(19) United States
(12) Patent Application Publication
(10) Pub. No.: US 2026/0248972 A1
(43) Pub. Date: Aug. 27, 2026

(54) DUAL RECEPTOR TARGETING RADIOLIGANDS AND USES THEREOF RELATED APPLICATIONS

(71) Applicant: Full-Life Technologies HK Limited, Hong Kong (CN)

(72) Inventors: Fa LIU, Lincolnshire, IL (US); Taishan HU, Shanghai (CN)

(21) Appl. No.: 19/150,510

(22) PCT Filed: Oct. 20, 2023
(86) PCT No.: PCT/CN2023/125675

§ 371 Date: Jul. 23, 2025

Related U.S. Application Data
(60) Provisional application No. 63/417,915, filed on Oct. 20, 2022.

(51) Int. Cl. A61K 51/08; A61P 35/00; C07K 14/595; C07K 14/655

(52) U.S. Cl. CPC A61K 51/083; A61K 51/088; A61P 35/00; C07K 14/595; C07K 14/655; A61K 2123/00

(57) ABSTRACT

The present invention discloses compounds of Formula (I) and their complexes with radionuclides that can recognize both Somatostatin type 2 receptor (SSTR2) and cholecystokinin 2 receptor (CCK2R) and can be used in the diagnosis and treatment of diseases where either or both receptors are overexpressed.

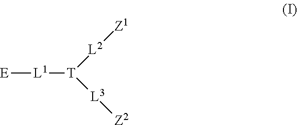

### RELATED APPLICATIONS

The present application claims the benefit of priority of co-pending U.S. provisional patent application No. 63/417,915 filed on Oct. 20, 2022, the contents of which are incorporated herein by reference in their entirety.

### FIELD

The present application relates generally to the field of radioligands that target cell surface receptors such as somatostatin receptors, cholecystokinin receptors or both. In particular, it relates to compounds and complexes of the compounds comprising radionuclides. The application also relates to methods of using the compounds and complexes for targeting and/or killing target cells.

### BACKGROUND

Somatostatin and its receptor family, consisting of five G-protein coupled receptors (GPCRs), play important roles in modulating the secretion of several essential hormones. The abnormality of this signaling pathway has been found to be associated with various diseases including cancers. It has been well-studied that the overexpression of Somatostatin type 2 receptor (SSTR2) is an important characteristic of neuroendocrine tumors. Consequently, SSTR2 has been explored extensively as a therapeutic target to deliver SSTR2-binding peptide complexed radionuclides to the tumor tissue, which enables PET or SPECT/CT-based diagnosis and internal α or β-radiation therapy. These efforts have led to the FDA-approved Netspot® (⁶⁸Ga-DOTATATE) and Lutathera® (¹⁷⁷Lu-DOTATATE), a theranostic pair being clinically used for detecting and treating gastroenteropancreatic neuroendocrine tumors (GEP-NETs). It has also been found that SSTR2 is overexpressed in other tumors, such as small cell lung cancer (SCLC); consequently, ¹⁷⁷Lu-DOTATATE and other SSTR2-targeting radioligands have been studied in multiple trails to treat SCLC although the desired outcome has yet to be achieved [1]. Similarly, ¹⁷⁷Lu-DOTATATE has been explored clinically for the treatment of medullary thyroid carcinoma (MTC) [2].

Cholecystokinin 2 receptor (CCK2R, also known as cholecystokinin B receptor or CCKBR) is also found to be overexpressed in various tumors including medullary thyroid carcinoma (MTC), small cell lung cancer (SCLC), astrocytoma, stromal ovarian and gastrointestinal stromal tumors (GIST) [3]. CCK2R is one of the two GPCR receptors in the cholecystokinin receptor (CCKR) family, whose endogenous ligands are cholecystokinin (CCK) and its N-terminal truncated versions. In the efforts to interfere with the CCK2R-mediated pathways, both native CCK or gastrin sequence-derived peptide agonists and small molecule-based antagonists have been developed. Recently, the targeted radioligand approach has also been investigated, most of them using peptide agonists as the vector with a F¹¹N-radionuclide complex [4-6] being a notable example. More recently, small molecule CCK2R antagonists have also been evaluated using a radionuclide vector [7-8].

Targeting tumor-surface overexpressed receptors has been a fruitful approach, which has resulted in a number of effective cancer treatments. However, it is also well recognized that the tumor heterogeneity has limited its potential, and additional challenges arise if the receptor has relatively low number of copies per cell.

An approach that would address these challenges has not been developed yet and is highly desired.

### SUMMARY

The Applicants have developed dual targeting compounds that can recognize both the Somatostatin type 2 receptor (SSTR2) and the cholecystokinin 2 receptor (CCK2R). When complexed to radionuclides, the compounds can be used, for example, for diagnostics and/or in the treatment of tumors, for examples tumors that overexpress either SSTR2 or CCK2R.

Accordingly, the present application includes a compound of Formula I or a pharmaceutically acceptable salt, solvate and/or prodrug thereof,

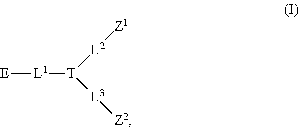

wherein
E is a chelating group;
T is a trivalent branching group;
Z¹ is a cholecystokinin-2 receptor (CCK2R) binding group;
Z² is a somatostatin receptor 2 (SSTR2) binding group; L¹, L² and L³ are each independently a direct bond or a divalent linker.

The present application also includes a radionuclide complex or a pharmaceutically acceptable salt and/or solvate thereof, comprising a compound of Formula I or a pharmaceutically acceptable salt and/or solvate thereof, and one or more radionuclides.

The present application also includes a composition comprising one or more compounds of Formula I, or one or more complexes thereof and a carrier.

The present application includes a kit comprising: (1) one or more compounds of Formula I, or a pharmaceutically acceptable salt and/or solvate thereof, and (2) instructions for administration of the one or more compounds of Formula I, or a pharmaceutically acceptable salt and/or solvate thereof, to a subject in need thereof.

The present application includes a kit comprising: (1) one or more compounds of Formula I, or a pharmaceutically acceptable salt and/or solvate thereof; (2) one or more radioisotope as defined above; and (3) optionally instructions for administration of the one or more compounds of Formula I, or a pharmaceutically acceptable salt and/or solvate thereof, to a subject in need thereof and the radioisotope to a subject in need thereof.

The present application includes a kit comprising: (1) one or more complexes of the application, as defined above, or a pharmaceutically acceptable salt and/or solvate thereof, and (2) instructions for administration of the one or more compounds complexes to a subject in need thereof.

The present application includes a method of treating a disease or disorder comprising administering a therapeutically effective amount of one or more compounds of Formula I or one or more complexes thereof to a subject in need thereof.

The present application includes a method of inhibiting proliferative activity in a cell, comprising administering a therapeutically effective amount of one or more compounds of any Formula I or one or more complexes thereof to the cell.

The present application includes a method of imaging a tissue in a subject by administering an imaging effective amount of one or more compounds of Formula I or one or more complexes thereof for use in imaging to a subject in need thereof and applying an imaging technique to detect emitted gamma rays.

The present application includes a method of diagnosing cancer in subject by administering a diagnostic effective amount of one or more compounds of Formula I or one or more complexes thereof to a subject in need thereof and applying an imaging technique to detect emitted gamma rays.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DRAWINGS

The embodiments of the application will now be described in greater detail with reference to the attached drawings in which:

FIG. 1 is a drawing showing a chelating group derived from DOTA (left side structure) and DOTAGA (right side structure) on a compound of Formula I or a pharmaceutically acceptable salt, solvate and/or prodrug thereof when complexed to Lu-177. A complex formed between a chelating group derived from DOTA group (left side structure) on a compound of Formula I or a pharmaceutically acceptable salt, solvate and/or prodrug thereof and Lu-177 has a net charge of 0. A complex formed between a chelating group derived from DOTAGA group (right side structure) on a compound of Formula I or a pharmaceutically acceptable salt, solvate and/or prodrug thereof and Lu-177 has a net charge of −1.

FIG. 2 is a graph showing the biodistribution (% ID/g) of ¹⁷⁷Lu-C-1 in AR42J tumor-bearing mice (n=3), The left most bar (darkest bar) at each organ/tissue point shows the biodistribution (% ID/g) at 4 hours, the middle bar at each organ/tissue point shows the biodistribution (% ID/g) at 24 hours, the right most bar at each organ/tissue shows the biodistribution (% ID/g) at 72 hours.

FIG. 3 is a graph showing the biodistribution of ¹⁷⁷Lu-C-4 in AR42J tumor-bearing mice (n=3). The left most bar (darkest bar) at each organ/tissue point shows the biodistribution (% ID/g) at 4 hours and the right most bar at each organ/tissue point shows the biodistribution (% ID/g) at 24 hours.

FIG. 4 is a graph showing the biodistribution of ¹⁷⁷Lu-1-13 in AR42J tumor-bearing mice (n=3). The left most bar (darkest bar) at each organ/tissue point shows the biodistribution (% ID/g) at 4 hours, the middle bar at each organ/tissue point shows the biodistribution (% ID/g) at 24 hours, the right most bar at each organ/tissue shows the biodistribution (% ID/g) at 72 hours.

FIG. 5 is a graph showing the biodistribution of ¹⁷⁷Lu-1-35 in AR42J tumor-bearing mice (n=3). The left most bar (darkest bar) at each organ/tissue point shows the biodistribution (% ID/g) at 4 hours, the middle bar at each organ/tissue point shows the biodistribution (% ID/g) at 24 hours, the right most bar at each organ/tissue shows the biodistribution (% ID/g) at 72 hours.

FIG. 6 is a graph showing the biodistribution of ¹⁷⁷Lu-1-36 in AR42J tumor-bearing mice (n=3). The left most bar (darkest bar) at each organ/tissue point shows the biodistribution (% ID/g) at 4 hours, the middle bar at each organ/tissue point shows the biodistribution (% ID/g) at 24 hours, the right most bar at each organ/tissue shows the biodistribution (% ID/g) at 72 hours.

FIG. 7 is a graph showing the biodistribution of ¹⁷⁷Lu-1-37 in AR42J tumor-bearing mice (n=3). The left most bar (darkest bar) at each organ/tissue point shows the biodistribution (% ID/g) at 4 hours, the middle bar at each organ/tissue point shows the biodistribution (% ID/g) at 24 hours, the right most bar at each organ/tissue shows the biodistribution.

FIG. 8 is a graph showing the biodistribution of ¹⁷⁷Lu-1-54 in AR42J tumor-bearing mice (n=3). The left most bar (darkest bar) at each organ/tissue point shows the biodistribution (% ID/g) at 4 hours, the middle bar at each organ/tissue point shows the biodistribution (% ID/g) at 24 hours, the right most bar at each organ/tissue shows the biodistribution.

### DESCRIPTION OF VARIOUS EMBODIMENTS

### I. Definitions

Unless otherwise indicated, the definitions and embodiments described in this and other sections are intended to be applicable to all embodiments and aspects of the present application herein described for which they are suitable as would be understood by a person skilled in the art.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “compound(s) of the application” or “compound(s) of the present application” and the like as used herein refers to a compound of Formula I or pharmaceutically acceptable salts and/or solvates thereof.

The term “complex of the application” or “complexes of the application” and the like as used herein refers to a complex comprising one or more compounds of Formula I or pharmaceutically acceptable salts and/or solvates thereof and one or more radionuclides.

The term “composition of the application” or “composition of the present application” and the like as used herein refers to a composition comprising one or more compounds or complexes of the application.

The term “radioligand” as used herein refers to compound comprising a targeting moiety and a radionuclide. The complexes of the application are examples of radioligands.

The term “radionuclide” as used herein refers to any atom capable of undergoing radioactive decay. The term radionuclide is used synonymously herein with radioactive nuclide, radioisotope, and radioactive isotope.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The present description refers to a number of chemical terms and abbreviations used by those skilled in the art. Nevertheless, definitions of selected terms are provided for clarity and consistency.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms. All alkyl groups are optionally fluoro-substituted unless otherwise indicated.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆alkylene means an alkylene group having 2, 3, 4, 5 or 6 carbon atoms. All alkylene groups are optionally fluoro-substituted unless otherwise indicated.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond. All alkenyl groups are optionally fluoro-substituted unless otherwise indicated.

The term “alkenylene”, whether it is used alone or as part of another group, means straight or branched chain, unsaturated alkyl groups containing at least one double bond that contains substituents on two of its ends. The number of carbon atoms that are possible in the referenced alkylene group are indicated by the prefix “Cₙ₁₋ₙ₂”. For example, the term C₂₋₆alkenylene means an alkenylene group having 2, 3, 4, 5 or 6 carbon atoms. All alkenylene groups are optionally fluoro-substituted unless otherwise indicated.

The term “aryl” as used herein, whether it is used alone or as part of another group, refers to carbocyclic groups containing at least one aromatic ring and contains 6 to 20 carbon atoms.

The term “cycloalkyl,” as used herein, whether it is used alone or as part of another group, means a saturated carbocyclic group containing from 3 to 20 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “heterocycloalkyl” as used herein, whether it is used alone or as part of another group, refers to cyclic groups containing at least one non-aromatic ring containing from 3 to 20 atoms in which one or more of the atoms are a heteroatom selected from O, S and N and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as selected from O, S and N and the remaining atoms are C. Heterocycloalkyl groups are optionally benzofused.

The term “heteroaryl” as used herein, whether it is used alone or as part of another group, refers to cyclic groups containing at least one heteroaromatic ring containing 5-20 atoms in which one or more of the atoms are a heteroatom selected from O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heteroaryl groups are optionally benzofused.

All cyclic groups, including aryl, heteroaryl, heterocycloalkyl and cycloalkyl groups, contain one or more than one ring (i.e. are polycyclic). When a cyclic group contains more than one ring, the rings may be fused, bridged, spirofused or linked by a bond.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “target binding group” are used herein refers to a moiety that is recognized by a target site to which it binds.

The term “target” or “target site” as used herein means a receptor, for example a cell surface receptor, antigen, for example Somatostatin type 2 receptor (SSTR2) and/or cholecystokinin 2 receptor (CCK2R) on a cell surface to which a first target binding group, a second targeting group or both can bind.

The term “chelating group” as used herein is chelator capable of complexing a radionuclide.

The term “trivalent branching group” as used herein refers to any molecular structure that comprises at least three terminal functional groups and each terminal functional group connects with another molecular structure. The at least three terminal functional groups can be the same or different.

The term “linker” as used herein refers to any molecular structure that connects two or more other molecular structures together.

The term “atom length” as used herein refers to the number of atoms in a chain. For example, an atom chain of “N—C—C” has an atom length of 3 atoms.

The term “length” with respect to a linker as used herein refers to the atom length of the backbone chain of the linker, L¹, L² and/or L³.

The term “combined length of L¹, L² and L³” as used herein refers to the combined atom length of the backbone chain of each bivalent linker, L¹, L² and L³, i.e., the number atoms in the backbone chain of the linker separating the chelating group (E), first target binding group (Z¹) and/or the second target binding group (Z²) and the trivalent branching group (T). For example, the length of a divalent linker comprising one glycine amino acid residue connecting the chelating group (E), first target binding group (Z¹) or the second target binding group (Z²) and the trivalent branching group (T) together through its amino and the carboxylic functional groups respectively is 3 atoms (i.e., a backbone atom chain of “N—C—C”.)

The term “amino acid residue” are used herein refers to an amino acid without the “—OH” of its carboxyl group and the “H” portion of an amino group.

The term “amino acid” as used herein is any compound comprising a carboxyl (—CO₂H) functional group and an amine (—NH₂) functional group.

The term “unnatural amino acid”, as used herein, refers to an amino acid that is not a naturally occurring amino acid and is obtained synthetically or by modification of a natural amino acid.

The term “naturally occurring amino acid” as used herein refers amino acids that occur naturally and are encoded by the genetic code, as well as those encoded amino acids that are later modified in vivo.

The term “net charge” as used herein in reference to the compound of Formula I or a pharmaceutically acceptable salt, solvate and/or prodrug thereof when complexed to a radionuclide is the balance of the number of positive charges and the number of negative charges of the complex. Net charges are measured at physiological pH.

The term “at least one” when preceding an item refers to a single member of the item or when preceding a list of items refers to a single member of the item and any combination of those items. For example at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. A subject with early cancer can be treated to prevent progression for example, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The terms “preventing”, “prevention” or “prophylaxis”, or synonyms thereto, as used herein refers to a reduction in the risk or probability of a patient becoming afflicted with a disease, disorder or condition or manifesting a symptom associated with a disease, disorder or condition.

As used herein, the term “therapeutically effective amount” means an amount of a compound, or one or more compounds, of the application or complex, or one or more complexes of the application, that is effective, at dosages and for periods of time necessary to achieve the desired result.

The term “imaging effective amount” when used in connection with a one or more complexes of the application, is an amount of the complex that is sufficient to produce a visible image when the complex is administered to a subject and the radiation emitted by the complex is detected using positron-emission tomography (“PET”) or single photon emission tomography (SPECT) or autoradiography or ex vivo or in vitro binding assays.

As used herein, the term “diagnostic effective amount” means an amount of a compound, or one or more compounds, of the application or complex, or one or more complexes of the application, that is effective, at dosages and for periods of time necessary to achieve the desired diagnostic effect including, for example, diagnosing a particular condition being assessed.

The term “administered” as used herein means administration of a therapeutically effective amount of one or more compounds, complexes or compositions of the application to a cell, tissue, organ or subject.

The term “cancer” as used herein refers to cellular-proliferative disease states.

The term “subject” as used herein includes all members of the animal kingdom including mammals, and suitably refers to humans. Thus, the methods and uses of the present application are applicable to both human therapy and veterinary applications.

The term “cell” as used herein refers to a single cell or a plurality of cells and includes a cell either in a cell culture or in a subject.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt and/or prodrug of a compound, wherein molecules of a suitable solvent are incorporated in the crystal lattice. A suitable solvent is physiologically tolerable at the dosage administered.

The symbol [symbol] when drawn perpendicularly across a bond indicates a point of covalent attachment of a chemical group.

When used, for example, with respect to the methods of treatment, uses, compositions, packages and/or kits of the application, a subject, for example a subject “in need thereof” is a subject that would benefit from administration of one or more compounds or complexes of the application, or a pharmaceutically acceptable salt and/or solvate thereof.

The term “DCM” as used herein refers to dichloromethane.

The term “DMF” as used herein refers to dimethylformamide.

The term “DIC” as used herein refers to N,N′-diisopropylcarbodiimide.

The term “DMAP” as used herein refers to 4-dimethylaminopyridine.

The term “PyBOP” as used herein refers to benzotriazol-1-yloxytripyrrolidinophosphonium hexafluorophosphate.

The term “HOBt” as used herein refers to hydroxybenzotriazole.

The term “DIEA” as used herein refers to diisopropylethylamine.

The term “HFIP” as used herein refers to hexafluoroisopropanol.

The term “TFA” as used herein refers to trifluoroacetic acid.

The term “TIS” as used herein refers to triisopropylsilane.

The term “MTBE” as used herein refers to methyl tert-butyl ether.

The term “ACN” as used herein refers to acetonitrile.

The term “BSA” as used herein refers to N,O-bis(trimethylsilyl)acetamide.

The term “PBS” as used herein refers to phosphate-buffered saline.

The term r.t. as used herein refers to room temperature.

### II. Compounds and Complexes of the Application

The Applicants have developed dual targeting radioligands that can recognize both the Somatostatin type 2 receptor (SSTR2) and the cholecystokinin 2 receptor (CCK2R). When complexed to radionuclides, the radioligands form radiopharmaceuticals that can be used, for example, as diagnostics and/or in the treatment of tumors, for example, tumors that overexpress either SSTR2 or CCK2R.

A person skilled in the art would appreciate that dual receptor targeting radioligands would need to maintain sufficient and balanced binding affinity towards both receptors. Further, since the dual targeting radioligand is built upon two mono-targeting receptor binding compounds, it would be appreciated that the dual targeting radioligand would possess physicochemical properties that are significantly different from either of the mono-targeting receptor binding parent compounds. As a result, the non-target driven biodistribution pattern of the mono-targeting receptor binding compounds can be dramatically changed. Moreover, the undesired distribution of the radioligand in vivo could be target-driven as well, therefore a dual targeting vector might worsen such target-directed normal organ accumulation.

The Applicants examined chemical and biophysical properties, such as molecular size, hydrophobicity, net charge, charge distribution, and hydrophobic patch distribution to determine factors which contributed to the biodistribution profile of the radioligand when complexed to the radionuclide. By modulating the net charge and/or hydrophobicity on the radioligand when complexed to the radionuclide, the Applicants have developed the dual targeting radioligand compounds of the application that show good binding affinity to both the Somatostatin type 2 receptor (SSTR2) and the cholecystokinin 2 receptor (CCK2R).

Further, exemplary dual targeting radioligand compounds of the application have been shown to have greater uptake of the radionuclide in the target cell while maintaining off-target normal organ accumulation to a minimum. The normal organs include but are not limited to the kidney and the liver.

Accordingly, the present application includes a compound of Formula I or a pharmaceutically acceptable salt, solvate and/or prodrug thereof,

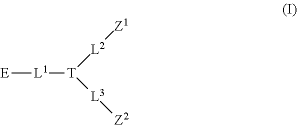

wherein
E is a chelating group;
T is a trivalent branching group;
Z¹ is a cholecystokinin-2 receptor (CCK2R) binding group;
Z² is a somatostatin receptor 2 (SSTR2) binding group;
L¹, L² and L³ are each independently a direct bond or a divalent linker.

In some embodiments, the CCK2R binding group is a benzodiazepine moiety of Formula II,

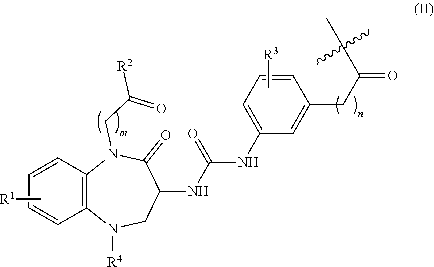

wherein
R¹ is selected from H, halo and C₁₋₆alkyl;
R² is selected from C₁₋₆alkyl, NH₂, NH(C₁₋₆alkyl) and N(C₁₋₆alkyl)₂;
R³ is selected from H, halo and C₁₋₆alkyl;
R⁴ is selected from C₅₋₆cycloalkyl and phenyl; and
m is 0, 1, 2 or 3; and
n is 0, 1, 2 or 3.

In some embodiments, the somatostatin receptor 2 (SSTR2) binding group is a somatostatin analogue. In some embodiments, Z² is selected from a D-Phe-c[Cys-Tyr-D-Trp-Lys-Thr-Cys]-Thr(ol) (Tyr 3-octreotide), a H-D-Phe-c[Cys-Phe-D-Trp-Lys-Thr-Cys]-Thr(ol) (octreotide) moiety and a D-Phe-c[Cys-Tyr-D-Trp-Lys-Thr-Cys]-Thr (Tyr3-octreotate) moiety.

Therefore, in some embodiments, Z² is a somatostatin receptor 2 (SSTR2) binding group and the somatostatin receptor 2 (SSTR2) binding group is a moiety of Formula III,

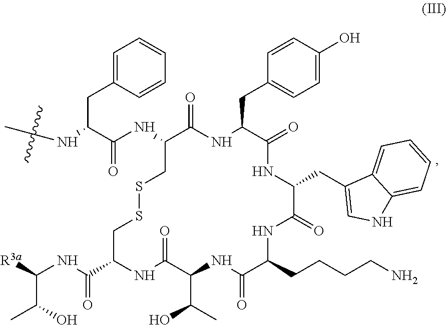

wherein:
R^{3a} is selected from CH₂OH, CO₂H and CONH₂.

Therefore, in some embodiments, the compound of Formula I is a compound of Formula I-A or a pharmaceutically acceptable salt, solvate and/or prodrug thereof,

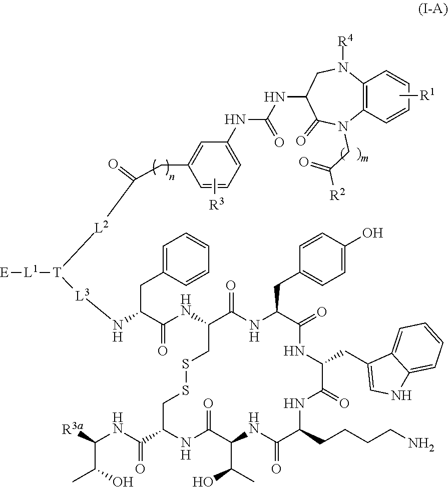

wherein
E is a chelating group;
T is a trivalent branching group;
L¹, L² and L³ are each independently a direct bond or a divalent linker;
R¹ is selected from H, halo and C₁₋₆alkyl;
R² is selected from C₁₋₆alkyl, NH₂, NH(C₁₋₆alkyl) and N(C₁₋₆alkyl)₂;
R³ is selected from H, halo and C₁₋₆alkyl;
R^{3a} is selected from CH₂OH, CO₂H and CONH₂;
R⁴ is selected from C₅₋₆cycloalkyl and phenyl; and
m is 0, 1, 2 or 3; and
n is 0, 1, 2 or 3.

In some embodiments, R¹ and R³ are independently selected from H, F, Cl, CH₃, CH₂CH₃, CH₂CH₂CH₃, CH₂CH₂CH₂CH₃, CH(CH₃)₂ and C(CH₃)₃. In some embodiments, R¹ and R³ are independently selected from H, CH₃, CH₂CH₃, CH₂CH₂CH₃, CH₂CH₂CH₂CH₃, CH(CH₃)₂ and C(CH₃)₃. In some embodiments, both R¹ and R³ are H.

In some embodiments, R² is selected from CH₃, CH₂CH₃, CH₂CH₂CH₃, CH₂CH₂CH₂CH₃, CH(CH₃)₂, C(CH₃)₃, NH₂, NH(CH₃) and N(CH₃)₂. In some embodiments, R² is selected from CH₃, CH₂CH₃, CH₂CH₂CH₃, CH₂CH₂CH₂CH₃, CH(CH₃)₂ and C(CH₃)₃. In some embodiments, R² is C(CH₃)₃.

In some embodiments, R^{3a} is selected from CH₂OH and CONH₂. In some embodiments, R^{3a} is CH₂OH. In some embodiments, R^{3a} is CONH₂. In some embodiments, R^{3a} is CO₂H.

In some embodiments, R⁴ is selected from cyclopentyl, cyclohexyl and phenyl. In some embodiment, R⁴ is selected from cyclohexyl and phenyl. In some embodiment, R⁴ is cyclohexyl.

In some embodiments, m is 0 or 1. In some embodiments, m is 1.

In some embodiments, n is 0 or 1. In some embodiments, n is 0.

In some embodiments, Z¹ is a CCK2R binding group and the CCK2R binding group is a Z360 moiety, Z360 having the following structure

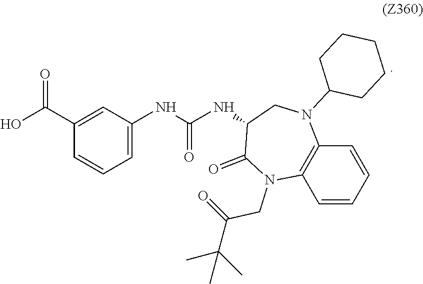 [SMILES: CC(C)(C)C(=O)CN1C(=O)[C@H](NC(=O)Nc2cccc(C(=O)O)c2)CN(C2CCCCC2)c2ccccc21]

Therefore, in some embodiments, the compound of Formula I has the following structure,

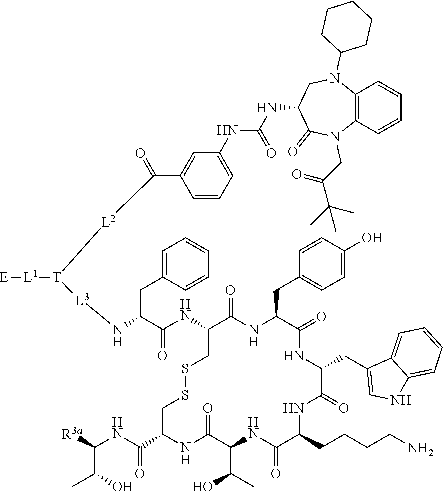

or a pharmaceutically acceptable salt, solvate and/or prodrug thereof wherein
E, T, L¹, L² and L³ are as defined in Formula I, and
R^{3a} is selected from CH₂OH, CO₂H and CONH₂.

In some embodiments, Z² is a SSTR2 binding group and the SSTR2 binding group is a moiety of Formula III wherein R^{a} is CO₂H and the compound of Formula III is Tyr 3-octreotate moiety, Tyr 3-octreotate having the following structure:

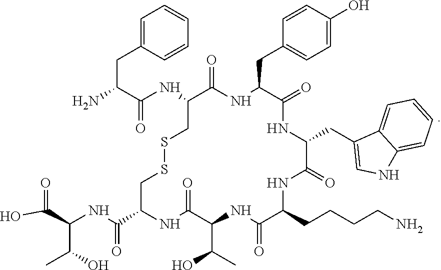 [SMILES: C[C@@H](O)[C@H](NC(=O)[C@@H]1CSSC[C@H](NC(=O)[C@H](N)Cc2ccccc2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Therefore, in some embodiments, the compound of Formula I is a compound of Formula I-A(i) or a pharmaceutically acceptable salt, solvate and/or prodrug thereof,

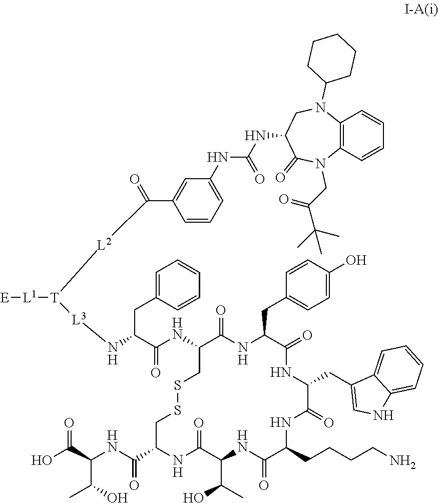

wherein
E, T, L¹, L² and L³ are as defined in Formula I.

In some embodiments, the compound of Formula I or a pharmaceutically acceptable salt, solvate and/or prodrug thereof when complexed to a radionuclide comprises a net charge of positive 2 (+2) to negative five (−5). In some embodiments, the compound of Formula I or a pharmaceutically acceptable salt, solvate and/or prodrug thereof when complexed to a radionuclide comprises a net charge of positive (+1) to negative five (−5). In some embodiments, the compound of Formula I or a pharmaceutically acceptable salt, solvate and/or prodrug thereof when complexed to a radionuclide comprises a net charge of positive (+1) to negative two (−2). In some embodiments, the compound of Formula I or a pharmaceutically acceptable salt, solvate and/or prodrug thereof when complexed to a radionuclide comprises a net charge of positive (+1) to negative one (−1). In some embodiments, the compound of Formula I or a pharmaceutically acceptable salt, solvate and/or prodrug thereof when complexed to a radionuclide comprises a net charge of zero (0) to negative five (−5). In some embodiments, the compound of Formula I or a pharmaceutically acceptable salt, solvate and/or prodrug thereof when complexed to a radionuclide comprises a net charge of zero (0) to negative four (−4). In some embodiments, the compound of Formula I or a pharmaceutically acceptable salt, solvate and/or prodrug thereof when complexed to a radionuclide comprises a net charge of zero (0) to negative three (−3). In some embodiments, the compound of Formula I or a pharmaceutically acceptable salt, solvate and/or prodrug thereof when complexed to a radionuclide comprises a net charge of zero (0) to negative two (−2). In some embodiments, the compound of Formula I or a pharmaceutically acceptable salt, solvate and/or prodrug thereof when complexed to a radionuclide comprises a net charge of zero (0) or negative one (−1). In some embodiments, the compound of Formula I or a pharmaceutically acceptable salt, solvate and/or prodrug thereof when complexed to a radionuclide comprises a net charge of 0. In some embodiments, the compound of Formula I or a pharmaceutically acceptable salt, solvate and/or prodrug thereof when complexed to a radionuclide comprises a net charge of −1.

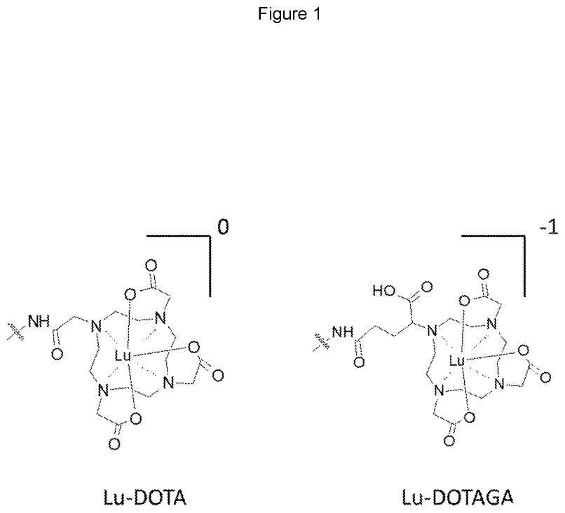

A person skilled in the art would appreciate that the net charge of the compound of Formula I or a pharmaceutically acceptable salt, solvate and/or prodrug thereof when complexed is presented as the radionuclide chelated state of the exemplary compound of Formula I or a pharmaceutically acceptable salt, solvate and/or prodrug thereof at physiological pH. For example, in an exemplary embodiment, when the compound of Formula I or a pharmaceutically acceptable salt, solvate and/or prodrug thereof comprises a chelating group, E, which comprises three free carboxylic acid groups (for example, when E is derived from 1,4,7,10-tetraazacyclododecane-1,4,7,10-tetraacetic acid (DOTA)), then the chelating group of the compound of Formula I or a pharmaceutically acceptable salt, solvate and/or prodrug thereof when complexed to a radionuclide having a positive three charge (for example, ¹⁷⁷Lu) would have a 0 net charge. In a further exemplary embodiment, when the compound of Formula I or a pharmaceutically acceptable salt, solvate and/or prodrug thereof comprises a chelating group, E, comprising four free carboxylic acid groups (for example, when E is derived from 1,4,7,10-tetraazacyclo-decane-1-glutamic acid-4,7,10-triacetic acid (DOTAGA) moiety), then the chelating group of the compound of Formula I or a pharmaceutically acceptable salt, solvate and/or prodrug thereof when complexed to a radionuclide having a positive three charge (for example, Lu-177) would have a −1 net charge. A representation of the chelating group (e.g., derived from DOTA or DOTAGA) of the compound of Formula I or a pharmaceutically acceptable salt, solvate and/or prodrug thereof when complexed to Lu-177 is shown in FIG. 1.

It would be further appreciated by a person skilled in the art that additional charged groups may be present in the compound of Formula I or a pharmaceutically acceptable salt, solvate and/or prodrug thereof. For example, additional charged groups may be present in the cholecystokinin-2 receptor (CCK2R) binding group, the somatostatin receptor 2 (SSTR2) binding group or in each of the divalent linkers. Any additional free carboxyl groups (for example, available from glutamic acid or gamma-glutamic acid residues) would be considered to contribute a net −1 charge for each free carboxyl group, and any additional free amino groups (for example, available from the epsilon amine of a lysine residue or the guanidine group of an arginine residue) would be considered to contribute a +1 charge for each free amine.

In some embodiments, the combined length of L¹, L² and L³ is 0 atoms to 80 atoms. In some embodiments, the combined length of L¹, L² and L³ is 0 atoms to 80 atoms, 0 atoms to 51 atoms, 0 atoms to 49 atoms, 0 atoms to 47 atoms, 0 atoms to 37 atoms, 0 atoms to 27 atoms, 0 atoms to 26 atoms, 0 atoms to 22 atoms, 0 atoms to 21 atoms, 0 atoms to 18 atoms, 0 atoms to 15 atoms, 0 atoms to 13 atoms, 0 atoms to 12 atoms, 0 atoms to 9 atoms, 0 atoms to 7 atoms, 0 atoms to 6 atoms, 0 atoms to 3 atoms. In some embodiments, the combined length of L¹, L² and L³ is 0 atoms to 70 atoms. In some embodiments, the combined length of L¹, L² and L³ is 0 atoms to 70 atoms, 0 atoms to 69 atoms, 0 atoms to 67 atoms, 0 atoms to 63 atoms, 0 atoms to 60 atoms, 0 atoms to 56 atoms, 0 atoms to 54 atoms, 0 atoms to 50 atoms, 0 atoms to 46 atoms, 0 atoms to 43 atoms, 0 atoms to 40 atoms, 0 atoms to 36 atoms, 0 atoms to 33 atoms, 0 atoms to 30 atoms, 0 atoms to 27 atoms, 0 atoms to 26 atoms, 0 atoms to 21 atoms, 0 atoms to 18 atoms, 0 atoms to 15 atoms, 0 atoms to 13 atoms, 0 atoms to 12 atoms, 0 atoms to 9 atoms, 0 atoms to 7 atoms, 0 atoms to 6 atoms, 0 atoms to 3 atoms, 3 atoms to 70 atoms, 3 atoms to 69 atoms, 3 atoms to 67 atoms, 3 atoms to 63 atoms, 3 atoms to 60 atoms, 3 atoms to 56 atoms, 3 atoms to 54 atoms, 3 atoms to 50 atoms, 3 atoms to 46 atoms, 3 atoms to 43 atoms, 3 atoms to 40 atoms, 3 atoms to 36 atoms, 3 atoms to 33 atoms, 3 atoms to 30 atoms, 3 atoms to 27 atoms, 3 atoms to 21 atoms, 3 atoms to 18 atoms, 3 atoms to 15 atoms, 3 atoms to 13 atoms, atoms to 12 atoms, 3 atoms to 9 atoms, 3 atoms to 7 atoms, 3 atoms to 6 atoms, 6 atoms to 70 atoms, 6 atoms to 69 atoms, 6 atoms to 67 atoms, 6 atoms to 63 atoms, 6 atoms to 60 atoms, 6 atoms to 56 atoms, 6 atoms to 54 atoms, 6 atoms to 50 atoms, 6 atoms to 46 atoms, 6 atoms to 43 atoms, 6 atoms to 40 atoms, 6 atoms to 36 atoms, 6 atoms to 33 atoms, 6 atoms to 30 atoms, 6 atoms to 27 atoms, 6 atoms to 21 atoms, 6 atoms to 18 atoms, 6 atoms to 15 atoms, 6 atoms to 13 atoms, 6 atoms to 12 atoms, 6 atoms to 9 atoms, 9 atoms to 70 atoms, 9 atoms to 69 atoms, 9 atoms to 67 atoms, 9 atoms to 63 atoms, 9 atoms to 60 atoms, 9 atoms to 56 atoms, 9 atoms to 54 atoms, 9 atoms to 50 atoms, 9 atoms to 46 atoms, 9 atoms to 43 atoms, 9 atoms to 40 atoms, 9 atoms to 36 atoms, 9 atoms to 33 atoms, 9 atoms to 30 atoms, 9 atoms to 27 atoms, 9 atoms to 21 atoms, 9 atoms to 18 atoms, 9 atoms to 15 atoms, 9 atoms to 12 atoms, 12 atoms to 70 atoms, 12 atoms to 69 atoms, 12 atoms to 67 atoms, 12 atoms to 63 atoms, 12 atoms to 60 atoms, 12 atoms to 56 atoms, 12 atoms to 54 atoms, 12 atoms to 50 atoms, 12 atoms to 46 atoms, 12 atoms to 43 atoms, 12 atoms to 40 atoms, 12 atoms to 36 atoms, 12 atoms to 33 atoms, 12 atoms to 30 atoms, 12 atoms to 27 atoms, 12 atoms to 21 atoms, 12 atoms to 18 atoms, 12 atoms to 15 atoms, 15 atoms to 70 atoms, 15 atoms to 69 atoms, 15 atoms to 67 atoms, 15 atoms to 63 atoms, 15 atoms to 60 atoms, atoms to 56 atoms, 15 atoms to 54 atoms, 15 atoms to 50 atoms, 15 atoms to 46 atoms, 15 atoms to 43 atoms, 15 atoms to 40 atoms, 15 atoms to 36 atoms, 15 atoms to 33 atoms, 15 atoms to 30 atoms, 15 atoms to 27 atoms, 15 atoms to 21 atoms, 15 atoms to 18 atoms, 18 atoms to 70 atoms, 18 atoms to 69 atoms, 18 atoms to 67 atoms, 18 atoms to 63 atoms, 18 atoms to 60 atoms, 18 atoms to 56 atoms, 18 atoms to 54 atoms, 18 atoms to 50 atoms, 18 atoms to 46 atoms, 18 atoms to 43 atoms, 18 atoms to 40 atoms, 18 atoms to 36 atoms, 18 atoms to 33 atoms, 18 atoms to 30 atoms, 18 atoms to 27 atoms, 18 atoms to 21 atoms, 21 atoms to 27 atoms 21 atoms to 70 atoms, 21 atoms to 67 atoms, 21 atoms to 60 atoms, 21 atoms to 54 atoms, 21 atoms to 46 atoms, 21 atoms to 27 atoms, 27 atoms to 70 atoms, 27 atoms to 67 atoms, 27 atoms to 60 atoms, 27 atoms to 54 atoms, 27 atoms to 46 atoms, 21 atoms to 35 atoms, 33 atoms to 70 atoms, 33 atoms to 67 atoms, 33 atoms to 60 atoms, 33 atoms to 54 atoms, 33 atoms to 46 atoms, 33 atoms to 36 atoms, 40 atoms to 70 atoms, 40 atoms to 67 atoms, 40 atoms to 60 atoms, 40 atoms to 54 atoms, 40 atoms to 46 atoms or 45 atoms to 60 atoms.

In some embodiments, the combined length of L¹, L² and L³ is 0 atoms to 47 atoms, 0 atoms to 46 atoms, 0 atoms to 43 atoms, 0 atoms to 41 atoms, 0 atoms to 40 atoms, 0 atoms to 36 atoms, 0 atoms to 37 atoms, 0 atoms to 33 atoms, 0 atoms to 30 atoms 0 atoms to 27 atoms, 0 atoms to 26 atoms, 0 atoms to 22 atoms, 0 atoms to 21 atoms, 0 atoms to 18 atoms, 0 atoms to 15 atoms, 0 atoms to 13 atoms, 0 atoms to 12 atoms, 0 atoms to 9 atoms, 0 atoms to 7 atoms, 0 atoms to 6 atoms, 0 atoms to 3 atoms. In some embodiments, the combined length of L¹, L² and L³ is 0 atoms to 47 atoms, 0 atoms to 46 atoms, 0 atoms to 43 atoms, 0 atoms to 41 atoms, 0 atoms to 40 atoms, 0 atoms to 36 atoms, 0 atoms to 37 atoms, 0 atoms to 33 atoms, 0 atoms to 30 atoms or 0 atoms to 27 atoms. In some embodiments, the combined length of L¹, L² and L³ is 0 atoms to 27 atoms.

In some embodiments, the combined length of L¹, L² and L³ is, 0 atoms to 26 atoms, 0 atoms to 21 atoms, 0 atoms to 18 atoms, 0 atoms to 15 atoms, 0 atoms to 12 atoms, 0 atoms to 9 atoms, 0 atoms to 7 atoms, 0 atoms to 6 atoms, 0 atoms to 3 atoms, 3 atoms to 27 atoms, 3 atoms to 21 atoms, 3 atoms to 18 atoms, 3 atoms to 15 atoms, 3 atoms to 12 atoms, 3 atoms to 9 atoms, 3 atoms to 6 atoms, 6 atoms to 27 atoms, 6 atoms to 21 atoms, 6 atoms to 18 atoms, 6 atoms to 15 atoms, 6 atoms to 12 atoms, 6 atoms to 9 atoms, 9 atoms to 27 atoms, 9 atoms to 21 atoms, 9 atoms to 18 atoms, 9 atoms to 15 atoms, 9 atoms to 12 atoms, 12 atoms to 27 atoms, 12 atoms to 21 atoms, 12 atoms to 18 atoms, 12 atoms to 15 atoms, 15 atoms to 27 atoms, 15 atoms to 21 atoms, 15 atoms to 18 atoms, 18 atoms to 27 atoms, 18 atoms to 21 atoms, or 21 atoms to 27 atoms.

In some embodiments, the combined length of L¹, L² and L³ is 0 atoms to 27 atoms, 0 atoms to 21 atoms, 0 atoms to 18 atoms, 0 atoms to 15 atoms, 0 atoms to 12 atoms, 0 atoms to 9 atoms, 0 atoms to 7 atoms, 0 atoms to 6 atoms, 0 atoms to 3 atoms, 3 atoms to 27 atoms, 3 atoms to 21 atoms, 3 atoms to 18 atoms, 3 atoms to 15 atoms, 3 atoms to 12 atoms, 3 atoms to 9 atoms, 3 atoms to 6 atoms, 6 atoms to 27 atoms, 6 atoms to 21 atoms, 6 atoms to 18 atoms, 6 atoms to 15 atoms, 6 atoms to 12 atoms, 6 atoms to 9 atoms, 9 atoms to 27 atoms, 9 atoms to 21 atoms, 9 atoms to 18 atoms, 9 atoms to 15 atoms, 9 atoms to 12 atoms, 18 atoms to 27 atoms, or 18 atoms to 21 atoms.

In some embodiments, the combined length of L¹, L² and L³ is 0 atoms, 3 atoms, 6 atoms, 7 atoms, 9 atoms, 12 atoms, 13 atoms, 15 atoms, 18 atoms, 21 atoms, 24 atoms or 27 atoms. In some embodiments, the combined length of L¹, L² and L³ is 0 atoms, 3 atoms, 6 atoms, 7 atoms, 9 atoms, 12 atoms, 13 atoms, 15 atoms or 18 atoms. In some embodiments, the combined length of L¹, L² and L³ is 9 atoms.

In some embodiments, each of L¹, L² and L³ independently have a length of 0 atoms to 80 atoms. In some embodiments, each of L¹, L² and L³ independently have a length of 0 atoms to 70 atoms.

In some embodiments, each of L¹, L² and L³ independently have a length of 0 atoms to 80 atoms. In some embodiments, each of L¹, L² and L³ independently have a length of 0 atoms to 70 atoms. In some embodiments, each of L¹, L² and L³ independently have a length of 0 atoms to 70 atoms, 0 atoms to 69 atoms, 0 atoms to 67 atoms, 0 atoms to 63 atoms, 0 atoms to 60 atoms, 0 atoms to 56 atoms, 0 atoms to 54 atoms, 0 atoms to 50 atoms, 0 atoms to 46 atoms, 0 atoms to 43 atoms, 0 atoms to 40 atoms, 0 atoms to 36 atoms, 0 atoms to 33 atoms, 0 atoms to 30 atoms, 0 atoms to 27 atoms, 0 atoms to 26 atoms, 0 atoms to 21 atoms, 0 atoms to 18 atoms, 0 atoms to 15 atoms, 0 atoms to 13 atoms, 0 atoms to 12 atoms, 0 atoms to 9 atoms, 0 atoms to 7 atoms, 0 atoms to 6 atoms, 0 atoms to 3 atoms, 3 atoms to 70 atoms, 3 atoms to 69 atoms, 3 atoms to 67 atoms, 3 atoms to 63 atoms, 3 atoms to 60 atoms, 3 atoms to 56 atoms, 3 atoms to 54 atoms, 3 atoms to 50 atoms, 3 atoms to 46 atoms, 3 atoms to 43 atoms, 3 atoms to 40 atoms, 3 atoms to 36 atoms, 3 atoms to 33 atoms, 3 atoms to 30 atoms, 3 atoms to 27 atoms, 3 atoms to 21 atoms, 3 atoms to 18 atoms, 3 atoms to 15 atoms, 3 atoms to 13 atoms, atoms to 12 atoms, 3 atoms to 9 atoms, 3 atoms to 7 atoms, 3 atoms to 6 atoms, 6 atoms to 70 atoms, 6 atoms to 69 atoms, 6 atoms to 67 atoms, 6 atoms to 63 atoms, 6 atoms to 60 atoms, 6 atoms to 56 atoms, 6 atoms to 54 atoms, 6 atoms to 50 atoms, 6 atoms to 46 atoms, 6 atoms to 43 atoms, 6 atoms to 40 atoms, 6 atoms to 36 atoms, 6 atoms to 33 atoms, 6 atoms to 30 atoms, 6 atoms to 27 atoms, 6 atoms to 21 atoms, 6 atoms to 18 atoms, 6 atoms to 15 atoms, 6 atoms to 13 atoms, 6 atoms to 12 atoms, 6 atoms to 9 atoms, 9 atoms to 70 atoms, 9 atoms to 69 atoms, 9 atoms to 67 atoms, 9 atoms to 63 atoms, 9 atoms to 60 atoms, 9 atoms to 56 atoms, 9 atoms to 54 atoms, 9 atoms to 50 atoms, 9 atoms to 46 atoms, 9 atoms to 43 atoms, 9 atoms to 40 atoms, 9 atoms to 36 atoms, 9 atoms to 33 atoms, 9 atoms to 30 atoms, 9 atoms to 27 atoms, 9 atoms to 21 atoms, 9 atoms to 18 atoms, 9 atoms to 15 atoms, 9 atoms to 12 atoms, 12 atoms to 70 atoms, 12 atoms to 69 atoms, 12 atoms to 67 atoms, 12 atoms to 63 atoms, 12 atoms to 60 atoms, 12 atoms to 56 atoms, 12 atoms to 54 atoms, 12 atoms to 50 atoms, 12 atoms to 46 atoms, 12 atoms to 43 atoms, 12 atoms to 40 atoms, 12 atoms to 36 atoms, 12 atoms to 33 atoms, 12 atoms to 30 atoms, 12 atoms to 27 atoms, 12 atoms to 21 atoms, 12 atoms to 18 atoms, 12 atoms to 15 atoms, 15 atoms to 70 atoms, 15 atoms to 69 atoms, 15 atoms to 67 atoms, 15 atoms to 63 atoms, 15 atoms to 60 atoms, 15 atoms to 56 atoms, 15 atoms to 54 atoms, 15 atoms to 50 atoms, 15 atoms to 46 atoms, 15 atoms to 43 atoms, 15 atoms to 40 atoms, 15 atoms to 36 atoms, 15 atoms to 33 atoms, 15 atoms to 30 atoms, 15 atoms to 27 atoms, 15 atoms to 21 atoms, 15 atoms to 18 atoms, 18 atoms to 70 atoms, 18 atoms to 69 atoms, 18 atoms to 67 atoms, 18 atoms to 63 atoms, 18 atoms to 60 atoms, 18 atoms to 56 atoms, 18 atoms to 54 atoms, 18 atoms to 50 atoms, 18 atoms to 46 atoms, 18 atoms to 43 atoms, 18 atoms to 40 atoms, 18 atoms to 36 atoms, 18 atoms to 33 atoms, 18 atoms to 30 atoms, 18 atoms to 27 atoms, 18 atoms to 21 atoms, 21 atoms to 27 atoms 21 atoms to 70 atoms, 21 atoms to 67 atoms, 21 atoms to 60 atoms, 21 atoms to 54 atoms, 21 atoms to 46 atoms, 21 atoms to 27 atoms, 27 atoms to 70 atoms, 27 atoms to 67 atoms, 27 atoms to 60 atoms, 27 atoms to 54 atoms, 27 atoms to 46 atoms, 21 atoms to 35 atoms, 33 atoms to 70 atoms, 33 atoms to 67 atoms, 33 atoms to 60 atoms, 33 atoms to 54 atoms, 33 atoms to 46 atoms, 33 atoms to 36 atoms, 40 atoms to 70 atoms, 40 atoms to 67 atoms, 40 atoms to 60 atoms, 40 atoms to 54 atoms, 40 atoms to 46 atoms or 45 atoms to 60 atoms.

In some embodiments, each of L¹, L² and L³ independently have a length of 0 atoms to 80 atoms. In some embodiments, each of L¹, L² and L³ independently have a length of 0 atoms to 70 atoms. In some embodiments, each of L¹, L² and L³ independently have a length of 0 atoms to 70 atoms, 0 atoms to 69 atoms, 0 atoms to 67 atoms, 0 atoms to 63 atoms, 0 atoms to 60 atoms, 0 atoms to 56 atoms, 0 atoms to 54 atoms, 0 atoms to 50 atoms, 0 atoms to 46 atoms, 0 atoms to 43 atoms, 0 atoms to 40 atoms, 0 atoms to 36 atoms, 0 atoms to 33 atoms, 3 atoms to 70 atoms, 3 atoms to 69 atoms, 3 atoms to 67 atoms, 3 atoms to 63 atoms, 3 atoms to 60 atoms, 3 atoms to 56 atoms, 3 atoms to 54 atoms, 3 atoms to 50 atoms, 3 atoms to 46 atoms, 3 atoms to 43 atoms, 3 atoms to 40 atoms, 3 atoms to 36 atoms, 3 atoms to 33 atoms, 3 atoms to 30 atoms, 6 atoms to 70 atoms, 6 atoms to 69 atoms, 6 atoms to 67 atoms, 6 atoms to 63 atoms, 6 atoms to 60 atoms, 6 atoms to 56 atoms, 6 atoms to 54 atoms, 6 atoms to 50 atoms, 6 atoms to 46 atoms, 6 atoms to 43 atoms, 6 atoms to 40 atoms, 6 atoms to 36 atoms, 6 atoms to 33 atoms, 9 atoms to 70 atoms, 9 atoms to 69 atoms, 9 atoms to 67 atoms, 9 atoms to 63 atoms, 9 atoms to 60 atoms, 9 atoms to 56 atoms, 9 atoms to 54 atoms, 9 atoms to 50 atoms, 9 atoms to 46 atoms, 9 atoms to 43 atoms, 9 atoms to 40 atoms, 9 atoms to 36 atoms, 9 atoms to 33 atoms, 9 atoms to 30 atoms, 12 atoms to 70 atoms, 12 atoms to 69 atoms, 12 atoms to 67 atoms, 12 atoms to 63 atoms, 12 atoms to 60 atoms, 12 atoms to 56 atoms, 12 atoms to 54 atoms, 12 atoms to 50 atoms, 12 atoms to 46 atoms, 12 atoms to 43 atoms, 12 atoms to 40 atoms, 12 atoms to 36 atoms, 12 atoms to 33 atoms, 15 atoms to 70 atoms, 15 atoms to 69 atoms, 15 atoms to 67 atoms, 15 atoms to 63 atoms, 15 atoms to 60 atoms, 15 atoms to 56 atoms, 15 atoms to 54 atoms, 15 atoms to 50 atoms, 15 atoms to 46 atoms, 15 atoms to 43 atoms, 15 atoms to 40 atoms, 15 atoms to 36 atoms, 15 atoms to 33 atoms, 18 atoms to 70 atoms, 18 atoms to 69 atoms, 18 atoms to 67 atoms, 18 atoms to 63 atoms, 18 atoms to 60 atoms, 18 atoms to 56 atoms, 18 atoms to 54 atoms, 18 atoms to 50 atoms, 18 atoms to 46 atoms, 18 atoms to 43 atoms, 18 atoms to 40 atoms, 18 atoms to 36 atoms, 21 atoms to 70 atoms, 21 atoms to 67 atoms, 21 atoms to 60 atoms, 21 atoms to 54 atoms, 21 atoms to 46 atoms, 21 atoms to 27 atoms, 27 atoms to 70 atoms, 27 atoms to 67 atoms, 27 atoms to 60 atoms, 27 atoms to 54 atoms, 27 atoms to 46 atoms, 21 atoms to 35 atoms, 33 atoms to 70 atoms, 33 atoms to 67 atoms, 33 atoms to 60 atoms, 33 atoms to 54 atoms, 33 atoms to 46 atoms, 33 atoms to 36 atoms, 40 atoms to 70 atoms, 40 atoms to 67 atoms, 40 atoms to 60 atoms, 40 atoms to 54 atoms, 40 atoms to 46 atoms or 45 atoms to 60 atoms.

In some embodiments, each of L¹, L² and L³ independently have a length of 0 atoms to 47 atoms, 0 atoms to 46 atoms, 0 atoms to 43 atoms, 0 atoms to 41 atoms, 0 atoms to 40 atoms, 0 atoms to 36 atoms, 0 atoms to 37 atoms, 0 atoms to 33 atoms, 0 atoms to 30 atoms 0 atoms to 27 atoms, 0 atoms to 26 atoms, 0 atoms to 22 atoms, 0 atoms to 21 atoms, 0 atoms to 18 atoms, 0 atoms to 15 atoms, 0 atoms to 13 atoms, 0 atoms to 12 atoms, 0 atoms to 9 atoms, 0 atoms to 7 atoms, 0 atoms to 6 atoms, 0 atoms to 3 atoms. In some embodiments, each of L¹, L² and L³ independently have a length of 0 atoms to 47 atoms, 0 atoms to 46 atoms, 0 atoms to 43 atoms, 0 atoms to 41 atoms, 0 atoms to 40 atoms, 0 atoms to 36 atoms, 0 atoms to 37 atoms, 0 atoms to 33 atoms, 0 atoms to 30 atoms or 0 atoms to 27 atoms In some embodiments, each of L¹, L² and L³ independently have a length of 0 atoms to 27 atoms.

In some embodiments, each of L¹, L² and L³ independently have a length of 0 atoms to 27 atoms, 0 atoms to 21 atoms, 0 atoms to 18 atoms, 0 atoms to 15 atoms, 0 atoms to 13 atoms, 0 atoms to 12 atoms, 0 atoms to 9 atoms, 0 atoms to 6 atoms, 3 atoms to 27 atoms, 3 atoms to 18 atoms, 6 atoms to 27 atoms, 6 atoms to 18 atoms, 9 atoms to 27 atoms, 9 atoms to 21 atoms, or 9 atoms to 18 atoms.

In some embodiments, each of L¹, L² and L³ independently have a length of 0 atoms, 3 atoms, 6 atoms, 7 atoms, 9 atoms, 12 atoms, 13 atoms, 15 atoms, 18 atoms, 21 atoms, 24 atoms or 27 atoms. In some embodiments, L¹, L² and L³ each have a length of 0 atoms, 3 atoms, 6 atoms, 7 atoms, 9 atoms, 12 atoms, 13 atoms, 15 atoms or 18 atoms.

In some embodiments, one of L¹, L² and L³ is a direct bond. In some embodiments, L¹ is a direct bond. In some embodiments, two of L¹, L² and L³ are direct bonds. In some embodiments, L² and L³ are both direct bonds. In some embodiments, L¹, L² and L³ are all direct bonds.

A person skilled in the art would appreciate that a length of L¹, L² and/or L³ of 0 atoms or a combined length of L¹, L² and L³ of 0 as used herein means that the L¹, L² and/or L³ is a direct bond or each of L¹, L² and L³ are direct bonds, respectively.

In some embodiment, the E is any chelating group that is capable of binding with and/or complexing a metal ion. In some embodiments, the E is any chelating group that is capable of binding with and/or complexing a metal ion to form a heterocyclic ring including the metal ion. In some embodiments, the E is any chelating group derived from any chelating agent known in the art, for example, as disclosed in Banerjee et al., Nucl. Med. Biol., 2005, 32, 1-20, Wadas et al., Chem. Rev., 2010, 110, 2858-2902, U.S. Pat. Nos. 5,367,080, 5,364,613, 5,021,556, 5,075,099, and 5,886,142,

Therefore, in some embodiments, E is a chelating group derived from a chelating agent. In some embodiments, the chelating agent is selected from a cyclic or an acyclic bifunctional chelating agent capable of binding with and/or complexing one or more radionuclides. In some embodiments, the chelating agent is selected from 1,4,7-Triazacyclononane (TACN); 1,4,7-triazacyclononane-triacetic acid (NOTA); 1,4,7-triazacyclononane-N-succinic acid-N′,N″-diacetic acid (NOTASA); 1,4,7-triazacyclononane-N-glutamic acid-N′,N″-diacetic acid (NODAGA); 1,4,7-triazacyclononane-N,N′,N″-tris(methylenephosphonic) acid (NOTP); 1,4,7,10-tetraazacyclododecane ([12]aneN4) (cyclen); 1,4,7,10-tetraazacyclotridecane ([13]aneN4); 1,4,7,11-tetraazacyclotetradecane (iso-cyclam); 1,4,7,10-tetraazacyclododecane-1,4,7,10-tetraacetic acid (DOTA); 2-(1,4,7,10-tetraazacyclododecan-1-yl)acetate (DO1A); 2,2′-(1,4,7,10-tetraazacyclododecane-1,7-diyl) diacetic acid (DO2A); 2,2′,2″-(1,4,7,10-tetraazacyclododecane-1,4,7-triyl) triacetic acid (DO3A); 1,4,7,10-tetraazacyclododecane-1,4,7,10-tetra(methanepnosphonic acid) (DOTP); 1,4,7,10-tetraazacyclododecane-1,7-di(methanephosphoriic acid) (DO2P); 1,4,7,10-tetraazacyclododecane-1,4,7-tri(methanephosphonic acid) (DO3P); 1,4,7,10-tetraazacyclo-decane-1-glutamic acid-4,7,10-triacetic acid (DOTAGA); 1,4,7,10-tetraazacyclodecane-1-succinic acid-4,7,10-triacetic acid (DOTASA); 1,4,8,11-tetraazacyclotetradecane ([14]aneN4) (cyclam); 1,4,8,12-tetraazacyclopentadecane ([15]aneN4); 1,5,9,13-tetraazacyclohexadecane ([16]aneN4); 1,4-ethano-1,4,8,11-tetraazacyclo-tetradecane (et-cyclam); 1,4,8,11-tetraazacyclotetradecane-1,4,8,11-tetraacetic acid (TETA); 2-(1,4,8,11-tetraazacyclotetradecane-1-yl) acetic acid (TE1A); 2,2′-(1,4,8,11-tetraazacyclotetradecane-1,8-diyl) diacetic acid (TE2A); 4,11-bis(carboxy methyl)-1,4,8,11-tetraazabicyclo[6.6.2]-hexadecane (CB-TE2A); 3,6,10,13,16,19-hexaazabicyclo[6.6.6]icosane (Sar); 1,4,7,10-tetra-(2-carbamoyl-methyl)-cyclododecane (TCMC); N,N′-bis[(6-carboxy-2-pyridil)methyl]-4,13-diaza-18-crown-6 (macropa), phthalocyanines; porphyrins; PCTA (3,6,9,15-tetraazabicyclo[9.3.1]pentadeca-1(15), 11,13-triene-3,6,9-triacetic acid); DEPA (7-[2-(biscarboxymethylamino)ethyl]-4,10-biscarboxymethyl-1,4,7,10-tetraazacyclododec-1-yl-acetic acid); DTPA (1,1,4,7,7-diethylenetriaminepentaacetic acid); CHX-DTPA (cyclohexane-1,2-diamineN,N,N′,N′-tetraacetate); BATPA (1,2-bis[2-aminophenoxy]ethane-N,N,N′,N′-tetraacetic acid); TTHA (triethylenetetramineN,N,N′,N″,N″′,N″′-hexaacetic acid); HBED (N,N′-bis[2-hydroxybenzyl]ethylenediamine-N,N′-diacetic acid); EGTA (ethylene glycol bis[2-aminoethyl ether]-N,N,N′,N′-tetraacetic acid); EDTMP (ethylenediamine tetra-[methylene phosphonic acid]); TRAP (triazacyclononate phosphinic acids); SHBED (N,N′-bis[2-hydroxy-5-sulfobenzyl]ethylenediaminediacetic acid); H6Sbbpen(N,N′-bis-[2-hydroxy-5-sulfonylbenzyl]-N,N′-bis[2-methylpyridyl]ethylenediamine); THP (Tris(3,4-hydroxypyridinone); DFO (deferoxamine); FSC (Fusarinine); 6SS (N,N′-bis[2,2-dimethyl-2-mercaptoethyl]ethylenediamine-N,N′-diacetic acid); ECC (ethylenecysteamine cysteine); ECD (ethyl cysteinate dimer); NETA ([2-{4,7-biscarboxymethyl(1,4,7)triazacyclonona-1-yl-ethyl}carbonylmethylamino]acetic acid; THPN (Tetrakis(3-Hydroxy-4-Pyridinone)); H2dedpa (1,2-[{6-(carboxylato-)pyridin-2-yl}methylamino]-ethane); H4octapa (N,N′-bis[6-carboxy-2-pyridylmethyl]-ethylenediamine-N,N′-diacetic acid); H2bispa2 (6,6′-[{9-hydroxy-1,5-bis-(methoxycarbonyl)-2,4-di(pyridin-2-yl)-3,7-diazabicyclo[3.3.1]nonane-3,7-diyl}bis(methylene)]dipicolinic acid); DOTMP(1,4,7,10-Tetraazacyclododecane-1,4,7,10-tetrayl-tetrakis(methylphosphonic acid)); PEPA (1,4,7,10,13-pentaazocyclopentadecane pentaacetic acid); HEHA (1,4,7,10,13,16-hexaazocyclooctadecane hexaacetic acid); H2hox; H2CHXhox; H2octox; H2pyhox; H4neunopa; TETPA; H4pypa; H4py4pa; DTPAm; EGTAm; ampam; Me-3,2-HOPO; 3,4,3-(LI-1,2-HOPO); macrocyclic tetrapthalimide; or any derivatives thereof. In some embodiments, E is selected from DOTA and DOTAGA. In some embodiments, E is DOTA. In some embodiments, E is DOTAGA.

A person skilled in the art would appreciate that “a chelating group derived from a chelating agent” as used herein refers to a chelating agent derivative formed after the chelating agent is connected to the compound of Formula I or a pharmaceutically acceptable salt, solvate and/or prodrug thereof, e.g., to connected to L¹ (or T when L¹ is a direct bond). For example, “a chelating group derived from a chelating agent” may be a chelating agent without the “—OH” (or ester thereof) of an available carboxyl group (or ester thereof) on the chelating agent, without the “H” portion of an available amino group on the chelating agent, without the “NCS” portion of an available isothiocyanate on the chelating agent, without the “H” portion of an available maleimide group on the chelating agent, a chelating agent after an available acetylene group on the chelating agent has been reacted to connect to the compound of Formula I or a pharmaceutically acceptable salt, solvate and/or prodrug thereof, or a chelating agent after an available tetrazole group on the chelating agent has been reacted to connect to the compound of Formula I or a pharmaceutically acceptable salt, solvate and/or prodrug thereof. For example, a person skilled in the art would appreciate that when E is a chelating group derived from a DOTA, one “—OH” from one of the four available carboxyl groups on DOTA is removed to form the connection, such as an amide bond, to L¹ (or T when L¹ is a direct bond) in the compound of Formula I or a pharmaceutically acceptable salt, solvate and/or prodrug thereof leaving three available carboxyl groups.

In some embodiments, the E is connected to L¹ through any one of the available functional groups. In some embodiments, E is a chelating group comprising two or more carboxyl groups, and E is connected to L¹ through a carboxyl functional group. In some embodiments, E is a chelating group derived from DOTA or DOTAGA and is connected to L¹ through any one of the available carboxyl functional groups.

In some embodiments, the one or more radionuclides is a radioactive isotope of C, N, O, F, P, S, Cl, Br, I, As, Se, At, K, Sc, Ti, Cr, Mn, Fe, Co, Ni, Cu, Ga, Ge, Rb, Sr, Y, Zr, Nb, Tc, Rh, Pd, In, Sn, Sb, Zn, Ta, W, Re, Os, Ir, Pt, Au, Hg, TI, Pb, Bi, Po, Fr, Pm, a lanthanide (such as La, Ce, Pr, Nd, Pm, Sm, Eu, Gd, Tb, Dy, Ho, Er, Tm, Yb, Lu), an actinide (such as Ac, Th, U), Mg, Al, Ca, Cd, or Ba.

In some embodiments, the lanthanide is Lu, Sm, Ho, or Tb.

In some embodiments, the actinide is Ac, Th, or U.

In some embodiments, the one or more radionuclides are selected from ¹⁴C, ¹⁵N, ¹⁸F, ⁷⁵Br, ⁷⁶Br, ⁷⁷Br, ¹²³I, ¹²⁴I, ¹²⁵I, ¹³¹I, ³⁵S ⁹⁹Tc, ⁹⁹mTc, ¹⁸⁸Re, ¹⁸⁶Re, ¹⁵³Sm, ⁶⁶Ga, ⁶⁷Ga, ⁶⁸Ga, ¹¹¹In, ¹²³In, ⁵⁹Fe, ⁶³Zn, ⁵²Fe, ⁵²Mn, ⁴⁵Ti ⁶⁰Cu, ⁶¹Cu, ⁶⁷Cu, ⁶⁴Cu, ⁶²Cu ⁸²Rb, ^{19m}Pt, ^{191m}Pt, ^{193m}Pt, ^{117m}Sn, ⁸⁹Zr, ¹⁷⁷Lu, ¹⁸F, ¹⁸⁸Re, ¹⁸⁶Re, ¹⁵³Sm, ⁶⁶Ho, ⁸⁶Y, ³⁷Y, ⁹⁰Y, ⁸⁹Sr, ¹⁵³Gd, ¹⁵⁹Gd, ²²⁵Ac, ²¹²Bi, ²¹³Bi, ²¹¹At, ¹⁹⁸Au, ¹⁹⁹Au, ^{193m}Pt, ¹⁹⁷Pt, ¹⁰³Pd, ¹⁰⁹Pd, ¹⁰⁵Rh, ^{101m}Rh, ^{103m}Rh, ²²³Ra, ²²⁴Ra, ⁹⁷Ru, ²²⁷Th, ²²⁹Th, ³²P ¹⁶¹Tb, ³³P ¹⁴⁹Tb, ¹²⁵I, ²⁰³Pb, ²¹²Pb, ²⁰¹Tl, ¹¹⁹Sb, ^{8m}Co, ⁵⁵Co, ⁵⁷Co, ⁴⁷Sc, ¹⁴⁹Pm, ¹⁴²Pr, ¹⁶¹Ho, ¹⁶⁶Ho, ¹⁷⁵Yb, or ⁵¹Cr.

In some embodiments, the one or more radionuclides are for use in imaging or for use in therapy.

In some embodiments, the one or more radionuclides for use in imaging are selected from ^{99m}Tc, ¹⁸⁸Re, ¹⁸⁶Re, ¹⁵³Sm, ⁶⁶Ga, ⁶⁷Ga, ⁶⁸Ga, ¹¹¹In, ⁵⁹Fe, ⁶³Zn, ⁵²Fe, ⁵²Mn, ⁴⁵Ti, ⁶⁰Cu, ⁶¹Cu, ⁶⁷Cu, ⁶⁴Cu, ⁶²Cu, ⁸²Rb, ¹⁹⁸Au, ¹⁹⁹Au, ^{199m}Pt, ^{191m}Pt, ^{193m}Pt, ^{117m}Sn, ⁸⁹Zr, ¹⁷⁷Lu ¹⁸F, ²⁰³Pb, ⁴⁴Sc, ⁵¹Cr, ^{101m}Rh, ¹⁶⁶Ho, or ¹²³I.

In some embodiments, the one or more radionuclides for use in therapy are selected from ¹⁸⁸Re, ¹⁸⁶Re, ¹⁵³Sm, ⁶⁶Ho, ⁹⁰Y, ⁸⁹Sr, ¹¹¹In, ¹⁵³Gd, ²²⁵Ac, ²¹²Bi, ²¹³Bi, ²¹¹At, ⁶⁰Cu, ⁶¹Cu, ⁶⁷Cu, ⁶⁴Cu, ⁶²Cu, ¹⁹⁸Au, ⁹⁹Au, ^{199m}Pt, ^{193m}Pt, ¹⁹⁷Pt, ^{117m}Sn, ¹⁰³Pd, ¹⁰⁵Rh, ^{103m}Rh, ¹⁷⁷Lu, ²²³Ra, ²²⁴Ra, ²²⁷Th, ²²⁹Th, ¹⁴⁹Tb, ³²P, ¹⁶¹Tb, 33P, ¹²⁵I, ²⁰³Pb, ²¹²Pb, ²⁰¹Tl, ¹¹⁹Sb, ^{58m}Co, ⁴⁷Sc, ¹⁴⁹Pm ¹⁶¹Ho, ¹⁵⁹Gd, ¹⁴²Pr, ¹⁶⁶Ho, or ¹⁷⁵Yb.

In some embodiments, the one or more radionuclides for use in therapy are selected from ¹⁷⁷Lu, ²¹²Pb, and ²²⁵Ac. In some embodiments, the radionuclides for use in therapy is ¹⁷Lu or ²²⁵Ac. In some embodiments, the one or more radionuclides for use in therapy is ¹⁷Lu. In some embodiments, the one or more radionuclides for use in therapy is ²²⁵Ac.

In some embodiments, the compound of Formula I or a pharmaceutically acceptable salt, solvate and/or prodrug thereof when complexed to ¹⁷⁷Lu comprises a net charge of positive 2 (+2) to negative five (−5). In some embodiments, the compound of Formula I or a pharmaceutically acceptable salt, solvate and/or prodrug thereof when complexed to ¹⁷⁷Lu comprises a net charge of positive (+1) to negative five (−5). In some embodiments, the compound of Formula I or a pharmaceutically acceptable salt, solvate and/or prodrug thereof when complexed to ¹⁷⁷Lu comprises a net charge of zero (O) to negative five (−5). In some embodiments the compound of Formula I or a pharmaceutically acceptable salt, solvate and/or prodrug thereof when complexed to ¹⁷⁷Lu comprises a net charge of zero (0) to negative four (−4). In some embodiments, the compound of Formula I or a pharmaceutically acceptable salt, solvate and/or prodrug thereof when complexed to ¹⁷⁷Lu comprises a net charge of zero (0) to negative three (−3). In some embodiments, the compound of Formula I or a pharmaceutically acceptable salt, solvate and/or prodrug thereof when complexed to ¹⁷⁷Lu comprises a net charge of zero (0) to negative two (−2). In some embodiments, the compound of Formula I or a pharmaceutically acceptable salt, solvate and/or prodrug thereof when complexed to ¹⁷⁷Lu comprises a net charge of zero (0) or negative one (−1). In some embodiments, the compound of Formula I or a pharmaceutically acceptable salt, solvate and/or prodrug thereof when complexed to ¹⁷⁷Lu comprises a net charge of 0. In some embodiments, the compound of Formula I or a pharmaceutically acceptable salt, solvate and/or prodrug thereof when complexed to ¹⁷⁷Lu comprises a net charge of −1.

In some embodiments, T is a trivalent branching group comprising at least three terminal functional groups. Therefore, in some embodiments, T is a trivalent branching group comprising at least a first terminal functional group, a second terminal functional group and a third terminal functional group, which are the same or different and bind to a complementary functional groups on L¹ (or alternatively E), L² (or alternatively Z¹) and L³ (or alternatively Z²), respectively. In some embodiments, T comprises at least a first terminal functional group, a second terminal functional group and a third terminal functional group, which are the same or different and bind to a complementary functional group on L¹ (or alternatively E), L² (or alternatively Z¹) and L³ (or alternatively Z²), respectively form an amide group, a urea group, a thiourea groups or a thioamide group.

In some embodiments, T comprises at least a first terminal functional group, a second terminal functional group and a third terminal functional group which are the same or different and when bonded to complementary functional groups on L¹ (or alternatively E), L² (or alternatively Z¹) and L³ (or alternatively Z²), respectively independently form an amide group.

In some embodiments, T is selected from an amino acid residue derived from lysine, ornithine, homo-lysine, 2,3-diaminopropionic acid (Dap), 2,4-diaminobutyric acid (Dab), cysteine, homo-cysteine, glutamate or glutamine. In some embodiments, T is an amino acid residue derived from lysine,

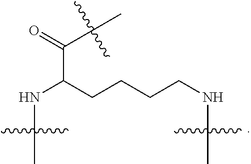 [SMILES: *NCCCCC(N*)C(*)=O]

Therefore, in some embodiments, the compound of Formula I is a compound of Formula I-B or a compound of Formula I-C or a pharmaceutically acceptable salt and/or solvate thereof:

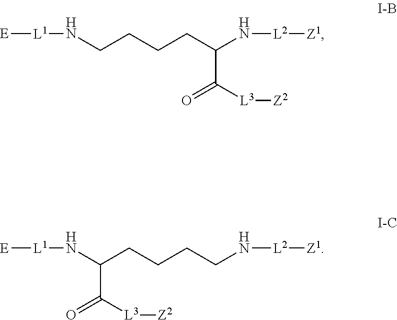

Accordingly, in some embodiments, the compound of Formula I is a compound of Formula I-B(i) or a compound of Formula I-C(i) or a pharmaceutically acceptable salt and/or solvate thereof:

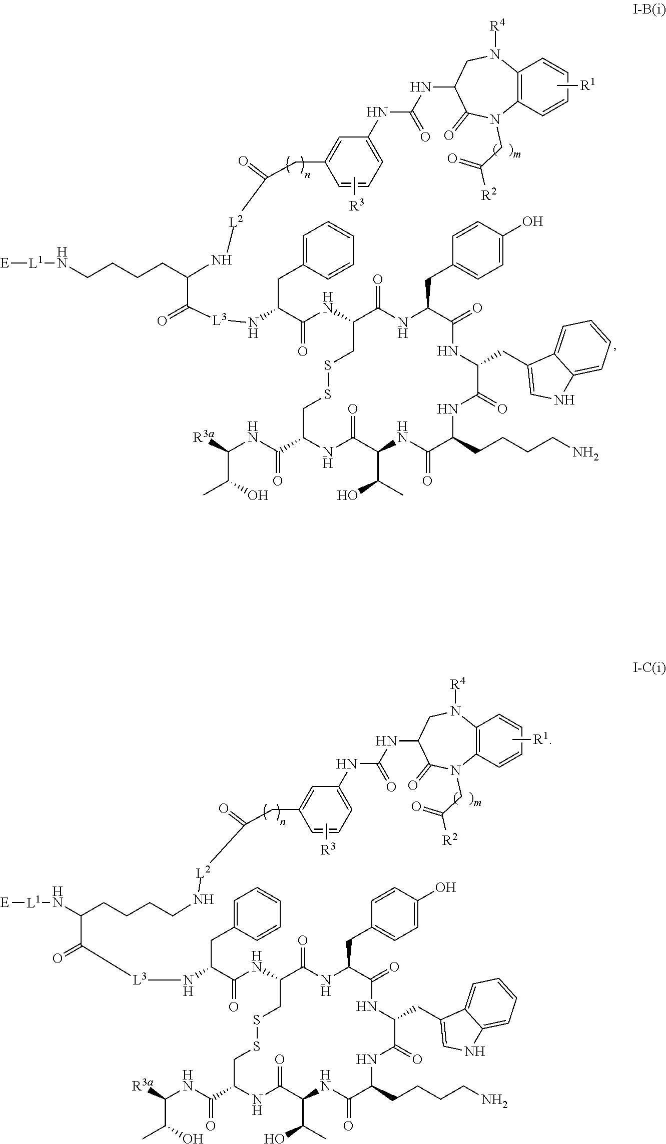

It would be appreciated by a person skilled in the art that when any of L¹, L² and L³ is a divalent linker, the divalent linker forms covalent bonds with E, Z¹ or Z² and T respectively. Therefore, in some embodiments, the divalent linker is any chemical moiety capable of forming a covalent bond with E, Z¹ or Z² and T respectively. In some embodiments, the divalent linker comprises linker moieties selected from amines, ethers, thioethers, carbonyl, thiocarbonyl, sulfones, sulfoxides, urea, thiourea, and amides. In some embodiments, the divalent linker comprises a functional group on an end terminus capable of forming a covalent bond with complementary functional group on E, Z¹ or Z² and T respectively. For example, in some embodiments, the divalent linker comprises an amine on an end terminus that reacts with a carboxylic acid group on E. In some embodiments, the divalent linker comprises an amine on an end terminus that reacts with a carboxylic acid group on E to form an amide.

In some embodiments, L¹, L² and L³ are each independently a direct bond or a divalent linker. In some embodiments, L¹, L² and L³ are each independently a direct bond or a divalent linker, and the divalent linkers comprise a combined total of 1 to 15 groups independently selected from amino acid residues, C₁₋₂₇alkylene, C₂₋₂₇alkenylene, and C₂₋₂₇alkynylene. In some embodiments, each C₁₋₂₇alkylene, C₂₋₂₇alkenylene, and C₂₋₂₇alkynylene is independently and optionally interrupted by one or more linker moieties selected from amines, ethers, thioethers, carbonyl, thiocarbonyl, sulfones, sulfoxides, urea, thiourea and amides.

Accordingly, in some embodiments, L¹, L² and L³ are each independently a direct bond or a divalent linker, and the divalent linkers comprise a combined total of 1 to 15 groups selected from amino acid residues, W^{a}, R^{a}, R^{a}-W^{a}, W^{a}-R^{b}, R^{a}-W^{a}-R^{b} and W^{a}-R^{b}-W^{b},
wherein each W^{a} and W^{b} is independently selected from O, S, S(O), SO₂, NR⁵, C(O), C(S), NR⁵C(O), NR⁵C(S), C(O)NR⁵, C(S)NR⁵, (C₁₋₆alkyleneY)ₚ and Y-(C₁₋₆alkyleneY′)ₚ;
each R^{a} and R^{b} is independently selected from C₁₋₂₀alkylene, C₂₋₂₀alkenylene, and C₂₋₂₀alkynylene;
each Y and Y′ is independently selected from O, S, C(O) and NR⁶;
each R⁵ is independently selected from H and C₁₋₆alkyl;
each R⁶ is independently selected from H and C₁₋₃alkyl; and
p is an integer selected from 1 to 8.

By “divalent linkers comprise a combined total of 1 to 15 groups” as used herein means that the combined total of groups from the 1, 2 or 3 divalent linkers is 0 to 15. For example, in a compound of Formula I wherein L¹ is a direct bond, L² is an amino acid residue, and L³ is a same or different amino acid residue, the combined total of groups is 2.

A person skilled in the art would appreciate that two adjacent groups in a divalent linker should be chosen so as to avoid a direct bond between two groups which would result in a partial structure that is not stable, for example, in an aqueous medium at room temperature such as about 18° C. to about 25° C.

In some embodiments, each R^{a} and R^{b} is independently selected from C₁₋₁₀alkylene, C₂₋₁₀alkenylene and C₂₋₁₀alkynylene. In some embodiments, each R^{a} and R^{b} is independently selected from C₁₋₂₀alkylene. In some embodiments, each R^{a} and R^{b} is independently selected from C₁₋₁₀alkylene.

In some embodiments, each W^{a} and W^{b} is independently selected from O, S, S(O), SO₂, NR⁵, C(O), C(S), C(S)NR⁵, NR⁵C(S), C(O)NR⁵, NR⁵C(O), (C₁₋₆alkyleneY)ₚ and Y-(C₁₋₆alkyleneY′)ₚ. In some embodiments, W^{a} and W^{b} are independently selected from O, S, S(O), SO₂, NR⁵, C(O), C(S), (C₁₋₆alkyleneY)ₚ and Y-(C₁₋₆alkyleneY′)ₚ.

In some embodiments, each Y and Y′ is independently selected from 0, S, C(O) and NR⁶. In some embodiments, each Y and Y′ is independently selected from O, C(O) and NR⁶. In some embodiments, each Y and Y′ is O. In some embodiments, each Y and Y′ is independently selected from 0, C(O) and NR⁶, and (C₁₋₆alkyleneY)ₚ in each W^{a} and W^{b} is independently selected from (C₁₋₆alkyleneO)ₚ, (C₁₋₆alkyleneC(O))ₚ and O—(C₁₋₆alkyleneNR⁶)ₚ.

In some embodiments, each Y and Y′ is independently selected from O, C(O) and NR⁶, and Y-(C₁₋₆alkyleneY′)ₚ, in each W^{a} and W^{b} is independently selected from O—(C₁₋₆alkyleneO)ₚ, O—(C₁₋₆alkyleneC(O))ₚ, O—(C₁₋₆alkyleneNR⁶)ₚ, C(O)—(C₁₋₆alkyleneO)ₚ, C(O)—(C₁₋₆alkyleneC(O))ₚ, C(O)—(C₁₋₆alkyleneNR⁶)ₚ, NR⁶—(C₁₋₆alkyleneO)ₚ, NR⁶—(C₁₋₆alkyleneC(O))ₚ and NR⁶—(C₁₋₆alkyleneNR⁶)ₚ.

In some embodiments, each Y and Y′ is independently selected from O, C(O) and NR⁶, and each W^{a} and W^{b} is independently selected from O, S, S(O), SO₂, NR⁵, C(O), C(S), C(S)NR⁵, NR⁵C(S), C(O)NR⁵, NR⁵C(O), (C₁₋₆alkyleneO)ₚ, (C₁₋₆alkyleneNR⁶)ₚ, (C₁₋₆alkyleneC(O))ₚ, O—(C₁₋₆alkyleneO)ₚ, O—(C₁₋₆alkyleneNR⁶)ₚ, O—(C₁₋₆alkyleneC(O))ₚ, NR⁶—(C₁₋₆alkyleneO)ₚ, NR⁶—(C₁₋₆alkyleneNR⁶)ₚ, NR⁶(C₁₋₆alkyleneC(O))ₚ, C(O)—(C₁₋₆alkyleneO)ₚ, C(O)—(C₁₋₆alkyleneNR⁶)ₚ, and C(O)—(C₁₋₆alkyleneC(O)ₚ. In some embodiments, each W^{a} and W^{b} is independently selected from O, S, S(O), SO₂, NR⁵, C(O), C(O)NR⁵, NR⁵C(O), (C₁₋₆alkyleneO)ₚ, (C₁₋₆alkyleneNR⁶)ₚ, (C₁₋₆alkyleneC(O))ₚ, O-(C₁₋₆alkyleneO)ₚ, O—(C₁₋₆alkyleneNR⁶)ₚ, O—(C₁₋₆alkyleneC(O))ₚ, NR⁶—(C₁₋₆alkyleneO)ₚ, NR⁶—(C₁₋₆alkyleneNR⁶)ₚ, NR⁶—(C₁₋₆alkyleneC(O))ₚ, C(O)—(C₁₋₆alkyleneO)ₚ, C(O)—(C₁₋₆alkyleneNR⁶)ₚ and C(O)—(C₁₋₆alkyleneC(O). In some embodiments, each Y and Y′ is selected from O, C(O) and NR⁶ and therefore each W^{a} and W^{b} is independently selected from O, S, S(O), SO₂, NR⁵, C(O), C(S), C(S)NR⁵, NR⁵C(S), C(O)NR⁵, NRSC(O), (C₁₋₆ alkyleneO)ₚ, (C₁₋₆alkyleneNR⁶)ₚ, (C₁₋₆alkyleneC(O))ₚ, O—(C₁₋₆alkyleneO)ₚ, O—(C₁₋₆alkyleneNR⁶)ₚ, O—(C₁₋₆alkyleneC(O))ₚ, NR⁶—(C₁₋₆alkyleneO)ₚ, NR⁶—(C₁₋₆alkyleneNH)ₚ, NR⁶—(C₁₋₆alkyleneN(CH₃))ₚ, NR⁶(C₁₋₆alkyleneC(O))ₚ, C(O)—(C₁₋₆alkyleneO)ₚ, C(O)—(C₁₋₆alkyleneNR⁶)ₚ, and C(O)—(C₁₋₆alkyleneC(O)ₚ. In some embodiments, each Y and Y′ is independently selected from 0, C(O) and NR⁶, and therefore each W^{a} and W^{b} is independently selected from O, S, S(O), SO₂, NR⁵, C(O), C(O)NR⁵, NR⁵C(O), (C₁₋₆alkyleneO)ₚ, (C₁₋₆alkyleneNR⁶)ₚ, (C₁₋₆alkyleneC(O))ₚ, O—(C₁₋₆alkyleneO)ₚ, O—(C₁₋₆alkyleneNR⁶)ₚ, O—(C₁₋₆alkyleneC(O))ₚ, NR⁶—(C₁₋₆alkyleneO)ₚ, NR⁶—(C₁₋₆alkyleneNH)ₚ, NR⁶—(C₁₋₆alkyleneN(CH₃))ₚ, NR⁶—(C₁₋₆alkyleneC(O))ₚ, C(O)—(C₁₋₆alkyleneO)ₚ, C(O)—(C₁₋₆alkyleneNR⁶)ₚ and C(O)—(C₁₋₆alkyleneC(O).

In some embodiments, the divalent linkers comprise a combined total of 1 to 15 groups selected from amino acid residues, W^{a}, R^{a}, R^{a}-W^{a}, W^{a}-R^{b}, and W^{a}-R^{b}-W^{b}. In some embodiments, the divalent linkers comprise a combined total of 1 to 15 groups selected from amino acid residues, W^{a}, R^{a} and W^{a}-R^{b}-W^{b}.

In some embodiments, each W^{a} and W^{b} is independently selected from O, S, S(O), SO₂, NR′, C(O), C(O)NR′, NRSC(O), (C₁₋₆alkyleneO)ₚ, (C₁₋₆alkyleneNR⁶)ₚ, (C₁₋₆alkyleneC(O))ₚ, O—(C₁₋₆alkyleneO)ₚ, O—(C₁₋₆alkyleneNR⁶)ₚ, O—(C₁₋₆alkyleneC(O))ₚ, NR⁶—(C₁₋₆alkyleneO)ₚ, NR⁶—(C₁₋₆alkyleneNH)ₚ, NR⁶—(C₁₋₆alkyleneN(CH₃))ₚ, NR⁶—(C₁₋₆alkyleneC(O))ₚ, C(O)—(C₁₋₆alkyleneO)ₚ, C(O)—(C₁₋₆alkyleneNR⁶)ₚ and C(O)—(C₁₋₆alkyleneC(O), and each R^{a} and R^{b} is independently selected from C₁₋₂₀alkylene, and the divalent linkers combined comprise a combined total of 1 to 15 groups selected from amino acid residues, 0, S, S(O), SO₂, NR⁵, C(O), C(O)NR′, NRSC(O), C₁₋₂₀alkylene, OC₁₋₂₀alkyleneO, OC₁₋₂₀alkyleneNR⁵, OC₁₋₂₀alkyleneC(O), NR⁵C₁₋₂₀alkyleneO, NR⁵C₁₋₂₀alkyleneNR⁵, NR⁵C₁₋₂₀alkyleneC(O), C(O)C₁₋₂₀alkyleneO, C(O)—C₁₋₂₀alkyleneC(O), C(O)C₁₋₂₀alkyleneNR⁵, O—(C₁₋₆alkyleneC(O))ₚC₁₋₂₀alkyleneO, O—(C₁₋₆alkyleneC(O))ₚC₁₋₂₀ alkyleneNR⁵, O—(C₁₋₆alkyleneC(O))ₚC₁₋₂₀alkyleneC(O), O—(C₁₋₆alkyleneNR⁶)ₚC₁₋₂₀alkyleneO, O—(C₁₋₆alkyleneNR⁶)ₚC₁₋₂₀alkyleneNR⁵, O—(C₁₋₆alkyleneNR⁶)ₚC₁₋₂₀alkyleneC(O), O—(C₁₋₆alkyleneC(O))ₚC₁₋₂₀alkyleneO, O—(C₁₋₆alkyleneC(O))ₚC₁₋₂₀alkyleneNR⁵, O—(C₁₋₆alkyleneC(O))ₚC₁₋₂₀alkyleneC(O), NR⁶—(C₁₋₆alkyleneO)ₚC₁₋₂₀alkyleneO, NR⁶—(C₁₋₆alkyleneO)ₚC₁₋₂₀alkyleneNR⁵, NR⁶—(C₁₋₆alkyleneO)ₚC₁₋₂₀alkyleneC(O), NR⁶—(C₁₋₆alkyleneNR⁶)ₚC₁₋₂₀alkyleneO, NR⁶—(C₁₋₆alkyleneNR⁶)ₚC₁₋₂₀alkyleneNR⁵, NR⁶—(C₁₋₆alkyleneNR⁶)ₚC₁₋₂₀alkyleneC(O), NR⁶(C₁₋₆alkyleneC(O))ₚC₁₋₂₀alkyleneO, NR⁶(C₁₋₆alkyleneC(O))ₚC₁₋₂₀alkyleneNR⁵, NR⁶(C₁₋₆alkyleneC(O))ₚC₁₋₂₀alkyleneC(O), C(O)—(C₁₋₆alkyleneO)ₚC₁₋₂₀alkyleneO, C(O)—(C₁₋₆alkyleneO)ₚC₁₋₂₀alkyleneNR⁵, C(O)—(C₁₋₆alkyleneO)ₚC₁₋₂₀alkyleneC(O), C(O)—(C₁₋₆alkyleneNR⁶)ₚC₁₋₂₀alkyleneO, C(O)—(C₁₋₆alkylene NR⁶)ₚC₁₋₂₀alkyleneNR⁵, C(O)—(C₁₋₆alkyleneNR⁶)ₚC₁₋₂₀alkyleneC(O), C(O)—(C₁₋₆alkyleneC(O)C₁₋₂₀alkyleneO, C(O)—(C₁₋₆alkyleneC(O)C₁₋₂₀alkyleneNR⁵ and C(O)—(C₁₋₆alkyleneC(O)C₁₋₂₀alkyleneC(O). In some embodiments, Y and Y′ are selected from O, C(O), NH and N(CH₃), W^{a} and W^{b} are as defined above, and R^{a} and R^{b} are independently selected from C₁₋₂₀alkylene, and therefore the divalent linkers combined comprise a combined total of 1 to 15 groups selected from amino acid residues, O, S, S(O), SO₂, NR⁵, C(O), C(O)NR⁵, NR⁵C(O), C₁₋₁₀alkylene, OC₁₋₂₀alkyleneO, OC₁₋₂₀alkyleneNR⁵, OC₁₋₂₀alkyleneC(O), NR⁵C₁₋₂₀alkyleneO, NR⁵C₁₋₂₀alkyleneNR⁵, NR⁵C₁₋₂₀alkyleneC(O), C(O)(C₁₋₂₀alkyleneO, C(O)—C₁₋₂₀alkyleneC(O), C(O)C₁₋₂₀alkyleneNR⁵, O—(C₁₋₆alkyleneC(O))ₚC₁₋₂₀alkyleneO, O—(C₁₋₆alkyleneC(O))ₚC₁₋₂₀ alkyleneNR⁵, O—(C₁₋₆alkyleneC(O))ₚC₁₋₂₀alkyleneC(O), O—(C₁₋₆alkyleneNR⁶)ₚC₁₋₂₀ alkyleneO, O—(C₁₋₆alkylene NR⁶)ₚC₁₋₂₀alkyleneNR⁵, O—(C₁₋₆alkyleneNR⁶)ₚC₁₋₂₀alkyleneC(O), O—(C₁₋₆alkyleneC(O))ₚC₁₋₂₀alkyleneO, O—(C₁₋₆alkyleneC(O))ₚC₁₋₂₀alkyleneNR⁵, O—(C₁₋₆alkyleneC(O))ₚC₁₋₂₀alkyleneC(O), NR⁶—(C₁₋₆alkyleneO)ₚC₁₋₂₀alkyleneO, NR⁶—(C₁₋₆alkyleneO)ₚC₁₋₂₀alkyleneNR⁵, NR⁶—(C₁₋₆alkyleneO)ₚC₁₋₂₀alkyleneC(O), NR⁶—(C₁₋₆alkyleneNR⁶)ₚC₁₋₂₀alkyleneO, NR⁶—(C₁₋₆alkyleneNR⁶)ₚC₁₋₂₀alkyleneNR⁵, NR⁶—(C₁₋₆alkyleneNR⁶)ₚC₁₋₂₀alkyleneC(O), NR⁶(C₁₋₆alkyleneC(O))ₚC₁₋₂₀alkyleneO, NR⁶(C₁₋₆alkyleneC(O))ₚC₁₋₂₀alkyleneNR⁵, NR⁶(C₁₋₆alkyleneC(O))ₚC₁₋₂₀alkyleneC(O), C(O)—(C₁₋₆alkyleneO)ₚC₁₋₂₀alkyleneO, C(O)—(C₁₋₆alkyleneO)ₚC₁₋₂₀alkyleneNR⁵, C(O)—(C₁₋₆alkyleneO)ₚC₁₋₂₀alkyleneC(O), C(O)—(C₁₋₆alkyleneNR⁶)ₚC₁₋₂₀alkyleneO, C(O)—(C₁₋₆alkylene NR⁶)ₚC₁₋₂₀alkyleneNR5, C(O)—(C₁₋₆alkyleneNR⁶)ₚC₁₋₂₀alkyleneC(O), C(O)—(C₁₋₆alkyleneC(O)C₁₋₂₀alkyleneO, C(O)—(C₁₋₆alkyleneC(O)C₁₋₂₀alkyleneNR⁵ and C(O)—(C₁₋₆alkyleneC(O)C₁₋₂₀alkyleneC(O).

in some embodiments, the divalent linkers combined comprise 1 to 15 groups independently selected from amino acid residues, O, S, S(O), SO₂, NR⁵, C(O), C(O)NR⁵, NR⁵C(O), C₁₋₁₂alkylene, OC₁₋₁₂alkyleneO, OC₁₋₁₂alkyleneNR⁵, OC₁₋₁₂ alkyleneC(O), NR⁵C₁₋₁₂alkyleneO, NR⁵C₁₋₁₂alkyleneNR⁵, NR⁵C₁₋₁₂alkyleneC(O), C(O)C₁₋₁₂alkyleneO, C(O)C₁₋₁₂alkyleneC(O), C(O)C₁₋₁₂alkyleneNR⁵, O—(C₁₋₆alkyleneC(O))ₚC₁₋₆alkyleneO, O—(C₁₋₆alkyleneC(O))ₚC₁₋₆alkyleneNR⁵, O—(C₁₋₆alkyleneC(O))ₚC₁₋₆alkyleneC(O), O—(C₁₋₆alkyleneNR⁶)ₚC₁₋₆alkyleneO, O—(C₁₋₆alkyleneNR⁶)ₚC₁₋₆alkyleneNR⁵, O—(C₁₋₆alkyleneNR⁶)ₚC₁₋₆alkyleneC(O), O—(C₁₋₆alkyleneC(O))ₚC₁₋₆alkyleneO, O—(C₁₋₆alkyleneC(O))ₚC₁₋₆alkyleneNR⁵, O—(C₁₋₆alkyleneC(O))ₚC₁₋₆alkyleneC(O), NR⁶—(C₁₋₆alkyleneO)ₚC₁₋₆alkyleneO, NR⁶—(C₁₋₆alkyleneO)ₚC₁₋₆alkyleneNR⁵, NR⁶—(C₁₋₆alkyleneO)ₚC₁₋₆alkyleneC(O), NR⁶—(C₁₋₆alkyleneNR⁶)ₚC₁₋₆alkyleneO, NR⁶—(C₁₋₆alkyleneNR⁶)ₚC₁₋₆alkyleneNR⁵, NR⁶—(C₁₋₆alkyleneNR⁶)ₚC₁₋₆alkyleneC(O), NR⁶(C₁₋₆alkyleneC(O))ₚC₁₋₆alkyleneO, NR⁶(C₁₋₆alkyleneC(O))ₚC₁₋₆alkyleneNR¹, NR⁶(C₁₋₆alkyleneC(O))ₚC₁₋₆alkyleneC(O), C(O)—(C₁₋₆ealkyleneO)ₚC₁₋₆alkyleneO, C(O)—(C₁₋₆alkyleneO)ₚC₁₋₆alkyleneNR⁵, C(O)—(C₁₋₆alkyleneO)ₚC₁₋₆alkyleneC(O), C(O)—(C₁₋₆alkyleneNR⁶)ₚC₁₋₆alkyleneO, C(O)—(C₁₋₆alkyleneNR⁶)ₚC₁₋₆alkyleneNR⁵, C(O)—(C₁₋₆alkyleneNR⁶)ₚC₁₋₂₀alkyleneC(O), C(O)—(C₁₋₆alkyleneC(O))ₚC₁₋₆alkyleneO, C(O)—(C₁₋₆alkyleneC(O))ₚC₁₋₆alkyleneNR⁵ and C(O)—(C₁₋₆alkyleneC(O)) pC₁₋₆alkyleneC(O). In some embodiments, the divalent linkers combined comprise 1 to 15 groups independently selected from amino acid residues, O, S, S(O), SO₂, NR⁵, C(O), C(O)NR⁵, NR⁵C(O), C₁₋₁₂alkylene, OC₁₋₁₂alkyleneO, OC₁₋₁₂alkyleneNR⁵, OC₁₋₁₂alkyleneC(O), NR⁵C₁₋₁₂alkyleneO, NR⁵C₁₋₁₂alkyleneNR⁵, NR⁵C₁₋₁₂alkyleneC(O), C(O)(C₁₋₁₂alkyleneO, C(O)—C₁₋₁₂alkyleneC(O), C(O)C₁₋₁₂alkyleneNR⁵, O—(C₁₋₆alkyleneC(O))ₚC₁₋₆alkyleneO, O—(C₁₋₆alkyleneC(O))ₚC₁₋₆alkyleneNR⁵, O—(C₁₋₆ealkyleneC(O))ₚC₁₋₆alkyleneC(O), O—(C₁₋₆alkyleneNR⁶)ₚC₁₋₆alkyleneO, O—(C₁₋₆alkyleneNR⁶)ₚC₁₋₆alkyleneNR⁵, O—(C₁₋₆alkyleneNR⁶)ₚC₁₋₆alkyleneC(O), O—(C₁₋₆alkyleneC(O))ₚC₁₋₆alkyleneO, O—(C₁₋₆alkyleneC(O))ₚC₁₋₆alkyleneNR⁵, O—(C₁₋₆alkyleneC(O))ₚC₁₋₆alkyleneC(O), NR⁶—(C₁₋₆alkyleneO)ₚC₁₋₆alkyleneO, NR⁶—(C₁₋₆alkyleneO)ₚC₁₋₆alkyleneNR⁵, NR⁶—(C₁₋₆alkyleneO)ₚC₁₋₆alkyleneC(O), NR⁶—(C₁₋₆alkyleneNR⁶)ₚC₁₋₆alkyleneO, NR⁶—(C₁₋₆alkyleneNR⁶)ₚC₁₋₆alkyleneNR⁵, NR⁶—(C₁₋₆alkyleneNR⁶)ₚC₁₋₆alkyleneC(O), NR⁶(C₁₋₆alkyleneC(O))ₚC₁₋₆alkyleneO, NR⁶(C₁₋₆alkyleneC(O))ₚC₁₋₆alkyleneNR¹, NR⁶(C₁₋₆alkyleneC(O))ₚC₁₋₆alkyleneC(O), C(O)—(C₁₋₆alkyleneO)ₚC₁₋₆alkyleneO, C(O)—(C₁₋₆alkyleneO)ₚC₁₋₆alkyleneNR⁵, C(O)—(C₁₋₆alkyleneO)ₚC₁₋₆alkyleneC(O), C(O)—(C₁₋₆alkyleneNR⁶)ₚC₁₋₆alkyleneO, C(O)—(C₁₋₆alkyleneNR⁶)ₚC₁₋₆alkyleneNR⁵, C(O)—(C₁₋₆alkyleneNR⁶)ₚC₁₋₂₀alkyleneC(O), C(O)—(C₁₋₆alkyleneC(O)C₁₋₆alkyleneO, C(O)—(C₁₋₆alkyleneC(O)C₁₋₆alkyleneNR⁵ and C(O)—(C₁₋₆alkyleneC(O)C₁₋₆alkyleneC(O).

In some embodiments, the divalent linkers combined comprise 1 to 15 groups independently selected from amino acid residues, NR⁵C₁₋₁₂alkyleneC(O), C(O)—C₁₋₁₂alkyleneC(O), C(O)C₁₋₁₂alkyleneNR⁵, NR⁵C₁₋₁₂alkyleneNR⁵, NR⁶—(C₁₋₆alkyleneO)ₚC₁₋₆alkyleneNR⁵, NR⁶—(C₁₋₆alkyleneO)ₚC₁₋₆alkyleneC(O), NR6—(C₁₋₆alkyleneNR⁶)ₚC₁₋₆alkyleneNR⁵, NR⁶—(C₁₋₆alkyleneNR⁶)ₚC₁₋₆alkyleneC(O), NR⁶(C₁₋₆alkyleneC(O))ₚC₁₋₆alkyleneNR¹, NR⁶(C₁₋₆alkyleneC(O))ₚC₁₋₆alkyleneC(O), C(O)—(C₁₋₆alkyleneO)ₚC₁₋₆alkyleneNR⁵, C(O)—(C₁₋₆alkyleneO)ₚC₁₋₆alkyleneC(O), C(O)—(C₁₋₆alkyleneNR⁶)ₚC₁₋₆alkyleneNR⁵, C(O)—(C₁₋₆alkyleneNR⁶)ₚC₁₋₂₀alkyleneC(O), C(O)—(C₁₋₆alkyleneC(O))ₚC₁₋₆alkyleneNR⁵ and C(O)—(C₁₋₆alkyleneC(O))ₚC₁₋₆alkyleneC(O). In some embodiments, the divalent linkers combined comprise 1 to 15 groups independently selected from amino acid residues, NR⁵C₁₋₁₂alkyleneC(O), C(O)—C₁₋₁₂alkyleneC(O), C(O)C₁₋₁₂alkyleneNR⁵, NR⁶—(C₁₋₆alkyleneO)ₚC₁₋₆alkyleneNR⁵, NR⁶—(C₁₋₆alkyleneO)ₚC₁₋₆alkyleneC(O), NR⁶—(C₁₋₆alkyleneNR⁶)ₚC₁₋₆alkyleneNR⁵, NR⁶—(C₁₋₆alkyleneNR⁶)ₚC₁₋₆alkyleneC(O), NR⁶(C₁₋₆alkyleneC(O))ₚC₁₋₆alkyleneNR⁵, NR⁶(C₁₋₆alkyleneC(O))ₚC₁₋₆alkyleneC(O), C(O)—(C₁₋₆alkyleneO)ₚC₁₋₆alkyleneNR⁵, C(O)—(C₁₋₆alkyleneO)ₚC₁₋₆alkyleneC(O), C(O)—(C₁₋₆alkyleneNR⁶)ₚC₁₋₆alkyleneNR⁵, C(O)—(C₁₋₆alkyleneNR⁶)ₚC₁₋₂₀alkyleneC(O), C(O)—(C₁₋₆alkyleneC(O)C₁₋₆alkyleneNR⁵ and C(O)—(C₁₋₆alkyleneC(O)C₁₋₆alkyleneC(O).

In some embodiments, the divalent linkers combined comprise 1 to 15 groups independently selected from amino acid residues, NR⁵C₁₋₁₂alkyleneC(O), C(O)C₁₋₁₂alkyleneNR⁵, NR⁵C₁₋₁₂alkyleneNR⁵, NR⁶—(C₁₋₆alkyleneO)ₚC₁₋₆alkyleneC(O), C(O)—(C₁₋₆alkyleneO)ₚC₁₋₆alkyleneNR⁵, C(O)—(C₁₋₆alkyleneNR⁶)ₚC₁₋₆alkyleneNR⁵, C(O)—(C₁₋₆alkyleneNR⁶)ₚC₁₋₂₀alkyleneC(O), C(O)—(C₁₋₆alkyleneC(O))ₚC₁₋₆alkyleneNR⁵ and C(O)—(C₁₋₆alkyleneC(O))ₚC₁₋₆alkyleneC(O). In some embodiments, the divalent linkers combined comprise 1 to 15 groups independently selected from amino acid residues, NR⁵C₁₋₁₂alkyleneC(O), C(O)C₁₋₁₂alkyleneNR⁵ and NR⁶—(C₁₋₆alkyleneO)ₚC₁₋₆ alkyleneC(O).

In some embodiments, the amino acid residues of the divalent linkers are amino acid residues derived from naturally occurring amino acids naturally occurring amino acids that have been modified to provide modified amino acids, D enantiomers of the naturally occurring amino acid residues or the modified amino acid residues, and amino acid residues derived from a β-amino acid or a γ-amino acid.

In some embodiments, the amino acid residues of the divalent linkers are amino acid residues derived from naturally occurring amino acids. In some embodiments, the naturally occurring amino acids are one or more naturally occurring amino acids selected from, but are not limited to, alanine (A, Ala), arginine (R, Arg), asparagine (N, Asn), aspartic acid (D, Asp), cysteine (C, Cys), glutamine (Q, Gln), glutamic acid (E, Glu), glycine (G, Gly), histidine (H, His), isoleucine (I, lie), leucine (L, Leu), Lysine (K, Lys), methionine (M, Met), phenylalanine (F, Phe), proline (P, Pro), serine (S, Ser), threonine (T, Thr), tryptophan (W, Trp), tyrosine (Y, Tyr), valine (V, Val), pyrrolysine (Pyl), selenocycleine (Sec) and pyrroline-carboxy-lysine (PCL).

In some embodiments, the amino acid residues derived from naturally occurring amino acids are naturally occurring amino acid residues derived from Glu. In some embodiments, the amino acid residues of the divalent linkers are further selected from naturally occurring amino acid residues derived from Glu. In some embodiments, the amino acid residues derived from Glu are connected through amino and the a-carboxy or the amino and the y-carboxy terminal. Therefore, in some embodiments, the amino acid residues derived from Glu are one or both selected from

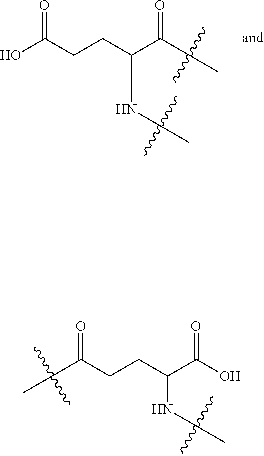 [SMILES: *NC(CCC(=O)O)C(*)=O ; *NC(CCC(*)=O)C(=O)O]

γGlu, gamma-Glu). In some embodiments, the amino acid residues derived from naturally occurring amino acids are naturally occurring amino acid residues derived from Asp. In some embodiments, the amino acid residues are further selected from amino acid residues derived from Asp. In some embodiments, the amino acid residues derived from Asp is connected through amino and the a-carboxy or is connected through the amino and the β-carboxy terminal. In some embodiments, the amino acid residues derived from naturally occurring amino acids are naturally occurring amino acid residues derived from Lys. In some embodiments, the amino acid residues are further selected from amino acid residues derived from Lys. In some embodiments, the amino acid residue derived from Lys are connected through amino and the a-carboxy or the amino and the E-amino terminal. Therefore, in some embodiments, the amino acid residues derived from Lys are one or both selected from

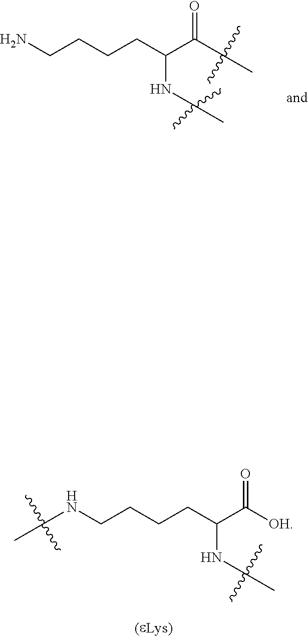 [SMILES: *NC(CCCCN)C(*)=O ; *NCCCCC(N*)C(=O)O]

In some embodiments, the amino acid residues of the divalent linkers are further selected from naturally occurring amino acids that have been modified to provide modified amino acids. Therefore, in some embodiments, the amino acid residues are further selected from one or more modified amino acids selected from, but not limited to, 4-carboxy-L-phenylalanine (Cbp), hydroxyproline, y-carboxyglutamate, O-phosphoserine, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine, 2,4-diaminoisobutyric acid, desmosine, 2,2′-diaminopimelic acid, 2,3-diaminoproprionic acid (Dap), N-ethylglycine, N-methylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid and thioproline. In some embodiments, the amino acid residues of the divalent linkers are further selected from naturally occurring amino acids that have been modified to provide modified amino acids. Therefore, in some embodiments, the amino acid residues are further selected from one or more modified amino acids selected from, but not limited to, hydroxyproline, y-carboxyglutamate, O-phosphoserine, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine, 2,4-diaminoisobutyric acid, desmosine, 2,2′-diaminopimelic acid, 2,3-diaminoproprionic acid, N-ethylglycine, N-methylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid and thioproline.

In some embodiments, the amino acid residues of the divalent linkers are further selected from the D enantiomers of the naturally occurring amino acid residues or the modified amino acid residues.

In some embodiments, the amino acid residues of the divalent linkers are further selected from amino acid residues derived from a β-amino acid or a γ-amino acid. In some embodiments, the β-amino acid is β-alanine.

By modulating the hydrophobicity and/or net charge on the radioligand when complexed to the radionuclide, the Applicants have provided radioligands that exhibit good binding affinity to both the Somatostatin type 2 receptor (SSTR2) and the cholecystokinin 2 receptor (CCK2R) and which also may exhibit greater uptake of the radionuclide in the target cell while maintaining off-target normal organ accumulation to a minimum.

Therefore, in some embodiments, the amino acid residues of the divalent linkers comprise zero negatively charged amino acid residues or at least one negatively charged amino acid residues. In some embodiments, the amino acid residues comprise zero negatively charged amino acid residues. In some embodiments, the amino acid residues comprise at least one negatively charged amino acid residue. In some embodiments, the amino acid residues are amino acids derived from naturally occurring amino acids and comprise at least one negatively charged amino acid residues. In some embodiments, the negatively charges amino acid residues are selected from D-Asp, L-Asp, D-Glu, L-Glu, D-γGlu and L-γGlu. In some embodiments, the negatively charges amino acid residues are selected from D-Glu, L-Glu, D-γGlu and L-γGlu.

Therefore, in some embodiments, the divalent linkers comprise a combined total of at least one amino acid residue selected from D-Asp, L-Asp, D-Glu, L-Glu, D-γGlu and L-γGlu. In some embodiments, the divalent linkers comprise a combined total of at least one to five amino acid residues selected from D-Asp, L-Asp, D-Glu, L-Glu, D-γGlu and L-γGlu. In some embodiments, the divalent linkers comprise a combined total of at least one to four amino acid residues selected from D-Asp, L-Asp, D-Glu, L-Glu, D-γGlu and L-γGlu. In some embodiments, the divalent linkers comprise a combined total of at least one to three amino acid residues selected from D-Asp, L-Asp, D-Glu, L-Glu, D-γGlu and L-γGlu. In some embodiments, the divalent linkers comprise a combined total of at least one to two amino acid residues selected from D-Asp, L-Asp, D-Glu, L-Glu, D-γGlu and L-γGlu. In some embodiments, the divalent linkers comprise a combined total of one amino acid residue selected from D-Asp, L-Asp, D-Glu, L-Glu, D-γGlu and L-γGlu. In some embodiments, the divalent linkers comprise a combined total of one to five amino acid residues selected from D-Asp, L-Asp, D-Glu, L-Glu, D-γGlu and L-γGlu. In some embodiments, the divalent linkers comprise a combined total of one to four amino acid residues selected from D-Asp, L-Asp, D-Glu, L-Glu, D-γGlu and L-γGlu. In some embodiments, the divalent linkers comprise a combined total of one to three amino acid residues selected from D-Asp, L-Asp, D-Glu, L-Glu, D-γGlu and L-γGlu. In some embodiments, the divalent linkers comprise a combined total of one to two amino acid residues selected from D-Asp, L-Asp, D-Glu, L-Glu, D-γGlu and L-γGlu.

In some embodiments, the amino acid residues are amino acids derived from naturally occurring amino acids and comprise at least one positively charged amino acid residue. In some embodiments, the amino acid residues further comprise positively charged amino acid residues. Therefore, in some embodiments, the one or more amino acid residues are further selected from D-His, L-His, D-Arg, L-Arg, D-Lys, L-Lys, D-εLys, and L-εLys.

It would be appreciated by a person skilled in the art that a net negative charge of the compound of Formula I or a pharmaceutically acceptable salt, solvate and/or prodrug thereof when complexed to a radionuclide can be achieved when the divalent linkers comprise a combined total of least one negatively charged amino acid residue, or alternatively, the divalent linkers comprise both positively charged amino acid residues and negatively charged amino acid residues, and the combined total number of negatively charged amino acid residues is greater than the combined total number of positively charged amino acid residues, and the net charge between the chelating group E of the compound of Formula I or a pharmaceutically acceptable salt, solvate and/or prodrug thereof and the radionuclide to which E is complexed is zero. Similarly, it would be appreciated by a person skilled in the art that a net negative charge of the compound of Formula I or a pharmaceutically acceptable salt, solvate and/or prodrug thereof when complexed to a radionuclide can also be achieved when the divalent linkers comprise a combined total of zero charged amino acid residue, or alternatively, the divalent linkers comprise the same number of positively charged amino acid residues and negatively charged amino acid residues, when the net charge between the chelating group E of the compound of Formula I or a pharmaceutically acceptable salt, solvate and/or prodrug thereof and the radionuclide to which E is complexed is negative.

In some embodiments, the amino acid residues comprise one or more hydrophilic amino acid residues. In some embodiments, the amino acid residues are selected from, D-Ser, and L-Ser.

In some embodiments, the amino acid residues comprise one or more neutral amino acid residues such as Gly, D-Pro and L-Pro.

Accordingly, in some embodiments, the divalent linkers comprise a combined total of 1 to 15 groups independently selected from 4-carboxy-L-phenylalaninel (Cbp), 2,3-diaminopropionic acid (Dap), Gly, D-His, L-His, D-Ala, L-Ala, D-Val, L-Val, D-Leu, L-Leu, D-Phe, L-Phe, D-Pro, L-Pro, D-Met, L-Met, D-Trp, L-Trp, D-Thr, L-Thr, D-Tyr, L-Tyr, D-Nle, L-Nle, D-Ser, L-Ser, D-Arg, L-Arg, D-Lys, L-Lys, D-εLys, L-εLys, D-Asp, L-Asp, D-Glu, L-Glu, D-γGlu, L-γGlu, NR⁵C₁₋₁₂alkyleneC(O), C(O)C₁₋₁₂ alkyleneNR⁵ and NR⁶—(C₁₋₆alkyleneO)ₚC₁₋₆alkyleneC(O). In some embodiments, the divalent linkers comprise a combined total of 1 to 15 groups independently selected from Gly, D-Ala, L-Ala, D-Val, L-Val, D-Leu, L-Leu, D-Phe, L-Phe, D-Pro, L-Pro, D-Met, L-Met, D-Trp, L-Trp, D-Thr, L-Thr, D-Tyr, L-Tyr, D-Nle, L-Nle, D-Ser, L-Ser, D-Arg, L-Arg, D-Lys, L-Lys, D-εLys, L-εLys, D-Asp, L-Asp, D-Glu, L-Glu, D-γGlu, L-γGlu, NR⁵C₁₋₁₂alkyleneC(O), C(O)C₁₋₁₂alkyleneNR⁵ and NR⁶—(C₁₋₆alkyleneO)ₚC₁₋₆alkyleneC(O).

In some embodiments, the divalent linkers comprise a combined total of 1 to 15 groups selected from Cbp, Dap, Gly, D-His, L-His, D-Ala, L-Ala, D-Val, L-Val, D-Leu, L-Leu, D-Phe, L-Phe, D-Pro, L-Pro, D-Ser, L-Ser, D-Arg, L-Arg, D-Lys, L-Lys, D-εLys, L-εLys, D-Asp, L-Asp, D-Glu, L-Glu, D-γGlu, L-γGlu, NR⁵C₁₋₁₂alkyleneC(O), C(O)C₁₋₁₂alkyleneNR⁵ and NR⁶—(C₁₋₆alkyleneO)ₚC₁₋₆alkyleneC(O). In some embodiments, the divalent linkers comprise a combined total of 1 to 15 groups selected from Gly, D-Ala, L-Ala, D-Val, L-Val, D-Leu, L-Leu, D-Phe, L-Phe, D-Pro, L-Pro, D-Ser, L-Ser, D-Lys, L-Lys, D-εLys, L-εLys, D-Asp, L-Asp, D-Glu, L-Glu, D-γGlu, L-γGlu, NR⁵C₁₋₁₂alkyleneC(O), C(O)C₁₋₁₂alkyleneNR⁵ and NR⁶—(C₁₋₆alkyleneO)ₚC₁₋₆alkyleneC(O).

In some embodiments, the divalent linkers comprise a combined total of 1 to 15 groups independently selected from Cbp, Dap, Gly, D-His, L-His, D-Ala, L-Ala, D-Val, L-Val, D-Ser, L-Ser, D-Pro, L-Pro, D-Arg, L-Arg, D-Lys, L-Lys, D-εLys, L-εLys, D-Asp, L-Asp, D-Glu, L-Glu, D-γGlu, L-γGlu, NR⁵C₁₋₁₂alkyleneC(O), C(O)C₁₋₁₂alkyleneNR⁵ and NR⁶—(C₁₋₆alkyleneO)ₚC₁₋₆alkyleneC(O). In some embodiments, the divalent linkers comprise a combined total of 1 to 15 groups independently selected from Gly, D-Ala, L-Ala, D-Val, L-Val, D-Ser, L-Ser, D-Pro, L-Pro, D-Lys, L-Lys, D-εLys, L-εLys, D-Asp, L-Asp, D-Glu, L-Glu, D-γGlu, L-γGlu, NR⁵C₁₋₁₂alkyleneC(O), C(O)C₁₋₁₂alkyleneNR⁵ and NR⁶—(C₁₋₆alkyleneO)ₚC₁₋₆alkyleneC(O).

In some embodiments, the divalent linkers comprise a combined total of 1 to 15 groups independently selected from Cbp, Dap, Gly, D-His, L-His D-Ala, L-Ala, D-Val, L-Val, D-Ser, L-Ser, D-Pro, L-Pro, D-Arg, L-Arg, D-Lys, L-Lys, D-εLys, L-εLys, D-Asp, L-Asp, D-Glu, L-Glu, D-γGlu, L-γGlu, NR⁵C₂₋₈alkyleneC(O), C(O)C₂₋₆alkyleneNR⁵ and NR⁶—(C₁₋₃alkyleneO)ₚC₁₋₃alkyleneC(O).

In some embodiments, the divalent linkers comprise a combined total of 1 to 15 groups independently selected from Cbp, Dap, Gly, D-Ala, D-His, L-His L-Ala, D-Val, L-Val, D-Ser, L-Ser, D-Pro, L-Pro, D-Arg, L-Arg, D-Lys, L-Lys, D-εLys, L-εLys, D-Asp, L-Asp, D-Glu, L-Glu, D-γGlu, L-γGlu, NR⁵C₅alkyleneC(O) (Ahx), NR⁵C₇alkyleneC(O) (Aoc), NR⁵C₁₀alkyleneC(O) (Aun), NR⁶—(C₂alkyleneO)ₚC₁alkyleneC(O) and NR⁶—(C₂alkyleneO)ₚC₂alkyleneC(O).In some embodiments, the divalent linkers comprise a combined total of 1 to 15 groups independently selected from Gly, D-Ala, L-Ala, D-Val, L-Val, D-Ser, L-Ser, D-Pro, L-Pro, D-Lys, L-Lys, D-εLys, L-εLys, D-Asp, L-Asp, D-Glu, L-Glu, D-γGlu, L-γGlu, NR⁵C₅alkyleneC(O) (Ahx) NR⁵C₇alkyleneC(O) (Aoc), NR⁵C₁₀alkyleneC(O) (Aun), NR⁶—(C₂alkyleneO)ₚC₁alkyleneC(O) and NR⁶—(C₂alkyleneO)ₚC₂alkyleneC(O).

In some embodiments, p is an integer selected from 1 to 7, 1 to 6 or 2 to 6.

In some embodiments, p is an integer selected from 1 to 6 and the divalent linkers comprise a combined total of 1 to 15 groups independently selected from Cbp, Dap, Gly, D-His, L-His, D-Ala, L-Ala, D-Ser, L-Ser, D-Pro, L-Pro, D-Arg, L-Arg, D-Lys, L-Lys, D-εLys, L-εLys, D-Asp, L-Asp, D-Glu, L-Glu, D-γGlu, L-γGlu,

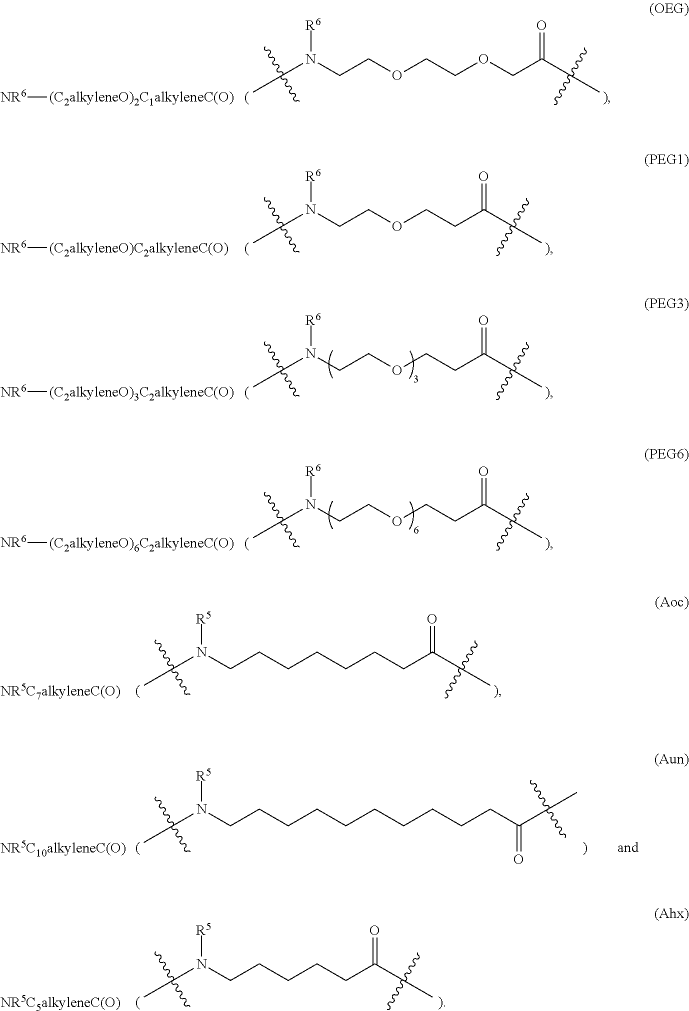

In some embodiments, p is an integer selected from 1 to 6 and the divalent linkers comprise a combined total of 1 to 15 groups independently selected from Gly, D-Ala, L-Ala, D-Ser, L-Ser, D-Pro, L-Pro, D-Lys, L-Lys, D-εLys, L-εLys, D-Asp, L-Asp, D-Glu, L-Glu, D-γGlu, L-γGlu,

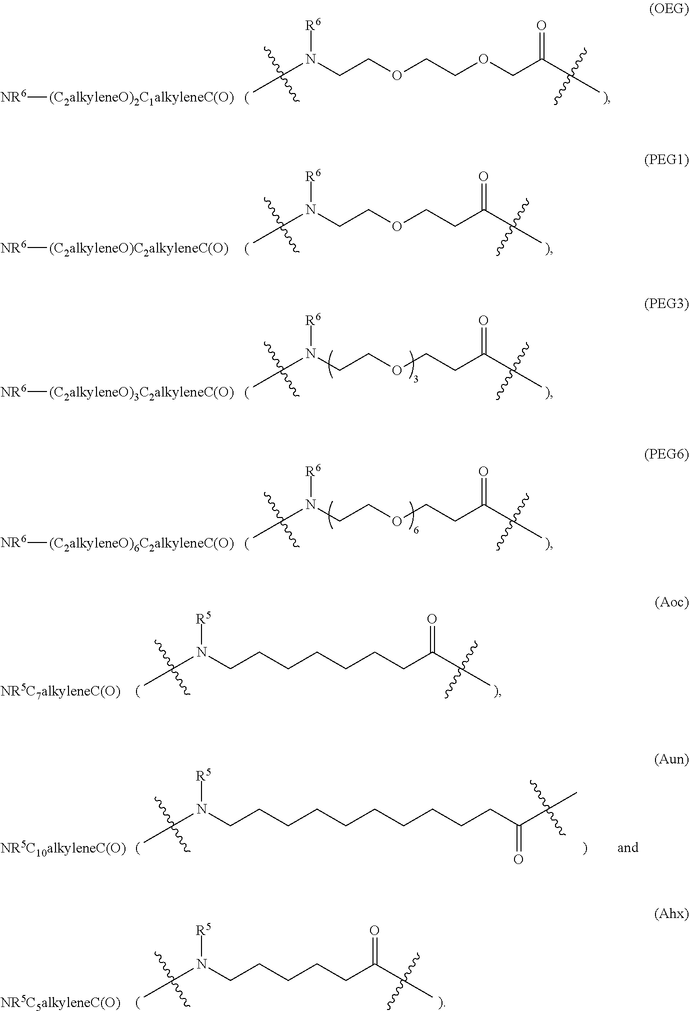

In some embodiments, the divalent linkers comprise a combined total of 1 to 15 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx. In some embodiments, the divalent linkers comprise a combined total of 1 to 15 groups independently selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx.

In some embodiments, the divalent linkers comprise a combined total of 1 to 14 groups, 1 to 12 groups, 1 to 10 groups, 1 to 9 groups, 1 to 8 groups, 1 to 7 groups, 1 to 6 groups, 1 to 5 groups, 1 to 4 groups, 1 to 3 groups, 1 to 2 groups, or 1 group. In some embodiments, the divalent linkers comprise a combined total of 1 to 7 groups, 1 to 6 groups, 1 to 5 groups, 1 to 4 groups, 1 to 3 groups, 1 to 2 groups, or 1 group. In some embodiments, the compound of Formula I or a pharmaceutically acceptable salt, solvate and/or prodrug thereof when complexed to a radionuclide comprises a net charge of negative five (−5). In some embodiments, the divalent linker groups comprise a combined total of at least 4 or 5 negatively amino acid residues selected from D-Glu, L-Glu, D-γGlu and L-γGlu. In some embodiments, the divalent linkers comprise a combined total 4 to 11 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, wherein at least 4 or 5 groups are selected from D-Glu, L-Glu, D-γGlu and L-γGlu. In some embodiments, the divalent linkers comprise a combined total 4 to 11 groups independently selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, wherein at least 4 or 5 groups are selected from D-Glu, L-Glu, D-γGlu and L-γGlu. In some embodiments, the divalent linkers comprise a combined total 4 to 11 groups independently selected from Cbp, Dap, Gly, D-Pro, L-Pro, D-Ser, L-Ser, D-Glu, L-Glu, D-γGlu, L-γGlu, OEG, PEG1, PEG3, PEG6, and Aoc, wherein at least 4 or 5 groups are selected from D-Glu, L-Glu, D-γGlu and L-γGlu. In some embodiments, the divalent linkers comprise a combined total 4 to 11 groups independently selected from Gly, D-Pro, L-Pro, D-Ser, L-Ser, D-Glu, L-Glu, D-γGlu, L-γGlu, OEG, PEG1, PEG3, PEG6, and Aoc, wherein at least 4 or 5 groups are selected from D-Glu, L-Glu, D-γGlu and L-γGlu. In some embodiments, the divalent linkers comprise a combined total of 5 to 11 groups independently selected from Gly, L-Pro, L-Glu, L-γGlu, OEG, PEG1, PEG3, PEG6, and Aoc wherein at least 4 or 5 groups are selected from L-Glu and L-γGlu. In some embodiments, the divalent linkers comprise a combined total of 4 to 11 groups independently selected from Gly, L-Glu, L-γGlu and OEG wherein at least 4 or 5 groups are selected from L-Glu and L-γGlu.

In some embodiments, the compound of Formula I or a pharmaceutically acceptable salt, solvate and/or prodrug thereof when complexed to a radionuclide comprises a net charge of negative four (−4). In some embodiments, the divalent linkers combined comprise at least 3 or 4 amino acid residues selected from D-Glu, L-Glu, D-γGlu and L-γGlu. In some embodiments, the divalent linkers combined comprise 3 to 11 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, wherein 3 or 4 groups are selected from D-Glu, L-Glu, D-γGlu and L-γGlu. In some embodiments, the divalent linkers combined comprise 3 to 11 groups independently selected from Cbp, Dap, Gly, D-Pro, L-Pro, D-Ser, L-Ser, D-Glu, L-Glu, D-γGlu, and L-γGlu, wherein at least 3 or 4 groups are selected from D-Glu, L-Glu, D-γGlu and L-γGlu. In some embodiments, the divalent linkers combined comprise 3 to 11 groups independently selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, wherein 3 or 4 groups are selected from D-Glu, L-Glu, D-γGlu and L-γGlu. In some embodiments, the divalent linkers combined comprise 3 to 11 groups independently selected from Gly, D-Pro, L-Pro, D-Ser, L-Ser, D-Glu, L-Glu, D-γGlu, and L-γGlu, wherein at least 3 or 4 groups are selected from D-Glu, L-Glu, D-γGlu and L-γGlu. In some embodiments, the divalent linkers combined comprise 3 to 11 groups independently selected from Gly, L-Pro, L-Glu, L-γGlu, OEG, PEG1, PEG3, PEG6 and Aoc, wherein 4 or 5 groups are selected from L-Glu and L-γGlu. In some embodiments, the divalent linkers combined comprise 3 or 4 groups independently selected from L-Glu and L-γGlu. In some embodiments, E is a chelating group comprising three free carboxylic acid groups such as a chelating group derived from DOTA, the divalent linkers combined comprise 3 to 11 groups independently selected from Gly, L-Pro, L-Glu, L-γGlu, OEG, PEG1, PEG3, PEG6, and Aoc, wherein 4 groups are selected from L-Glu and L-γGlu and the net charge of the compound of Formula I or a pharmaceutically acceptable salt, solvate and/or prodrug when complexed to ¹⁷⁷Lu is negative four (−4). In some embodiments, E is a chelating group comprising four free carboxylic acid groups such as a chelating group derived from DOTAGA, the divalent linkers combined comprise 3 to 11 groups independently selected from Gly, L-Pro, L-Glu, L-γGlu, OEG, PEG1, PEG3, PEG6, and Aoc, wherein 3 groups are selected from L-Glu and L-γGlu and the net charge of the compound of Formula I or a pharmaceutically acceptable salt, solvate and/or prodrug when complexed to ¹⁷⁷Lu is negative four (−4).

In some embodiments, the compound of Formula I or a pharmaceutically acceptable salt, solvate and/or prodrug thereof when complexed to a radionuclide comprises a net charge of negative three (−3). In some embodiments, the divalent linkers comprise a combined total of at least 2 or 3 amino acid residues selected from D-Glu, L-Glu, D-γGlu and L-γGlu. In some embodiments, the divalent linkers comprise a combined total of 2 to 9 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, wherein at least 2 or 3 groups are selected from D-Glu, L-Glu, D-γGlu and L-γGlu. In some embodiments, the divalent linkers comprise a combined total of 2 to 9 groups independently selected from Cbp, Dap, Gly, D-Pro, L-Pro, D-Ser, L-Ser, D-Glu, L-Glu, D-γGlu, L-γGlu, OEG, PEG1, PEG3, PEG6, and Aoc, wherein at least 2 or 3 groups are selected from D-Glu, L-Glu, D-γGlu and L-γGlu. In some embodiments, the divalent linkers comprise a combined total of 2 to 9 groups independently selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, wherein at least 2 or 3 groups are selected from D-Glu, L-Glu, D-γGlu and L-γGlu. In some embodiments, the divalent linkers comprise a combined total of 2 to 9 groups independently selected from Gly, D-Pro, L-Pro, D-Ser, L-Ser, D-Glu, L-Glu, D-γGlu, L-γGlu, OEG, PEG1, PEG3, PEG6, and Aoc, wherein at least 2 or 3 groups are selected from D-Glu, L-Glu, D-γGlu and L-γGlu. In some embodiments, the divalent linkers combined comprise a combined total of 2 to 9 groups independently selected from Gly, L-Pro, L-Glu, L-γGlu, OEG, PEG1, PEG3, PEG6 and Aoc, wherein 2 or 3 groups are selected from L-Glu and L-γGlu. In some embodiments, the divalent linker groups comprise a combined total of 2 to 9 groups independently selected from Gly, L-Glu, L-γGlu and OEG, wherein 2 or 3 groups are selected from L-Glu and L-γGlu. In some embodiments, E is a chelating group comprising three free carboxylic acid groups such a chelating group derived from as DOTA, the divalent linkers combined comprise 2 to 9 groups independently selected from Gly, L-Pro, L-Glu, L-γGlu, OEG, PEG1, PEG3, PEG6, and Aoc, wherein 3 groups are selected from L-Glu and L-γGlu and the net charge of the compound of Formula I or a pharmaceutically acceptable salt, solvate and/or prodrug when complexed to a ¹⁷⁷Lu is negative three (−3). In some embodiments, E is a chelating group comprising four free carboxylic acid groups such as a chelating group derived from DOTAGA, the divalent linkers combined comprise 2 to 9 groups independently selected from Gly, L-Pro, L-Glu, L-γGlu, OEG, PEG1, PEG3, PEG6, and Aoc wherein 2 groups are selected from L-Glu and L-γGlu and compound of Formula I or a pharmaceutically acceptable salt, solvate and/or prodrug when complexed to a ¹⁷⁷Lu comprises a net charge of negative three (−3).

In some embodiments, the compound of Formula I or a pharmaceutically acceptable salt, solvate and/or prodrug thereof when complexed to a radionuclide comprises a net charge of negative two (−2). In some embodiments, the divalent linker groups comprise a combined total at least 1 or 2 amino acid residues selected from D-Glu, L-Glu, D-γGlu and L-γGlu. In some embodiments, the divalent linkers comprise a combined total of 1 to 9 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, wherein at least 1 or 2 groups are selected from D-Glu, L-Glu, D-γGlu and L-γGlu. In some embodiments, the divalent linkers comprise a combined total of 2 to 9 groups independently selected from Cbp, Dap, Gly, D-His, L-His, D-Arg, L-Arg, D-Pro, L-Pro, D-Ser, L-Ser, D-Glu, L-Glu, D-γGlu, L-γGlu, OEG, PEG1, PEG3, PEG6, and Aoc, wherein at least 1 or 2 groups are selected from D-Glu, L-Glu, D-γGlu and L-γGlu. In some embodiments, the divalent linkers comprise a combined total of 1 to 9 groups independently selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, wherein at least 1 or 2 groups are selected from D-Glu, L-Glu, D-γGlu and L-γGlu. In some embodiments, the divalent linkers comprise a combined total of 2 to 9 groups independently selected from Gly, D-Pro, L-Pro, D-Ser, L-Ser, D-Glu, L-Glu, D-γGlu, L-γGlu, OEG, PEG1, PEG3, PEG6, and Aoc, wherein at least 1 or 2 groups are selected from D-Glu, L-Glu, D-γGlu and L-γGlu. In some embodiments, the divalent linkers comprise a combined total of 2 to 9 groups independently selected from Gly, L-Pro, L-Glu, L-γGlu, OEG, PEG1, PEG3, PEG6, and Aoc, wherein at least 2 or 3 groups are selected from L-Glu and L-γGlu. In some embodiments, the divalent linkers comprise a combined total of 1 or 2 groups independently selected from Gly, L-Glu, L-γGlu and OEG, wherein at least 1 or 2 groups are selected from L-Glu and L-γGlu. In some embodiments, E is a chelating group comprising three free carboxylic acid groups such as a chelating group derived from DOTA, the divalent linkers combined comprise 2 to 9 groups independently selected from Gly, L-Pro, L-Glu, L-γGlu, OEG, PEG1, PEG3, PEG6, and Aoc, wherein at least 2 groups are selected from L-Glu and L-γGlu and the compound of Formula I or a pharmaceutically acceptable salt, solvate and/or prodrug when complexed to ¹⁷⁷Lu comprises a net charge of negative two (−2). In some embodiments, E is a chelating group comprising four free carboxylic acid groups such as a chelating group derived from DOTAGA, the divalent linkers combined comprise 2 to 9 groups independently selected from Gly, L-Pro, L-Glu, L-γGlu, OEG, PEG1, PEG3, PEG6, and Aoc, wherein at least 1 group is selected from L-Glu and L-γGlu and the compound of Formula I or a pharmaceutically acceptable salt, solvate and/or prodrug when complexed to ¹⁷⁷Lu comprises a net charge of negative two (−2).

In some embodiments, the compound of Formula I or a pharmaceutically acceptable salt, solvate and/or prodrug thereof when complexed to a radionuclide comprises a net charge of negative one (−1). In some embodiments, the divalent linkers comprise a combined total of 0 or 1 amino acid residues selected from D-Glu, L-Glu, D-γGlu and L-γGlu. In some embodiments, divalent linkers comprise a combined total of 0 to 9 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, wherein 0 or 1 groups are selected from D-Glu, L-Glu, D-γGlu and L-γGlu. In some embodiments, the divalent linkers comprise a combined total of 2 to 9 groups independently selected from Cbp, Dap, Gly, D-His, L-His, D-Pro, L-Pro, D-Ser, L-Ser, D-Glu, L-Glu, D-Arg, L-Arg, D-γGlu, L-γGlu, OEG, PEG1, PEG3, PEG6, and Aoc, wherein 0 or 1 groups are selected from D-Glu, L-Glu, D-γGlu and L-γGlu. In some embodiments, divalent linkers comprise a combined total of 1 to 9 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, wherein 0 or 1 groups are selected from D-Glu, L-Glu, D-γGlu and L-γGlu. In some embodiments, the divalent linkers comprise a combined total of 2 to 9 groups independently selected from Cbp, Dap, Gly, D-His, L-His, D-Pro, L-Pro, D-Ser, L-Ser, D-Glu, L-Glu, D-Arg, L-Arg, D-γGlu, L-γGlu, OEG, PEG1, PEG3, PEG6, and Aoc, wherein 0 or 1 groups are selected from D-Glu, L-Glu, D-γGlu and L-γGlu. In some embodiments, divalent linkers comprise a combined total of 0 to 9 groups independently selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, wherein 0 or 1 groups are selected from D-Glu, L-Glu, D-γGlu and L-γGlu. In some embodiments, the divalent linkers comprise a combined total of 2 to 9 groups independently selected from Gly, D-Pro, L-Pro, D-Ser, L-Ser, D-Glu, L-Glu, D-γGlu, L-γGlu, OEG, PEG1, PEG3, PEG6, and Aoc, wherein 0 or 1 groups are selected from D-Glu, L-Glu, D-γGlu and L-γGlu. In some embodiments, the divalent linkers comprise a combined total of 2 to 9 groups independently selected from Gly, L-Pro, L-Glu, L-γGlu, OEG, PEG1, PEG3, PEG6, and Aoc, wherein 0 or 1 groups are selected from L-Glu and L-γGlu. In some embodiments, the divalent linkers comprise a combined total of 2 to 9 groups independently selected from Gly, L-Glu, L-γGlu, OEG and Aoc, wherein 0 or 1 groups are selected from L-Glu and L-γGlu. In some embodiments, E is a chelating group comprising three free carboxylic acid groups such as DOTA, the divalent linkers combined comprise 2 to 9 groups independently selected from Gly, L-Pro, L-Glu, L-γGlu, OEG, PEG1, PEG3, PEG6, and Aoc, wherein at least 1 group is selected from L-Glu and L-γGlu and the compound of Formula I or a pharmaceutically acceptable salt, solvate and/or prodrug when complexed to ¹⁷⁷Lu comprises a net charge of negative one (−1). In some embodiments, E is a chelating group comprising four free carboxylic acid groups such as DOTAGA, the divalent linkers combined comprise 2 to 9 groups independently selected from Gly, L-Pro, L-Glu, L-γGlu, OEG, PEG1, PEG3, PEG6, and Aoc, wherein 0 groups are selected from L-Glu and L-γGlu and the compound of Formula I or a pharmaceutically acceptable salt, solvate and/or prodrug when complexed to ¹⁷⁷Lu comprises a net charge of negative one (−1). In some embodiments, E is a chelating group comprising four free carboxylic acid groups such as DOTAGA, the divalent linkers combined comprise 2 to 9 groups independently selected from Gly, L-Pro, OEG, PEG1, PEG3, PEG6, and Aoc, wherein 0 groups are selected from L-Glu and L-γGlu and the compound of Formula I or a pharmaceutically acceptable salt, solvate and/or prodrug when complexed to ¹⁷⁷Lu comprises a net charge of negative one (−1).

In some embodiments, the compound of Formula I or a pharmaceutically acceptable salt, solvate and/or prodrug thereof when complexed to a radionuclide comprises a net charge of zero (0). In some embodiments, the divalent linkers comprise a combined total of 0 or 1 amino acid residues selected from D-Lys, L-Lys, D-εLys, L-εLys. In some embodiments, the divalent linkers comprise a combined total of 0 to 9 groups independently selected from Cbp, Dap, Gly, D-His, L-His, D-Pro, L-Pro, D-Arg, L-Arg D-Ser, L-Ser, D-Lys, L-Lys, D-εLys, L-εLys, D-Glu, L-Glu, D-γGlu, L-γGlu, OEG, PEG1, PEG3, PEG6, Aoc Aun and Ahx, wherein 0 or 1 groups are selected from D-Lys, L-Lys, D-εLys, L-εLys. In some embodiments, the divalent linkers comprise a combined total of 1 to 9 groups independently selected from Cbp, Dap, Gly, D-His, L-His, D-Pro, L-Pro, D-Arg, L-Arg D-Ser, L-Ser, D-Lys, L-Lys, D-εLys, L-εLys, D-Glu, L-Glu, D-γGlu, L-γGlu, OEG, PEG1, PEG3, PEG6, Aoc Aun and Ahx, wherein 0 or 1 groups are selected from D-Lys, L-Lys, D-εLys and L-εLys. In some embodiments, the divalent linkers comprise a combined total of 1 to 9 groups independently selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc Aun and Ahx, wherein 0 or 1 groups are selected from D-Lys, L-Lys, D-εLys, L-εLys. In some embodiments, the divalent linkers comprise a combined total of 0 to 9 groups independently selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc Aun and Ahx, wherein 0 or 1 groups are selected from D-Lys, L-Lys, D-εLys, L-εLys. In some embodiments, the divalent linkers comprise a combined total of 1 to 9 groups independently selected from Gly, D-Pro, L-Pro, D-Ser, L-Ser, D-Lys, L-Lys, D-εLys, L-εLys, OEG, PEG1, PEG3, PEG6 and Aoc, wherein 0 or 1 groups are selected from L-Lys and L-εLys. In some embodiments, the divalent linkers comprise a combined total of 0 to 9 groups independently selected from Gly, D-Pro, L-Pro, D-Ser, L-Ser, D-Lys, L-Lys, D-εLys, L-εLys, OEG, PEG1, PEG3, PEG6 and Aoc, wherein 0 or 1 groups are selected from L-Lys and L-εLys. In some embodiments, the divalent linkers comprise a combined total of 1 to 9 groups

In some embodiments, E is a chelating group comprising four free carboxylic acid groups such as a chelating group derived from DOTAGA, the divalent linkers combined comprise 1 to 9 groups independently selected from Gly, D-Pro, L-Pro, D-Ser, L-Ser, D-Lys, L-Lys, D-εLys, L-εLys, OEG, PEG1, PEG3, PEG6 and Aoc, wherein 1 group is selected from L-Lys and L-εLys and the compound of Formula I or a pharmaceutically acceptable salt, solvate and/or prodrug thereof when complexed to ¹⁷⁷Lu comprises a net charge of zero (0).

In some embodiments, E is a chelating group comprising three free carboxylic acid groups such as a chelating group derived from DOTA, the divalent linkers combined comprise 0 to 9, or 1 to 9 groups independently selected from Cbp, Dap, Gly, D-His, L-His, D-Pro, L-Pro, D-Ser, L-Ser, D-Arg, L-Arg, D-Lys, L-Lys, D-εLys, L-εLys, OEG, PEG1, PEG3, PEG6 and Aoc, wherein 0 groups are selected from L-Lys and L-εLys and the compound of Formula I or a pharmaceutically acceptable salt, solvate and/or prodrug thereof when complexed to ¹⁷⁷Lu comprises a net charge of zero (0). Therefore, in some embodiments, E is a chelating group comprising three free carboxylic acid groups such as a chelating group derived from DOTA, the divalent linkers combined comprise 0 to 9 groups or 1 to 9 groups independently selected from Gly, D-Pro, L-Pro, D-Ser, L-Ser, OEG, PEG1, PEG3, PEG6, and Aoc, and the compound of Formula I or a pharmaceutically acceptable salt, solvate and/or prodrug thereof when complexed to ¹⁷⁷Lu comprises a net charge of zero (0). In some embodiments, the divalent linkers comprise a combined total of 0 to 9 groups independently selected from Cbp, Dap, D-His, L-His, D-Pro, L-Pro, D-Ser, L-Ser, D-Arg, L-Arg, OEG, PEG1, PEG3, PEG6, Aoc, Aun and Ahx. In some embodiments, E is a chelating group comprising three free carboxylic acid groups such as a chelating group derived from DOTA, the divalent linkers combined comprise 0 to 9 groups independently selected from Gly, D-Pro, L-Pro, D-Ser, L-Ser, D-Lys, L-Lys, D-εLys, L-εLys, OEG, PEG1, PEG3, PEG6 and Aoc, wherein 0 groups are selected from L-Lys and L-εLys and the compound of Formula I or a pharmaceutically acceptable salt, solvate and/or prodrug thereof when complexed to ¹⁷⁷Lu comprises a net charge of zero (0). Therefore, in some embodiments, E is a chelating group comprising three free carboxylic acid groups such as a chelating group derived from DOTA, the divalent linkers combined comprise 0 to 9 groups independently selected from Gly, D-Pro, L-Pro, D-Ser, L-Ser, OEG, PEG1, PEG3, PEG6, and Aoc, and the compound of Formula I or a pharmaceutically acceptable salt, solvate and/or prodrug thereof when complexed to ¹⁷⁷Lu comprises a net charge of zero (0). In some embodiments, the divalent linkers comprise a combined total of 0 to 9 groups independently selected from Gly, D-Pro, L-Pro, D-Ser, L-Ser, OEG, PEG1, PEG3, PEG6, Aoc, Aun and Ahx.

In some embodiments, the divalent linkers comprise a combined total of 1 to 7 groups independently selected from Gly, D-γGlu, L-γGlu, OEG and Gly.

In some embodiments, the divalent linkers comprise a combined total of 1 to 7 groups independently selected from Gly, D-γGlu, L-γGlu, D-Lys, L-Lys, D-εLys and L-εLys and OEG.

In some embodiments, the divalent linkers comprise a combined total of 1 to 7 groups independently selected from Cbp, Dap, Gly, D-γGlu, L-γGlu, D-Ser, L-Ser, D-His, L-His and OEG.

In some embodiments, the divalent linkers comprise a combined total of 2 to 6 groups independently selected from Gly and L-Pro. In some embodiments, the divalent linkers comprise a combined total of 2 to 5 groups independently selected from Gly and L-Pro.

In some embodiments, the divalent linkers comprise a combined total of 1 to 6 groups independently selected from Gly and L-Ser. In some embodiments, the divalent linkers comprise a combined total of 1 to 4 groups independently selected from Gly and L-Ser.

In some embodiments, the divalent linkers comprise a combined total of 1 to 6 groups and the 1 to 6 groups are Gly. In some embodiments, the divalent linkers comprise a combined total of 1 to 4 groups and the 1 to 4 groups are Gly.

In some embodiments, the divalent linkers comprise a combined total of 1 to 4 groups independently selected from Gly and L-Ser.

In some embodiments, the divalent linkers comprise a combined total of 1 to 2 wherein the 1 to 2 groups are selected from PEG1, PEG3 or PEG6.

In some embodiments, the divalent linkers comprise a combined total of 1 to 2 and the 1 to 2 groups are Aoc.

In some embodiments, the divalent linkers comprise a combined total of 1 to 2 and the 1 to 2 groups are Ahx.

In some embodiments, the divalent linkers comprise a combined total of 1 to 4 groups and the 1 to 4 groups are OEG. In some embodiments, the divalent linkers comprise a combined total of 1 to 3 groups and the 1 to 3 groups are OEG. In some embodiments, the divalent linkers comprise a combined total of 1 to 2 groups and the 1 to 2 groups are OEG. In some embodiments, the divalent linkers comprise a combined total of 1 group and the 1 group is OEG.

In some embodiments, one of L¹, L² and L³ is a direct bond. In some embodiments, L¹ is a direct bond. In some embodiments, two of L¹, L² and L³ are direct bonds. In some embodiments, L¹ and L² are both direct bonds. In some embodiments, L¹, L² and L³are all direct bonds.

In some embodiments, one or two of L¹, L² and L³ are divalent linkers, and the divalent linkers comprise a combined total of 1 to 15 groups selected from Cbp, Dap, Gly, D-His, L-His, D-Pro, L-Pro, D-Arg, L-Arg D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, and the other two or one of L¹, L² and L³ respectively are direct bonds. In some embodiments, one or two of L¹, L² and L³ are divalent linkers, and the divalent linkers comprise a combined total of 1 to 11 groups selected from Cbp, Dap, D-His, L-His, Gly, D-Pro, L-Pro, D-Arg, L-Arg, D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, and the other two or one of L¹, L² and L³ respectively are direct bonds. In some embodiments, one or two of L¹, L² and L³are divalent linkers, and the divalent linkers comprise a combined total of 1 to 6 groups selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, and the other two or one of L¹, L² and L³ respectively are direct bonds. In some embodiments, one or two of L¹, L² and L³ are divalent linkers, and the divalent linkers comprise a combined total of 1 to 15 groups selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, and the other two or one of L¹, L² and L³ respectively are direct bonds. In some embodiments, one or two of L¹, L² and L³ are divalent linkers, and the divalent linkers comprise a combined total of 1 to 11 groups selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, and the other two or one of L¹, L² and L³ respectively are direct bonds. In some embodiments, one or two of L¹, L² and L³ are divalent linkers, and the divalent linkers comprise a combined total of 1 to 6 groups selected from Gly, D-Pro, L-Pro, D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, and the other two or one of L¹, L² and L³ respectively are direct bonds.

In some embodiments, one of L¹, L² and L³ is a divalent linker, and the divalent linker comprises 1 to 3 OEG groups, and the other two of L¹, L² and L³ are direct bonds. In some embodiments, one of L¹, L² and L³ is a divalent linker, and the divalent linker comprises 1 to 2 OEG groups, and the other two of L¹, L² and L³ are direct bonds. In some embodiments, one of L¹, L² and L³ is a divalent linker, and the divalent linker comprises 1 OEG group, and the other two of L¹, L² and L³ are direct bonds. In some embodiments, L³ is a divalent linker, and the divalent linker comprises 1 to 3 OEG groups, and L¹ and L² are direct bonds. In some embodiments, L³ is a divalent linker, and the divalent linker comprises 1 to 2 OEG groups, and L¹ and L² are direct bonds. In some embodiments, L³ is a divalent linker, and the divalent linker comprises 1 OEG group, and L¹ and L² are both direct bonds.

In some embodiments, E is a chelating group derived from DOTA, one of L¹, L² and L³ is a divalent linker, and the divalent linker comprises 1 OEG group, and the other two of L¹, L² and L³ are direct bonds and the compound of Formula I or a pharmaceutically acceptable salt, solvate and/or prodrug thereof when complexed to ¹⁷⁷Lu comprises a net charge of zero (0).

In some embodiments, each R⁵ is independently selected from H and C₁₋₄ alkyl. In some embodiments, each R⁵ is independently selected from H, CH₃, CH₂CH₃, CH₂CH₂CH₃, CH(CH₂)₂ and C(CH₂)₃. In some embodiments, each R⁵ is independently selected from H and CH₃.

In some embodiments, each R⁶ is independently selected from H and C. ₂alkyl. In some embodiments, each R⁶ is independently selected from H, CH₃ and CH₂CH₃. In some embodiments, each R⁶ is independently selected from H and CH₃.

In some embodiments, the compound of Formula I is selected from the following list of compounds, or a pharmaceutically acceptable salt and/or solvate thereof:

| | Compound | |
|---|---|---|
| I.D | Name | Structure |
| I-1 | DOTA- K(-gE- G- Z360)- OEG- f[CYwKTC]T- OH | 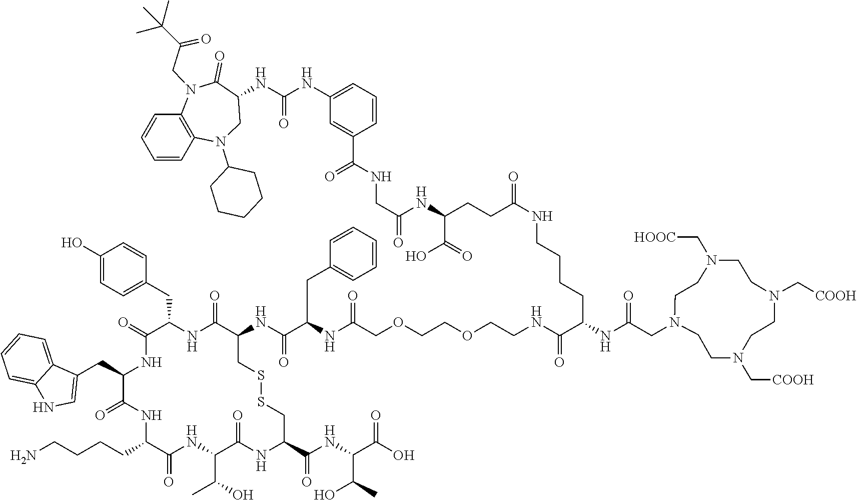 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-2 | DOTA- K(-gE- G- Z360)- OEG- OEG- f[CYwKTC]T- OH | 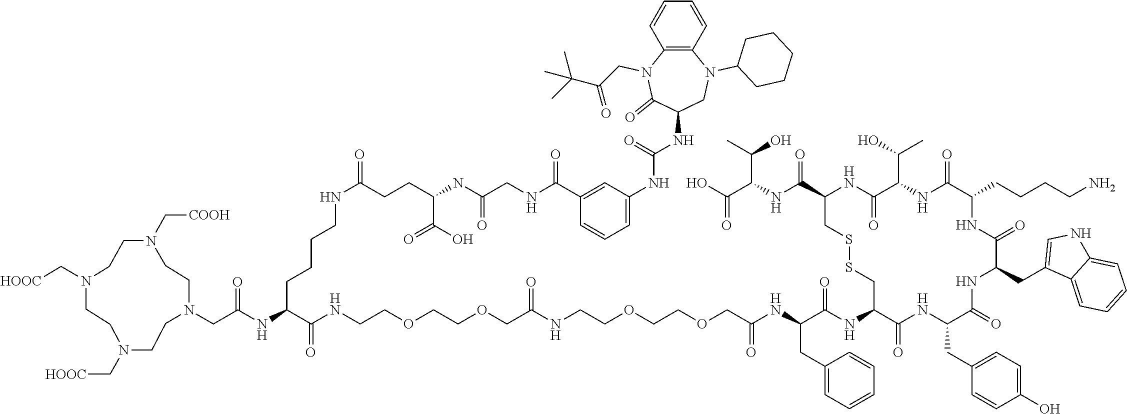 |
| I-3 | DOTA- K(-E- Z360)- OEG- f[CYwKTC]T- NH₂ | 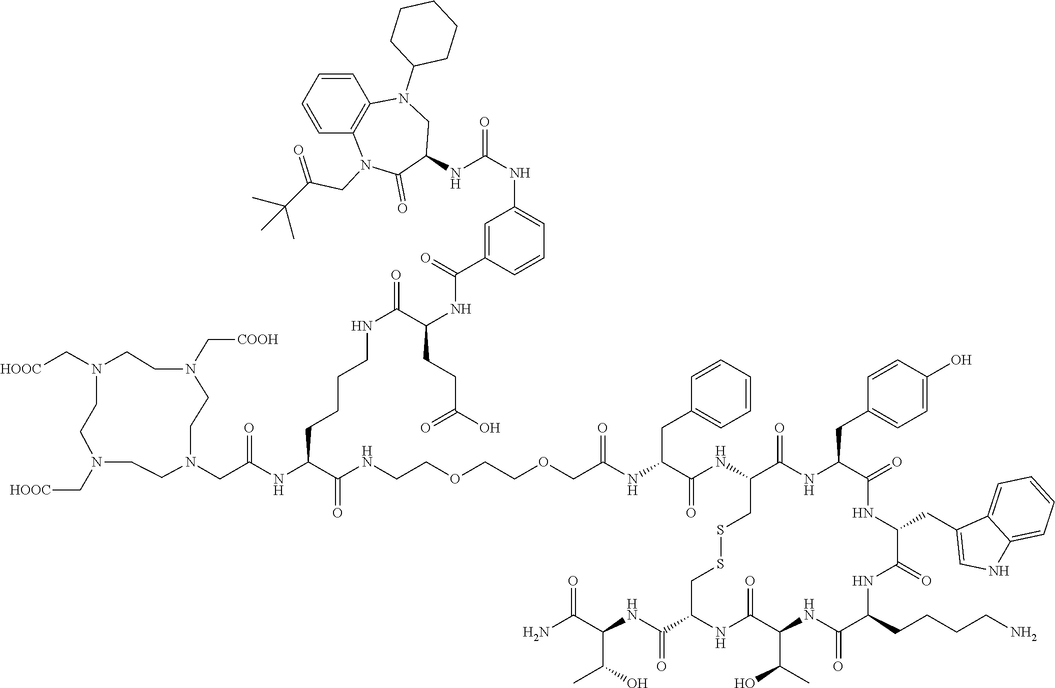 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(N)=O] |
| I-4 | DOTA- K(-E- Z360)- f[CYwKTC]T- NH₂ | 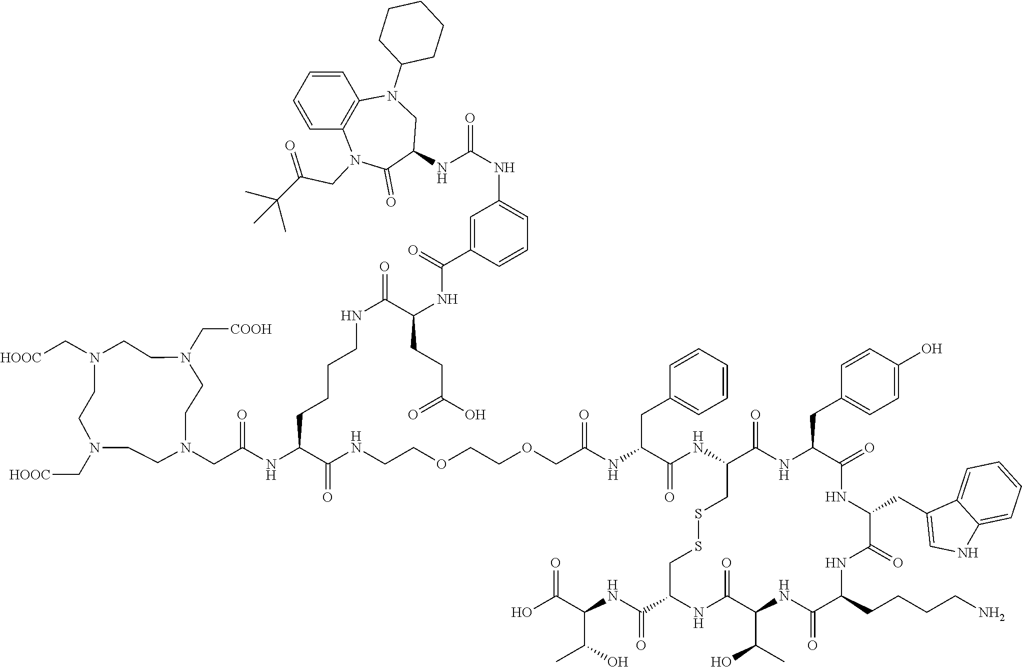 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-5 | DOTA- K(-E- Z360)- OEG- f[CYwKTC]- Thr(ol) | 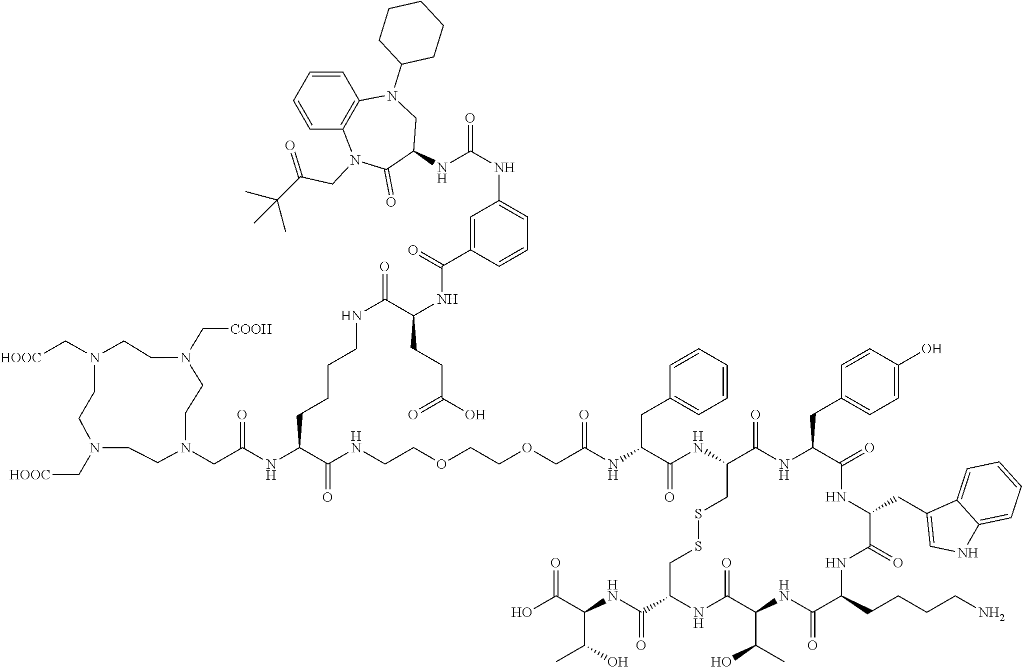 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-6 | DOTA- K(-E- Z360)- f[CYwKTC]- Thr(ol) | 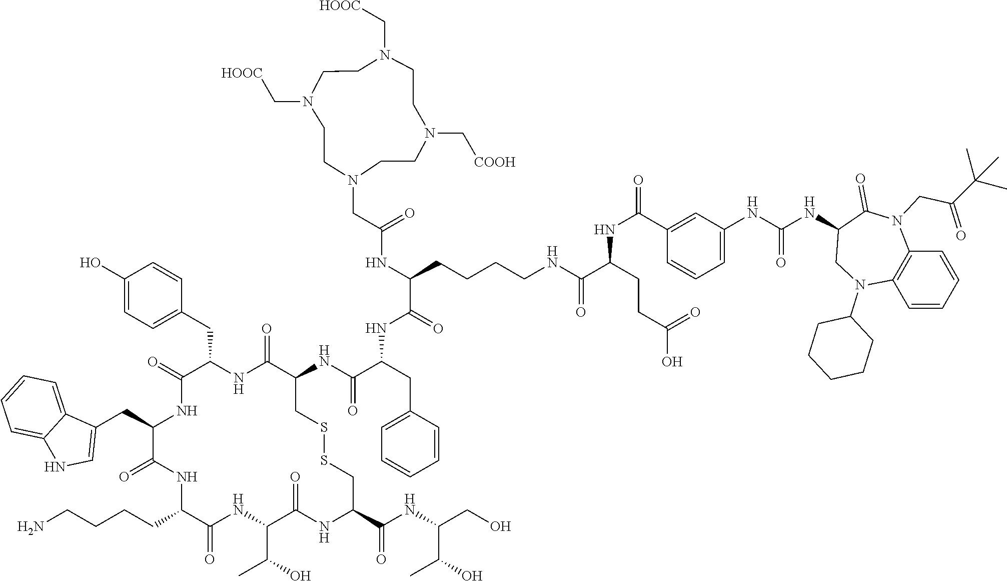 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)CSSC[C@@H](C(=O)N[C@H](CO)[C@@H](C)O)NC1=O] |
| I- 7 | DOTA- gE-gE- gE-gE- OEg- K(-gE- G- Z360)- OEG- f[CYwKTC]T- OH) | 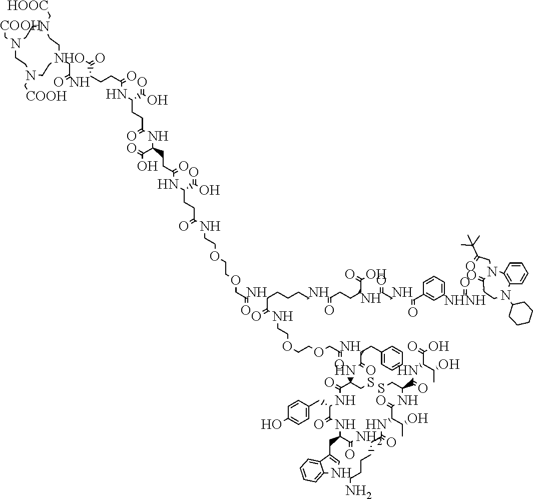 |
| I-8 | DOTA- gE-gE- gE- OEG- K(-gE- G- Z360)- OEG- f[CYwKTC]T- OH | 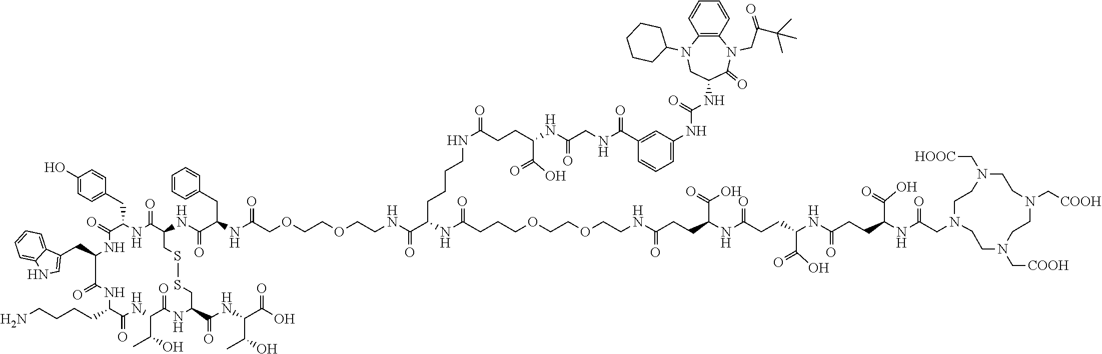 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CCCOCCOCCNC(=O)CC[C@H](NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-9 | DOTA- gE-K(-gE- G- Z360)- gE- f[CYwKTC]T- OH | 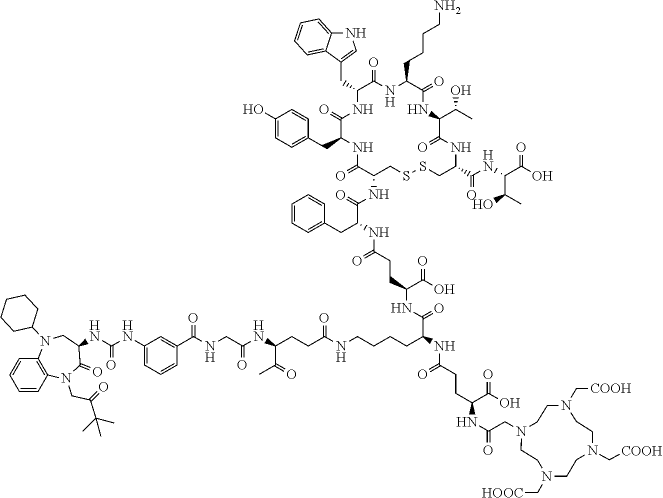 [SMILES: CC(=O)[C@H](CCC(=O)NCCCC[C@H](NC(=O)CC[C@H](NC(=O)CN1CCN(CC(=O)O)CCN(CC(=O)O)CCN(CC(=O)O)CC1)C(=O)O)C(=O)N[C@@H](CCC(=O)N[C@H](Cc1ccccc1)C(=O)N[C@H]1CSSC[C@@H](C(=O)N[C@H](C(=O)O)[C@@H](C)O)NC(=O)[C@H]([C@@H](C)O)NC(=O)[C@H](CCCCN)NC(=O)[C@@H](Cc2c[nH]c3ccccc23)NC(=O)[C@H](Cc2ccc(O)cc2)NC1=O)C(=O)O)NC(=O)CNC(=O)c1cccc(NC(=O)N[C@@H]2CN(C3CCCCC3)c3ccccc3N(CC(=O)C(C)(C)C)C2=O)c1] |
| I- 10 | DOTA- gE-gE- K(-gE- G- Z360)- OEG- f[CYwKTC]T- OH | 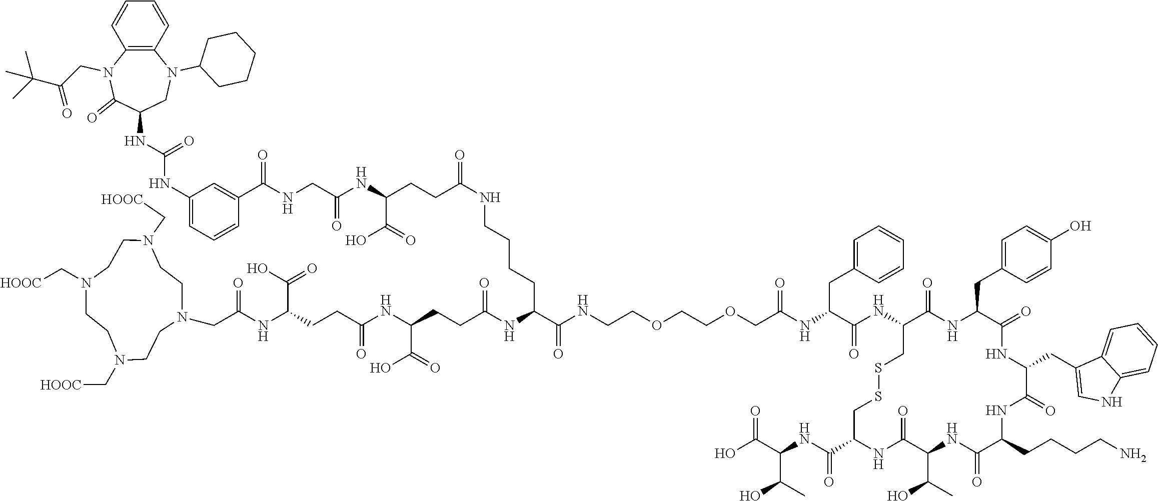 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 11 | DOTA- gE-gE- OEG- K(-E- Z360)- OEG- f[CYwKTC]T- OH | 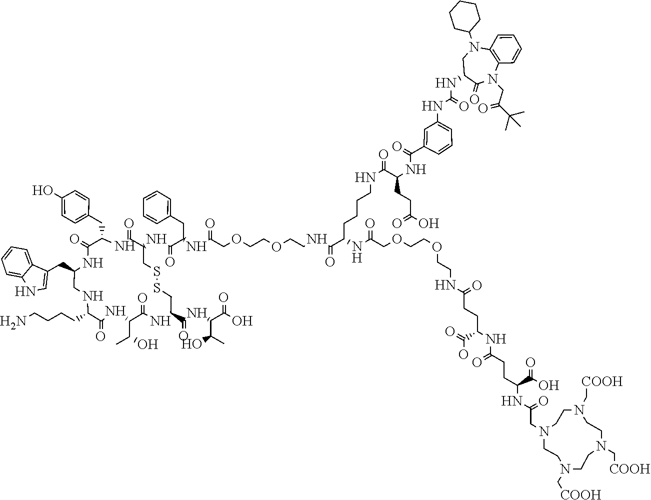 |
| I- 12 | (Z360)- E-gE- OEG- gE- K(DOTA)- OEG- f[CYwKTC]T- OH | 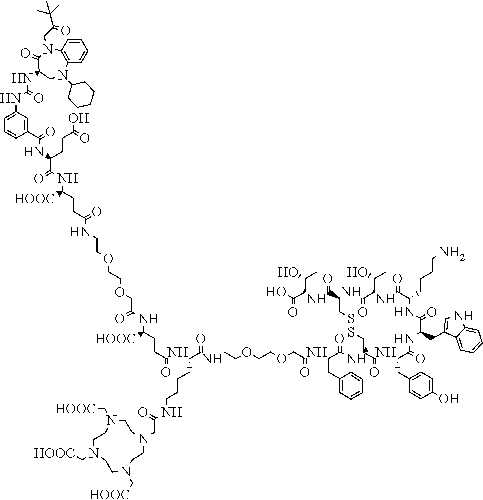 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CN2CCN(CC(=O)O)CCN(CC(=O)O)CCN(CC(=O)O)CC2)NC(=O)CC[C@H](NC(=O)COCCOCCNC(=O)CC[C@H](NC(=O)[C@H](CCC(=O)O)NC(=O)c2cccc(NC(=O)N[C@@H]3CN(C4CCCCC4)c4ccccc4N(CC(=O)C(C)(C)C)C3=O)c2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 13 | (Z360)- gE- gE- OEG- K(DOTAGA)- OEG- f[CYwKTC]T- OH | 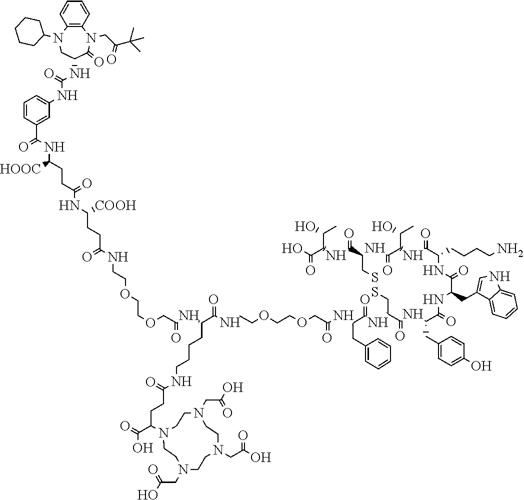 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CCC(C(=O)O)N2CCN(CC(=O)O)CCN(CC(=O)O)CCN(CC(=O)O)CC2)NC(=O)COCCOCCNC(=O)CC[C@H](NC(=O)CC[C@H](NC(=O)c2cccc(NC(=O)N[C@@H]3CN(C4CCCCC4)c4ccccc4N(CC(=O)C(C)(C)C)C3=O)c2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 14 | DOTA- gE-gE- OEG- K(-gE- G- Z360)- OEG- f[CYwKTC]T- OH | 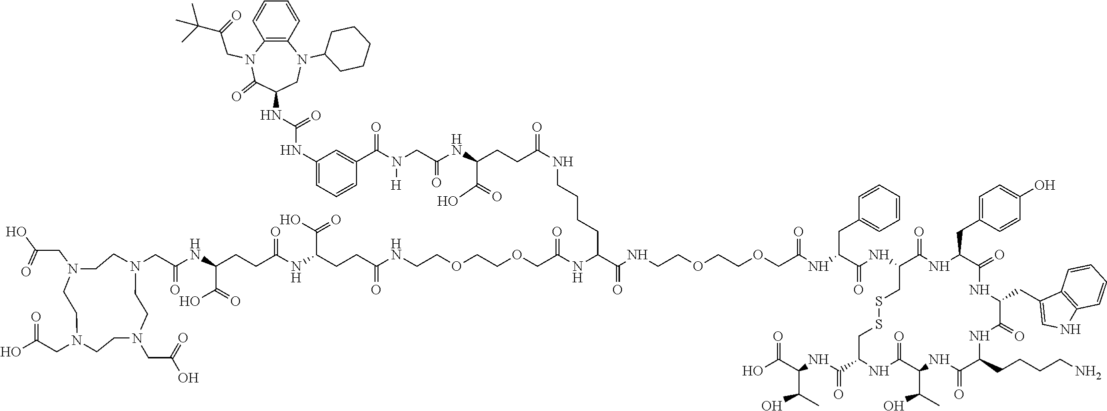 [SMILES: C[C@@H](O)[C@H](NC(=O)[C@@H]1CSSC[C@H](NC(=O)[C@@H](Cc2ccccc2)NC(=O)COCCOCCNC(=O)C(CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 15 | DOTA- gE- gE- OEG- OEG- K(-gE- G- Z360)- OEG- f[CYwKTC]T- OH | 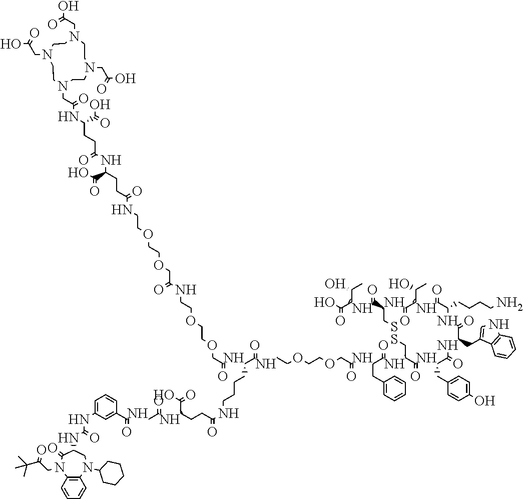 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 16 | DOTA- gE-gE- OEG- OEg- K(-gE- G- Z360)- OEG- OEG- f[CYwKTC]T- OH | 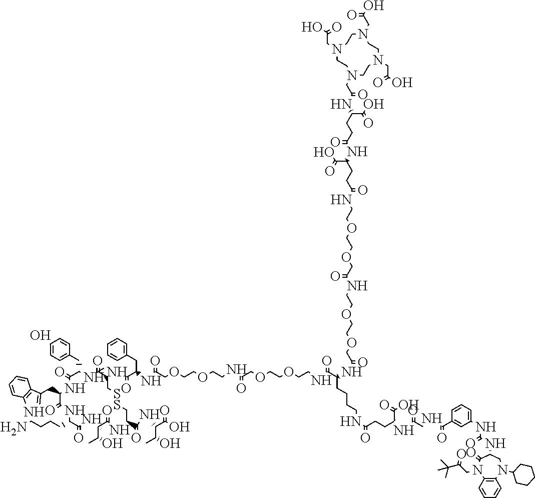 [SMILES: C[C@@H](O)[C@H](NC(=O)[C@@H]1CSSC[C@H](NC(=O)[C@@H](Cc2ccccc2)NC(=O)COCCOCCNC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 17 | (Z360)- gE- gE- OEG- OEG- K(DOTAGA)- OEG- OEG- f[CYwKTC]T- OH ( | 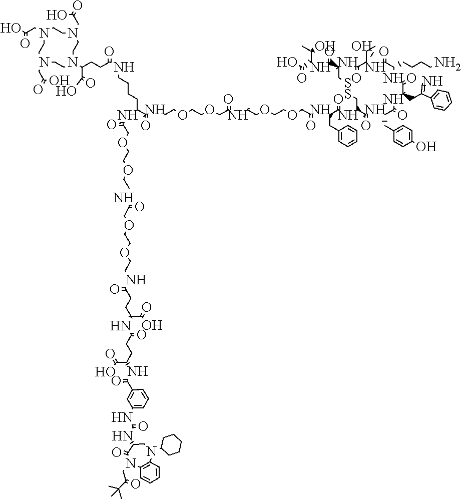 [SMILES: C[C@@H](O)[C@H](NC(=O)[C@@H]1CSSC[C@H](NC(=O)[C@@H](Cc2ccccc2)NC(=O)COCCOCCNC(=O)COCCOCCNC(=O)[C@H](CCCCNC(=O)CCC(C(=O)O)N2CCN(CC(=O)O)CCN(CC(=O)O)CCN(CC(=O)O)CC2)NC(=O)COCCOCCNC(=O)COCCOCCNC(=O)CC[C@H](NC(=O)CC[C@H](NC(=O)c2cccc(NC(=O)N[C@@H]3CN(C4CCCCC4)c4ccccc4N(CC(=O)C(C)(C)C)C3=O)c2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 18 | DOTA- gE- OEG- OEG- OEG- OEG- K(-gE- G- Z360)- OEG- gE- OEG- OEG- f[CYwKTC]T- OH | 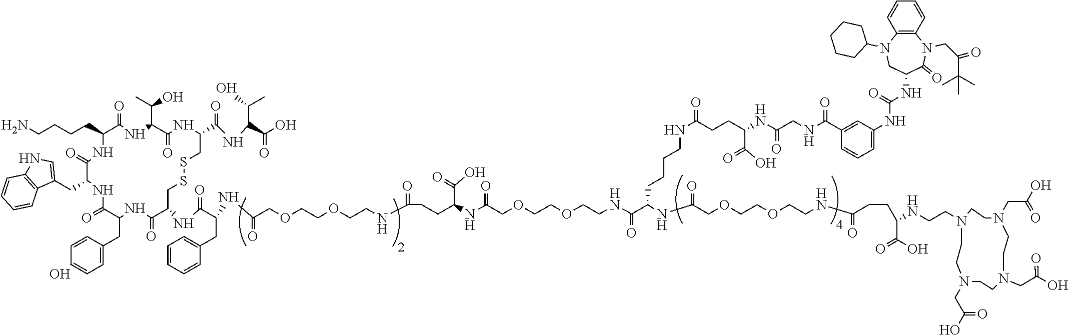 |
| I- 19 | DOTAGA- OEG- K(-gE- G- Z360)- f[CYwKTC]T- OH | 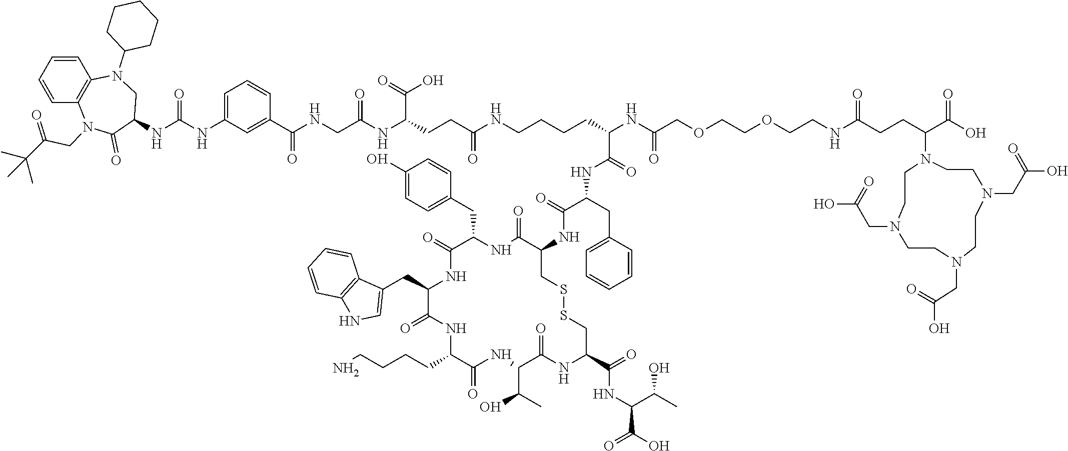 [SMILES: C[C@@H](O)[C@H](NC(=O)[C@@H]1CSSC[C@H](NC(=O)[C@@H](Cc2ccccc2)NC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CCC(C(=O)O)N2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 20 | DOTAGA- OEG- K(-gE- G- Z360)- OEG- f[CYwKTC]T- OH | 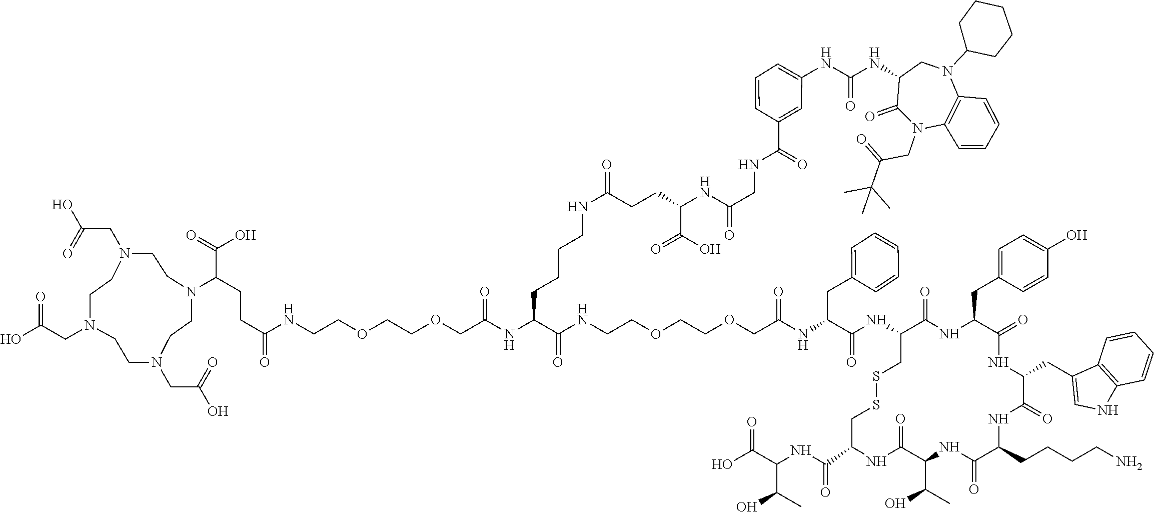 [SMILES: C[C@@H](O)C(NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CCC(C(=O)O)N2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 21 | DOTA- gE- OEG- OEG- K(-gE- G- Z360)- OEG- f[CYwKTC]T- OH | 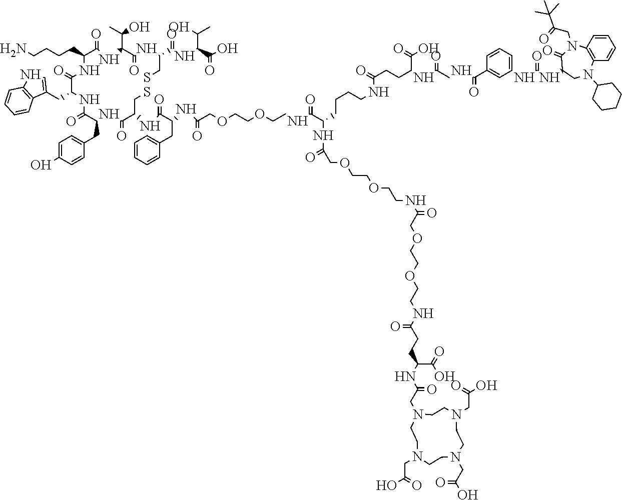 [SMILES: CC(O)[C@H](NC(=O)[C@@H]1CSSC[C@H](NC(=O)[C@@H](Cc2ccccc2)NC(=O)COCCOCCNC(=O)[C@H](CCCCNC(=O)CCC(NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)CC[C@H](NC(=O)CN2CCN(CC(=O)O)CCN(CC(=O)O)CCN(CC(=O)O)CC2)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 22 | DOTA- gE- OEG- OEG- OEG- OEG- OEG- K(-gE- G- Z360)- OEG- f[CYwKTC]T- OH) | 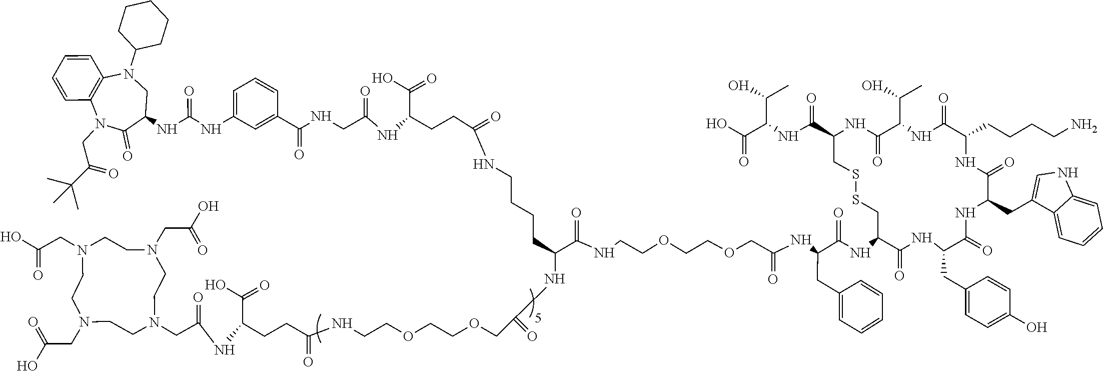 |
| I- 23 | DOTA- OEG- OEG- OEG- K(-gE- G- Z360)- OEG- gE- OEG- OEG- f[CYwKTC]T- OH | 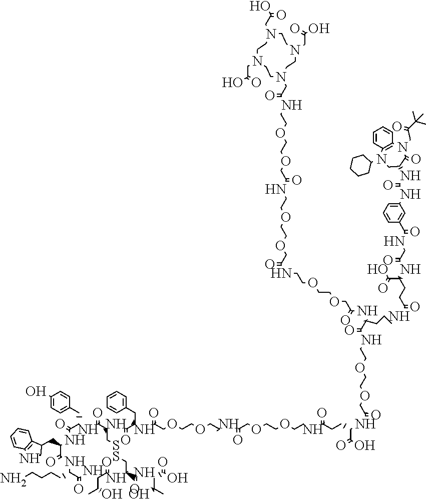 [SMILES: C[C@@H](O)C1NC(=O)[C@H](CCCCN)NC(=O)[C@@H](CC2C=Nc3ccccc32)NC(=O)[C@H](Cc2ccc(O)cc2)NC(=O)[C@@H](NC(=O)[C@@H](Cc2ccccc2)NC(=O)COCCOCCNC(=O)COCCOCCNC(=O)CC[C@H](NC(=O)COCCOCCNC(=O)[C@H](CCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)COCCOCCNC(=O)CN2CCN(CC(=O)O)CCN(CC(=O)O)CCN(CC(=O)O)CC2)C(=O)O)CSSC[C@@H](C(=O)N[C@H](C(=O)O)[C@@H](C)O)NC1=O] |
| I- 24 | DOTA- gE- OEG- OEG- OEG- OEG- K(-gE- G- Z360)- OEG- OEG- OEG- OEG- f[CYwKTC]T- OH | 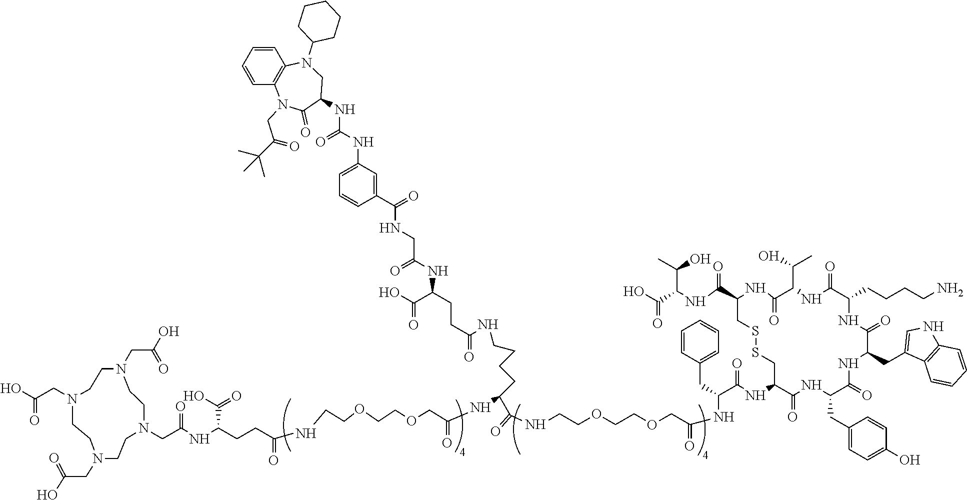 |
| I- 25 | DOTA- K(-E- Z360)- f[CYwKTC]T- OH | 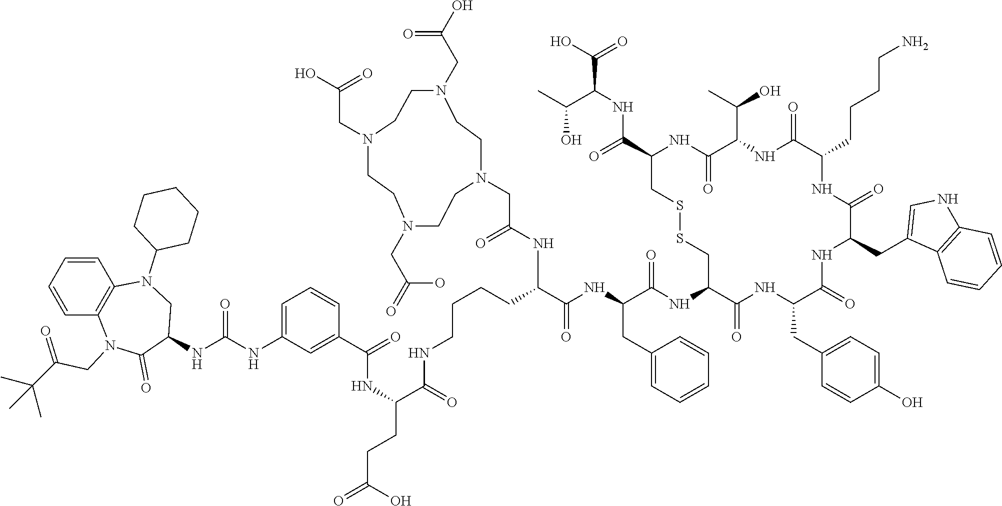 |
| I- 26 | DOTA- K(-gE- G- Z360)- f[CYwKTC]T- OH | 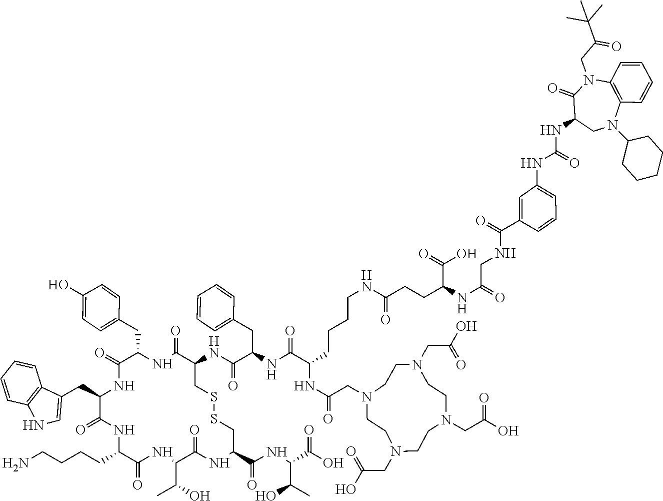 [SMILES: C[C@@H](O)[C@H](NC(=O)[C@@H]1CSSC[C@H](NC(=O)[C@@H](Cc2ccccc2)NC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 27 | DOTA- K(-E- Z360)- OEG- f[CYwKTC]T- OH | 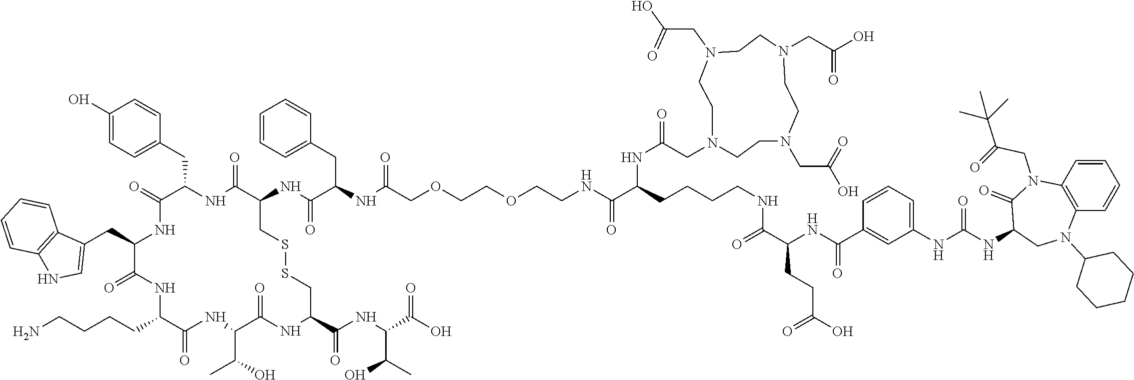 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 28 | DOTA- K(-OEG- E- Z360)- f[CYwKTC]T- OH | 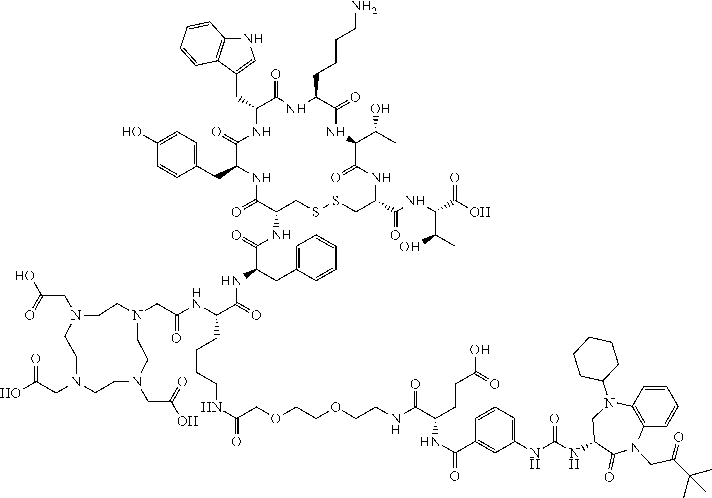 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 29 | DOTA- K(-OEG- OEG- E- Z360)- f[CYwKTC]T- OH | 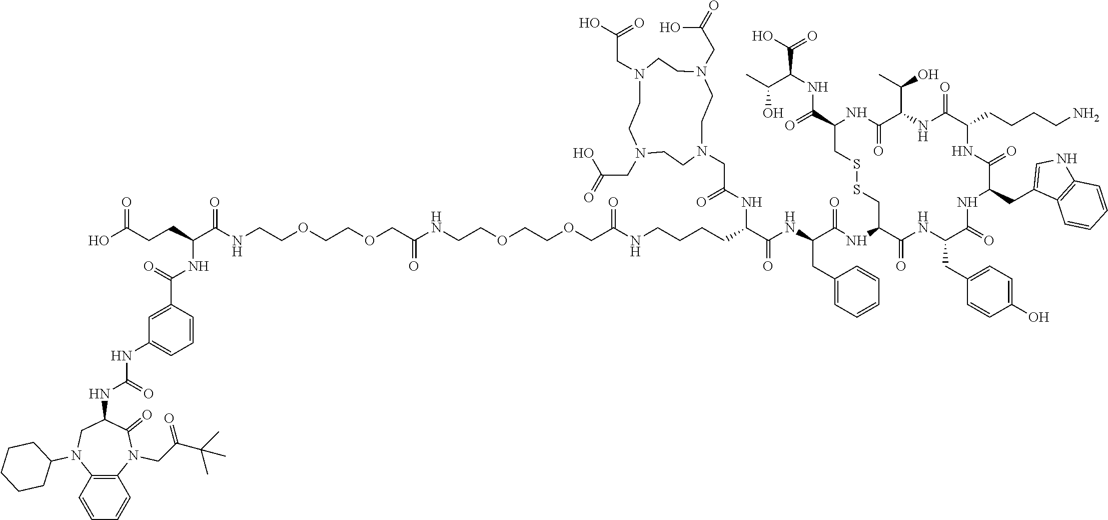 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)COCCOCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 30 | DOTA- K(-OEG- E- Z360)- OEG- f[CYwKTC]T- OH | 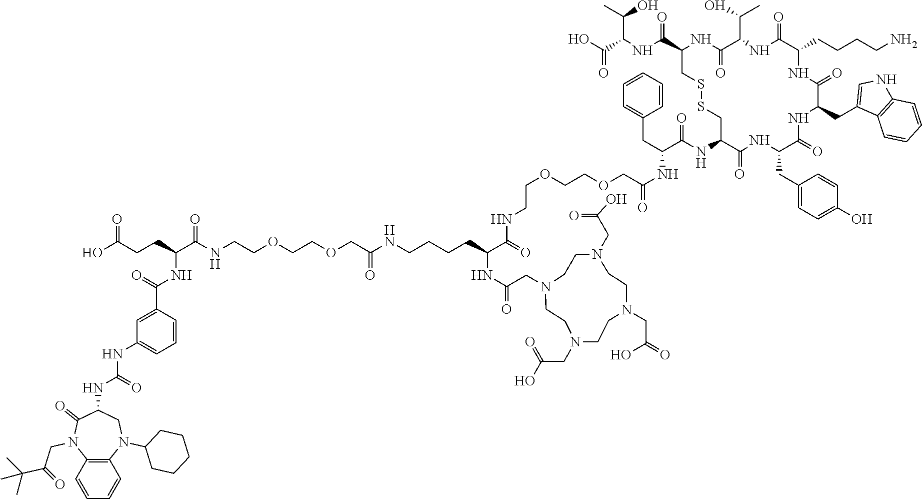 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)COCCOCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 31 | Z360- OEG- K(DOTAGA)- OEG- f[CYwKTC]T- OH | 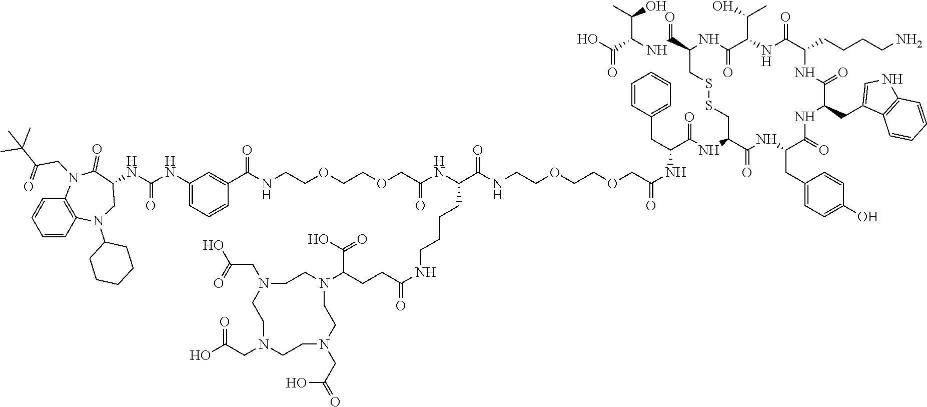 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CCC(C(=O)O)N2CCN(CC(=O)O)CCN(CC(=O)O)CCN(CC(=O)O)CC2)NC(=O)COCCOCCNC(=O)c2cccc(NC(=O)N[C@@H]3CN(C4CCCCC4)c4ccccc4N(CC(=O)C(C)(C)C)C3=O)c2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 32 | Z360- G-gE- OEG- OEG- OEG- K(DOTA)- OEG- f[CYwKTC]T- OH | 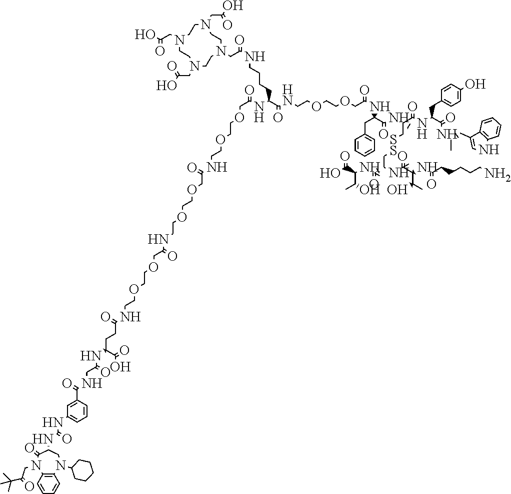 |
| I- 33 | DOTA- OEG- OEG- OEG- OEG- OEG- OEG- K(-gE- G- Z360)- OEG- f[CYwKTC]T- OH | 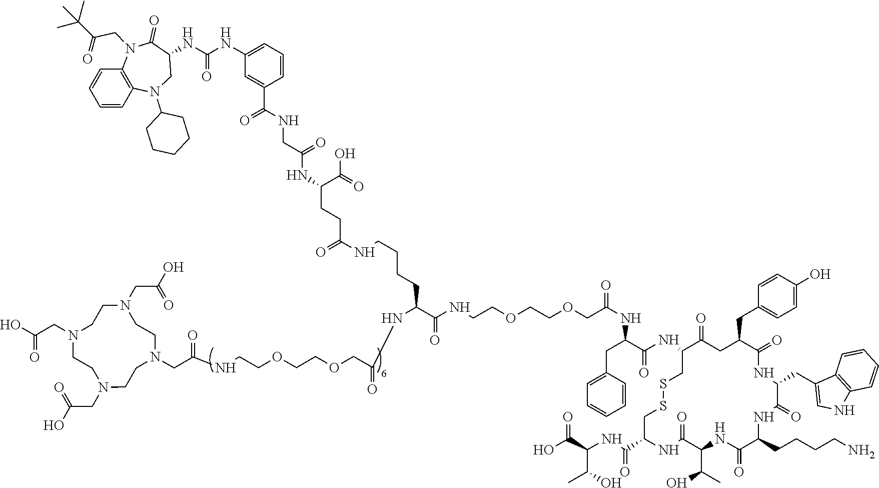 |
| I- 34 | DOTA- K(Z360)- f[CYwKTC]T- OH | 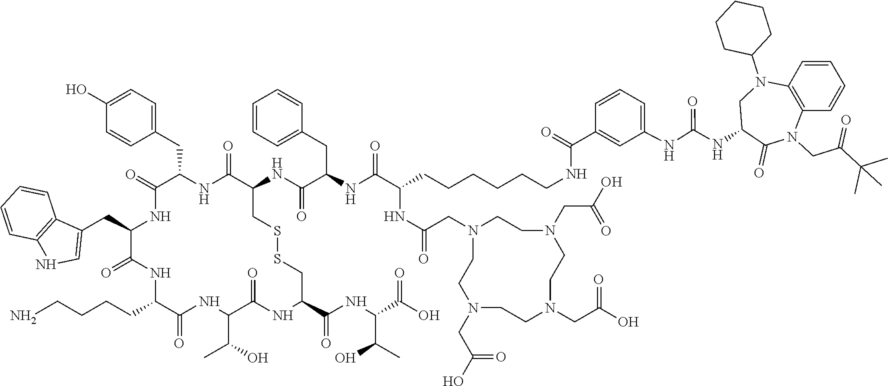 [SMILES: C[C@@H](O)C1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)[C@H](CCCCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC1=O] |
| I- 35 | DOTA- OEG- K(Z360)- f[CYwKTC]T- OH | 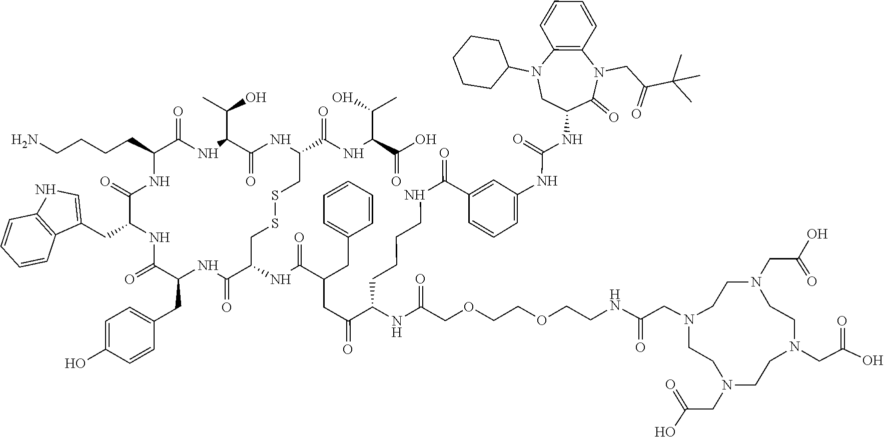 [SMILES: C[C@@H](O)[C@H](NC(=O)[C@@H]1CSSC[C@H](NC(=O)C(CC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)COCCOCCNC(=O)CN2CCN(CC(=O)O)CCN(CC(=O)O)CCN(CC(=O)O)CC2)Cc2ccccc2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 36 | DOTA- K(-OEG- Z360)- f[CYwKTC]T- OH | 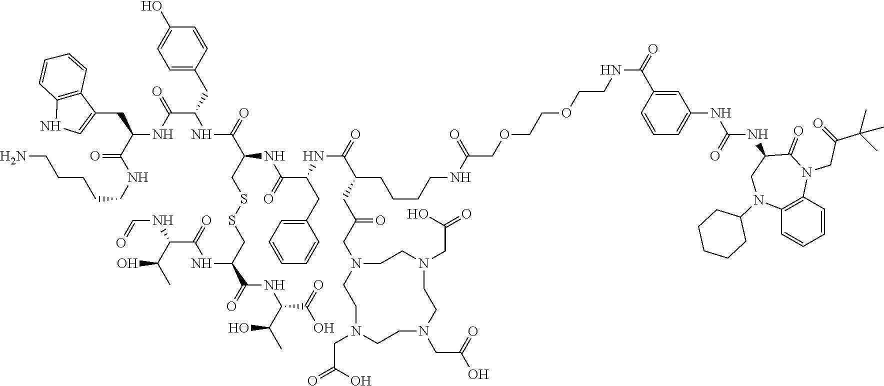 |
| I- 37 | DOTA- K(Z360)- OEG- f[CYwKTC]T- OH | 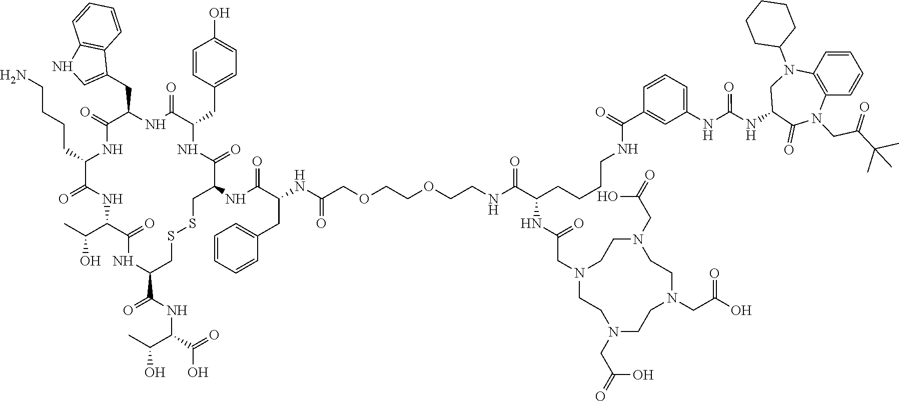 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 38 | DOTA- K(Z360)- PEG3- f[CYwKTC]T- OH | 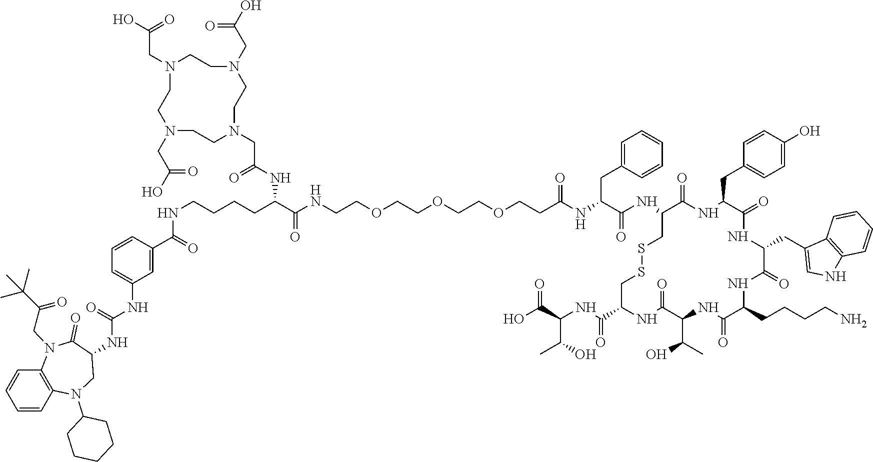 [SMILES: C[C@@H](O)[C@H](NC(=O)[C@@H]1CSSC[C@H](NC(=O)[C@@H](Cc2ccccc2)NC(=O)CCOCCOCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 39 | DOTA- K(Z360)- OEG- OEG- f[CYwKTC]T- OH | 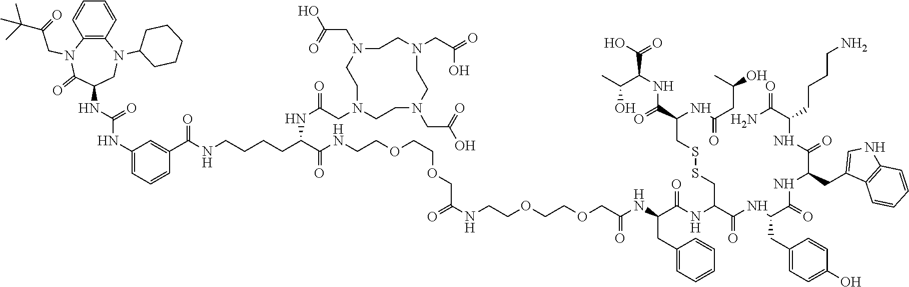 [SMILES: C[C@@H](O)CC(=O)N[C@@H](CSSCC(NC(=O)[C@@H](Cc1ccccc1)NC(=O)COCCOCCNC(=O)COCCOCCNC(=O)[C@H](CCCCNC(=O)c1cccc(NC(=O)N[C@@H]2CN(C3CCCCC3)c3ccccc3N(CC(=O)C(C)(C)C)C2=O)c1)NC(=O)CN1CCN(CC(=O)O)CCN(CC(=O)O)CCN(CC(=O)O)CC1)C(=O)N[C@@H](Cc1ccc(O)cc1)C(=O)N[C@H](Cc1c[nH]c2ccccc12)C(=O)N[C@@H](CCCCN)C(N)=O)C(=O)N[C@H](C(=O)O)[C@@H](C)O] |
| I- 40 | DOTA- K(Z360)- PEG6- f[CYwKTC]T- OH | 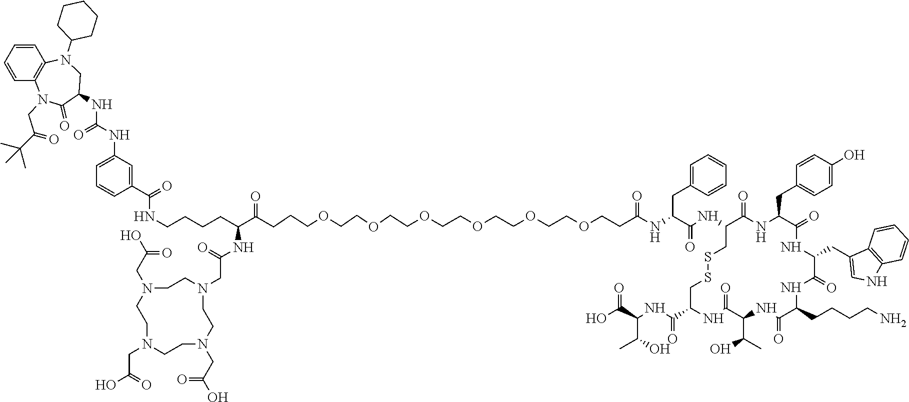 [SMILES: C[C@@H](O)[C@H](NC(=O)[C@@H]1CSSC[C@H](NC(=O)[C@@H](Cc2ccccc2)NC(=O)CCOCCOCCOCCOCCOCCOCCCC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 41 | DOTA- OEG- OEG- K(Z360)- f[CYwKTC]T- OH | 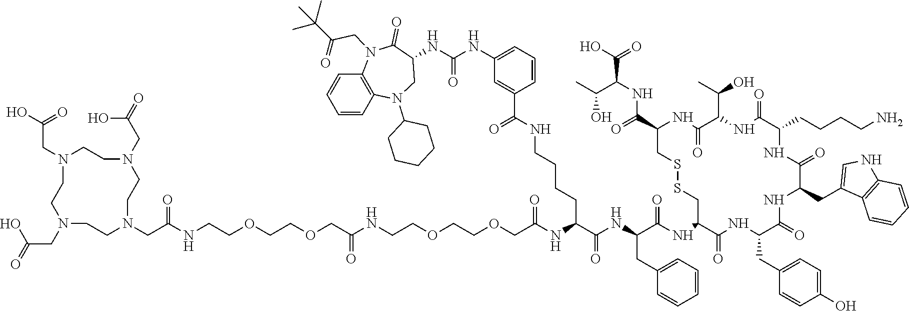 |
| I- 42 | DOTA- K(-OEG- OEG- Z360)- f]CYwKTC]T- OH | 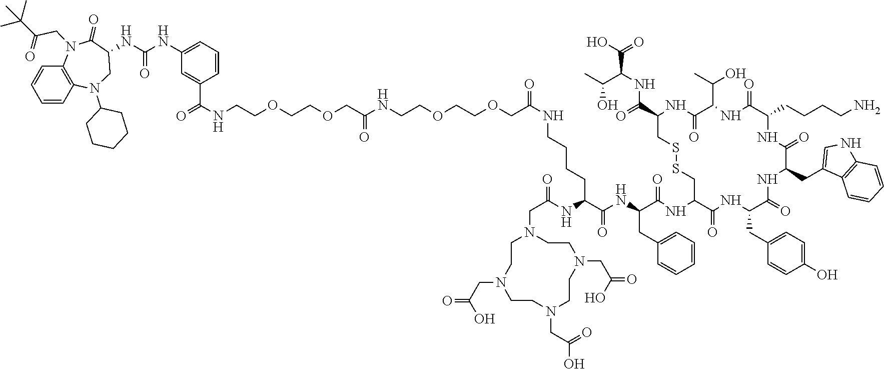 [SMILES: CC(O)[C@@H]1NC(=O)[C@H](CCCCN)NC(=O)[C@@H](Cc2c[nH]c3ccccc23)NC(=O)[C@H](Cc2ccc(O)cc2)NC(=O)C(NC(=O)[C@@H](Cc2ccccc2)NC(=O)[C@H](CCCCNC(=O)COCCOCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC1=O] |
| I- 43 | DOTA- K(-OEG- Z360)- OEG- f[CYwKTC]T- OH | 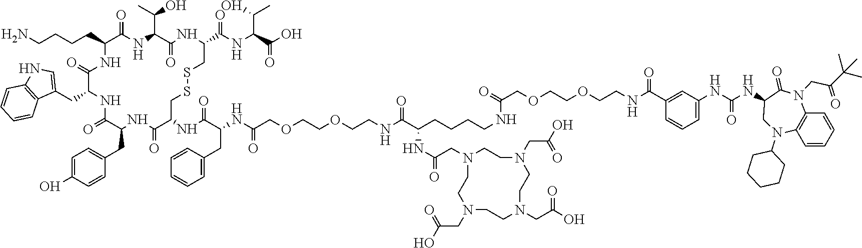 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 44 | DOTA- OEG- K(-OEG- Z360)- f[CYwKTC]T- OH | 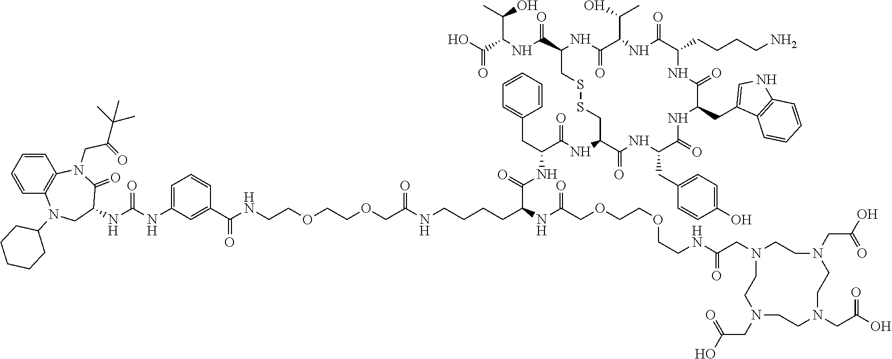 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)COCCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 45 | DOTA- K(Z360)- OEG- OEG- OEG- f[CYwKTC]T- OH | 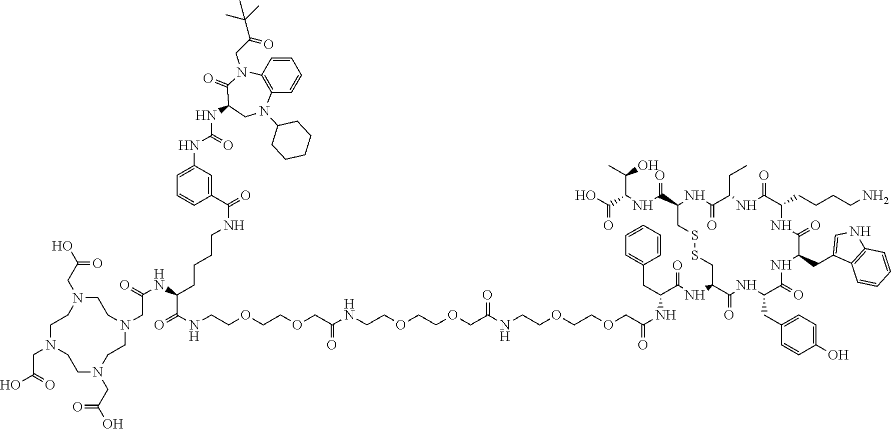 [SMILES: CC[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)COCCOCCNC(=O)COCCOCCNC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)CSSC[C@@H](C(=O)N[C@H](C(=O)O)[C@@H](C)O)NC1=O] |
| I- 46 | DOTA- K(-OEG- OEG- OEG- Z360)- f[CYwKTC]T- OH | 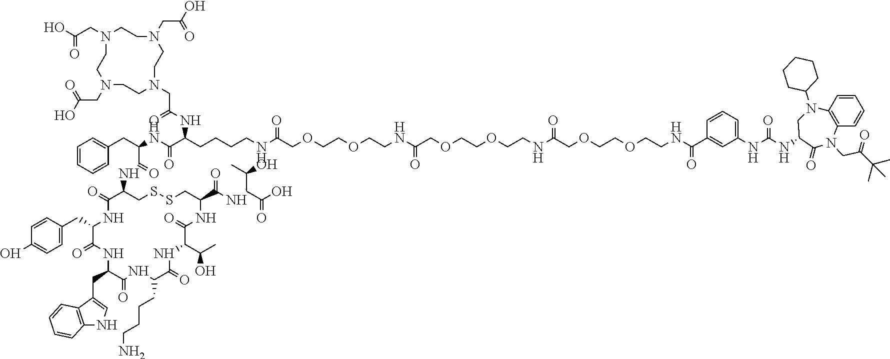 |
| I- 47 | DOTA- eK- OEG- OEG- OEG- K(-gE- G- Z360)- OEG- f[CYwKTC]T- OH | 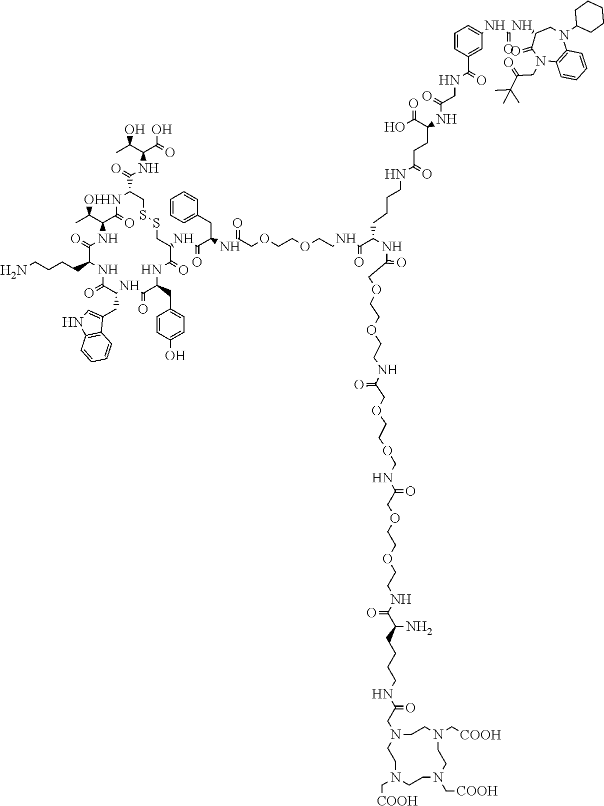 |
| I- 48 | DOTA- PEG3- K(Z360)- f[CYwKTC]T- OH | 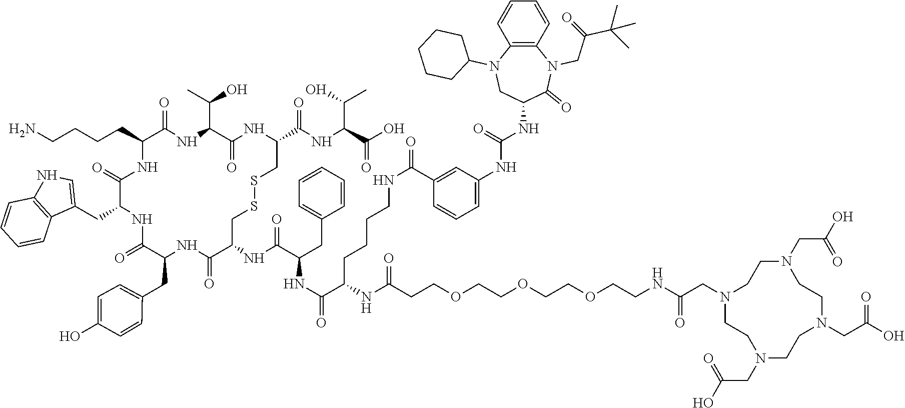 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CCOCCOCCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 49 | DOTA- G-G- K(Z360)- f[CYwKTC]T- OH | 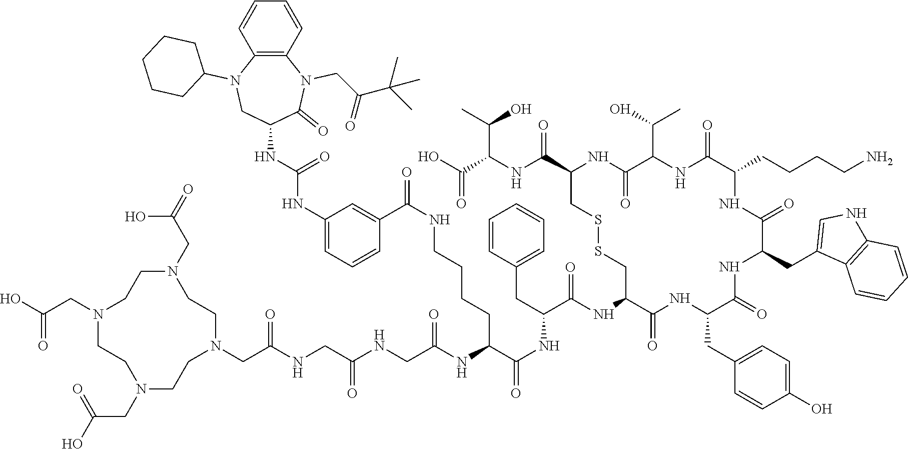 [SMILES: C[C@@H](O)C1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CNC(=O)CNC(=O)CN2CCN(CC(=O)O)CCN(CC(=O)O)CCN(CC(=O)O)CC2)CSSC[C@@H](C(=O)N[C@H](C(=O)O)[C@@H](C)O)NC1=O] |
| I- 50 | DOTA- S-S- K(Z360)- f[CYwKTC]T- OH | 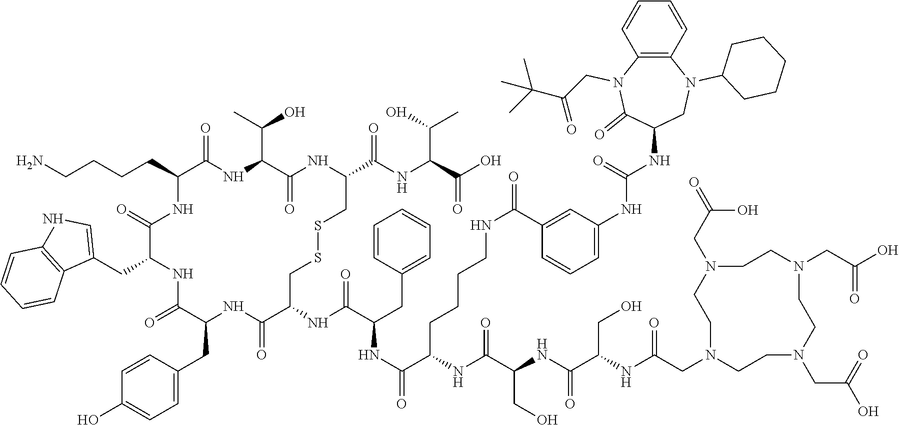 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)[C@H](CO)NC(=O)[C@H](CO)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 51 | DOTA- PEG1- K(Z360)- f[CYwKTC]T- OH | 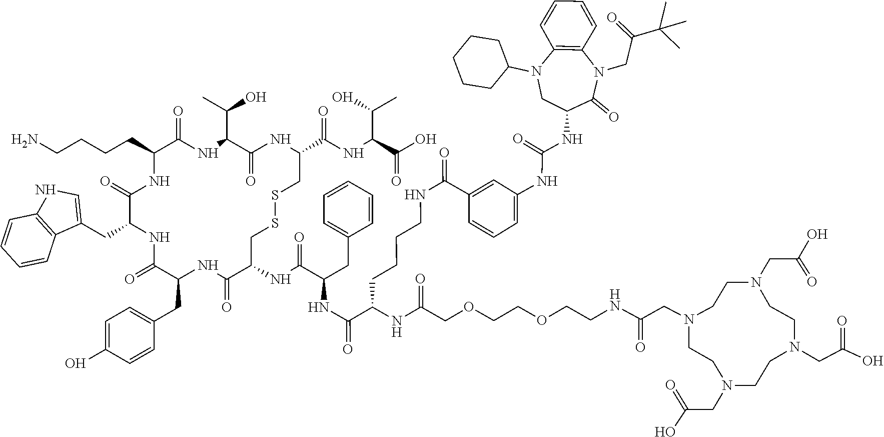 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)COCCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 52 | DOTA- OEG- K(Z360)- OEG- f[CYwKTC]T- OH | 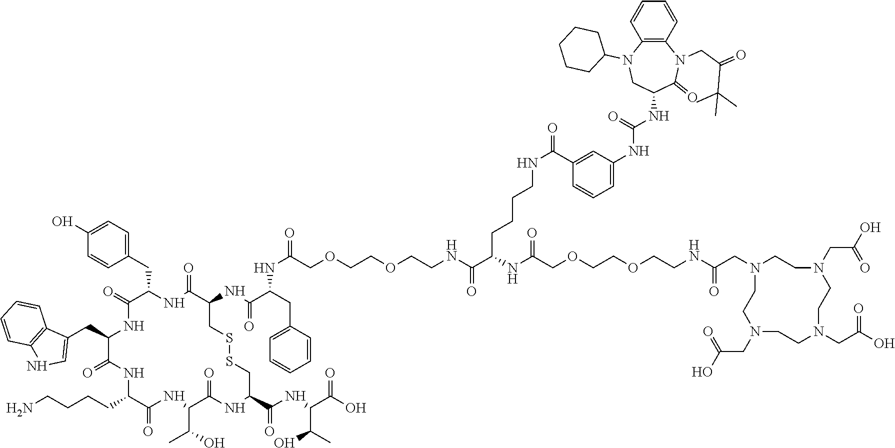 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)COCCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 53 | DOTA- K(Z360)- OEG- G- f[CYwKTC]T- OH | 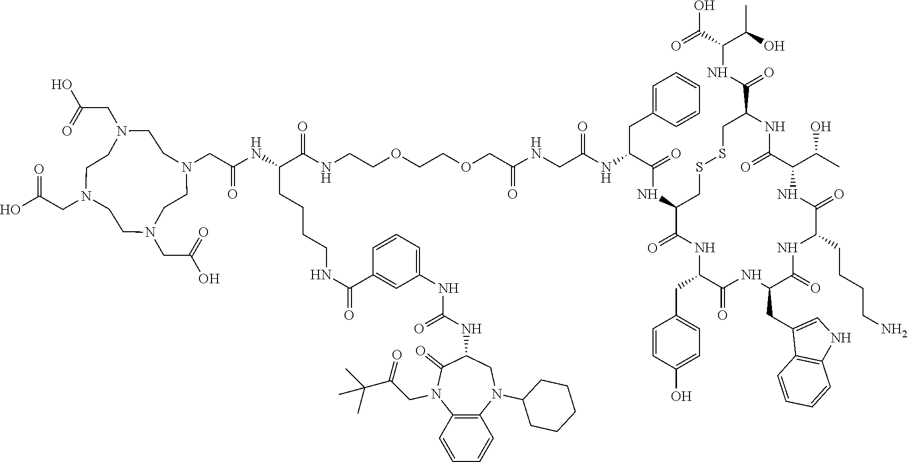 [SMILES: C[C@@H](O)[C@H](NC(=O)[C@@H]1CSSC[C@H](NC(=O)[C@@H](Cc2ccccc2)NC(=O)CNC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 54 | DOTA- K(Z360)- G- S-G- f[CYwKTC]T- OH | 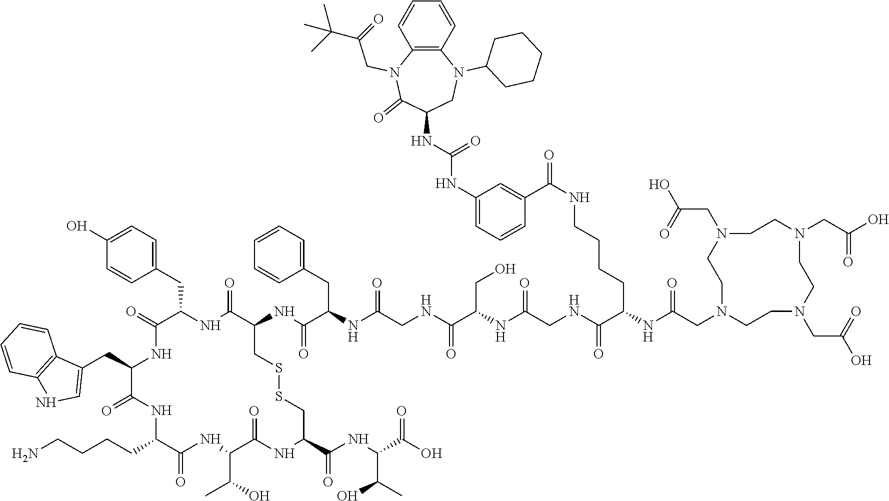 [SMILES: C[C@@H](O)[C@H](NC(=O)[C@@H]1CSSC[C@H](NC(=O)[C@@H](Cc2ccccc2)NC(=O)CNC(=O)[C@H](CO)NC(=O)CNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 55 | DOTA- K(Z360)- G- p-G-p- G- f[CYwKTC]T- OH | 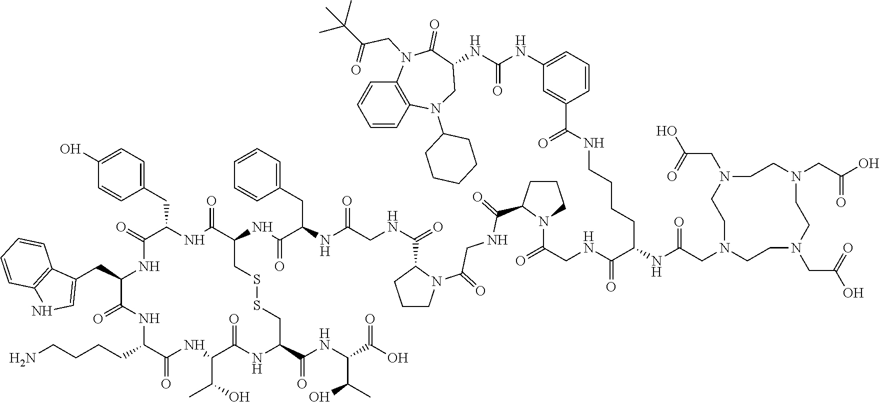 |
| I- 56 | DOTA- G-p-G- K(Z360)- f[CYwKTC]T- OH | 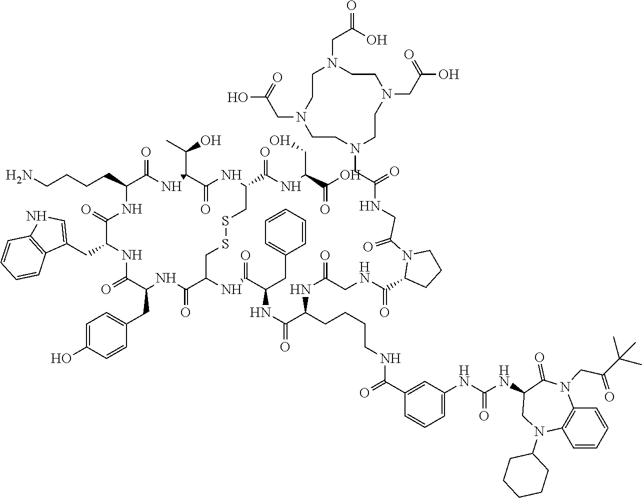 |
| I- 57 | DOTA- p-G-p- G- K(Z360)- f[CYwKTC]T- OH | 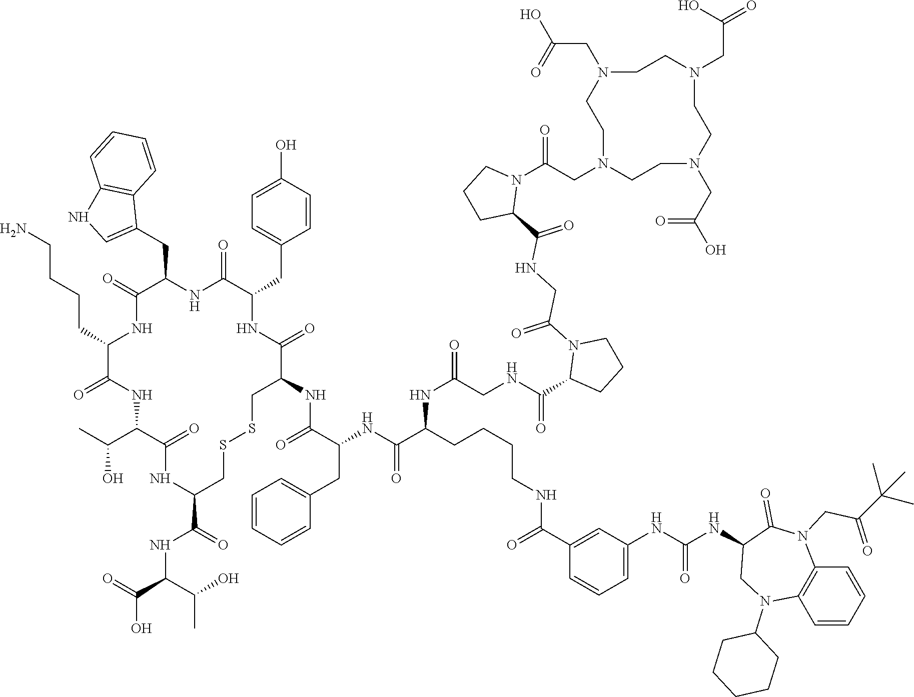 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CNC(=O)[C@H]2CCCN2C(=O)CNC(=O)[C@H]2CCCN2C(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 58 | DOTA- G-S- K(Z360)- G- S- f[CYwKTC]T- OH | 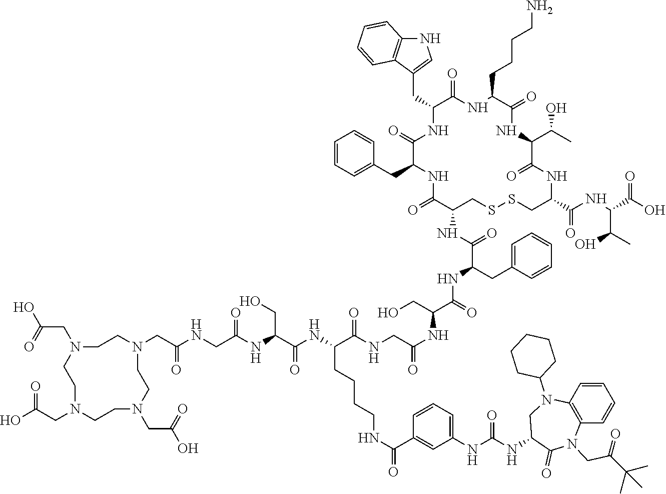 [SMILES: C[C@@H](O)[C@H](NC(=O)[C@@H]1CSSC[C@H](NC(=O)[C@@H](Cc2ccccc2)NC(=O)[C@H](CO)NC(=O)CNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)[C@H](CO)NC(=O)CNC(=O)CN2CCN(CC(=O)O)CCN(CC(=O)O)CCN(CC(=O)O)CC2)C(=O)N[C@@H](Cc2ccccc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 59 | DOTA- Aoc- K(Z360)- f[CYwKTC]T- OH | 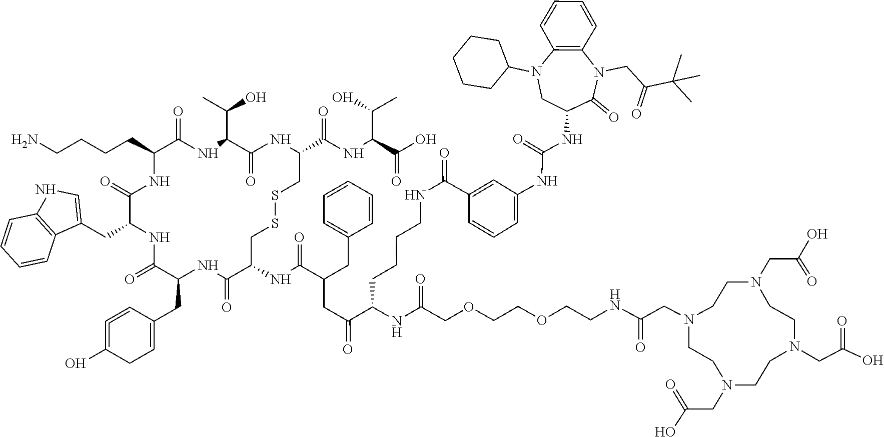 [SMILES: C[C@@H](O)[C@H](NC(=O)[C@@H]1CSSC[C@H](NC(=O)C(CC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)COCCOCCNC(=O)CN2CCN(CC(=O)O)CCN(CC(=O)O)CCN(CC(=O)O)CC2)Cc2ccccc2)C(=O)N[C@@H](CC2=CCC(O)=C=C2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 60 | DOTA- K(-G- G-G- Z360)- f[CYwKTC]T- OH | 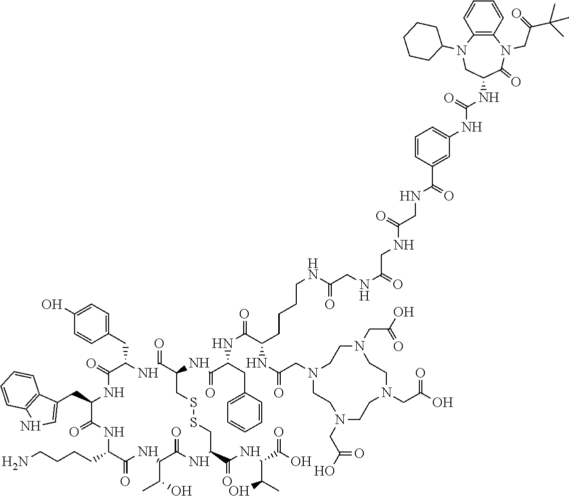 [SMILES: C[C@@H](O)[C@H](NC(=O)[C@@H]1CSSC[C@H](NC(=O)[C@@H](Cc2ccccc2)NC(=O)[C@H](CCCCNC(=O)CNC(=O)CNC(=O)CNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 61 | DOTA- K(Z360)- Aoc- f[CYwKTC]T- OH | 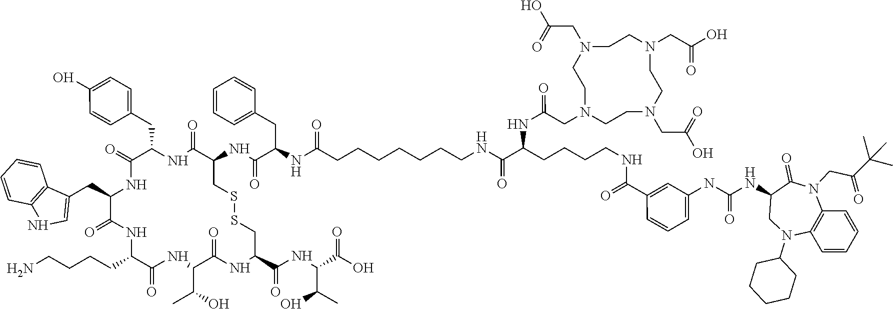 |
| I- 62 | DOTA- R- K(Z360)- f[CYwKTC]T- OH | 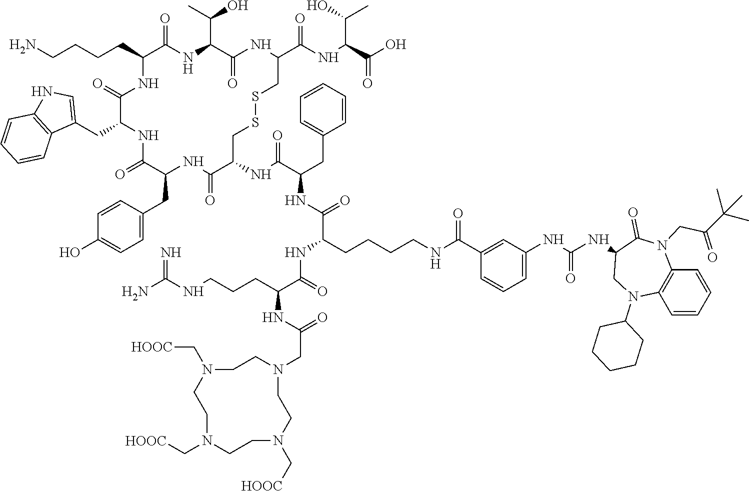 [SMILES: C[C@@H](O)[C@H](NC(=O)C1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)[C@H](CCCNC(=N)N)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 63 | DOTA- K(Z360)- R- f]CYwKTC]T- OH | 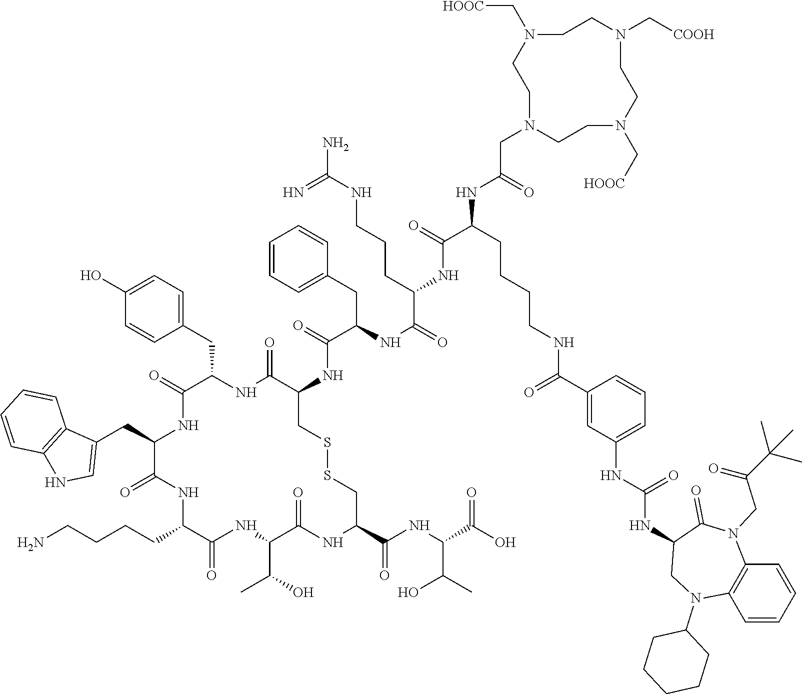 [SMILES: CC(O)[C@H](NC(=O)[C@@H]1CSSC[C@H](NC(=O)[C@@H](Cc2ccccc2)NC(=O)[C@H](CCCNC(=N)N)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I- 64 | DOTA- K(-OEG- Z360)- f[CYwKTC]T- NH₂ | 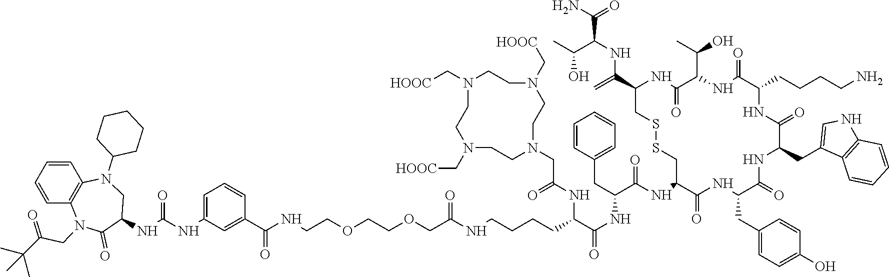 |
| I- 65 | DOTA- eK-eK- OEG- OEG- K(-gE- G- Z360)- OEG- f[CYwKTC]T- OH | 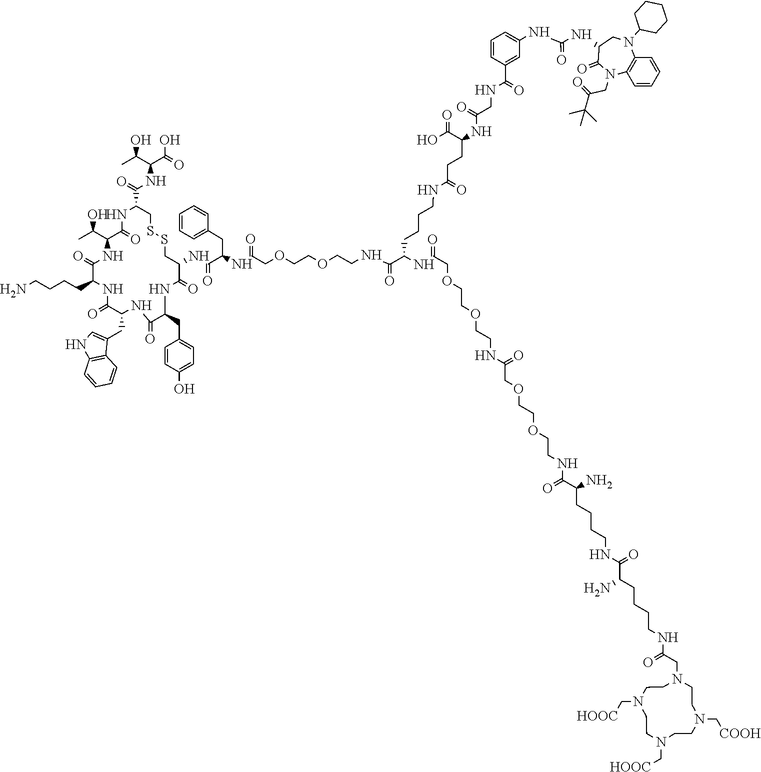 |
| I- 66 | DOTA- eK-eK- eK- OEG- K(-gE- G- Z360)- OEG- f]CYwKTC]T- OH | 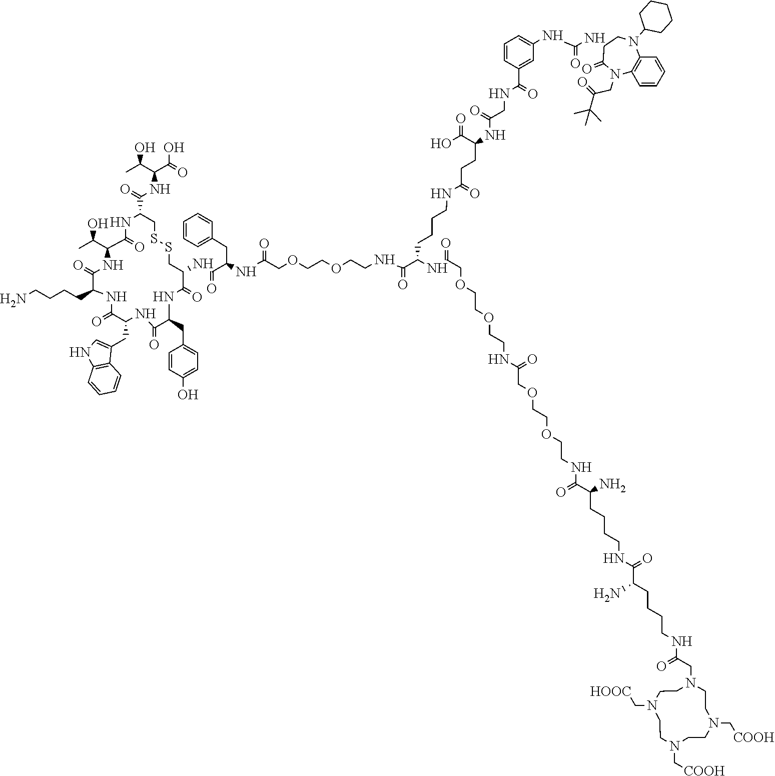 |
| I- 67 | DOTAGA- K(Z360)- OEG- f[CYwKTC]T- OH | 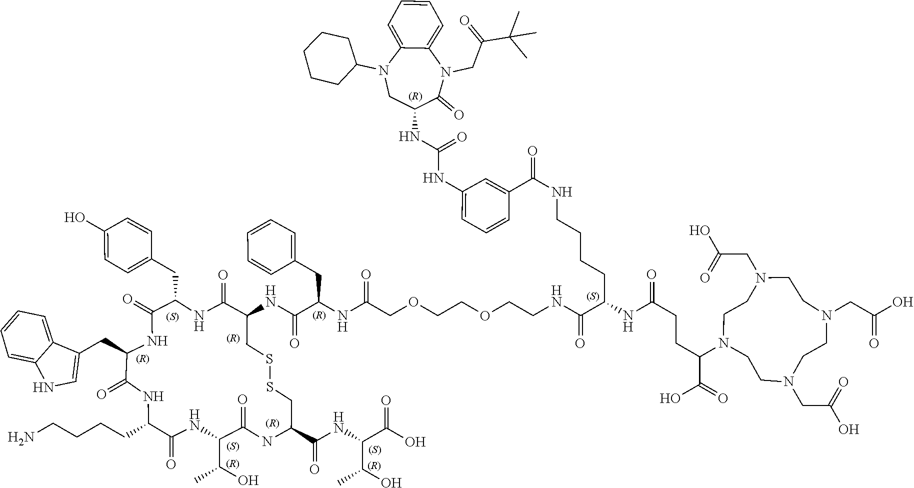 |
| I- 68 | DOTAGA- K(-Glu- Z360)- OEG- f[CYwKTC]T- OH | 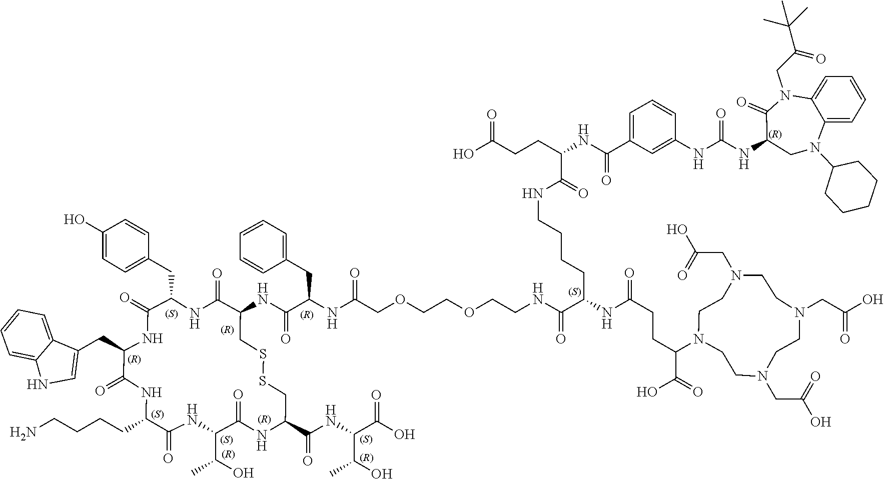 |
| I- 69 | DOTAGA- K(-Glu- Z360)- f[CYwKTC]T- OH | 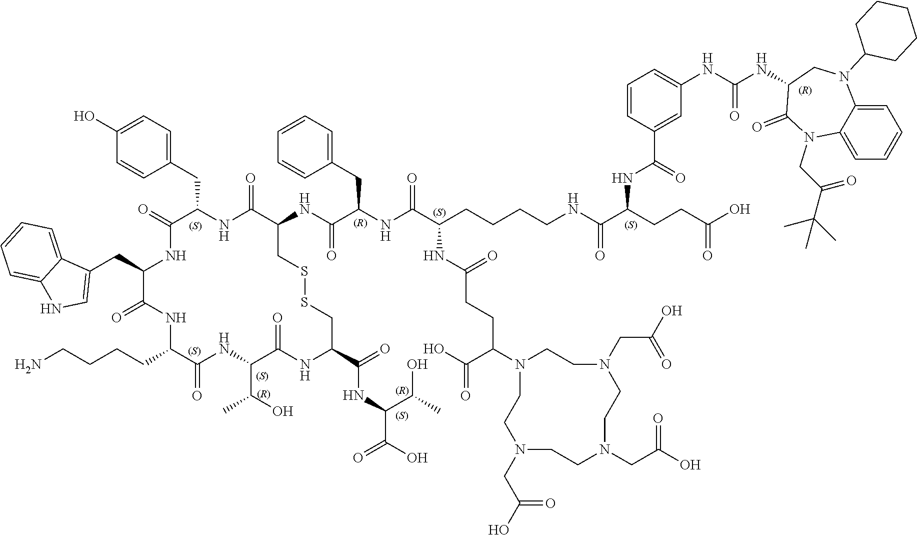 |
| I- 70 | DOTA- K(-Glu- Z360)- Ahx- f[CYwKTC]T- OH | 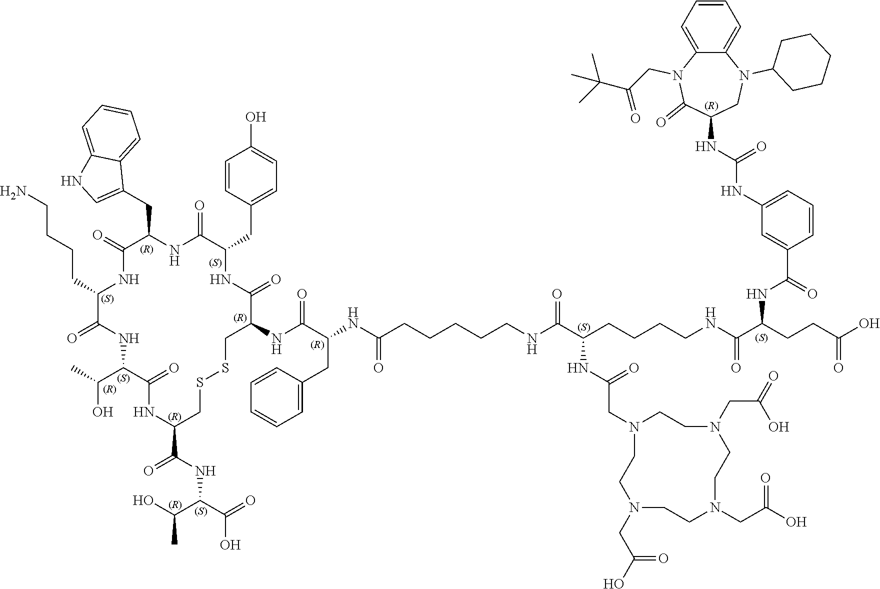 |
| I- 71 | DOTAGA- K(-Glu- Z360)- Ahx- f[CYwKTC]T- OH | 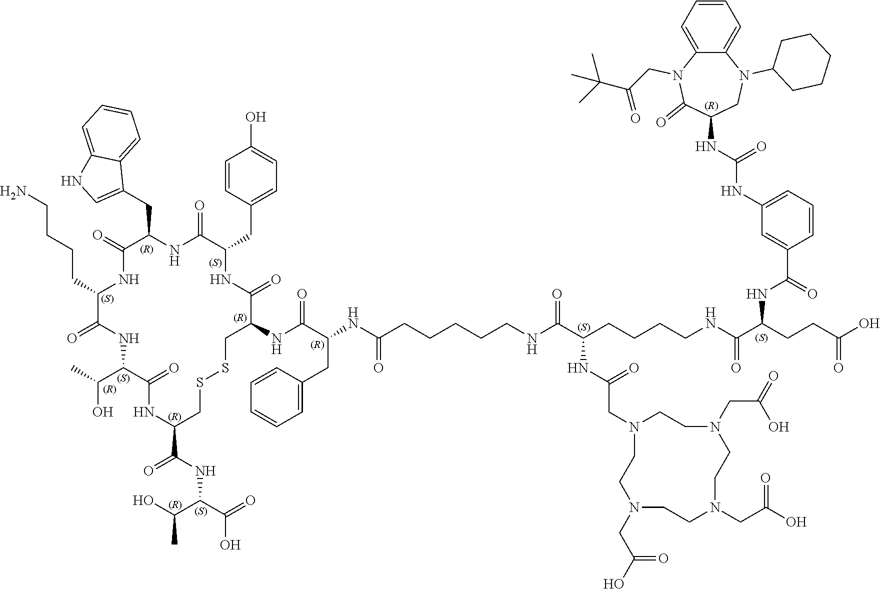 |
| I- 72 | DOTAGA- K(-Glu- Z360)- Ahx- Ahx- f[CYwKTC]T- OH | 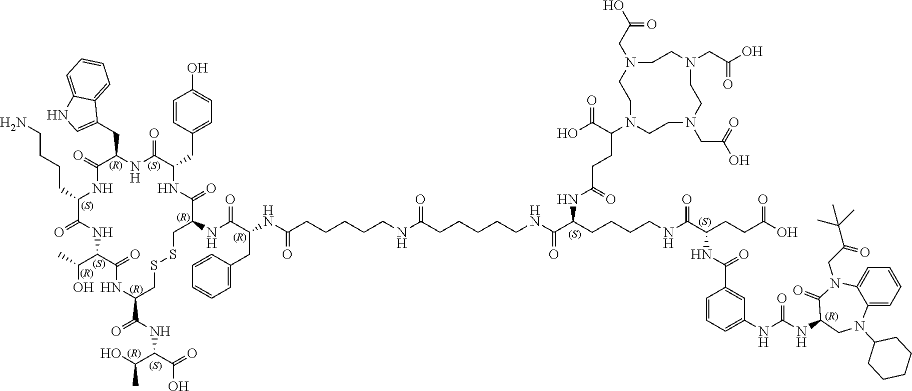 |
| I- 73 | DOTAGA- K(-Glu- Z360)- Aoc- Aoc- f[CYwKTC]T- OH | 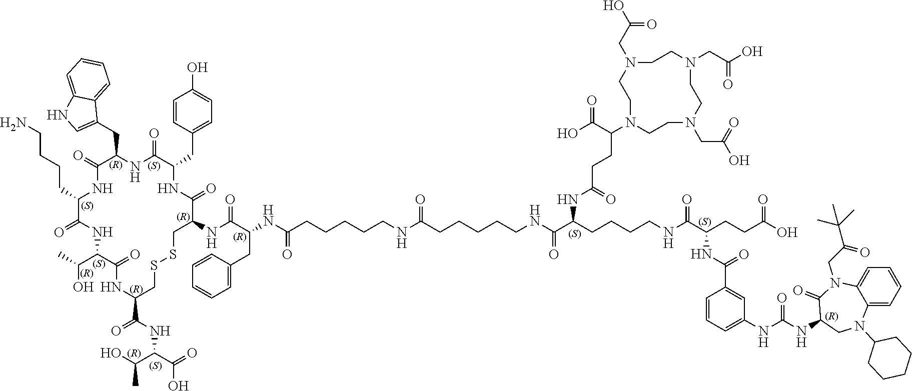 |
| I- 74 | DOTA- K(Z360)- gGlu- OEG- OEG- f]CYwKTC]T- OH | 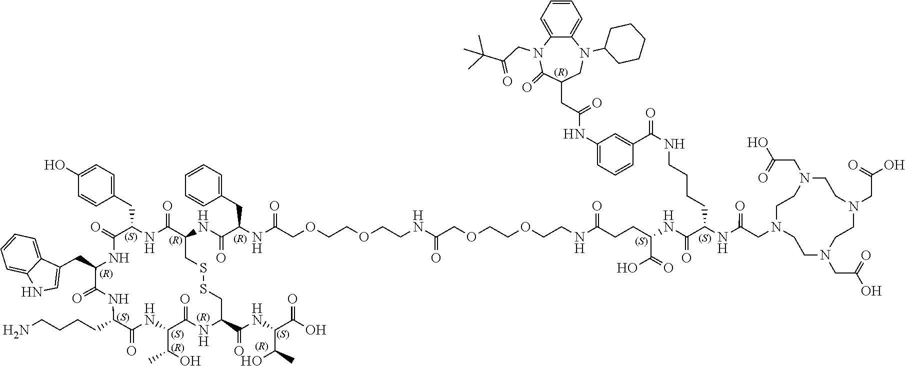 |
| I- 75 | DOTA- K(Z360)- Glu- gGlu- OEG- OEG- f[CYwKTC]T- OH | 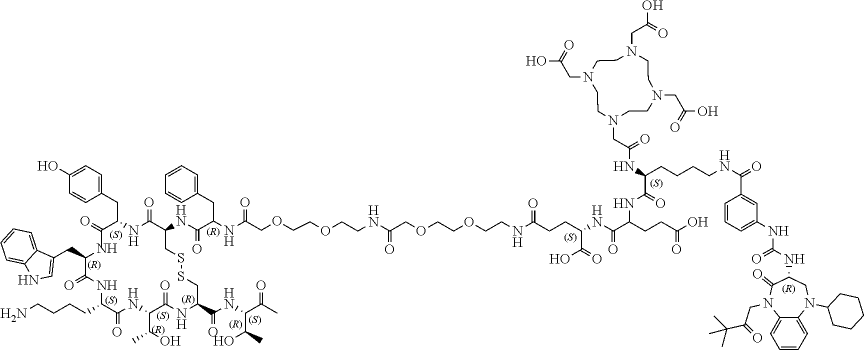 |
| I- 76 | DOTA- K(-OEG- OEG- Glu- Z360)- OEG- f[CYwKTC]T- OH | 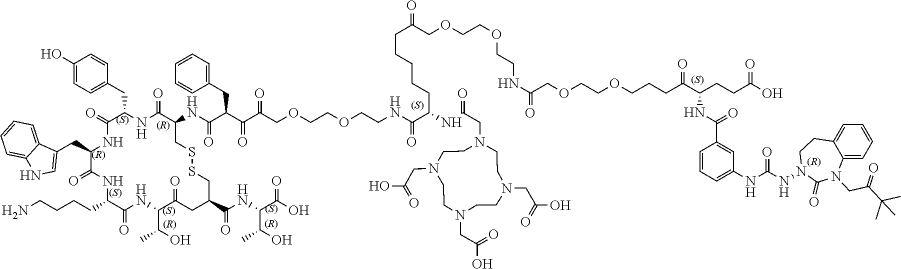 |
| I- 77 | DOTA- K(-OEG- gGlu- Ser- Z360)- OEG- f]CYwKTC]T- OH | 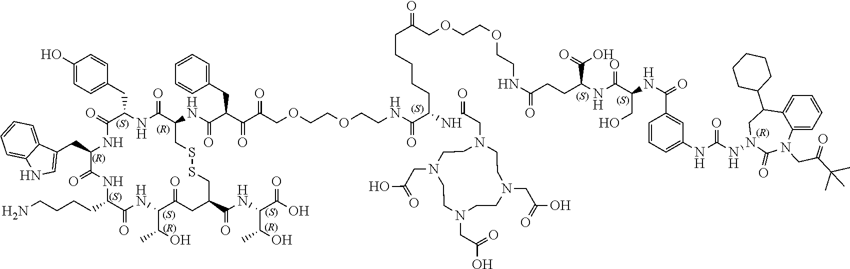 |
| I- 78 | DOTA- K(-OEG- gGlu- Dap- Z360)- OEG- f[CYwKTC]T- OH | 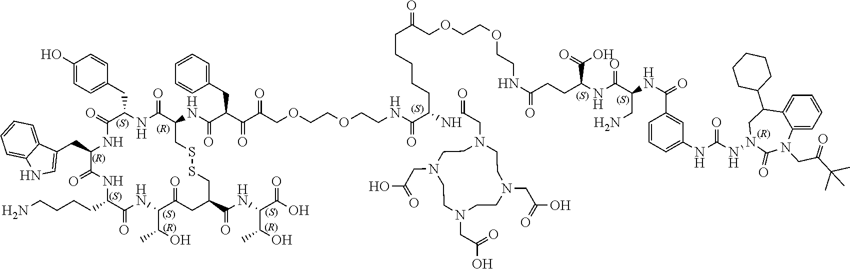 |
| I- 79 | DOTA- K(-OEG- His- Glu- Z360)- OEG- f[CYwKTC]T- OH | 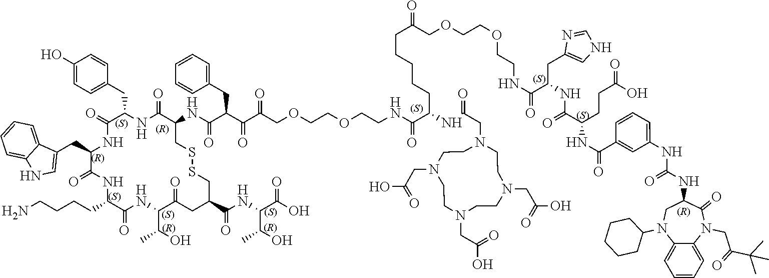 |
| | | and |
| I- 80 | DOTA- K(-OEG- OEG- Cbp- Z360)- OEG- f[CYwKTC]T- OH | 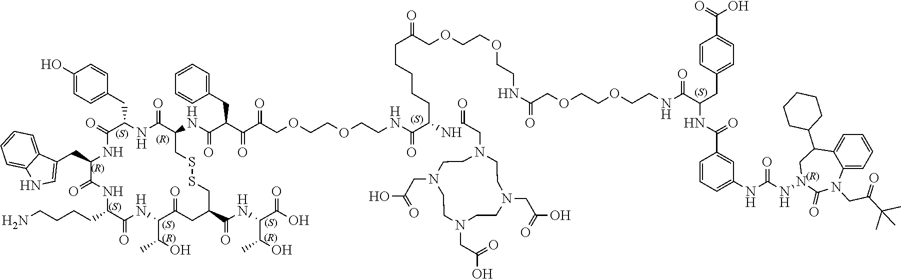 |

Abbreviations:
T: Threonine;
C: Cysteine;
K: Lysine;
w: D-Tryptophan;
Y: Tyrosine;
f: D-Phenylalanine;
A: Alanine;
G: Glycine;
W: Tryptophan;
Nle: Non-leucine;
D: Aspartic acid;
S: Serine;
p: D-proline;
R: Arginine;
H: Histine:
gE: gamma-Glutamic acid;
eK: epsilon-Lysine-OH: C-terminal acid;
—NH2: C-terminal amide;
Thr(ol): C-terminal alcohol
[CXXXXC]: Disulfide bond
DOTA: 1,4,7,10-Tetraazacyclododecane-1,4,7,10-tetraacetic acid;
DOTAGA: 2-[1,4,7,10-Tetraazacyclododecane-4,7,10-trisacetic acid]-pentanedioic acid
OEG: H2N—[CH2HC2O]2—CH2CO2H
PEG1: H2N—CH2CH2O—CH2CH2CO2H
PEG3: H2N—[CH2HC2O]3—CH2CH2CO2H
PEG6: H2N—[CH2CH2O]6—CH2CH2CO2H
Aoc: 8-amino-octanoic acid
Cbp: 4-Carboxy-L-phenylalanine
Dap: 2,3-diaminoproprionic acid
Z360: Nastorazepide; CAS NO. 343326-69-2

The chelating binding group is capable of binding with and/or complexing a radionuclide. Therefore, in some embodiments, the compound of Formula I further comprises a radionuclide complexed to the chelating binding group.

Accordingly, the present application also includes a radionuclide complex (radioligand) or a pharmaceutically acceptable salt and/or solvate thereof, comprising a compound of the application or a pharmaceutically acceptable salt and/or solvate thereof, and one or more radionuclides.

In some embodiments, the radionuclide is selected from a transition metal, rare-earth metal, lanthanide, actinide and metalloid.

In some embodiments, the one or more radionuclides is a radioactive isotope of C, N, O, F, P, S, Cl, Br, I, As, Se, At, K, Sc, Ti, Cr, Mn, Fe, Co, Ni, Cu, Ga, Ge, Rb, Sr, Y, Zr, Nb, Tc, Rh, Pd, In, Sn, Sb, Zn, Ta, W, Re, Os, Ir, Pt, Au, Hg, Tl, Pb, Bi, Po, Fr, Pm, a lanthanide (such as La, Ce, Pr, Nd, Pm, Sm, Eu, Gd, Tb, Dy, Ho, Er, Tm, Yb, Lu), an actinide (such as Ac, Th, U), Mg, Al, Ca, Cd, or Ba.

In some embodiments, the lanthanide Lu, Sm, Ho, or Tb.

In some embodiments, the actinide is Ac Th, or U.

In some embodiments, the radionuclide is selected from ²²⁵Ac, ²²⁶Ac, ²²⁸Ac, ¹⁰⁵Ag, ¹⁰⁶mAg, ¹¹⁰mAg, ¹¹¹Ag, ¹¹²Ag, ¹¹³Ag, ²³⁹Am, ²⁴⁰Am, ²⁴²Am, ²⁴⁴Am, ³⁷Ar, ⁷¹As, ⁷²As, ⁷³As, ⁷⁴As ⁷⁶As, ⁷⁷As, ²⁰⁹At, ²¹⁰At, ¹⁹¹Au, ¹⁹²Au, ¹⁹³Au, ¹⁹⁴Au, ¹⁹⁵Au, ¹⁹⁶Au, ¹⁹⁶mAu, ¹⁹⁸Au, ¹⁹⁸mAu, ¹⁹⁹Au, ²⁰⁰mAu, ¹²⁸Ba, ¹³¹Ba, ¹³³mBa, ¹³⁵mBa, ¹⁴⁰Ba, ⁷Be, ²⁰³Bi, ²⁰⁴Bi, ²⁰⁵Bi, ²⁰⁶Bi, ²¹⁰Bi, ²¹²Bi, ²⁴³Bk, ²⁴⁴Bk ²⁴⁵Bk, ²⁴⁶Bk, ²⁴⁸mBk, ²⁵⁰Bk, ⁷⁶Br, ⁷⁷Br, ⁸⁰mBr, ⁸²Br, ¹¹C, ¹⁴C, ⁴⁵Ca, ⁴⁷Ca, ¹⁰⁷Cd, ¹¹⁵Cd, ¹¹⁵mCd, ¹¹⁷mCd, ¹³²Ce, ¹³³mCe, ¹³⁴Ce, ¹³⁵Ce, ¹³⁷Ce, ¹³⁷mCe, ¹³⁹Ce, ¹⁴¹Ce, ¹⁴³Ce, ¹⁴⁴Ce, ²⁴⁶Cf, ²⁴⁷Cf, ²⁵³Cf ²⁵⁴Cf, 240 Cm, ²⁴¹Cm, ²⁴²C, ²⁵²M, ⁵⁵Co, ⁵⁶Co, ⁵⁷Co, ⁵⁸Co, ⁵⁸mCo, ⁶⁰Co, ⁴⁸Cr, ⁵¹Cr, ¹²⁷Cs, ¹²⁹Cs, ¹³¹Cs, ¹³²Cs, ¹³⁶Cs, ¹³⁷Cs, ⁶¹Cu, ⁶²Cu ⁶⁴Cu, ⁶⁷Cu, ¹⁵³Dy, ¹⁵⁵Dy, ¹⁵⁷Dy, ¹⁵⁹Dy, ¹⁶⁵Dy, ¹⁶⁶Dy, ¹⁶⁰Er 161 Er, ¹⁶⁵Er, ¹⁶⁹Er, ¹⁷¹Er, ¹⁷²Er, ²⁵⁰Es, ²⁵¹Es, ²⁵³Es, ²⁵⁴Es, ²⁵⁴mEs, ²⁵⁵Es, ²⁵⁶mEs, ¹⁴⁵Eu, ¹⁴⁶Eu, ¹⁴⁷Eu ¹⁴⁸Eu, ¹⁴⁹Eu, ¹⁵⁰mEu, ¹⁵²mEu, ¹⁵⁶Eu, ¹⁵⁷Eu, ⁵²Fe, ⁵⁹Fe, 251 Fm, ²⁵²Fm, ²⁵³Fm, ²⁵⁴Fm, ²⁵⁵Fm, ²⁵⁷Fm ⁶⁶Ga, ⁶⁷Ga, ⁶⁸Ga, ⁷²Ga, ⁷³Ga, ¹⁴⁶Gd, ¹⁴⁷Gd, ¹⁴⁹Gd, ¹⁵¹Gd, ¹⁵³Gd, ¹⁵⁹Gd, ⁶⁸Ge, ⁶⁹Ge, ⁷¹Ge, ⁷⁷Ge ¹⁷⁰Hf, ¹⁷¹Hf, ¹⁷³Hf, ¹⁷⁵Hf, ¹⁷⁹mHf, ¹⁸⁰mHf, 181 Hf, ¹⁸⁴Hf, ¹⁹²Hg, ¹⁹³Hg, ¹⁹³mHg, ¹⁹⁵Hg, ¹⁹⁵mHg, ¹⁹⁷Hg, ¹⁹⁷mHg, ²⁰³Hg, ¹⁶⁰mHo, ¹⁶⁶Ho, ¹⁶⁷Ho, ¹²³I, ¹²⁴I, ¹²⁶I, ¹³⁰I, ¹³²I, ¹³³I, ¹³⁵I, ¹⁰⁹In, ¹¹⁰I, ¹¹¹I, In, ¹¹⁴ mln, ¹¹⁵mln, ¹⁸⁴Ir, ¹⁸⁵Ir, ¹⁸⁶Ir, ¹⁸⁷Ir, ¹⁸⁸Ir, ¹⁸⁹Ir, ¹⁹⁰r, ¹⁹⁰mlr, ¹⁹²Ir, ¹⁹³mIr, ¹⁹⁴Ir, ¹⁹⁴mIr, ¹⁹⁵ mIr, ⁴²K, ⁴³K ⁷⁶, Kr⁷⁹, Kr⁸¹mKr, ⁸⁵mKr, ¹³²La, ¹³³La, ¹³⁵La, ¹⁴⁰La, 141 La, ²⁶²Lr, ¹⁶⁹Lu, ¹⁷⁰Lu, ¹⁷¹Lu, ¹⁷²Lu, ¹⁷⁴mLu, ¹⁷⁶mLu ¹⁷⁷Lu, ¹⁷⁷mLu, ¹⁷⁹Lu, ²⁵⁷Md, ²⁵8Md, ²⁶⁰Md, ²⁸Mg, ⁵²Mn, ⁹⁰Mo, ⁹³mMo, Mo, ¹³N, ²⁴Na, ⁹⁰Nb, 91 mNb, ⁹²mNb, ⁹⁵Nb, ⁹⁵mNb, ⁹⁶Nb, ¹³⁸Nd, ¹³⁹mNd, ¹⁴⁰Nd, ¹⁴⁷Nd, ⁵⁶Ni, ⁵⁷Ni, ⁶⁶Ni, ²³⁴Np, ²³⁶mNp, ²³⁸Np ²³⁹Np, ¹⁸²0s, ¹⁸³0s, ¹⁸³mOs, ¹⁸⁵0s, ¹⁸⁹mOs, ¹⁹¹0s, 191 mOs, ¹⁹³0s, 3²p 33p 228 Pa, 229 Pa 230 Pa, 232 Pa, 233 Pa, 234 Pa ²⁰⁰Pb, 201 Pb, ²⁰²mPb, ²⁰³Pb, ²⁰⁹Pb, ²¹²Pb, ¹⁰⁰Pd, 101 Pd, ¹⁰³Pd, ¹⁰⁹Pd, ¹¹¹mPd, ¹¹²Pd, ¹⁴³Pm, ¹⁴⁸Pm, ¹⁴⁸mPm, ¹⁴⁹Pm, 151 Pm, ²⁰⁴P0, ²⁰⁶Po, ²⁰⁷Po, ²¹⁰Po, ¹³⁹Pr, ¹⁴²Pr, ¹⁴³Pr. ¹⁴⁵Pr, ¹⁸⁸Pt, 189Pt 191 Pt, ¹⁹³mPt, ¹⁹⁵mPt, ¹⁹⁷Pt, ²⁰⁰Pt, ²⁰²Pt, ²³⁴Pu, ²³⁷Pu, ²⁴³Pu, ²⁴⁵Pu, ²⁴⁶Pu, ²⁴⁷Pu, ²²³Ra, ²²⁴Ra, ²²⁵Ra, ⁸¹Rb, ⁸²Rb, ⁸²mRb, ⁸³Rb, ⁸⁴Rb, ⁸⁶Rb, ¹⁸¹Re, ¹⁸²Re, ¹⁸²mRe, ¹⁸³Re, ¹84Re, ¹⁸⁴mRe, ¹⁸⁶Re, ¹⁸⁸Re, ¹89Re, ¹⁹⁰mRe, ⁹⁹Rh, ⁹⁹mRh, ¹⁰⁰Rh, 101 mRh, ¹⁰²Rh, ¹⁰³mRh, ¹⁰⁵Rh, 211 Rn ²²²Rn, ⁹⁷Ru, ¹⁰³Ru, ¹⁰⁵Ru, ³⁵S, ¹¹⁸mSb, ¹¹⁹Sb, ¹²⁰Sb, ¹²⁰mSb, ¹²²Sb, ¹²⁴Sb, ¹²⁶Sb, ¹²⁷Sb, ¹²⁸Sb ¹²⁹Sb, ⁴³SC, 4SC, 4mSC, ⁴⁶SC, ⁴⁷SC, ⁴⁸SC, ⁷²Se ⁷³Se, ⁷⁵Se ¹⁵³SM, ¹⁵⁶SM, ¹¹⁰Sn, ¹¹³Sn, ¹¹⁷mSn, ¹¹⁹mSn, ¹²¹Sn, ¹²³Sn, ¹²⁵Sn, ⁸²Sr, ⁸³Sr, ⁸⁵Sr, ⁸⁹Sr, 91 Sr, ¹⁷³Ta, ¹⁷⁵Ta, ¹⁷⁶Ta, ¹⁷⁷Ta ¹⁸⁰Ta, ¹⁸²Ta ¹⁸³Ta, ¹⁸⁴Ta ¹⁴⁹Tb, ¹⁵⁰Tb, ¹⁵¹Tb, ¹⁵²Tb, ¹⁵³Tb, ¹⁵⁴Tb, ¹⁵⁴mTb, ¹⁵⁴m2Tb, ¹⁵⁵Tb, ¹⁵⁶Tb, ¹⁵⁶mTb, ¹⁵⁶m2Tb, ¹⁶⁰Tb, ¹⁶¹Tb, ⁹⁴Tc, ⁹⁵Tc, ⁹⁵mTc, ⁹⁶Tc, ⁹⁷mTc, ⁹⁹mTc, ¹¹⁸Te ¹¹⁹Te, ¹¹⁹mTe, ¹²¹Te, 121 mTe, ¹²³mTe, ¹²⁵mTe, ¹²⁷Te, ¹²⁷mTe, ¹²⁹mTe, ¹³¹mTe, ¹³²Te, 227Th ²³¹Th, 234Th, ⁴⁵Ti, ¹⁹⁸Tl, ¹⁹⁹T, ²⁰⁰Tl, ²⁰¹Tl, ²⁰²Tl, ²⁰⁴Tl, ¹⁶⁵Tm, ¹⁶⁶Tm, ¹⁶⁷Tm, ¹⁶⁸TM, ¹⁷⁰Tm ¹⁷²Tm, ¹⁷³Tm, ²³⁰U, ²³¹U, ²³⁷U, ²⁴⁰U, ⁴⁸V, ¹⁷⁸W, ¹⁸¹W, ¹⁸⁵W, ⁸⁷W, ¹⁸⁸W, ¹²²Xe, ¹²⁵Xe, ¹²⁷Xe, ¹²⁹mXe, ¹³¹ mXe, ¹³³Xe¹³³mXe, ¹³⁵Xe⁸⁵mY, ⁸⁶Y, ⁸⁷Y⁸⁷mY, ⁸⁸Y, ⁹⁰Y, ⁹⁰mY, ⁹¹Y, ⁹²Y, ⁹³Y, ¹⁶⁶Yb, ¹⁶⁹Yb, ¹⁷⁵Yb, ⁶²Zn, ⁶⁵Zn, ⁶⁹mZn, ⁷¹mZn, ⁷²Zn, ⁸⁶Zr, ⁸⁸Zr ⁸⁹Zr, ⁹⁵Zr, and ⁹⁷Zr.

In some embodiments, the one or more radionuclides are selected from ¹⁴C, ¹⁵N, ¹⁸F, ⁷⁵Br, ⁷⁶Br, ⁷⁷Br, ¹²³I, ¹²⁴I, ¹²⁵I, ¹³¹I, ³⁵S, ⁹⁹Tc, ^{99m}Tc, ¹⁸⁸Re, ¹⁸⁶Re, ¹⁵³Sm, ⁶⁶Ga, ⁶⁷Ga, ⁶⁸Ga, ¹¹¹In, ¹²³In, ⁵⁹Fe, ⁶³Zn, ⁵²Fe, ⁵²Mn, ⁴⁵Ti, ⁶⁰Cu, ⁶¹Cu, ⁶⁷Cu, ⁶⁴Cu, ⁶²Cu, ⁸²Rb, ^{195m}Pt, ^{191m}Pt, ^{193m}Pt, ^{117m}Sn, ⁸⁹Zr, ¹⁷⁷Lu, ¹⁸F, ¹⁸⁸Re, ¹⁸⁶Re, ¹⁵³Sm, ⁶⁶Ho, ³⁷Y, ⁹⁰Y, ⁸⁹Sr, ¹⁵³Gd, ¹⁵⁹Gd, ²²⁵Ac, ²¹²Bi, ²¹³Bi, ²¹¹At, ¹⁹⁸Au, ¹⁹⁹Au, ^{193m}Pt, ¹⁹⁷Pt ¹⁰³Pd, ¹⁰⁹Pd, ¹⁰⁵Rh, ^{101m}Rh, ^{103m}Rh, ²²³Ra, ²²⁴Ra, ⁹⁷Ru, ²²⁷Th, ²²⁹Th, ³²P, ¹⁶¹Tb, ³³P, ¹⁴⁹Tb, ¹²⁵I, ²⁰³Pb, ²¹²Pb, ²⁰¹TI, ¹¹⁹Sb, ^{58m}Co, ⁵⁵Co, ⁵⁷Co, ⁴⁷Sc, ¹⁴⁹Pm, ¹⁴²Pr, ¹⁶¹Ho, ¹⁶⁶Ho, ¹⁷⁵Yb, or ⁵¹Cr.

In some embodiments, the one or more radionuclide are for use in imaging or diagnosing, or for use in therapy.

In some embodiments, the one or more radionuclide for use in imaging or diagnosing is selected from ^{99m}Tc, ¹⁸⁸Re, ¹⁸⁶Re, ¹⁵³Sm, ⁶⁶Ga, ⁶⁷Ga, ⁶⁸Ga, ¹¹¹In, ⁵⁹Fe, ⁶³Zn, ⁵²Fe, ⁵²Mn, ⁴⁵Ti, ⁶⁰Cu, ⁶¹Cu, ⁶⁷Cu, ⁶⁴Cu, ⁶²Cu, ⁸²Rb, ¹⁹⁸Au, ¹⁹⁹Au, ^{195m}Pt, ^{191m}Pt, ^{193m}Pt, ^{117m}Sn, ⁸⁹Zr, ¹⁷⁷Lu, ¹⁸F, ²⁰³Pb, ⁴⁴Sc, ⁵¹Cr, ^{11m}Rh, ¹⁶⁶Ho, or ¹²³I.

In some embodiments, one or more radionuclides for use in therapy are selected from ¹⁸⁸Re, ¹⁸⁶Re, ¹⁵³Sm, ⁶⁶Ho, ⁹⁰Y, ⁸⁹Sr, ¹¹¹In, ¹⁵³Gd, ²²⁵Ac, ²¹²Bi, ²¹³Bi, ²¹¹At, ⁶⁰Cu, ⁶¹Cu, ⁶⁷Cu, ⁶⁴Cu, ⁶²Cu, ¹⁹⁸Au, ⁹⁹Au, ^{195m}Pt, ^{193m}Pt, ¹⁹⁷Pt, ^{117m}Sn, ¹⁰³Pd, ¹⁰⁵Rh, ^{103m}Rh, ¹⁷⁷Lu, ²²³Ra, ²²⁴Ra, ²²⁷Th, ²²⁹Th, ¹⁴⁹Tb, ³²P, ¹⁶¹Tb, ³³P, ¹²⁵I, ²⁰³Pb, ²¹²Pb, ²⁰¹Tl, ¹¹⁹Sb, ^{58m}Co, ⁴⁷Sc, ¹⁴⁹Pm, ¹⁶¹Ho, ¹⁵⁹Gd, ¹⁴²Pr, ¹⁶⁶Ho, or ¹⁷⁵Yb.

In some embodiments, the radionuclides for use in therapy are selected from ¹⁷⁷Lu, ¹⁵³Sm, ²¹²Pb, ⁹⁰Y, and ²²⁵Ac. In some embodiments, the radionuclides for use in therapy is ¹⁷⁷Lu or ²²⁵Ac. In some embodiments, the radionuclides for use in therapy is ¹⁷⁷Lu. In some embodiments, the radionuclides for use in therapy is ²²⁵Ac.

Accordingly, in an exemplary embodiment, the present application includes a radionuclide complex or a pharmaceutically acceptable salt and/or solvate thereof, comprising a compound of the application or a pharmaceutically acceptable salt and/or solvate thereof and a lanthanide radionuclide. In some embodiments, the lanthanide radionuclide is ¹⁷⁷Lu.

In an embodiment the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of compounds of the application include, for example, those made with solvents that are pharmaceutically acceptable. Examples of such solvents include water (resulting solvate is called a hydrate) and ethanol and the like. Suitable solvents are physiologically tolerable at the dosage administered.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

### III. Compositions and Kits of the Application

The compounds and complexes of the present application are suitably formulated in a conventional manner into compositions using one or more carriers. Accordingly, the present application also includes a composition comprising one or more compounds or complexes of the application and a carrier. The compounds or complexes of the application are suitably formulated into pharmaceutical compositions for administration to subjects in a biologically compatible form suitable for administration in vivo. Accordingly, the present application further includes a pharmaceutical composition comprising one or more compounds or complexes of the application and a pharmaceutically acceptable carrier. In embodiments of the application the pharmaceutical compositions are used in the treatment of any of the diseases, disorders or conditions described herein.

The present application also includes a kit comprising
one or more compounds of Formula I as defined above, or a pharmaceutically acceptable salt and/or solvate thereof, and
instructions for administration of the one or more compounds of Formula I, or a pharmaceutically acceptable salt and/or solvate thereof, to a subject in need thereof.

The present application also includes a kit comprising
one or more compounds of Formula I as defined above, or a pharmaceutically acceptable salt and/or solvate thereof, and
one or more radioisotope as defined above, and
optionally instructions for administration of the one or more compounds of Formula I, or a pharmaceutically acceptable salt and/or solvate thereof, to a subject in need thereof and the radioisotope to a subject in need thereof.

The present application also includes a kit comprising one or more complexes of the application as defined above, or a pharmaceutically acceptable salt and/or solvate thereof, and instructions for administration of the one or more compounds complexes to a subject in need thereof.

In some embodiments, the one or more compounds of Formula I or a pharmaceutically acceptable salt and/or solvate thereof as defined above, the one or more complexes or a pharmaceutically acceptable salt and/or solvate thereof as defined above, or the one or more radioisotope as defined above and are each present in the kits in one or more pharmaceutical compositions.

In some embodiments, the pharmaceutical compositions comprising the one or more compounds of Formula I or a pharmaceutically acceptable salt and/or solvate thereof as defined above, the one or more complexes or a pharmaceutically acceptable salt and/or solvate thereof as defined above, or the one or more radioisotope as defined above are formulated for parenteral administration as described below. In some embodiments, the parenteral administration is by injection.

In some embodiments, the kit further comprises a pharmaceutically acceptable buffer such as bacteriostatic water for injection (BWFI), phosphate-buffered saline, Ringer's solution or dextrose solution. In some embodiments, the pharmaceutically acceptable buffer is present in the kits in one or more containers such as vial or ampoule.

In some embodiments, the kits are for use in imaging. In some embodiments, the kits are for use in therapy. In some embodiments, the kits are for use in treating cancer. In some embodiments, the kits are adapted and/or arranged to carry out any method of the present application. Therefore, the present application also includes pharmaceutical packages or kits adapted and arranged to carry out any method of the present application.

The compounds or complexes of the application are administered to a subject in a variety of forms depending on the selected route of administration, as will be understood by those skilled in the art. For example, a compound of the application is administered by oral, inhalation, parenteral, buccal, sublingual, nasal, rectal, vaginal, patch, pump, minipump, topical or transdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20^{th} edition) and in The United States Pharmacopeia: The National Formulary (USP 24 NF19) published in 1999.

Parenteral administration includes systemic delivery routes other than the gastrointestinal (GI) tract, and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound or complex of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound or complex of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiments, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, a compound or complex of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, the compounds or complexes of the application are suitably in a sterile powder form for reconstitution with a suitable vehicle, e.g., sterile pyrogen-free water, before use.

In some embodiments, compositions for nasal administration are conveniently formulated as aerosols, drops, solutions, gels and powders.

Compositions suitable for buccal or sublingual administration include tablets, lozenges, and pastilles, wherein a compound or complex of the application is formulated with a carrier such as sugar, acacia, tragacanth, or gelatin and glycerine.

Suppository forms of the compounds or complexes of the application are useful for vaginal, urethral and rectal administrations.

In some embodiments a compound or complex of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

A compound or complex of the application including pharmaceutically acceptable salts and/or solvates thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### IV. Methods and Uses of the Application

Accordingly, the present application also includes a method of treating a disease or disorder comprising administering a therapeutically effective amount of one or more compounds or complexes of the application to a subject in need thereof. The present application also includes a use of one or more compounds or complexes of the application for treatment of a disease or disorder as well as a use of one or more compounds or complexes of the application for the preparation of a medicament for treatment of a disease or disorder. The application further includes one or more compounds or complexes of the application for use in treating a disease or disorder.

In one embodiment, a target of the target binding group of the compound or complex is present on disease cells. Indeed, the presence and/or overexpression of receptors on the cell surface is a hallmark of many disease associated cells including cancer cells. According, in another embodiment, a target of the target binding group (for example a cell surface receptor) is present on cancer cells and disease or disorder is cancer. In some embodiments, a first targeting group, a second targeting group or both are present on disease cells. For example, in one embodiment, the target of the first target binding group is Cholecystokin receptor, and the disease or disorder is medullary thyroid carcinoma (MTC), small cell lung cancer (SCLC), astrocytoma, stromal ovarian or gastrointestinal stromal tumors (GIST). In another embodiment, the target of the second target binding group is Somatostatin receptor 2 (SSTR2) and the disease or disorder is a neuroendocrine tumor, a small cell lung cancer (LSCL) or medullary thyroid carcinoma (MTC). In some embodiments, the target of the first and second targeting binding groups are both Cholecystokin receptor (CCK2R) and Somatostatin receptor 2 (SSTR2), and the disease or disorder is MTC or SCLC, where both SSTR2 and CCK2R are overexpressed.

In one embodiment, the disease or disorder is cancer.

In an embodiment, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; medullary thyroid carcinoma; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; Wilms' Tumor and a neuroendocrine tumor. Metastases of the aforementioned cancers can also be treated in accordance with the methods described herein.

In another embodiment, the cancer is a cancer that overexpresses SSTR2, CCK2R or both. As used herein, a cancer that “overexpresses” a cell surface receptor is a cancer that has higher expression levels of the cell surface levels of the receptor compared to a non-cancerous cell of the same tissue type. A cancer that “overexpresses” includes a cancer that expresses the cell surface receptor, where a non-cancerous cell of the same tissue type does not express the cell surface receptor.

In an embodiment, the cancer is medullary thyroid carcinoma (MTC), a small cell lung cancer (SCLC) or a neuroendocrine tumor. In an embodiment, the cancer is medullary thyroid carcinoma (MTC), a small cell lung cancer (SCLC) or a neuroendocrine tumor, where SSTR2, CCK2R or both SSTR2 and CCK2R are expressed.

Efficaciousness of treatment is determined in association with any known method for diagnosing or treating the particular cancer. Alleviation of one or more symptoms of the cancer indicates that the compound or complex confers a clinical benefit.

The compound or complex may be administered in combination with at least one additional cancer therapy, including chemotherapy, radiation and/or immuno-oncology therapy. The other cancer therapy may be administered in any order with the at least one additional cancer therapy, for example simultaneously, sequentially or separately.

As used herein, “treating a cancer” includes, but is not limited to, reversing, alleviating or inhibiting the progression of the cancer or symptoms or conditions associated with the cancer. “Treating the cancer” also includes extending survival in a subject. Survival is optionally extended by at least 1, 2, 3, 6 or 12 months, or at least 2, 3, 4, 5 or 10 years over the survival that would be expected without treatment with a cytotoxic agent or composition as described herein. “Treating the cancer” also includes reducing tumor mass and/or reducing tumor. Optionally, tumor mass and/or tumor burden is reduced by at least 5, 10, 25, 50, 75 or 100% following treatment with a cytotoxic agent or composition as described herein. “Treating the cancer” also includes reducing the aggressiveness, grade and/or invasiveness of a tumor.

The application also includes a method of inhibiting proliferative activity in a cell, comprising administering an effective amount of one or more compounds or complexes of the application to the cell. The present application also includes a use of one or more compounds or complexes of the application for inhibition of proliferative activity in a cell as well as a use of one or more compounds one or more compounds or complexes of the application for the preparation of a medicament for inhibition of proliferative activity in a cell. The application further includes one or more compounds one or more compounds or complexes of the application for use in inhibiting proliferative activity in a cell. In one embodiment, the one or more compounds or complexes comprises a radionuclide for use in therapy, optionally ¹⁸⁸Re, ¹⁸⁶Re, ¹⁵³Sm, ⁶⁶Ho, ⁹⁰Y, ⁸⁹Sr, ¹¹¹In, ¹⁵³Gd, ²²⁵Ac, ²¹²Bi, ²¹³Bi, ²¹¹At, ⁶⁰Cu, ⁶¹Cu, ⁶⁷Cu, ⁶⁴Cu, ⁶²Cu, ¹⁹⁸Au, ⁹⁹Au, ^{195m}Pt, ^{193m}Pt, ¹⁹⁷Pt, ^{117m}Sn, ¹⁰³Pd, ¹⁰⁵Rh, ^{103m}Rh, ¹⁷Lu, ²²³Ra, ²²⁴Ra, ²²⁷Th, ²²⁹Th, ¹⁴⁹Tb, ³²P, ¹⁶¹Tb, ³³P, ¹²⁵I, ²⁰³Pb, ²¹²Pb, ²⁰¹Tl, ¹¹⁹Sb, ^{58m}Co, ⁴⁷Sc, ¹⁴⁹Pm ¹⁶¹Ho, ¹⁵⁹Gd, ¹⁴²Pr, ¹⁶⁶Ho, or ¹⁷⁵Yb.

The present application also includes a method of imaging a tissue in a subject by administering an imaging effective amount of one or more compounds or complexes of the application for use in imaging to a subject in need thereof and applying an imaging technique to detect emitted gamma rays. The present application also includes a use of one or more compounds or complexes of the application for use in imaging for imaging a tissue well as a use of one or more compounds or complexes of the application comprising a radionuclide for use in imaging for the preparation of a medicament for imaging a tissue. The application further includes one or more compounds or complexes of the application comprising a radionuclide for use in imaging for use in imaging a tissue. In some embodiments, the use further includes applying an imaging technique to detect emitted gamma rays. In one embodiment, the one or more compounds or complexes comprises a radionuclide for use in imaging, optionally ⁹⁹mTc, ¹⁸⁸Re, ¹⁸⁶Re, ¹⁵³Sm, ⁶⁶Ga, ⁶⁷Ga, ⁶⁸Ga, ¹¹¹In, ⁵⁹Fe, ⁶³Zn, ⁵²Fe, ⁵²Mn, ⁴⁵Ti, ⁶⁰Cu, ⁶¹Cu, ⁶⁷Cu, ⁶⁴Cu, ⁶²Cu, ⁸²Rb, ¹⁹⁸Au, ¹⁹⁹Au, ^{195m}Pt, ^{191m}Pt, ^{193m}Pt, ^{117m}Sn, ⁸⁹Zr, ¹⁷⁷Lu, ¹⁸F, ²⁰³Pb, ⁴⁴Sc, ⁵¹Cr, ^{101m}Rh, ¹⁶⁶Ho, or ¹²³I.

The present application also includes a method of diagnosing cancer in subject by administering a diagnostic effective amount of one or more compounds or complexes of the application to a subject in need thereof and applying an imaging technique to detect emitted gamma rays. The present application also includes a use of one or more compounds or complexes of the application for diagnosing cancer in well as a use of one or more compounds or complexes of the application for diagnosing cancer. The application further includes one or more compounds or complexes of the application comprising a radionuclide for use in imaging for use in diagnosing cancer. In some embodiments, the use further includes applying an imaging technique to detect emitted gamma rays. In one embodiment, the one or more compounds or complexes comprises a radionuclide for use in imaging, optionally ^{99m}Tc, ¹⁸⁸Re, ¹⁸⁶Re, ¹⁵³Sm, ⁶⁶Ga, ⁶⁷Ga, ⁶⁸Ga, ¹¹¹In, ⁵⁹Fe, ⁶³Zn, ⁵²Fe, ⁵²Mn, ⁴⁵Ti, ⁶⁰Cu, ⁶¹Cu, ⁶⁷Cu, ⁶⁴Cu, ⁶²Cu, ⁸²Rb, ¹⁹⁸Au, ¹⁹⁹Au, ^{19m}Pt, ^{191m}Pt, ^{193m}Pt, ^{117m}Sn, ⁸⁹Zr, ¹⁷⁷Lu, ¹⁸F, ²⁰³Pb, ⁴⁴Sc, ⁶¹Cr, ^{101m}Rh, ¹⁶⁶Ho, or ¹²³I.

In an embodiment, effective amounts vary according to factors such as the disease state, age, sex and/or weight of the subject. In a further embodiment, the amount of a given compound or complex that will correspond to an effective amount will vary depending upon factors, such as the given compound or complex, the pharmaceutical formulation, the route of administration, the type of condition, disease or disorder, the identity of the subject being treated, and the like, but can nevertheless be routinely determined by one skilled in the art. In an embodiment, the effective amount is one that following treatment therewith manifests as an improvement in or reduction of any disease symptom.

In an embodiment, the compound or complex is administered at least once a week. However, in another embodiment, the compound or complex is administered to the subject from about one time per two weeks, three weeks or one month. In another embodiment, the compound or complex is administered about one time per week to about once daily. In another embodiment, the compound or complex is administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound or complex is used for the treatment may increase or decrease over the course of a particular treatment regime. Changes in dosage may result and become apparent by standard diagnostic assays known in the art. In some instances, chronic administration is required. For example, the compound or complex is administered to the subject in an amount and for duration sufficient to treat the subject.

In an embodiment, the subject is a mammal. In another embodiment, the subject is human. In an embodiment, the subject is a non-human animal. In an embodiment, the subject is canine. In an embodiment, the subject is feline. Accordingly, the compounds, methods and uses of the present application are directed to both human and veterinary diseases, disorders and conditions.

The dosage of a compound or complex of the application varies depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, a compound or complex of the application is administered initially in a suitable dosage that is adjusted as required, depending on the clinical response. Dosages will generally be selected to maintain a serum level of the compound of the application from about 0.01 pg/cc to about 1000 pg/cc, or about 0.1 pg/cc to about 100 pg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg.

### V. Methods of Preparing the Compounds of the Application

Compounds and/or complexes of the present application or comparator compounds can be prepared by various synthetic processes. The choice of particular structural features and/or substituents may influence the selection of one process over another. The selection of a particular process to prepare a given compound and/or complexes of the present application or comparator compounds is within the purview of the person of skill in the art. Some starting materials for preparing compounds of the present application are available from commercial chemical sources. Other starting materials, for example as described below, are readily prepared from available precursors using straightforward transformations that are well known in the art.

In some embodiments, the compound of Formula I or comparator compound is prepared all or in part using solid phase peptide synthesis (SPPS) or solution phase coupling techniques known in the art, for example, using the synthetic procedures found in Stewart and Young, 1984, Solid Phase Synthesis, Second Edition, Pierce Chemical Co., Rockford, Ill.; Fields and Noble, 1990, “Solid phase peptide synthesis utilizing 9-fluorenylmethyloxycarbonyl amino acids,” Int. J. Pept. Protein Res. 35:161-214; Geysen et al., 1987, J. Immunol. Methods 102:259-274.

Accordingly, in some embodiments, in SPPS, an Nα-protected linker group, such as a tert-butoxycarbonyl (Boc) or 9-fluorenylmethyloxycarbonyl (Fmoc) amino acid linker group, is activated at the α-carbonyl and coupled with the deprotected Nα functionality of the solid phase support. The newly added Nα-protected linker group is then deprotected and coupled to the next Nα-protected linker group if necessary until the final cleavage step. It would be appreciated by the person skilled in the art the chemistry of the coupling, deprotection, and final cleavage step of the linker from the solid phase support depends on choice of a N-protecting group. In some embodiments, the cleavage is accomplished by treatment with acid, for example trifluoro acetic acid (TFA) optionally in the presence of scavenger reagents such as triisopropylsilane. In some embodiments, when the a N-protecting group is Fmoc, cleavage in acid will also result in deprotection of the side chains.

Therefore, in an exemplary embodiment, the compounds of Formula I or comparator compounds are prepared using fluorenylmethyloxycarbonyl (Fmoc) solid-phase peptide synthesis chemistry known in the art. Accordingly, in some embodiments, the compounds of Formula I or fragments therefore are prepared, manually or by using automated multiple solid-phase peptide synthesizer, using a Wang resin, Rink Amide-MBHA or equivalent resin and Fmoc-protected linker group derivatives with suitable side-chain protections such as Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-Lys(Mtt)-OH, Fmoc-Lys(ivDde)-OH, Fmoc-Nle-OH, Fmoc-8-amino-3,6-dioxaoctanoic acid (Fmoc-OEG-OH), Fmoc-Glu-OtBu. The resin is swelled using a suitable solvent such as combination of dichloromethane (DCM) and dimethylformamide (DMF). Prior to each coupling step the base-labile Nα-protecting group Fmoc is cleaved off from the Fmoc protected linker groups using a suitable base such as piperidine in a suitable solvent such as DMF for time to cleave to cleave the Fmoc protecting group, for example, about 10-15 min. The resin is then subsequently washed with a suitable solvent such as the DMF to, for example, remove piperidine. An excess amount of the Fmoc-linker group (e.g., 4 to 8 molar equivalent) is then coupled using coupling agents known in the art, for example, N,N′-diisopropylcarbodiimide (DIC) and ethyl cyanohydroxyiminoacetate (Oxyma, e.g Oxyma Pure®) or (Benzotriazol-1-yloxy)tripyrrolidinophosphonium hexafluorophosphate (PyBOP) and (1-Hydroxybenzotriazole (HOBt), in a suitable solvent such as DMF for about 1 to about 2 hours and then further washed with a suitable solvent, such as DMF. The coupling step is repeated once for each linker group.

When necessary, the methyltrityl (Mtt) group of the Fmoc-Lys(Mtt)-OH (i.e., N-α-Fmoc-N-ε-4-methyltrityl-L-lysine) linker group or the deprotected Lys(Mtt)-residue in the linker fragment is removed by treating the group or residue with hexafluoroisopropanol (HFIP) in a suitable solvent such as dichloromethane (DCM) (e.g. about 30% v/v) for suitable amount of time, for example, about 1 hour, followed by washing the resin with the suitable solvent and repeating the treatment with HFIP in DCM with a final washing with DCM after treatment.

After coupling, the compound of Formula I, comparator compound or fragment thereof is cleaved from the solid phase by treatment with a suitable acid for example, trifluoroacetic acid (TFA) and optionally in the presence of a trialkylsilane such as triisopropylsilane (TIP) and water and then precipitated with a suitable solvent such as diethyl ether. The product is dissolved in a suitable solvent such as water and acetonitrile and purified using high-performance liquid chromatography (HPLC) such as reversed phase HPLC using a suitable solvent or solvent mixture such as water with acetonitrile and TFA with an increasing gradient of acetonitrile. Relevant fractions are checked by analytical UPLC. Fractions containing the pure target compounds are pooled and freeze-dried.

The chelating group such as DOTA is conjugated to the linker fragment, for example, E-amine of a lysine residue of the linker fragment or the linker fragment attached to the tumour binding group and/or circulation enhancing group using active ester chemistry known in the art. For example, DOTA is combined with the linker fragment in the presence of a base such as an amine.

The chelating groups can be synthesized through methods known in the art or are commercially available. For example, DOTA is available from Sigma-Aldrich (St. Louis, Missouri, United States).

The formation of a desired compound salt is achieved using standard techniques. For example, the neutral compound is treated with an acid or base in a suitable solvent and the formed salt is isolated by filtration, extraction or any other suitable method.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T.W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

The following non-limiting examples are illustrative of the present application.

### EXAMPLES

The following non-limiting examples are illustrative of the present application.

### 332. Synthesis of Exemplary Compounds of Formula I

### General Methods

### Reagents

The Fmoc-protected amino acid derivatives used, unless specifically stated otherwise, were the standard recommended: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Leu-OH, Fmoc-Met-OH, Fmoc-Phe-OH, Fmoc-D-Phe-OH, Fmoc-Pro-OH, Fmoc-D-Pro-OH, Fmoc-Ser(tBu)-OH, Fmoc-Thr(tBu)-OH, Fmoc-Trp(Boc)-OH, Fmoc-D-Trp(Boc)-OH, Fmoc-Tyr(tBu)-OH, Fmoc-Val-OH, Fmoc-Lys(Mtt)-OH, Fmoc-Lys(ivDde)-OH, Fmoc-Glu-OtBu, Fmoc-Thr(tBu)-ol (CAS #189337-28-8), Fmoc-8-amino-3,6-dioxaoctanoic acid (Fmoc-OEG-OH), Fmoc-8-amino-octanoic acid (Fmoc-Aoc-OH), Fmoc-PEG₁-OH (Fmoc-HN—CH₂CH₂O—CH₂CH₂CO₂H), Fmoc-PEG₃-OH (Fmoc-HN-[CH₂CH₂O]₃-CH₂CH₂CO₂H), Fmoc-PEG₆-OH (Fmoc-HN-[CH₂CH₂O]₆-CH₂CH₂CO₂H) etc. Other reagents used included DOTA-tris(tert-butyl ester), DOTAGA-tetra (t-Bu ester), nastorazepide (Z360, CAS NO. 343326-69-2) etc.

### Loading of C-Terminal Residues

### C-Terminal Acid Peptides

The C-terminal acid peptides were prepared using Wang resin. The coupling of the first C-terminal residue started with swelling Wang resin (loading 1.1 mmol/g, 5.0 mmol) in DCM (50 mL) in a SPPS reaction vessel with N₂ bubbling for 30 min. The resin was then drained and washed with DMF (50 mL) 3 times. In a separate flask, a mixture of Fmoc-Thr(tBu)-OH (15 mmol), DIC (15 mmol) and 4-dimethylaminopyridine (DMAP) (0.5 mmol) in DMF (60 mL) was stirred for 15 min at r.t. before being transferred to the above reaction vessel. The resulting mixture was bubbled with N₂ for 4 h, then drained and washed with DMF (50 mL) 6 times, followed by the addition of DMF (60 mL), acetic anhydride (50 mmol) and DMAP (0.50 mmol). The resulting mixture was bubbled with N₂ for 2 h, then drained and the resin was washed with DMF (50 mL) 6 times.

### C-Terminal Amide Peptides

The C-terminal amide peptides were prepared using Rink Amide-MBHA resin. The first C-terminal residue (Fmoc-Lys(Mtt)-OH or Fmoc-Thr(tBu)-OH) was attached following a standard amide bond coupling conditions using PyBOP/HOBt/DIEA as coupling reagents. The resin was washed with DMF 6 times before the coupling of the 2^{nd} residue.

### C-Terminal Alcohol Peptides

The C-terminal alcohol peptides were prepared using Chlorotrityl Chloride (CTC) resin, and the first C-terminal residue, Fmoc-Thr(tBu)-ol, was attached to CTC resin by mixing 2 eq. of Fmoc-Thr(tBu)-ol with the resin in the presence of 3 eq. of DIEA in DMF at r.t. for 4 h, followed by washing and blocking with 50% MeOH/DMF at. R.t. for 1 h. The resin was then washed with DMF 6 times.

### Standard Solid-Phase Assembly Protocol

The synthesis was performed using Fmoc-based chemistry manually. The stepwise assembly was conducted following the below steps:
1) Pre-swell the resin with DCM and DMF;
2) Remove the Fmoc group by 20% piperidine; 2 treatments, 10 min each;
3) Wash the resin with DMF to remove piperidine;
4) add Fmoc-amino acid (1 mmol), PyBOP (1 mmol), HOBt (0.2 mmol) and DMF (5 mL) to a reaction vessel containing 0.20 mmol resin followed by addition of DIEA (2 mmol), the resulting mixture was mixed by bubbling nitrogen for 1-2 h;
5) Drain the resin, and wash the resin with DMF 6 times;
6) When needed, the N-epsilon-lysine Mtt protective group can be removed by treating the resin with 30% (v/v) HFIP in DCM for 1 h twice; drain the resin and wash it with DCM and DMF;
8) The final wash before resin cleavage was done with DMF (3 times), DCM (3 times) and MeOH (3 times), respectively.

### Resin Cleavage Condition and Purification

After solid-phase peptide assembly was completed, the resin was subject to a 1.5-3 h treatment of TFA/triisopropylsilane (TIS)/H₂O (95:2.5:2.5, v/v/v). The resin was filtered off and washed one time with TFA, the combined filtrate was treated with methyl tert-butyl ether (MTBE) to precipitate the crude peptide out of the solution. The precipitate was collected by centrifugation and washed 3 times with diethyl ether, briefly dried, then re-dissolved in acetonitrile (ACN) and H₂O to a concentration of 1 mM or lower. The volume ratio of ACN to H₂O can be adjusted to a relatively low percentage of ACN while ensuring a completely clear solution can still be achieved. The pH of the resulting solution was adjusted to 4-5 by the addition of acetic acid, then a solution of iodine in methanol (1.0 gram iodine in 100 mL methanol) was added dropwise until the iodine purple color stayed. The slightly purple mixture was then gently stirred for another 30 min before the addition of ascorbic acid solid (a few mg each portion) until the purple color completely disappeared. The resulting crude product was subjected to purification by using a reverse phase preparative HPLC system (Waters Delta Prep 4000) with a C18-reverse phase column. Mobile phases: A: 0.1% TFA/H₂O; B: 0.1% TFA/ACN. Relevant fractions were analyzed by analytical ultra-performance liquid chromatography (UPLC). The pure fractions were pooled and freeze-dried affording the product as a white lyophilized powder.

LC-MS condition
Instrument: Agilent prime-6125B_2LCMS
Column: Boltimate EXT C18 CoreShell 4.6×50 mm, 2.7 μm
Detection: UV (254 nm 214 nm 280 nm) and MS (ESI, 100 to 2000 amu)
Mobile Phase: A: H₂O (0.05% formic acid); B: ACN (0.05% formic acid)
Flow Rate: 2.0 mL/min
Column Temperature: 45° C.
Gradient: 10% to 95% B within 1.5 min, followed by 95% B for 1.0 min

### Analytical HPLC Condition

Instrument: WATERS ARC UPLC
Column: XBridge BEH peptide BEH C18, 3.5 μm, 2.1 mm×150 mm
Detection: UV 254 nm, 214 nm, 280 nm
Mobile Phase: A: H₂O (0.1% TFA); B: ACN (0.1% TFA)
Column Temperature: 40° C.
Flow Rate: 0.6 mL/min
Gradient:

| Time (min) | 0 | 1 | 11 | 13 | 13.5 | 15 |
|---|---|---|---|---|---|---|
| A % | 90 | 90 | 5 | 5 | 90 | 90 |
| B % | 10 | 10 | 95 | 95 | 10 | 10 |

### Radiochemistry Methods

### ¹²⁵I-Complex Synthesis

To a 38 μL of exemplary or comparator compound in solution (20 μM in 100 mM pH 7.5 sodium phosphate), was added Na ¹²⁵I solution (2.0 mCi, Perkin Elmer), then added 14 μL of a Chloramine-T solution (fresh 500 μM in 100 mM pH 7.5 sodium phosphate). The resulting solution was mixed well then left at room temperature for 5 min before the addition of 2 μL of the sodium ascorbate solution (fresh 50 mM in H₂O). The resulting crude product was mixed at room temperature for 2 min before loaded to Oasis HLB cartridge (10 mg) for purification. The cartridge was first washed with 0.80 mL×3 H₂O, then the product was eluted by 0.40 mL×3-80% aqueous ethanol. The fractions were analyzed by radio-thin-layer chromatography (TLC) on a polyamide film using methanol and 1M ammonium acetate as the mobile phase (v/v: 4:1) and detected by Mini Scan (Eckert & Ziegler Radiopharma Inc.). The selected fractions were pooled and diluted by 1% bovine serum albumin (BSA) and 5 mg/mL sodium ascorbate-containing pH 7.5 100 mM sodium phosphate buffer to a final activity of ~50-100 μCi/mL. The aliquoted products were stored at −80° C. until being used for the radioligand binding assay and expired at 4 weeks after synthesis.

¹²⁵I-C1 and ¹²⁵I-C3 were prepared using the above protocol.

### ¹⁷⁷Lu Complex Synthesis

To a 0.5 M NaOAc buffer (20-50 μL, pH=4.5) was added 4 μL of exemplary or comparator compound in DMSO stock solution (2000 μM) and 2 mCi ¹⁷⁷Lu (ITM Isotope Technologies Munich), the resulting mixture was heated to 95° C. for 15 min. The resulting product was analyzed by radio-TLC and radio-HPLC (column: Shim-pack GIST 5 μm 4.6*150 mm; buffer A: 0.2% formic acid H₂O; buffer B: 0.1% formic acid acetonitrile; flow rate: 1 mL/min; gradient: 0-5 min: 10% B to 95% B; then 5-8 min: 95% B). The labelled products were used immediately or diluted by a PBS buffer containing freshly added 3 mg/mL ascorbate.

¹⁷⁷Lu complexes of the application were prepared using protocols described above.

### Synthesis of Exemplary Compounds of Formula I

The following exemplary compounds of Formula I were prepared using the above methods:

### Example A1: DOTA-K(-gE-G-Z360)-OEG-f[CYwKTC]T-OH (I-1)

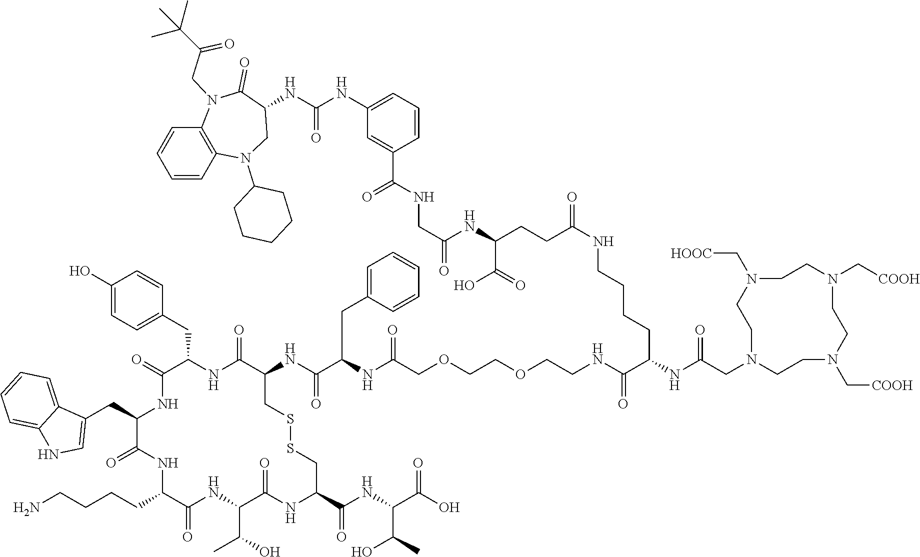 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2397.8 g/mol.

Determined by LC-MS: (M+2H)2+: 1199.7; (M+3H)_{3+: 799.9}; (M+4H)4+: 600.4.

Purity by UPLC (214 nm): >99.0%.

### Example A2: DOTA-K(-gE-G-Z360)-OEG-OEG-f[CYwKTC]T-OH (I-2)

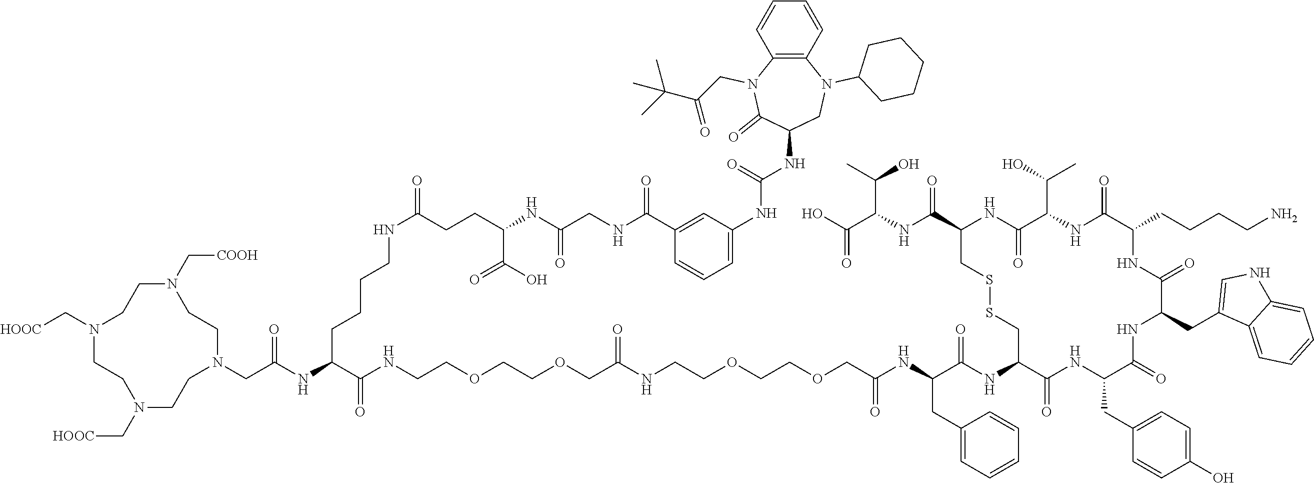 [SMILES: C[C@@H](O)[C@H](NC(=O)[C@@H]1CSSC[C@H](NC(=O)[C@@H](Cc2ccccc2)NC(=O)COCCOCCNC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2542.9 g/mol.

Determined by LC-MS: (M+2H)2+: 1271.8; (M+3H)_{3+: 848.3}; (M+4H)4+: 636.7.

Purity by UPLC (214 nm): 98.8%.

### Example A3: DOTA-K(-E-Z360)-OEG-f[CYwKTC]T-NH₂ (1-3)

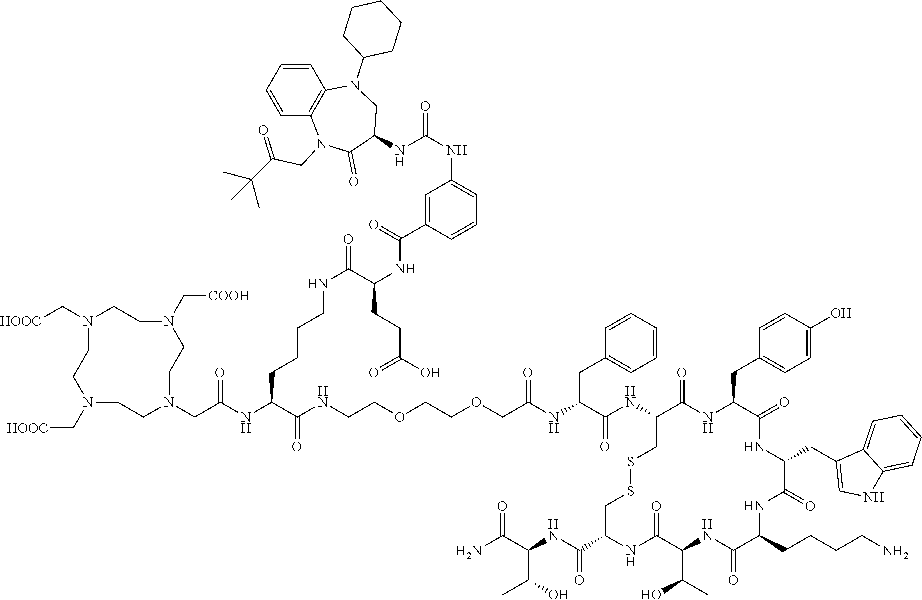 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(N)=O]

Molecular weight (average) calculated: 2339.7 g/mol

Determined by LC-MS: (M+2H)2+: 1170.4; (M+3H)_{3+: 780.8}; (M+4H)4+: 585.8

Purity by UPLC (214 nm): >99.0%

### Example A4: DOTA-K(-E-Z360)-f[CYwKTC]T-NH₂ (1-4)

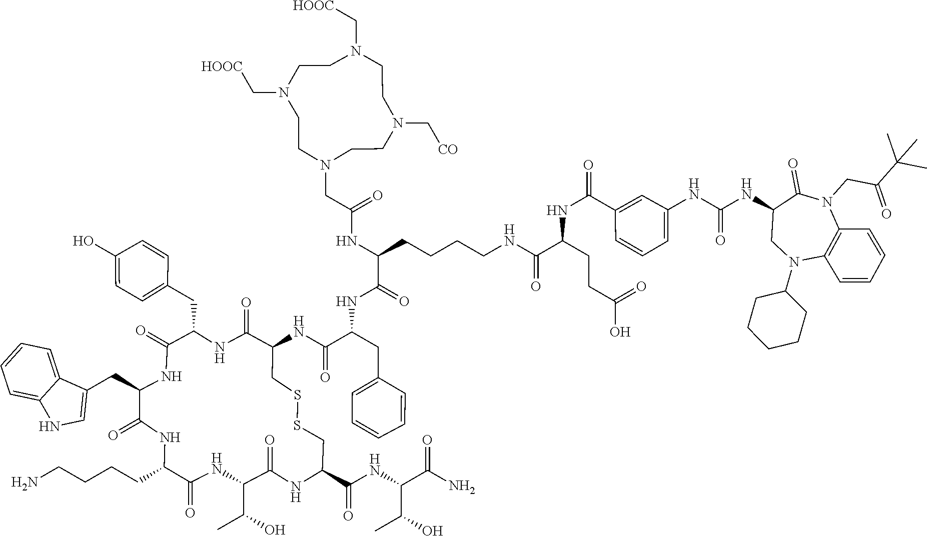

Molecular weight (average) calculated: 2194.6 g/mol

Determined by LC-MS: (M+2H)2+: 1097.9; (M+3H)_{3+: 732.2}; (M+4H)4+: 549.5

Purity by UPLC (214 nm): 90.3%

### Example A5: DOTA-K(-E-Z360)-OEG-f[CYwKTC]-Thr(ol) (1-5)

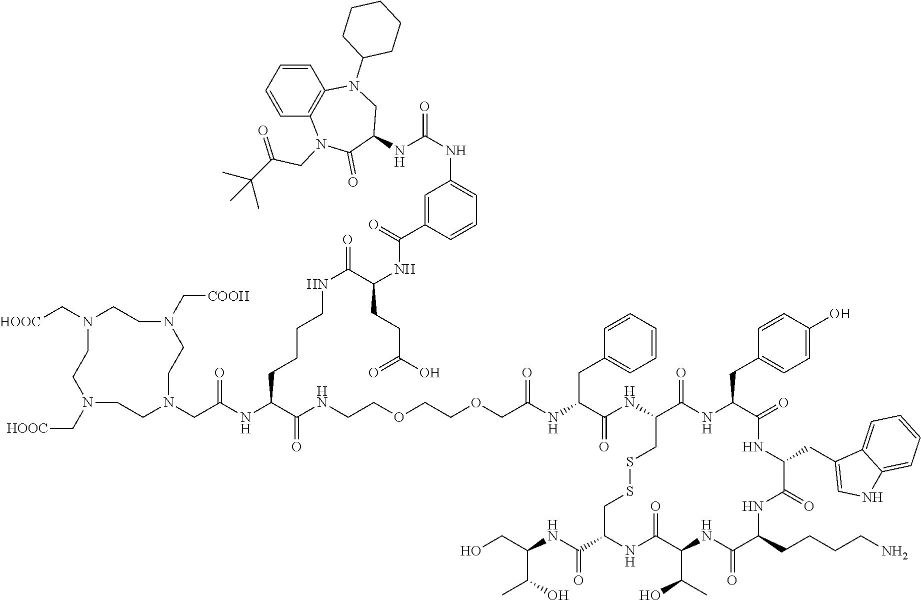 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)COCCOCCNC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)CSSC[C@@H](C(=O)N[C@H](CO)[C@@H](C)O)NC1=O]

Molecular weight (average) calculated: 2326.7 g/mol

Determined by LC-MS: (M+2H)2+: 1163.9; (M+3H)_{3+: 776.3}; (M+4H)4+: 582.7

Purity by UPLC (214 nm): >99.0%

### Example A6: DOTA-K(-E-Z360)-f[CYwKTC]-Thr(ol) (1-6)

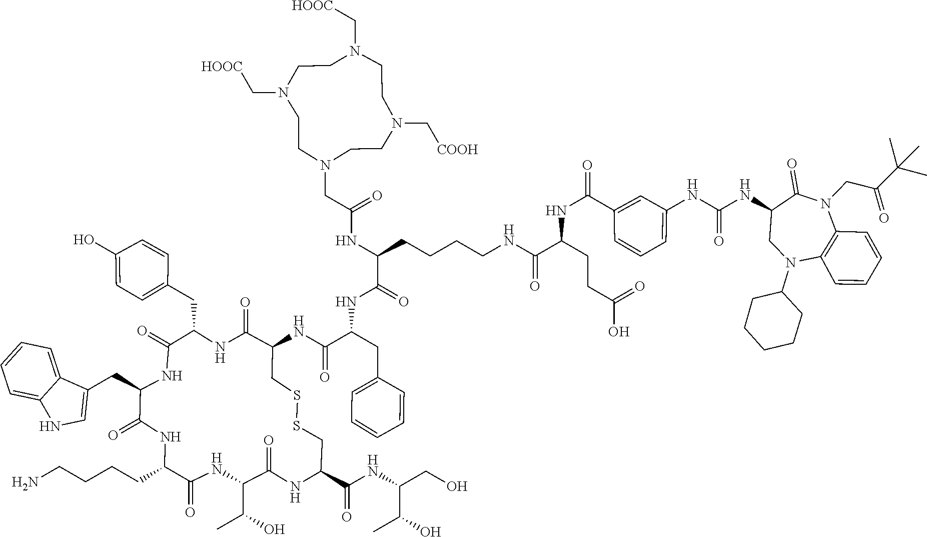 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)CSSC[C@@H](C(=O)N[C@H](CO)[C@@H](C)O)NC1=O]

Molecular weight (average) calculated: 2181.6 g/mol

Determined by LC-MS: (M+2H)2+: 1091.4; (M+3H)_{3+: 728.0}; (M+4H)4+: 546.3

Purity by UPLC (214 nm): 93.6%

### Example A7: DOTA-gE-gE-gE-gE-OEG-K(-gE-G-Z360)-OEG-f[CYwKTC]T-OH (I-7)

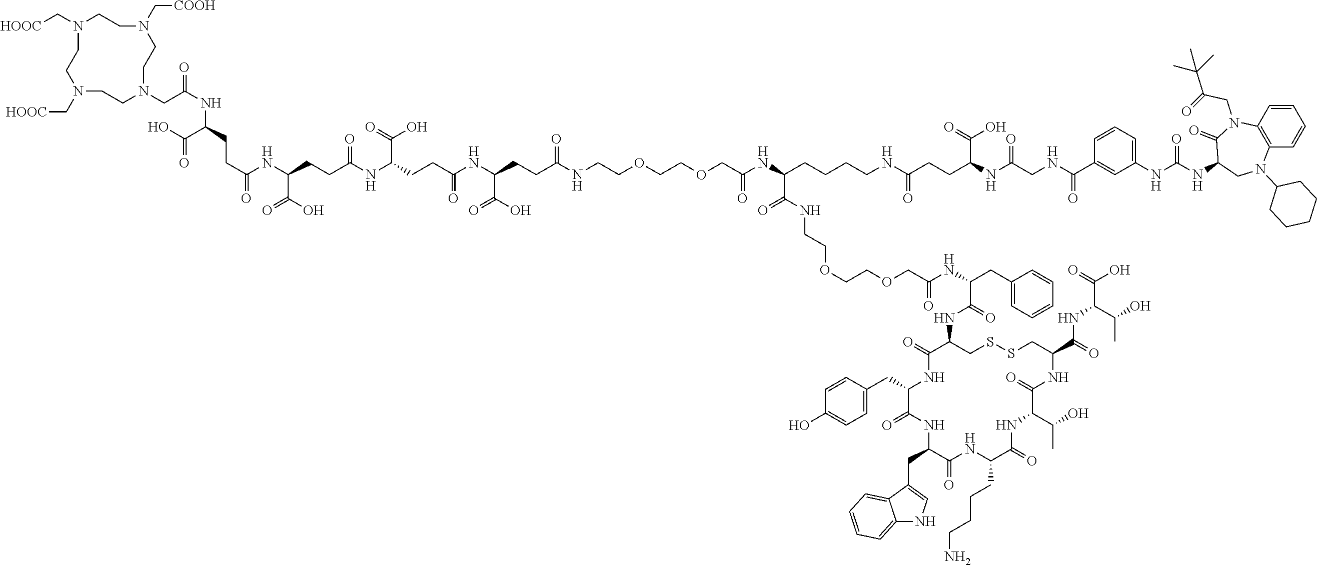 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CC[C@H](NC(=O)CC[C@H](NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 3059.4 g/mol

Determined by LC-MS: (M+2H)2+: 1530.0; (M+3H)_{3+: 1020.4}; (M+4H)4+: 765.7

Purity by UPLC (214 nm): 94.7%

### Example A8: DOTA-gE-gE-gE-OEG-Lys(-gE-G-Z360)-OEG-f[CYwKTC]T-OH (I-8)

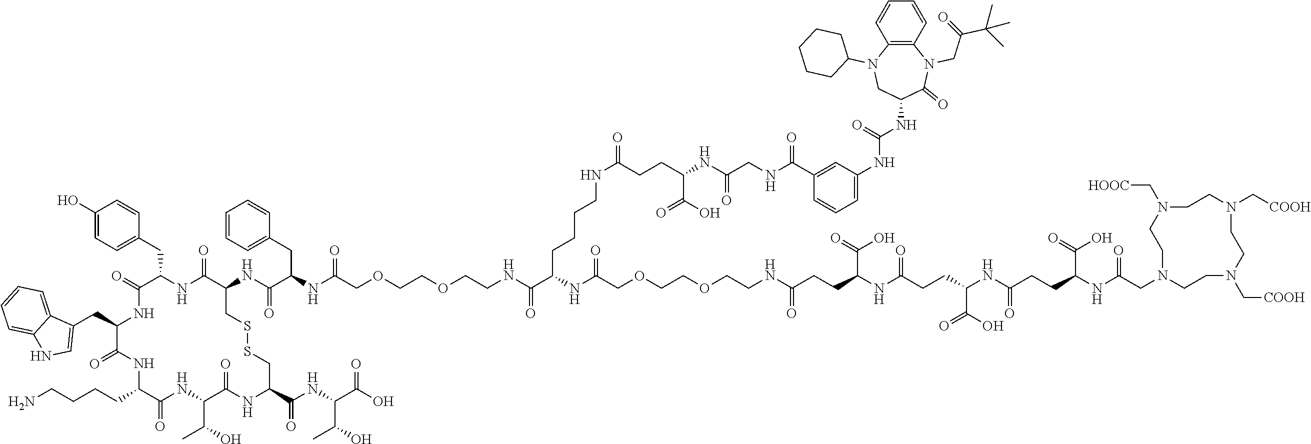 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CC[C@H](NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2930.3 g/mol

Determined by (M+2H)2+: 1465.4; (M+3H)_{3+: 977.3}; (M+4H)4+: 733.4

Purity by UPLC (214 nm): >99.0%

### Example A9: DOTA-gE-K(-gE-G-Z360)-gE-f[CYwKTC]T-OH (1-9)

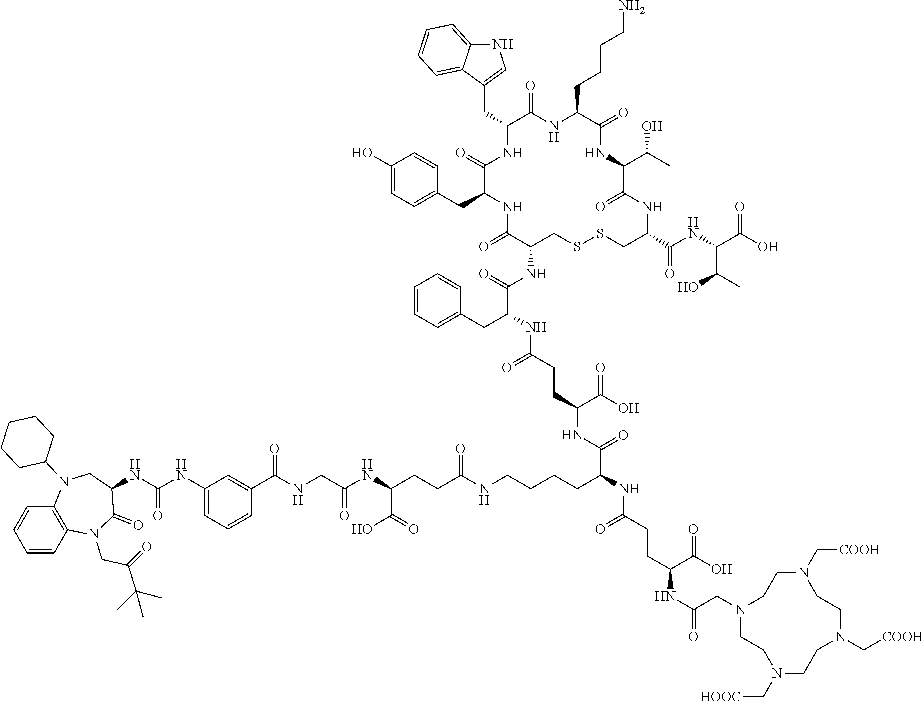 [SMILES: C[C@@H](O)[C@H](NC(=O)[C@@H]1CSSC[C@H](NC(=O)[C@@H](Cc2ccccc2)NC(=O)CC[C@H](NC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2510.8 g/mol

Determined by LC-MS: (M+2H)2+: 1255.8; (M+3H)_{3+: 837.7}; (M+4H)4+: 628.6

Purity by UPLC (214 nm): >99.0%

### Example A10: DOTA-gE-gE-K(-gE-G-Z360)-OEG-f[CYwKTC]T-OH (I-10)

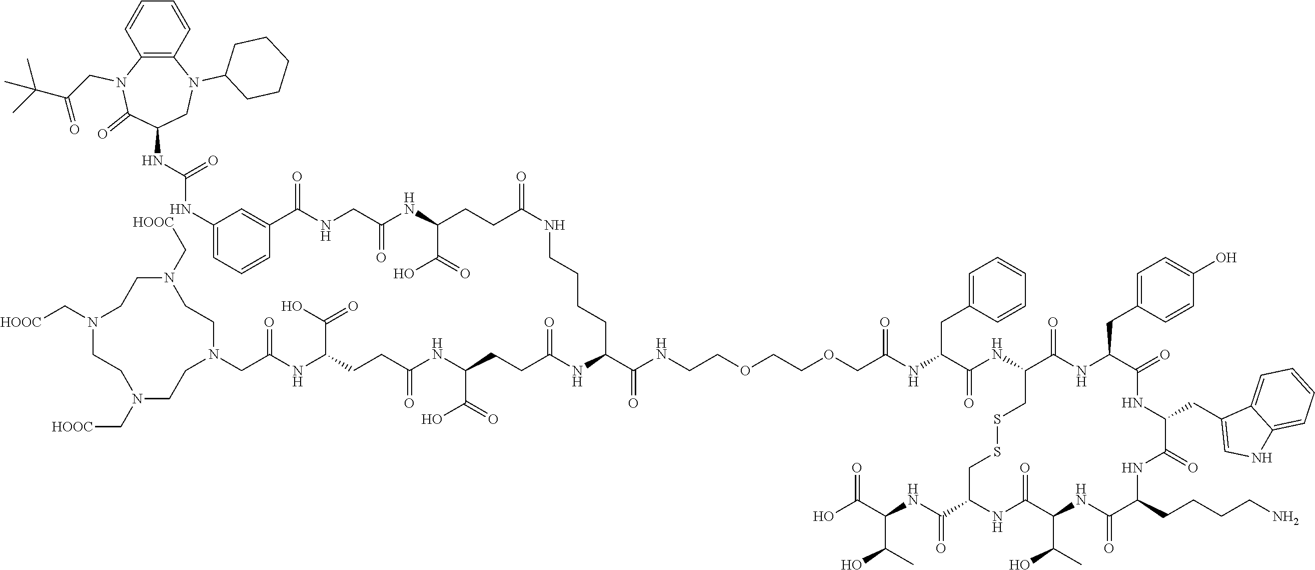 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2656.0 g/mol

Determined by LC-MS: (M+2H)2+: 1328.2; (M+3H)_{3+: 886.0}; (M+4H)4+: 664.8

Purity by UPLC (214 nm): 91.4%

### Example A11: DOTA-gE-gE-OEG-K(-E-Z360)-OEG-f[CYwKTC]T-OH (I-11)

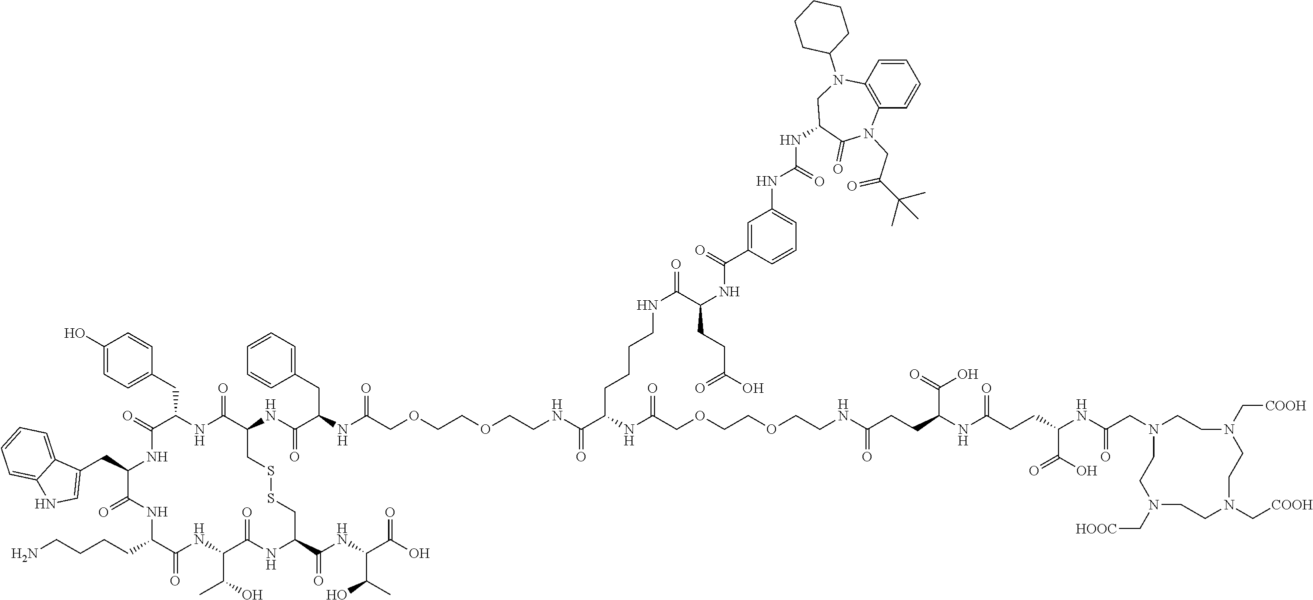 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)COCCOCCNC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2744.1 g/mol

Determined by LC-MS: (M+2H)2+: 1372.4; (M+3H)_{3+: 915.5}; (M+4H)4+: 686.8

Purity by UPLC (214 nm): 95.5%

### Example A12: (Z360)-E-gE-OEG-gE-K(DOTA)-OEG-f[CYwKTC]T-OH (I-12)

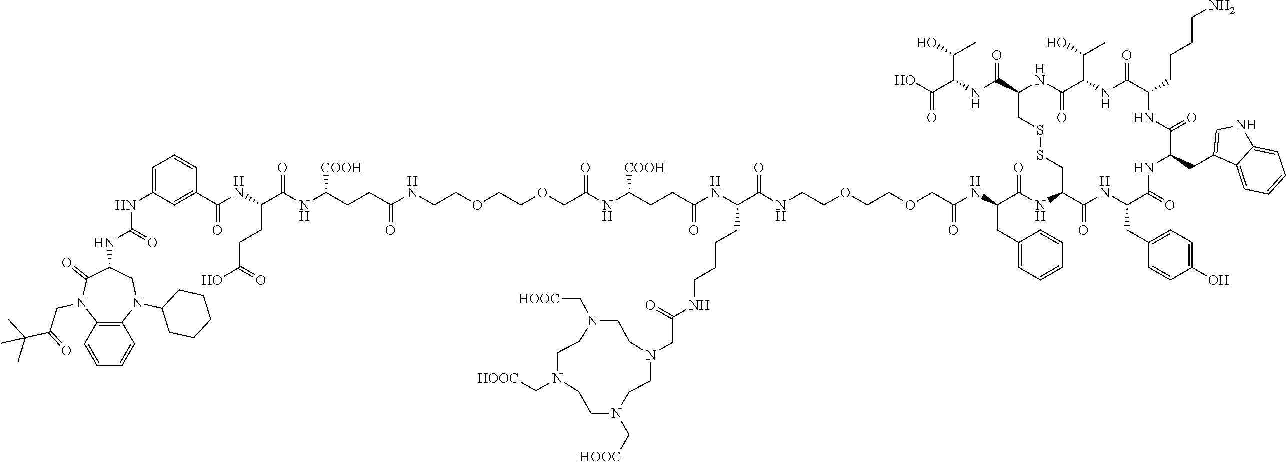 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CN2CCN(CC(=O)O)CCN(CC(=O)O)CCN(CC(=O)O)CC2)NC(=O)CC[C@H](NC(=O)COCCOCCNC(=O)CC[C@H](NC(=O)[C@H](CCC(=O)O)NC(=O)c2cccc(NC(=O)N[C@@H]3CN(C4CCCCC4)c4ccccc4N(CC(=O)C(C)(C)C)C3=O)c2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2744.1 g/mol

Determined by LC-MS: (M+2H)2+: 1372.4; (M+3H)_{3+: 915.4}; (M+4H)4+: 687.0

Purity by UPLC (214 nm): 97.4%

### Example A13: (Z360)-gE-gE-OEG-K(DOTAGA)-OEG-f[CYwKTC]T-OH (I-13)

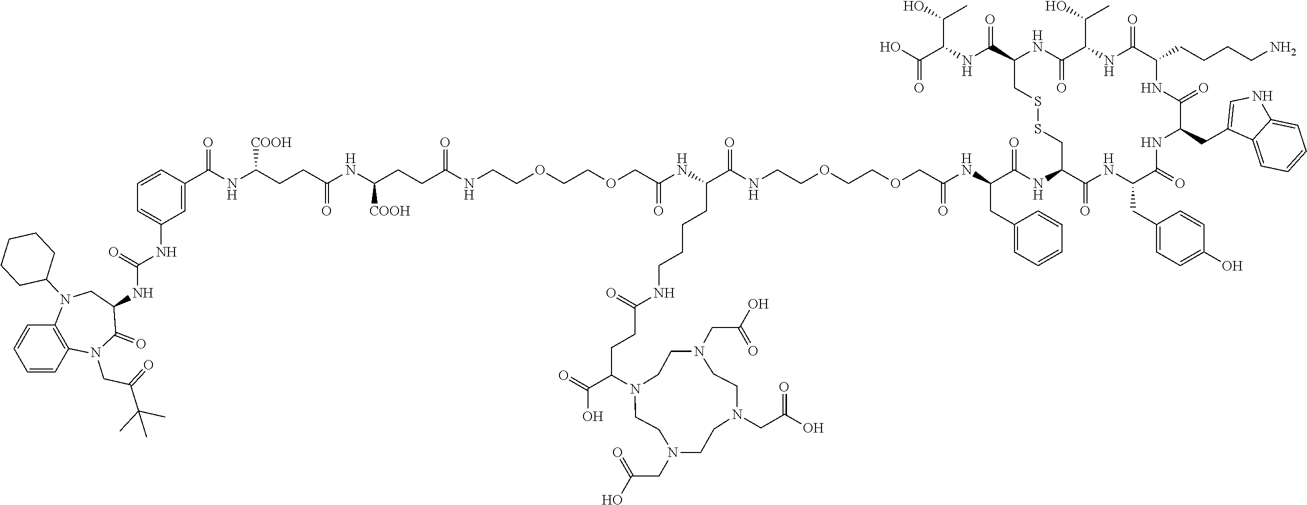 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CCC(C(=O)O)N2CCN(CC(=O)O)CCN(CC(=O)O)CCN(CC(=O)O)CC2)NC(=O)COCCOCCNC(=O)CC[C@H](NC(=O)CC[C@H](NC(=O)c2cccc(NC(=O)N[C@@H]3CN(C4CCCCC4)c4ccccc4N(CC(=O)C(C)(C)C)C3=O)c2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2687.0 g/mol

Determined by LC-MS: (M+3H)_{3+: 896.3}; (M+4H)4+: 672.6

Purity by UPLC (214 nm): 87.1%

### Example A14: DOTA-gE-gE-OEG-K(-gE-G-Z360)-OEG-f[CYwKTC]T-OH (I-14)

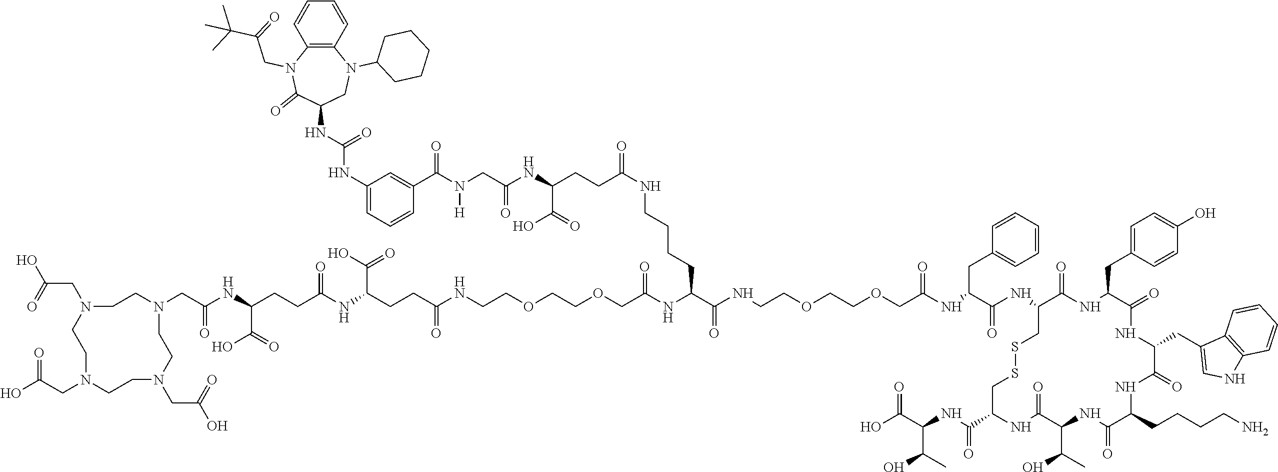 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2801.1 g/mol

Determined by LC-MS: (M+2H)2+: 1401.0; (M+3H)_{3+: 934.2}; (M+4H)4+: 701.2

Purity by UPLC (214 nm): >99.0%

### Example A15: DOTA-gE-gE-OEG-OEG-K(-gE-G-Z360)-OEG-f[CYwKTC]T-OH (l-15)

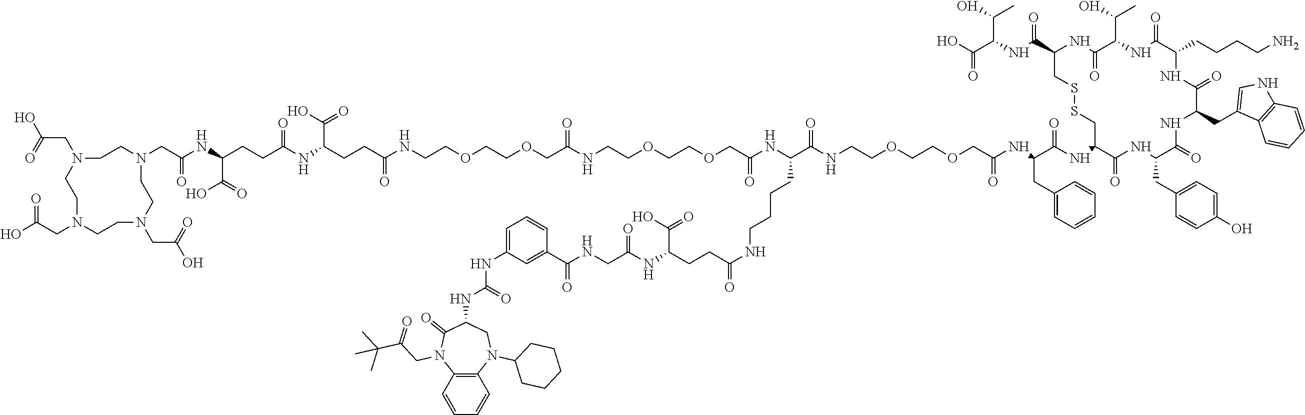 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2946.3 g/mol

Determined by LC-MS: (M+3H)_{3+: 982.8}; (M+4H)4+: 737.4

Purity by UPLC (214 nm): >99.0%

### Example A16: DOTA-gE-gE-OEG-OEG-K(-gE-G-Z360)-OEG-OEG-f[CYwKTC]T-OH (I-16)

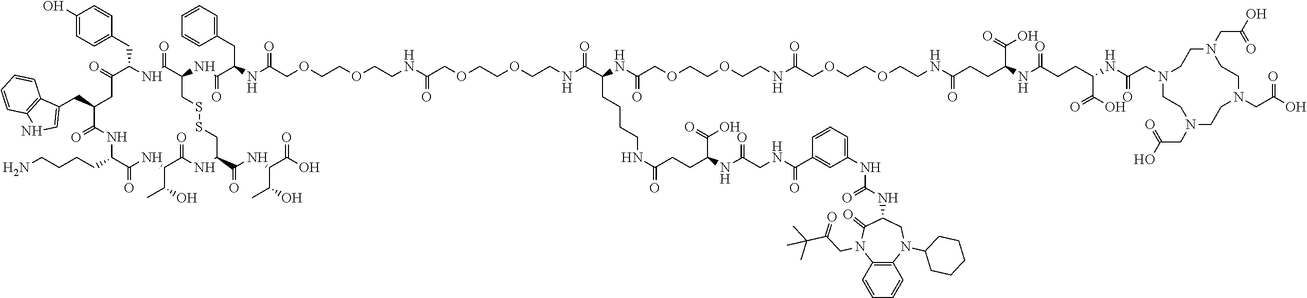

Molecular weight (average) calculated: 3091.5 g/molPG-3,l

Determined by LC-MS: (M+3H)_{3+: 1031.2}; (M+4H)4+: 773.7

Purity by UPLC (214 nm): 91.8%

### Example A17: (Z360)-gE-gE-OEG-OEG-K(DOTAGA)-OEG-OEG-f[CYwKTC]T-OH (I-17)

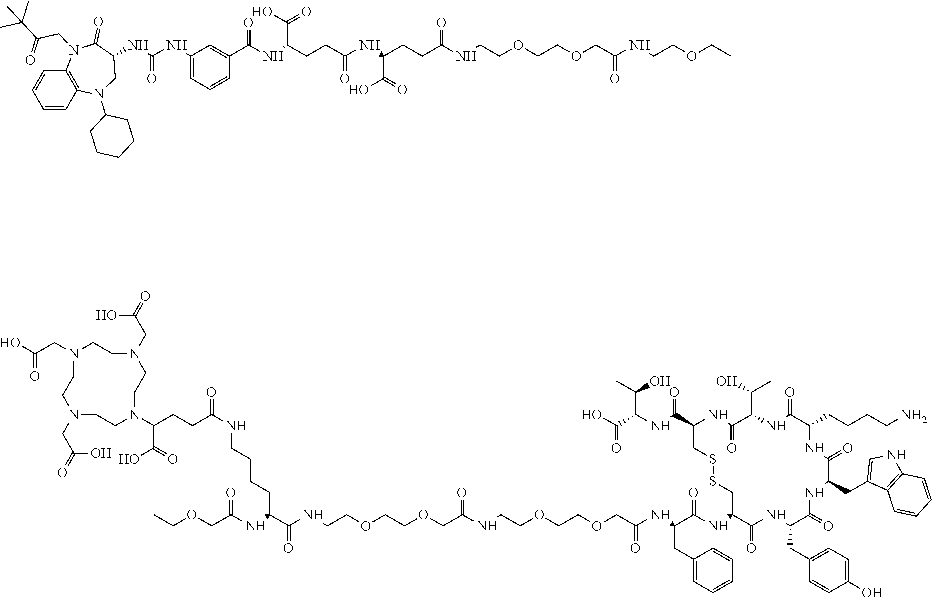 [SMILES: CCOCCNC(=O)COCCOCCNC(=O)CC[C@H](NC(=O)CC[C@H](NC(=O)c1cccc(NC(=O)N[C@@H]2CN(C3CCCCC3)c3ccccc3N(CC(=O)C(C)(C)C)C2=O)c1)C(=O)O)C(=O)O ; CCOCC(=O)N[C@@H](CCCCNC(=O)CCC(C(=O)O)N1CCN(CC(=O)O)CCN(CC(=O)O)CCN(CC(=O)O)CC1)C(=O)NCCOCCOCC(=O)NCCOCCOCC(=O)N[C@H](Cc1ccccc1)C(=O)N[C@H]1CSSC[C@@H](C(=O)N[C@H](C(=O)O)[C@@H](C)O)NC(=O)[C@H]([C@@H](C)O)NC(=O)[C@H](CCCCN)NC(=O)[C@@H](Cc2c[nH]c3ccccc23)NC(=O)[C@H](Cc2ccc(O)cc2)NC1=O]

Molecular weight (average) calculated: 2977.4 g/mol

Determined by LC-MS: (M+3H)_{3+: 993.0}; (M+4H)4+: 745.2

Purity by UPLC (214 nm): 87.5%

### Example A18: DOTA-gE-OEG-OEG-OEG-OEG-K(-gE-G-Z360)-OEG-gE-OEG-OEG-f[CYwKTC]T-OH (I-18)

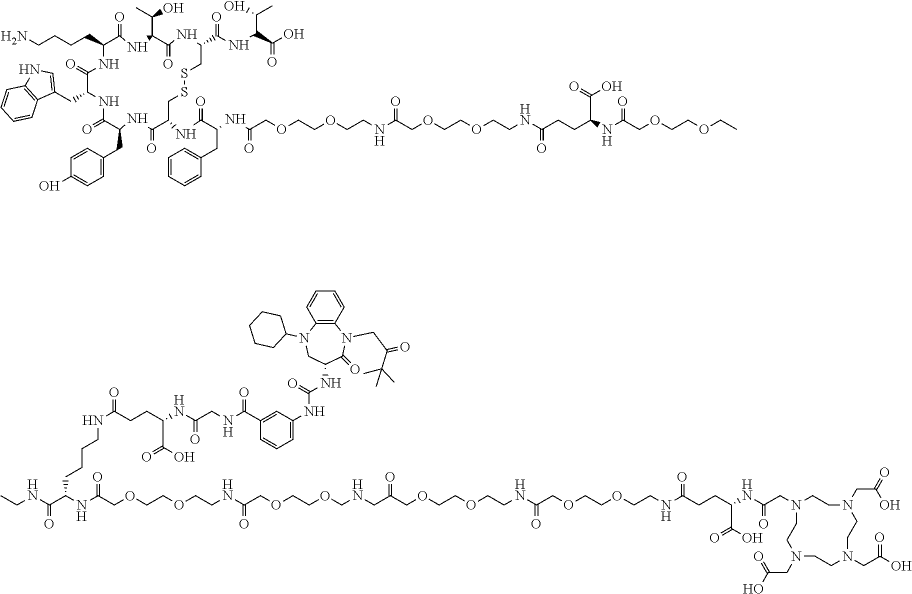

Molecular weight (average) calculated: 3526.9 g/mol

Determined by LC-MS: (M+2H)2+: 1176.3; (M+3H)_{3+: 882.5}; (M+4H)4+: 706.3

Purity by UPLC (214 nm): >99.0%

### Example A19: DOTAGA-OEG-K(-gE-G-Z360)-f[CYwKTC]T-OH (I-19)

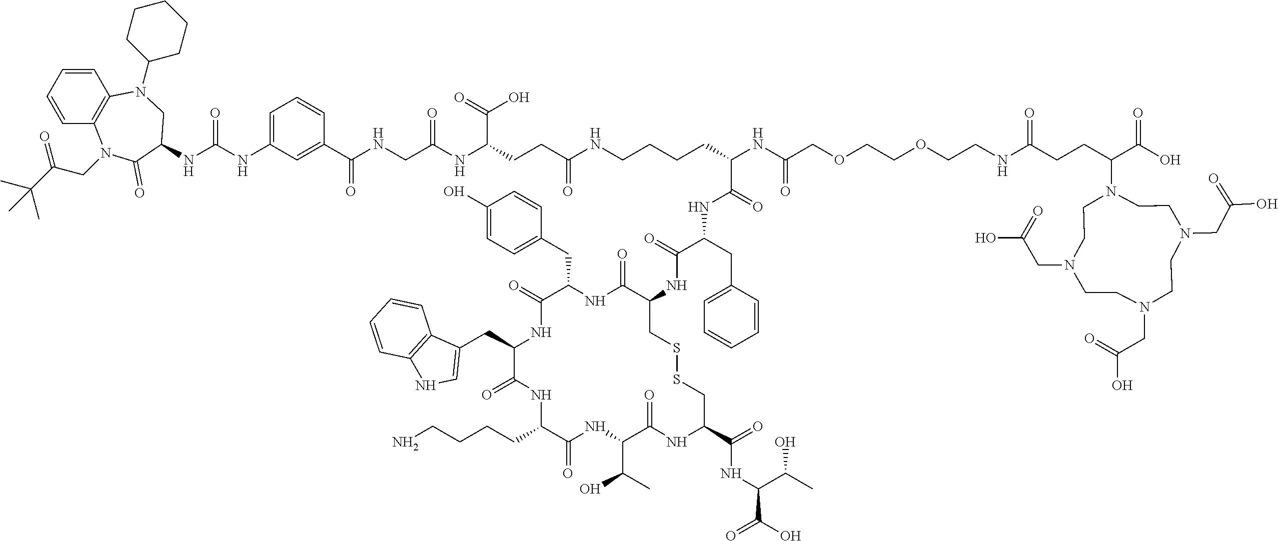 [SMILES: C[C@@H](O)[C@H](NC(=O)[C@@H]1CSSC[C@H](NC(=O)[C@@H](Cc2ccccc2)NC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CCC(C(=O)O)N2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2469.8 g/mol

Determined by LC-MS: (M+2H)2+: 1235.6; (M+3H)_{3+: 823.9}; (M+4H)4+: 618.4

Purity by UPLC (214 nm): 93.7%

### Example A20: DOTAGA-OEG-K(-gE-G-Z360)-OEG-f[CYwKTC]T-OH (1-20)

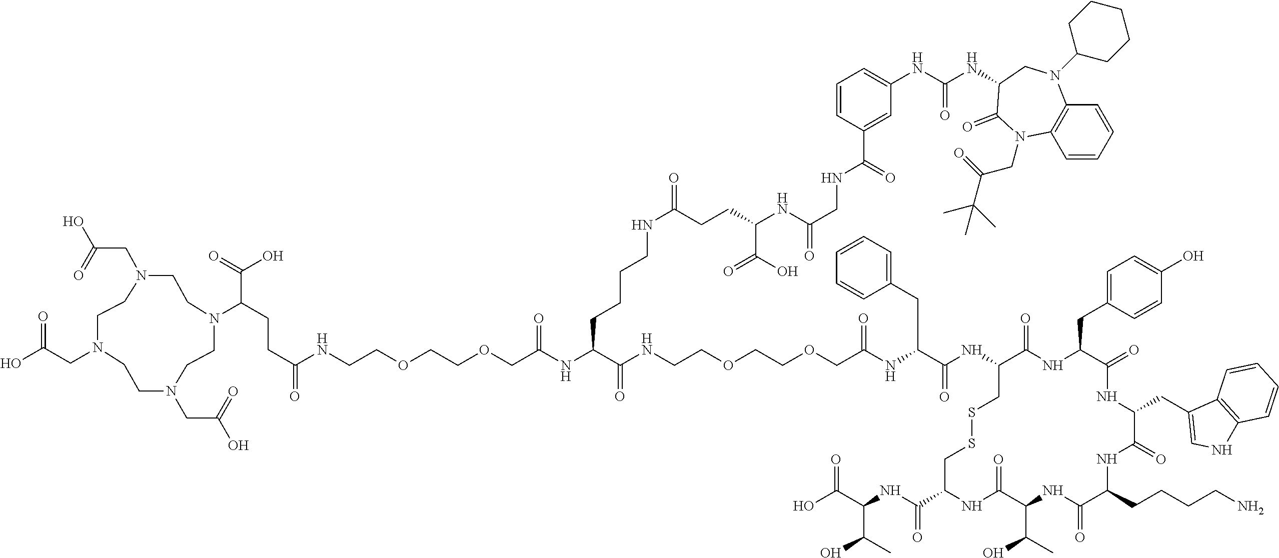 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CCC(C(=O)O)N2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2615.0 g/mol PGP-139′I1I

Determined by LC-MS: (M+2H)2+: 1307.9; (M+3H)_{3+: 872.3}; (M+4H)4+: 654.7

Purity by UPLC (214 nm): 93.9%

### Example A21: DOTA-gE-OEG-OEG-K(-gE-G-Z360)-OEG-f[CYwKTC]T-OH (I-21)

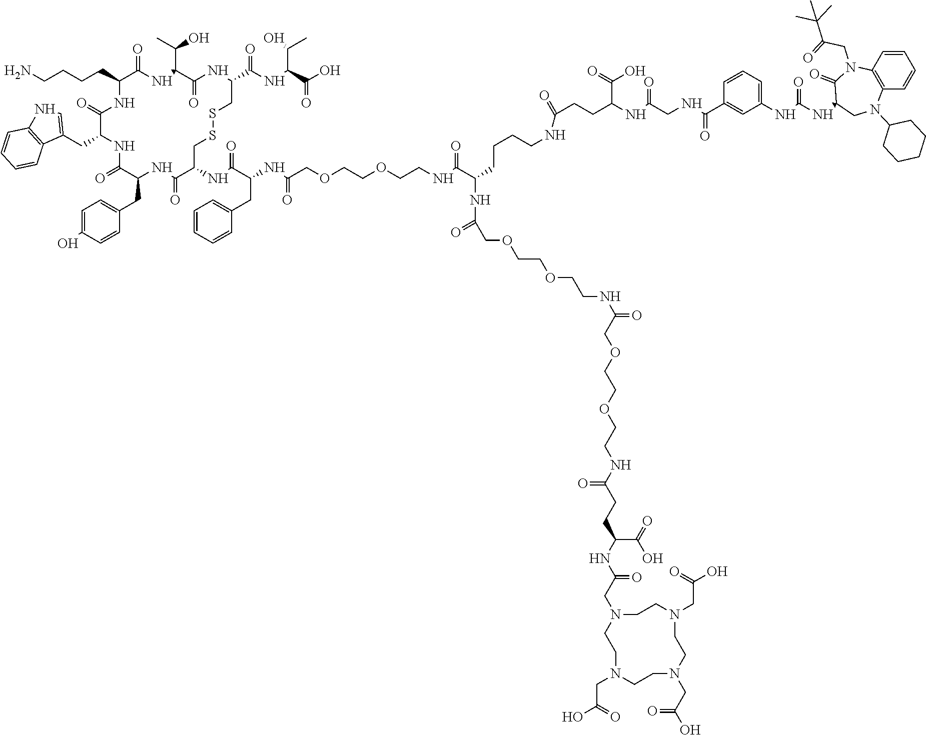 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CCC(NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)CC[C@H](NC(=O)CN2CCN(CC(=O)O)CCN(CC(=O)O)CCN(CC(=O)O)CC2)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2817.2 g/mol

Determined by LC-MS: (M+2H)2+: 1409.1; (M+3H)_{3+: 939.8}; (M+4H)4+: 705.3

Purity by UPLC (214 nm): >99.0%

### Example A22: DOTA-gE-OEG-OEG-OEG-OEG-OEG-K(-gE-G-Z360)-OEG-f[CYwKTC]T-OH (I-22)

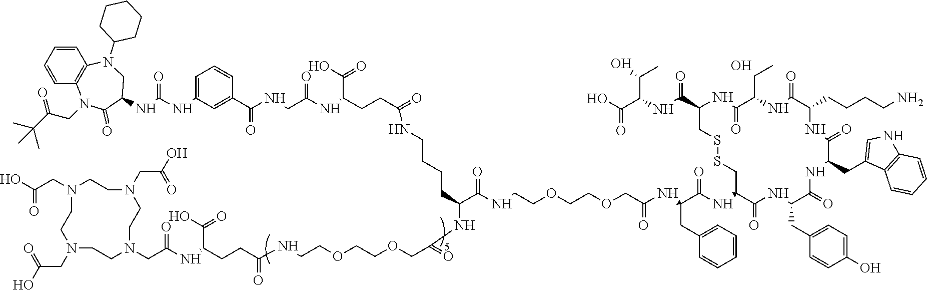

Molecular weight (average) calculated: 3252.7 g/mol

Determined by LC-MS: (M+3H)_{3+: 1084.9}; (M+4H)4+: 814.0; (M+5H)5+: 651.4

Purity by UPLC (214 nm): >99.0%

### Example A23: DOTA-OEG-OEG-OEG-K(-gE-G-Z360)-OEG-gE-OEG-OEG-f[CYwKTC]T-OH (I-23)

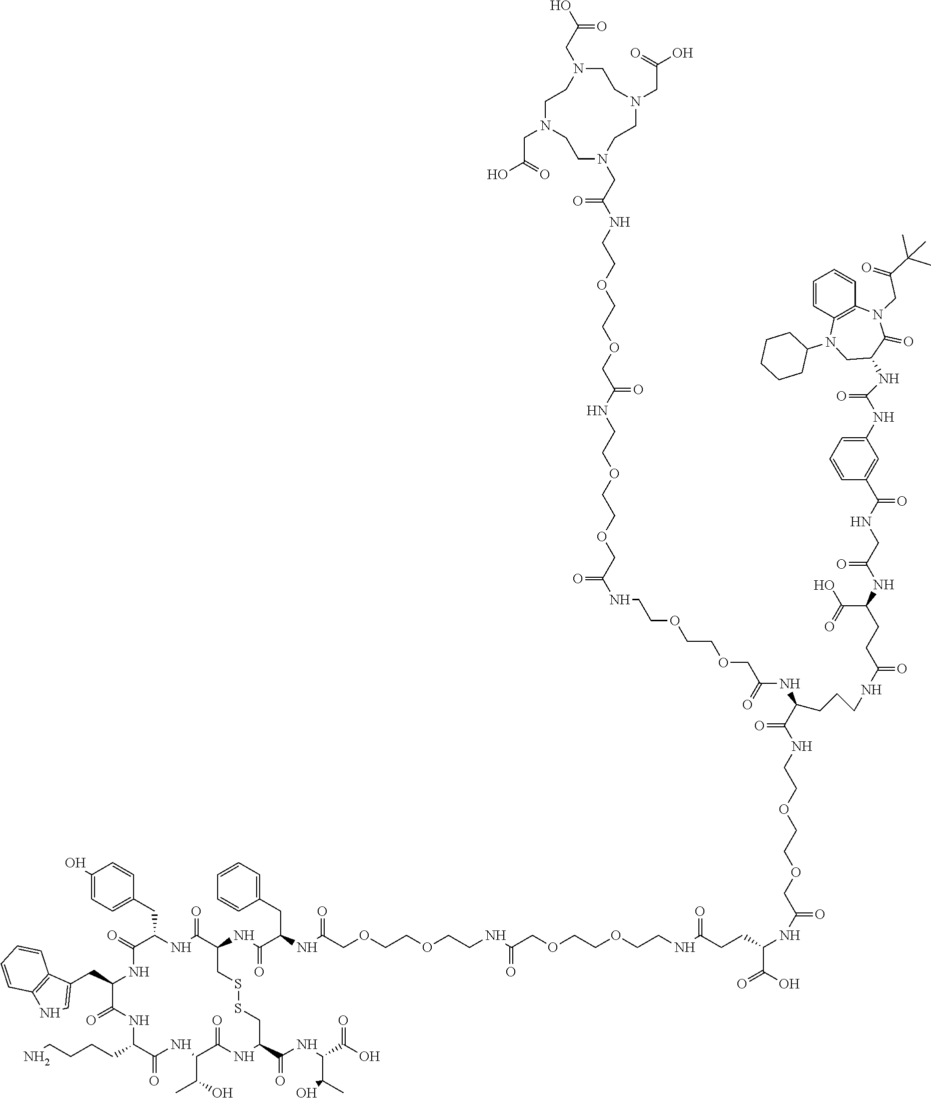 [SMILES: C[C@@H](O)[C@H](NC(=O)[C@@H]1CSSC[C@H](NC(=O)[C@@H](Cc2ccccc2)NC(=O)COCCOCCNC(=O)COCCOCCNC(=O)CC[C@H](NC(=O)COCCOCCNC(=O)[C@H](CCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)COCCOCCNC(=O)CN2CCN(CC(=O)O)CCN(CC(=O)O)CCN(CC(=O)O)CC2)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 3252.7 g/mol

Determined by LC-MS: (M+2H)2+: 1626.6; (M+3H)_{3+: 1084.9}; (M+4H)4+: 814.0

Purity by UPLC (214 nm): >99.0%

### Example A24: DOTA-gE-OEG-OEG-OEG-OEG-K(-gE-G-Z360)-OEG-OEG-OEG-OEG-f[CYwKTC]T-OH (I-24)

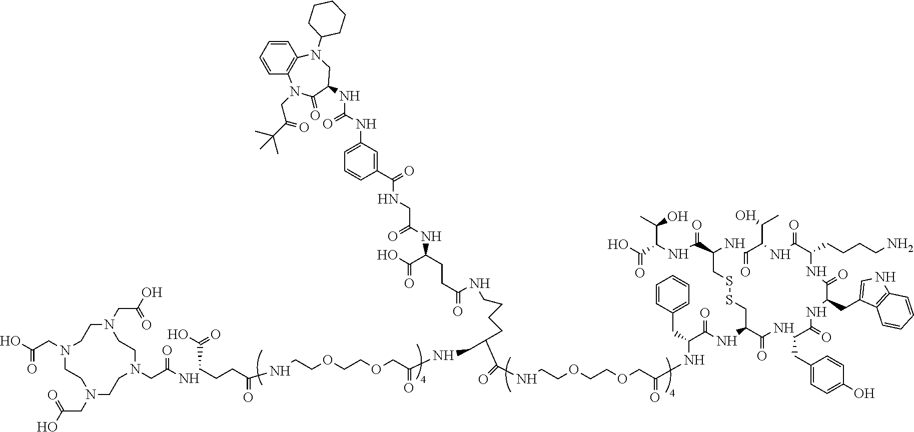

Molecular weight (average) calculated: 3543.0 g/mol

Determined by LC-MS: (M+3H)_{3+: 1181.6}; (M+4H)4+: 886.5; (M+5H)5+: 709.4

Purity by UPLC (214 nm): >99.0%

### Example A25: DOTA-K(-E-Z360)-f[CYwKTC]T-OH (I-25)

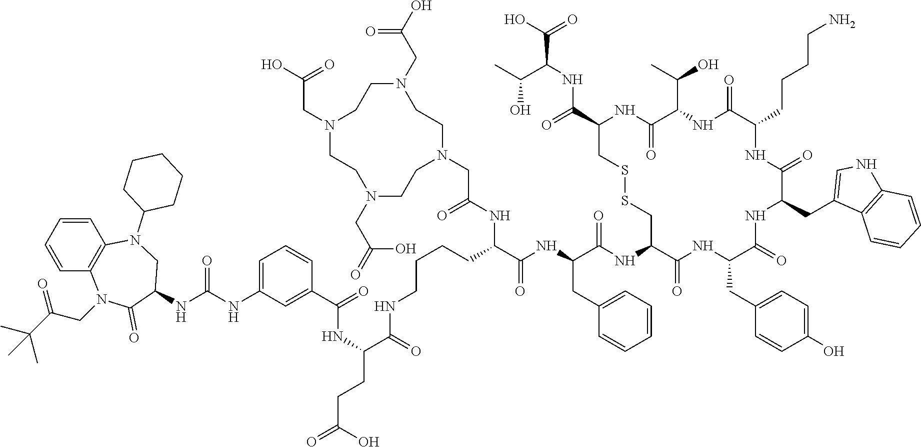 [SMILES: C[C@@H](O)[C@H](NC(=O)[C@@H]1CSSC[C@H](NC(=O)[C@@H](Cc2ccccc2)NC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

### Example A26: DOTA-K(-gE-G-Z360)-f[CYwKTC]T-OH (I-26)

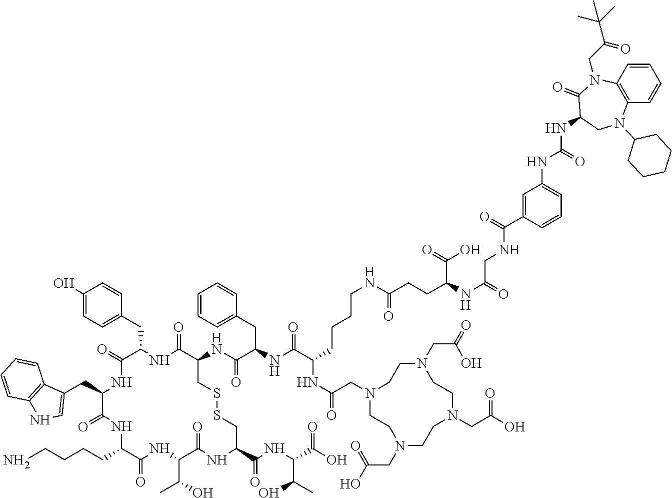 [SMILES: C[C@@H](O)[C@H](NC(=O)[C@@H]1CSSC[C@H](NC(=O)[C@@H](Cc2ccccc2)NC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2252.6 g/mol

Determined by LC-MS: (M+2H)2+: 1126.9; (M+3H)_{3+: 751.6}; (M+4H)4+: 564.2

Purity by UPLC (214 nm): 96.6%

### Example A27: DOTA-K(-E-Z360)-OEG-f[CYwKTC]T-OH (I-27)

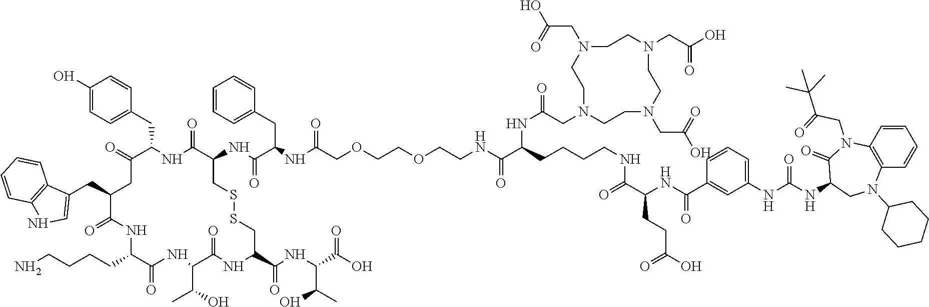

Molecular weight (average) calculated: 2340.7 g/mol

Determined by LC-MS: (M+2H)2+: 1170.2; (M+3H)_{3+: 780.8}; (M+4H)4+: 586.1

Purity by UPLC (214 nm): 99.0%

### Example A28: DOTA-K(-OEG-E-Z360)-f[CYwKTC]T-OH (I-28)

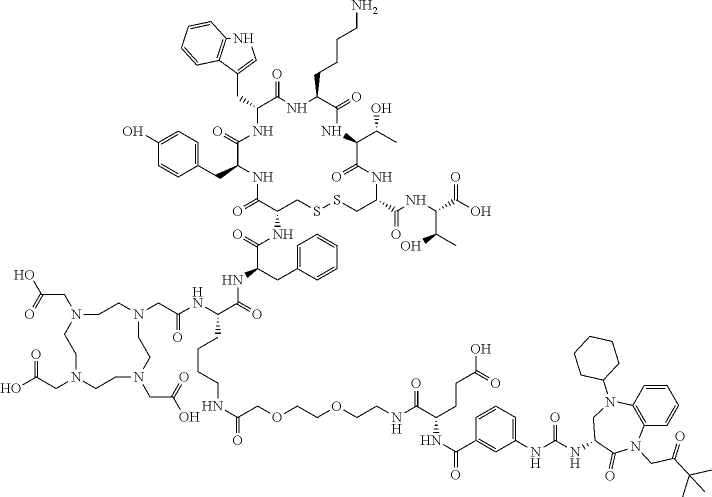

Molecular weight (average) calculated: 2340.7 g/mol

Determined by LC-MS: (M+2H)2+: 1170.9; (M+3H)_{3+: 781.1}; (M+4H)4+: 586.0

Purity by UPLC (214 nm): 90.2%

### Example A29: DOTA-K(-OEG-OEG-E-Z360)-f[CYwKTC]T-OH (I-29)

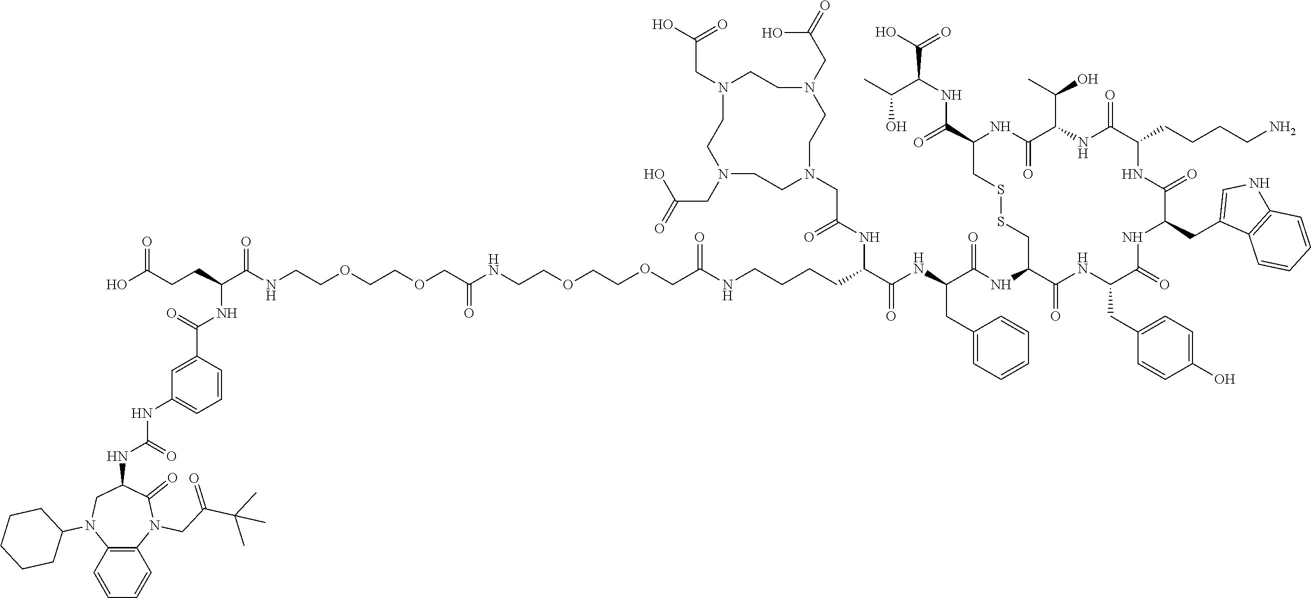 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)COCCOCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2485.9 g/mol

Determined by LC-MS: (M+2H)2+: 1244.0; (M+3H)_{3+: 829.4}; (M+4H)4+: 622.4

Purity by UPLC (214 nm): 95.7%

### Example A30: DOT A-K(-OEG-E-Z360)-OEG-f[CYwKTC]T-OH (I-30)

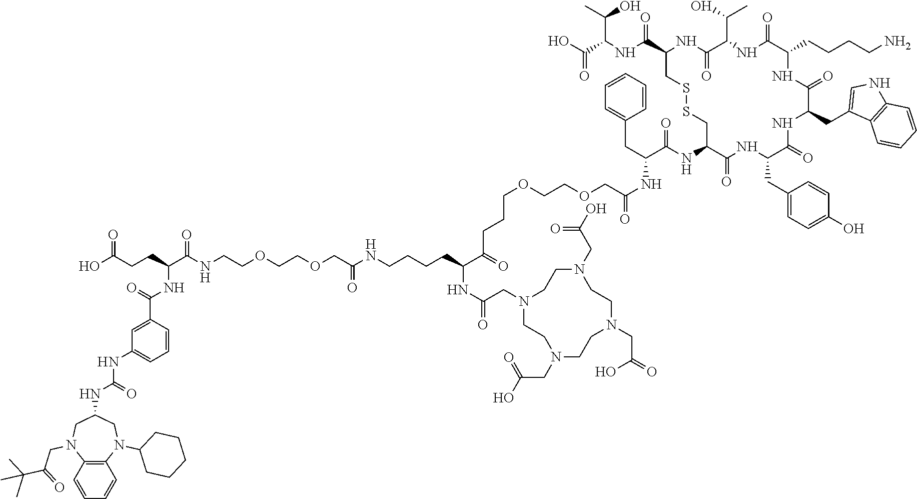 [SMILES: C[C@@H](O)[C@H](NC(=O)[C@@H]1CSSC[C@H](NC(=O)[C@@H](Cc2ccccc2)NC(=O)COCCOCCCC(=O)[C@H](CCCCNC(=O)COCCOCCNC(=O)[C@H](CCC(=O)O)NC(=O)c2cccc(NC(=O)N[C@H]3CN(CC(=O)C(C)(C)C)c4ccccc4N(C4CCCCC4)C3)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2485.9 g/mol

Determined by LC-MS: (M+2H)2+: 1243.5; (M+3H)_{3+: 829.4}; (M+4H)4+: 622.3

Purity by UPLC (214 nm): 98.4%

### Example A31: Z360-OEG-K(DOTAGA)-OEG-f[CYwKTC]T-OH (I-31)

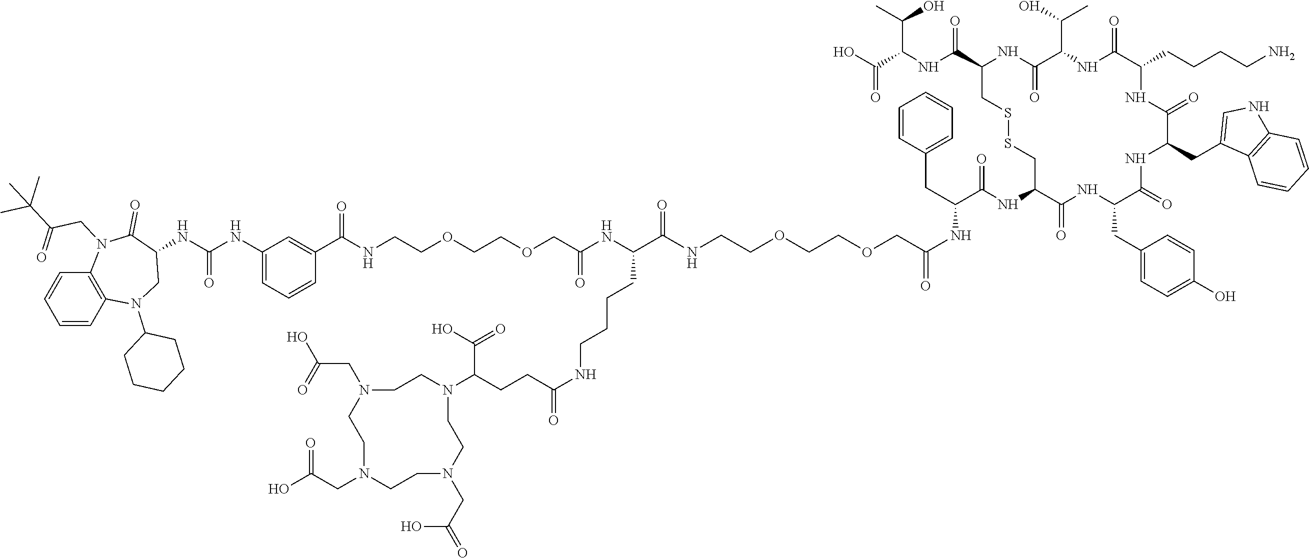 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CCC(C(=O)O)N2CCN(CC(=O)O)CCN(CC(=O)O)CCN(CC(=O)O)CC2)NC(=O)COCCOCCNC(=O)c2cccc(NC(=O)N[C@@H]3CN(C4CCCCC4)c4ccccc4N(CC(=O)C(C)(C)C)C3=O)c2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2428.8 g/mol

Determined by LC-MS: (M+2H)2+: 1215.1; (M+3H)_{3+: 810.4}; (M+4H)4+: 608.1

Purity by UPLC (214 nm): >99.0%

### Example A32: Z360-G-gE-OEG-OEG-OEG-K(DOTA)-OEG-f[CYwKTC]T-OH (I-32)

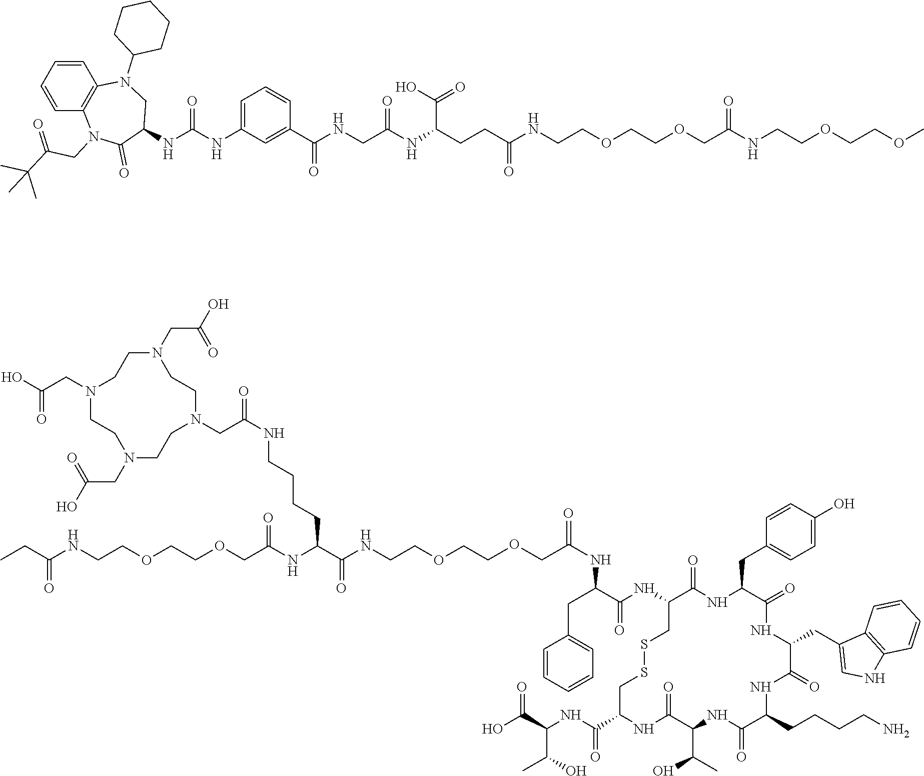

Molecular weight (average) calculated: 2833.2 g/mol

Determined by LC-MS: (M+2H)2+: 1417.4; (M+3H)_{3+: 945.2}; (M+4H)4+: 709.2

Purity by UPLC (214 nm): >99.0%

### Example A33: DOTA-OEG-OEG-OEG-OEG-OEG-OEG-K(-gE-G-Z360)-OEG-f[CYwKTC]TO-H (I-33)

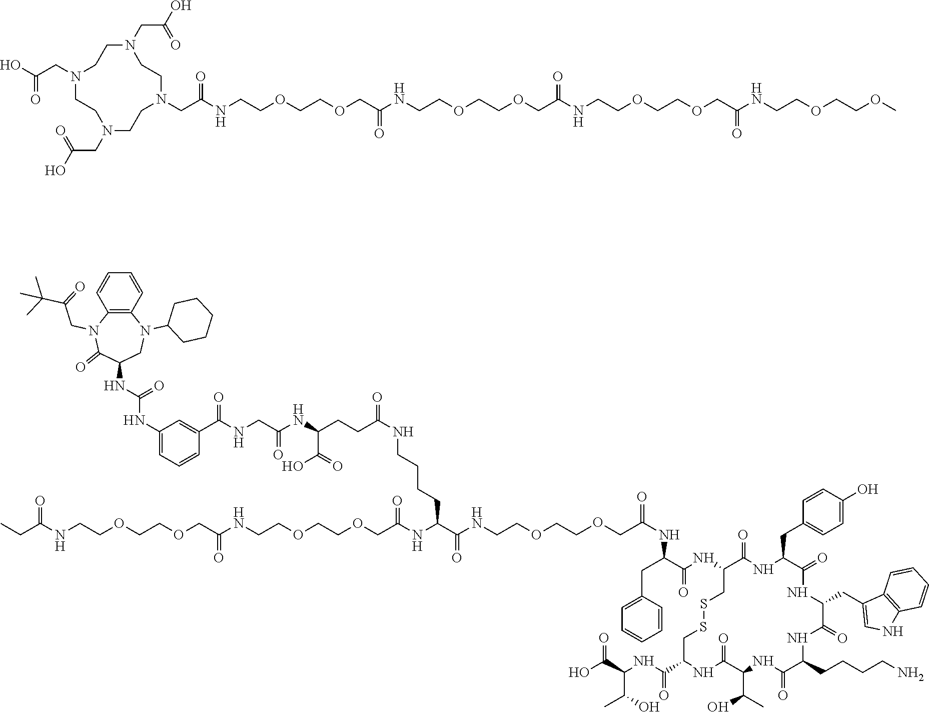

Molecular weight (average) calculated: 3268.7 g/mol

Determined by LC-MS: (M+3H)_{3+: 1090.3}; (M+4H)4+: 816.0; (M+5H)5+:654.7

Purity by UPLC (214 nm): >99.0%

### Example A34: DOTA-K(Z360)-f[CYwKTC]T-OH (I-34)

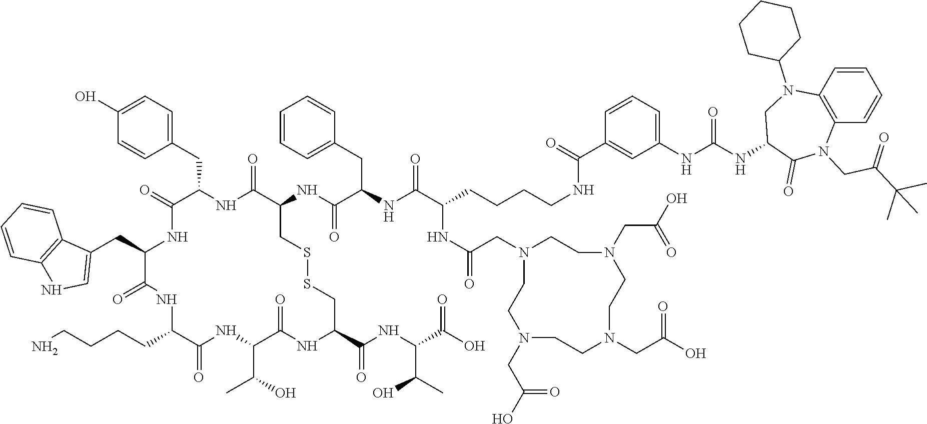 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2066.4 g/mol

Determined by LC-MS: (M+2H)2+: 1033.7; (M+3H)_{3+: 689.6}

Purity by UPLC (214 nm): 99.3%

### Example A35: DOTA-OEG-K(Z360)-f[CYwKTC]T-OH (I-35)

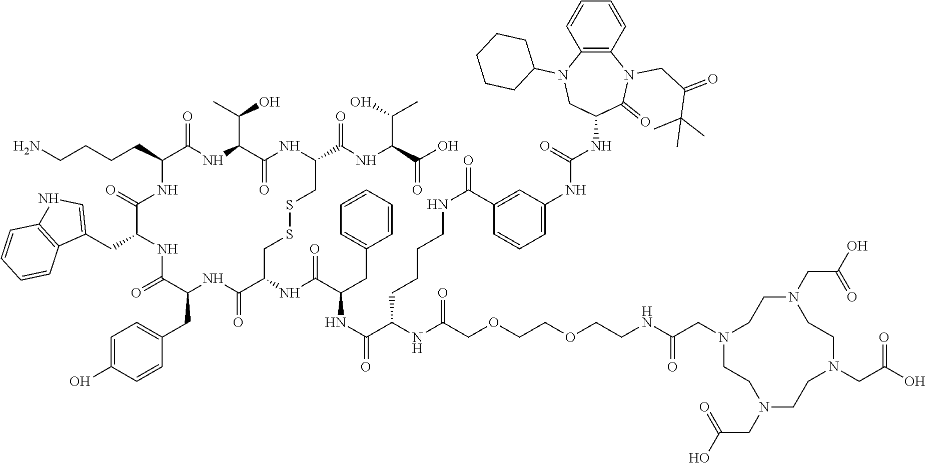 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)COCCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2211.6 g/mol

Determined by LC-MS: (M+2H)2+: 1106.4; (M+3H)_{3+: 738.0}; (M+4H)4+:553.8

Purity by UPLC (214 nm): >99.0%

### Example A36: DOTA-K(-OEG-Z360)-f[CYwKTC]T-OH (I-36)

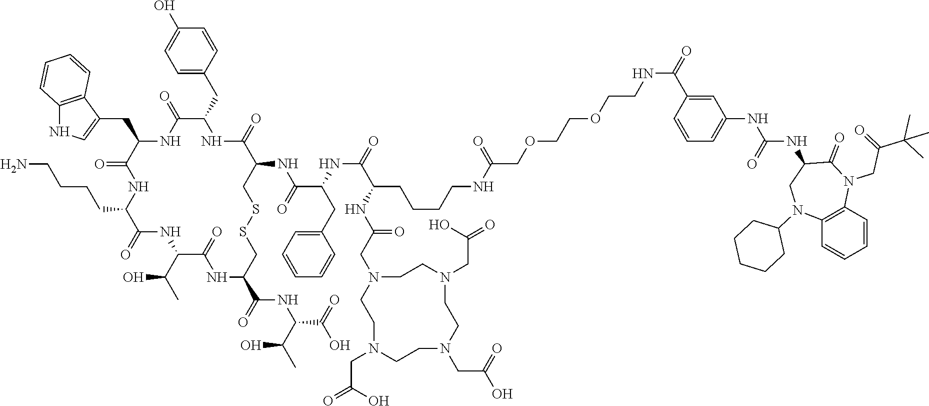 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2211.6 g/mol

Determined by LC-MS: (M+2H)2+: 1106.2; (M+3H)_{3+: 738.0}; (M+4H)4+: 553.9

Purity by UPLC (214 nm): >99.0%

### Example A37: DOTA-K(Z360)-OEG-f[CYwKTC]T-OH (I-37)

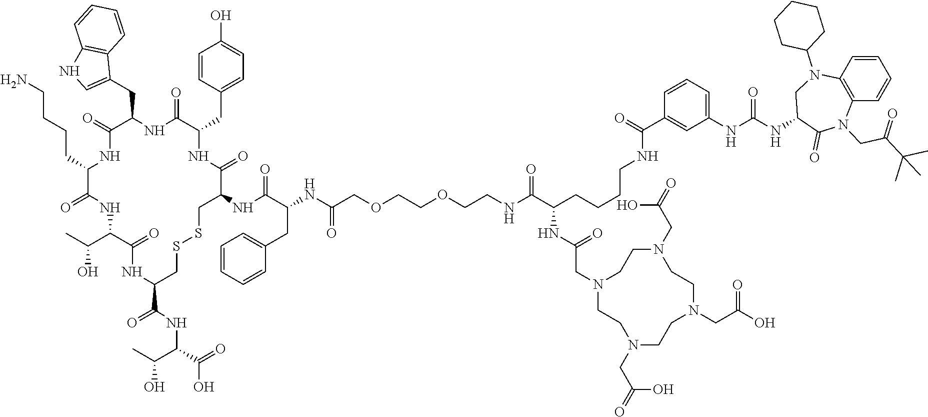 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2211.6 g/mol

Determined by LC-MS: (M+2H)2+: 1106.0; (M+3H)_{3+: 738.0}; (M+4H)4+: 553.8

Purity by UPLC (214 nm): >99.0%

### Example A38: DOTA-K(Z360)-PEG3-f[CYwKTC]T-OH (I-38)

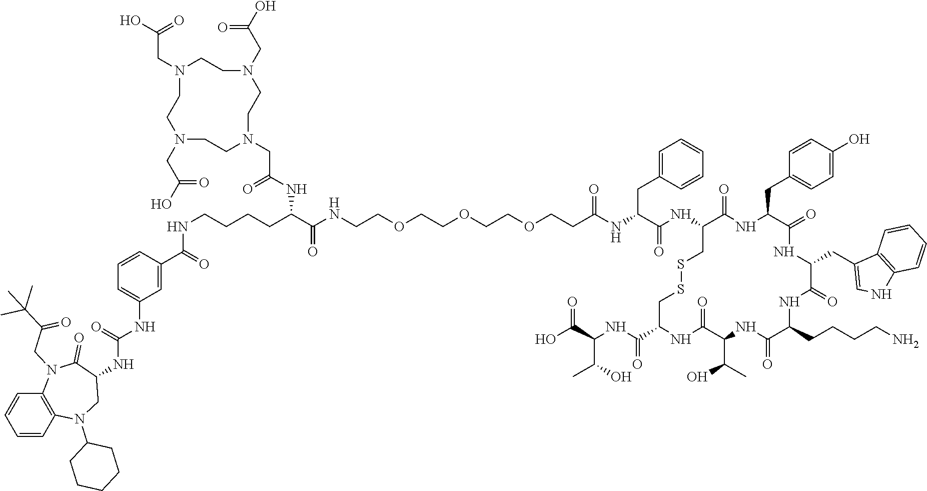 [SMILES: C[C@@H](O)[C@H](NC(=O)[C@@H]1CSSC[C@H](NC(=O)[C@@H](Cc2ccccc2)NC(=O)CCOCCOCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2269.7 g/mol

Determined by LC-MS: (M+2H)2+: 1135.4; (M+3H)_{3+: 757.3}; (M+4H)4+: 568.3

Purity by UPLC (214 nm): 99.3%

### Example A39: DOTA-K(Z360)-OEG-OEG-f[CYwKTC]T-OH (I-39)

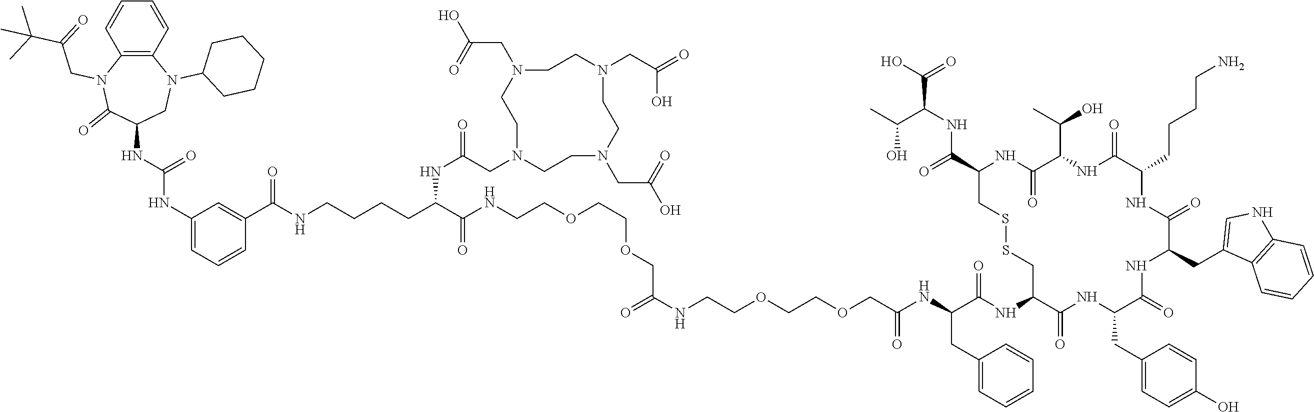 [SMILES: C[C@@H](O)[C@H](NC(=O)[C@@H]1CSSC[C@H](NC(=O)[C@@H](Cc2ccccc2)NC(=O)COCCOCCNC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2356.7 g/mol PGPp-158,TI1

Determined by LC-MS: (M+2H)2+: 1178.8; (M+3H)_{3+: 786.5}; (M+4H)4+: 590.2

Purity by UPLC (214 nm): >99.0%

### Example A40: DOTA-K(Z360)-PEG6-f[CYwKTC]T-OH (I-40)

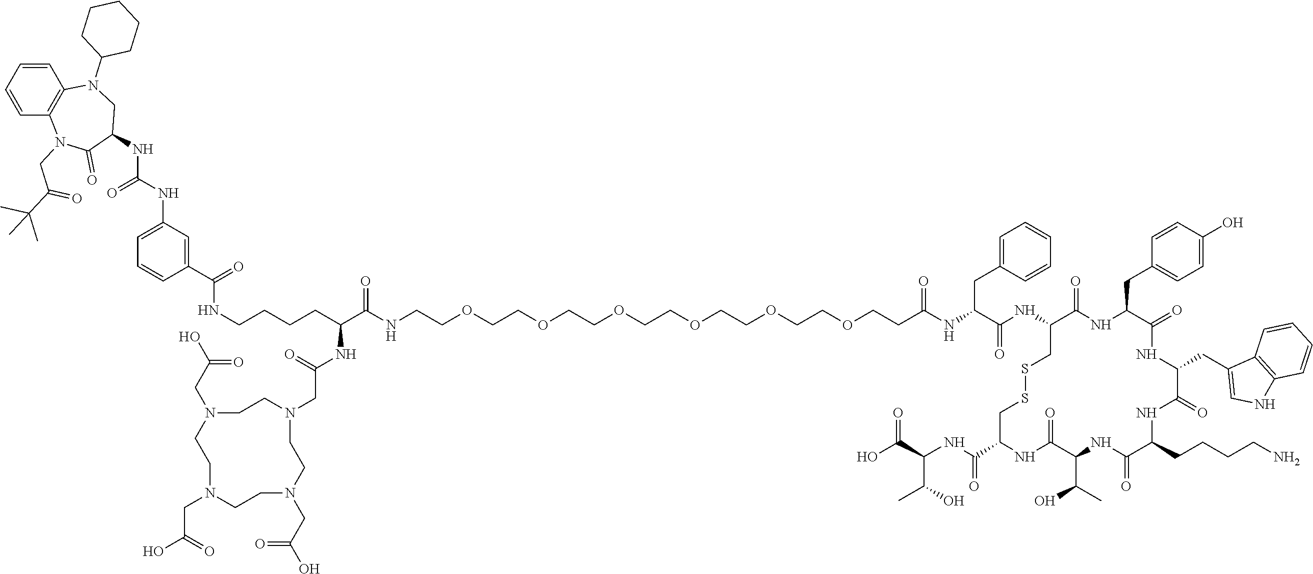 [SMILES: C[C@@H](O)[C@H](NC(=O)[C@@H]1CSSC[C@H](NC(=O)[C@@H](Cc2ccccc2)NC(=O)CCOCCOCCOCCOCCOCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2401.8 g/mol

Determined by LC-MS: (M+2H)2+: 1201.4; (M+3H)_{3+: 801.4}; (M+4H)4+: 601.4

Purity by UPLC (214 nm): 97.6%

### Example A41: DOTA-OEG-OEG-K(Z360)-f[CYwKTC]T-OH (I-41)

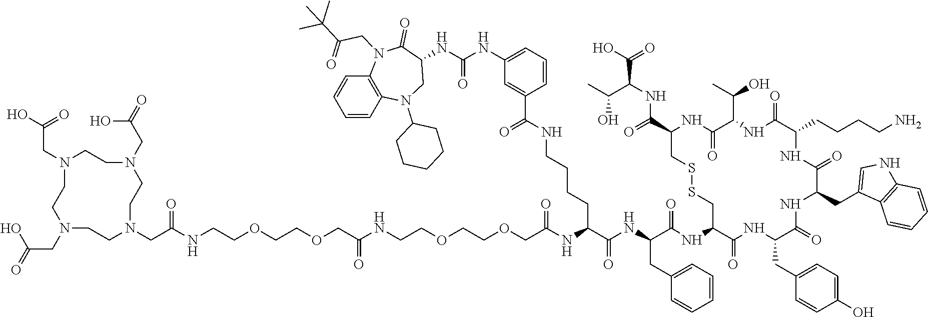 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)COCCOCCNC(=O)COCCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2356.7 g/mol

Determined by LC-MS: (M+2H)2+: 1179.1; (M+3H)_{3+: 786.3}; (M+4H)4+: 590.0

Purity by UPLC (214 nm): >99.0%

### Example A42: DOTA-K(-OEG-OEG-Z360)-f[CYwKTC]T-OH (I-42)

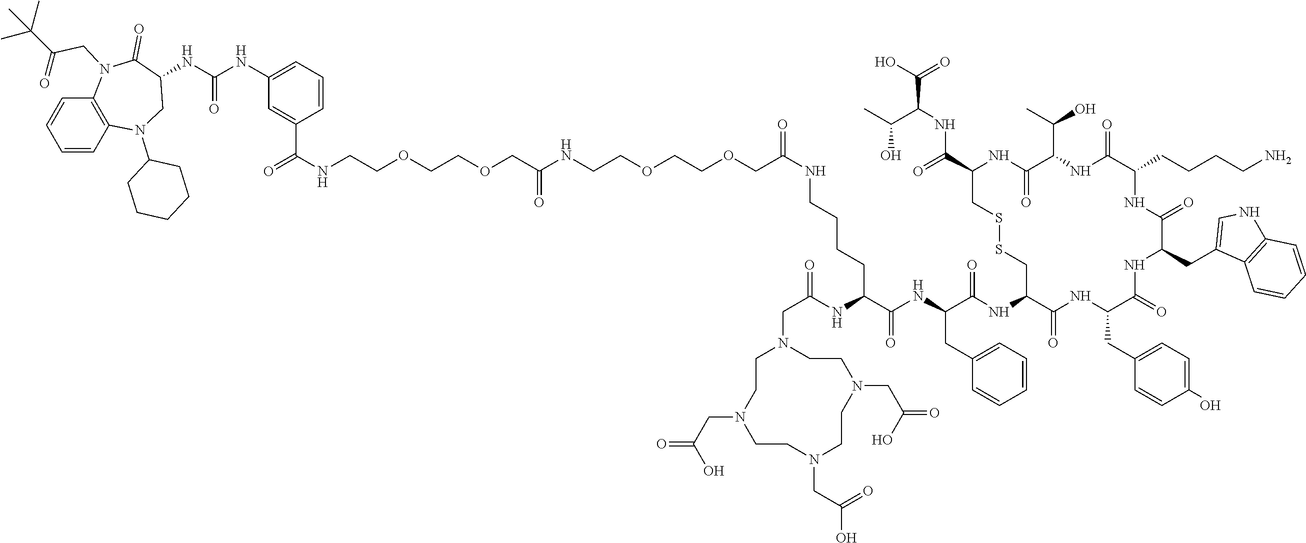 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2356.7 g/mol

Determined by LC-MS: (M+2H)2+: 1178.9; (M+3H)_{3+: 786.3}; (M+4H)4+: 590.1

Purity by UPLC (214 nm): 91.0%

### Example A43: DOTA-K(-OEG-Z360)-OEG-f[CYwKTC]T-OH (I-43)

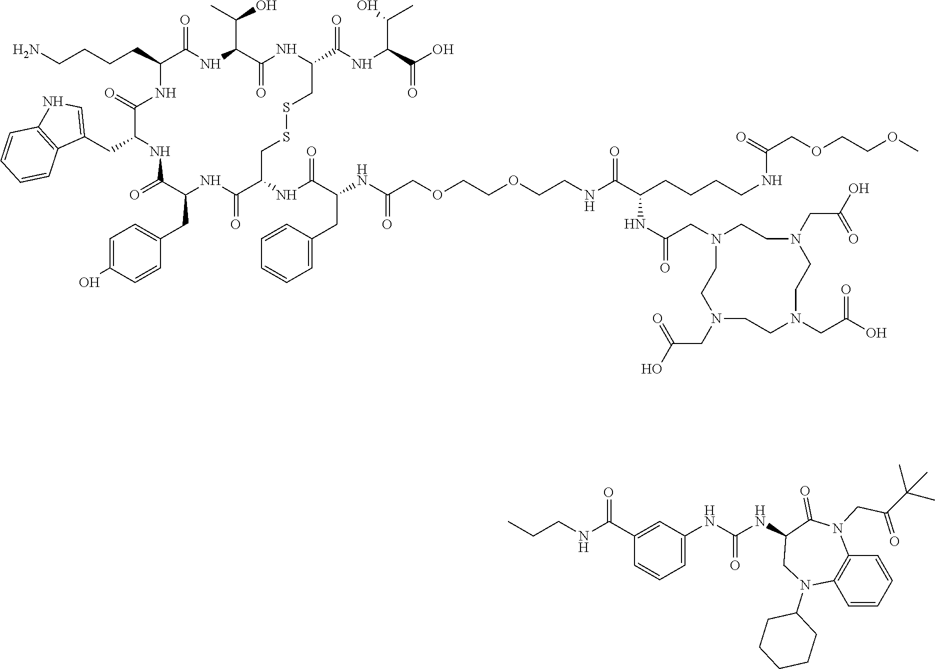

Molecular weight (average) calculated: 2356.7 g/mol

Determined by LC-MS: (M+2H)2+: 1178.8; (M+3H)_{3+: 786.2}; (M+4H)4+: 590.0

Purity by UPLC (214 nm): >99.0%

### Example A44: DOTA-OEG-K(-OEG-Z360)-f[CYwKTC]T-OH (I-44)

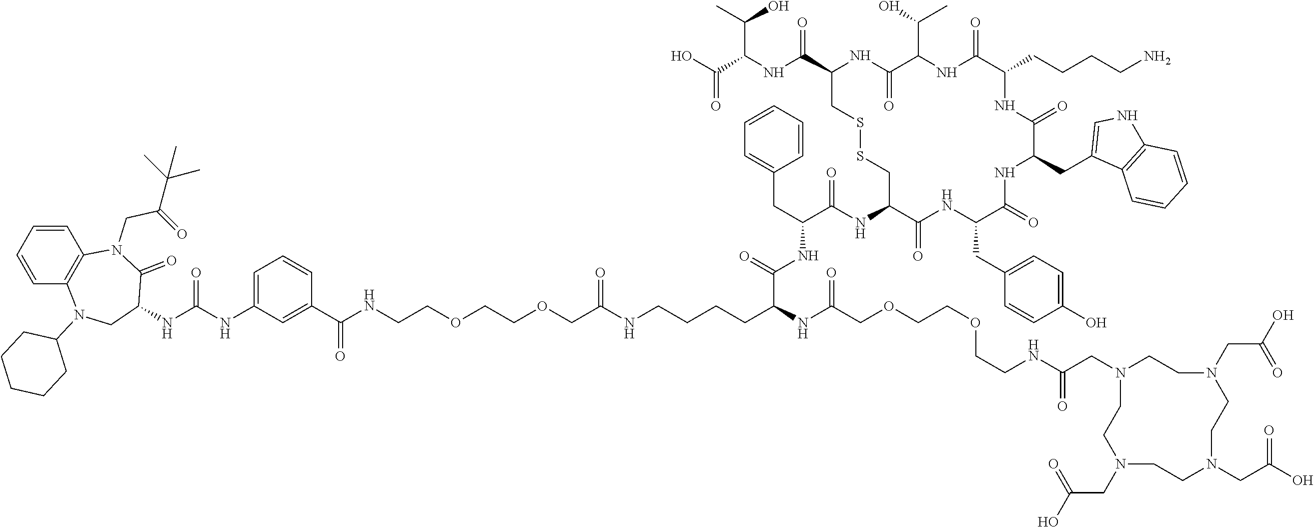 [SMILES: C[C@@H](O)C1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)[C@H](CCCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)COCCOCCNC(=O)CN2CCN(CC(=O)O)CCN(CC(=O)O)CCN(CC(=O)O)CC2)CSSC[C@@H](C(=O)N[C@H](C(=O)O)[C@@H](C)O)NC1=O]

Molecular weight (average) calculated: 2356.7 g/mol

Determined by LC-MS: (M+2H)2+: 1179.4; (M+3H)_{3+: 786.4}; (M+4H)4+: 590.1

Purity by UPLC (214 nm): 98.4%

### Example A45: DOTA-K(Z360)-OEG-OEG-OEGf[CYwKTC]T-OH (I-45)

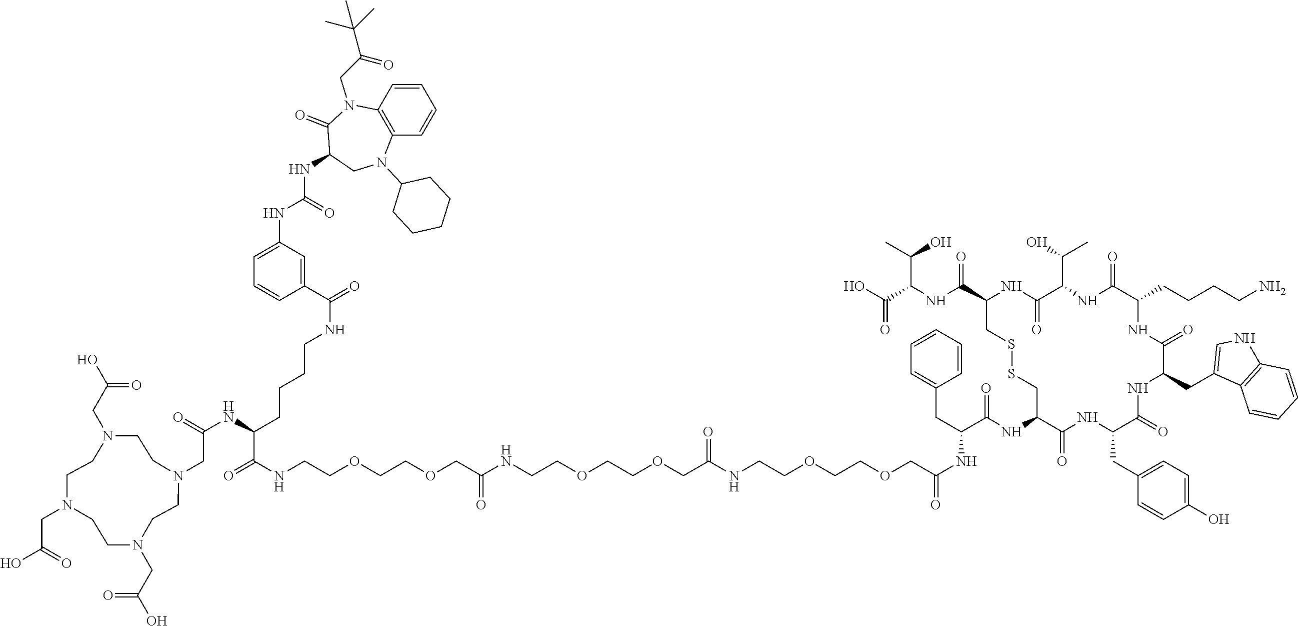 [SMILES: C[C@@H](O)[C@H](NC(=O)[C@@H]1CSSC[C@H](NC(=O)[C@@H](Cc2ccccc2)NC(=O)COCCOCCNC(=O)COCCOCCNC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2501.9 g/mol

Determined by LC-MS: (M+2H)2+: 1251.9; (M+3H)_{3+: 834.8}; (M+4H)4+: 626.4

Purity by UPLC (214 nm): >99.0%

### Example A46: DOTA-K(-OEG-OEG-OEG-Z360)-f[CYwKTC]T-OH (I-46)

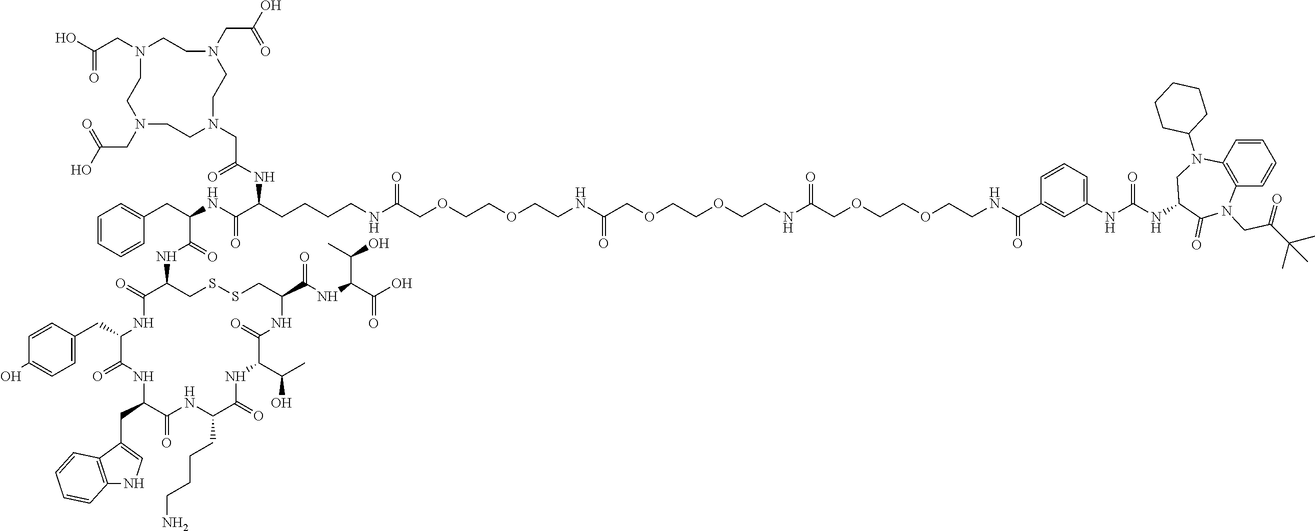 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)COCCOCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2501.9 g/mol

Determined by LC-MS: (M+2H)2+: 1251.4; (M+3H)_{3+: 834.7}; (M+4H)4+: 626.3

Purity by UPLC (214 nm): 90.2%

### Example A47: DOTA-eK-OEG-OEG-OEG-K(-gE-G-Z360)-OEG-f[CYwKTC]T-OH (I-47)

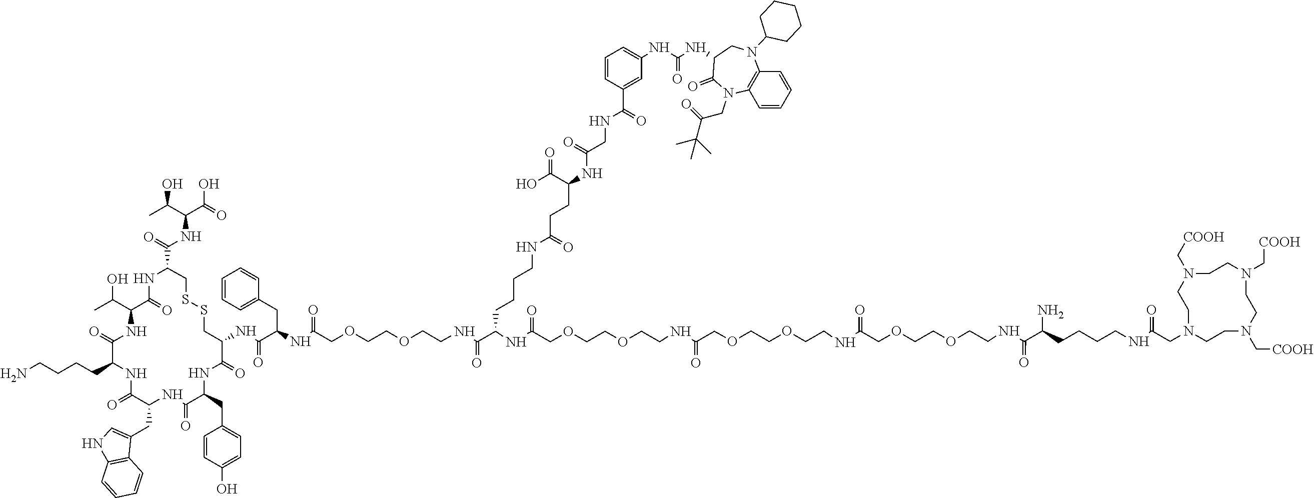 [SMILES: CC(O)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)COCCOCCNC(=O)[C@@H](N)CCCCNC(=O)CN2CCN(CC(=O)O)CCN(CC(=O)O)CCN(CC(=O)O)CC2)CSSC[C@@H](C(=O)N[C@H](C(=O)O)[C@@H](C)O)NC1=O]

Molecular weight (average) calculated: 2961.4 g/mol

Determined by LC-MS: (M+3H)_{3+: 987.8}; (M+4H)4+: 741.2; (M+5H)5+: 593.2

Purity by UPLC (214 nm): 92.0%

### Example A48: DOTA-PEG3-K(Z360)-f[CYwKTC]T-OH (I-48)

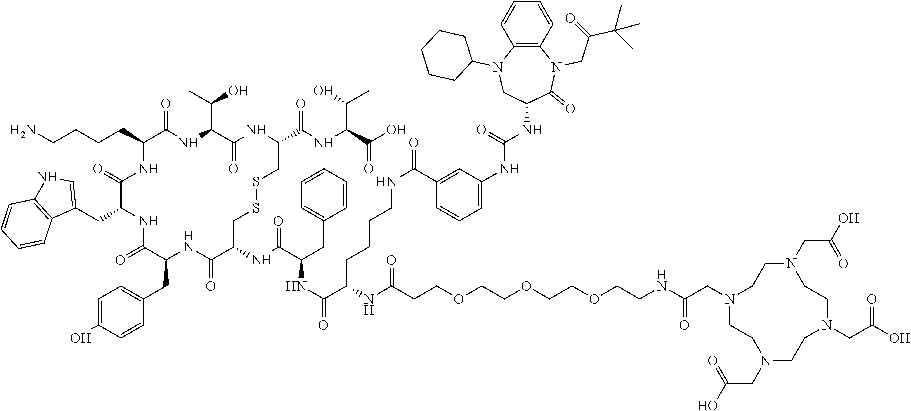 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CCOCCOCCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2269.7 g/mol PGPI167,T1

Determined by LC-MS: (M+2H)2+: 1135.3; (M+3H)_{3+: 757.3}; (M+4H)4+: 568.3

Purity by UPLC (214 nm): 96.1%

### Example A49: DOTA-G-G-K(Z360)-f[CYwKTC]T-OH (I-49)

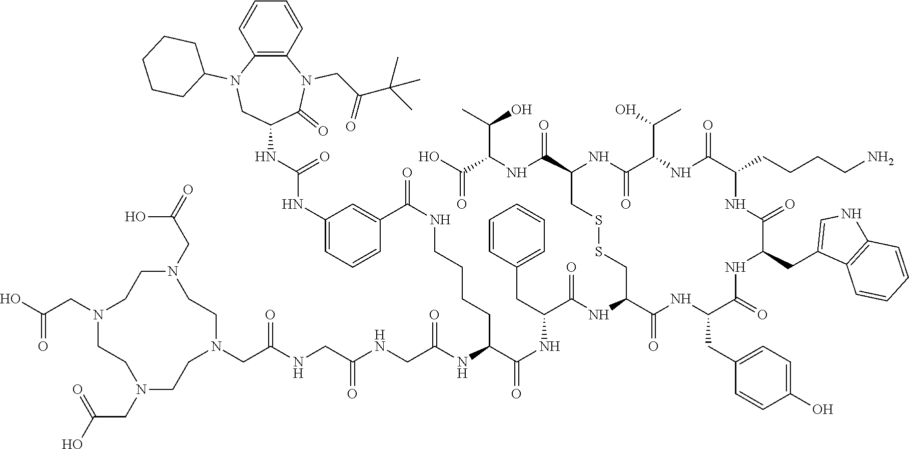 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CNC(=O)CNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2180.5 g/mol

Determined by LC-MS: (M+2H)2+: 1090.7; (M+3H)_{3+: 727.6}; (M+4H)4+: 546.1

Purity by UPLC (214 nm): 98.8%

### Example A50: DOTA-S—S-K(Z360)-f[CYwKTC]T-OH (I-50)

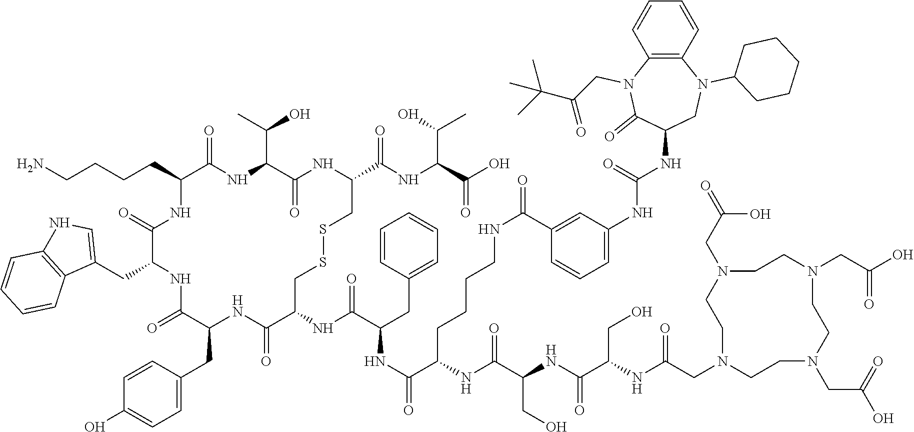 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)[C@H](CO)NC(=O)[C@H](CO)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2240.6 g/mol

Determined by LC-MS: (M+2H)2+: 1120.7; (M+3H)_{3+: 747.7}; (M+4H)4+: 561.0

Purity by UPLC (214 nm): 97.9%

### Example A51: DOTA-PEG1-K(Z360)-f[CYwKTC]T-OH (I-51)

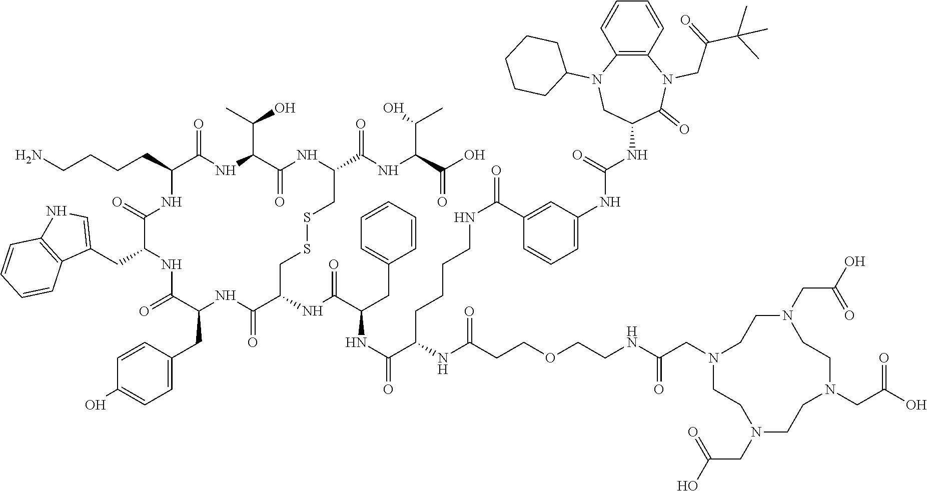 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2181.6 g/mol

Determined by LC-MS: (M+2H)2+: 1091.5; (M+3H)_{3+: 728.0}; (M+4H)4+: 546.4

Purity by UPLC (214 nm): 97.2%

### Example A52: DOTA-OEG-K(Z360)-OEG-f[CYwKTC]T-OH (I-52)

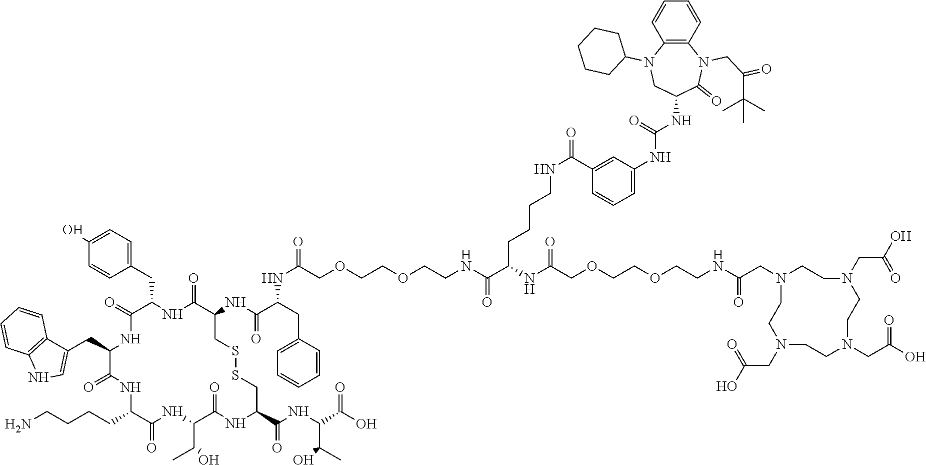

Molecular weight (average) calculated: 2356.7 g/mol

Determined by LC-MS: (M+2H)2+: 1178.9; (M+3H)_{3+: 786.4}; (M+4H)4+: 590.0

Purity by UPLC (214 nm): >99.0%

### Example A53: DOTA-K(Z360)-OEG-G-f[CYwKTC]T-OH (I-53)

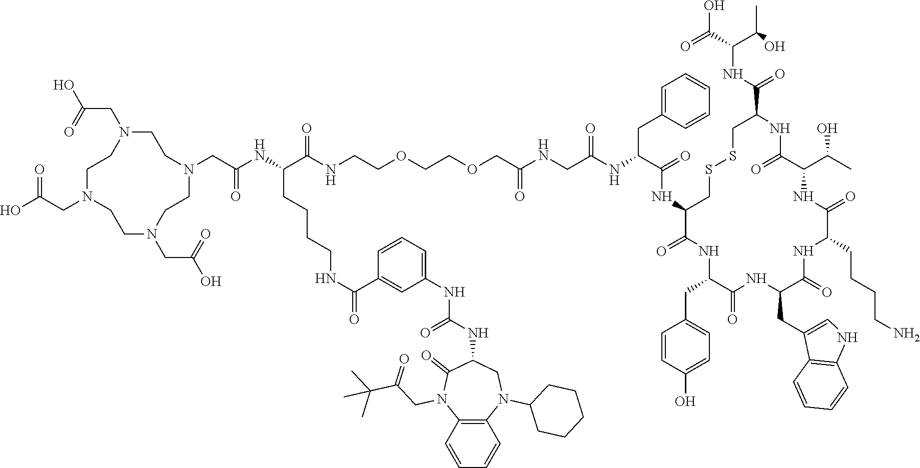 [SMILES: C[C@@H](O)[C@H](NC(=O)[C@@H]1CSSC[C@H](NC(=O)[C@@H](Cc2ccccc2)NC(=O)CNC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2268.6 g/mol

Determined by LC-MS: (M+2H)2+: 1134.7; (M+3H)_{3+: 757.0}; (M+4H)4+: 568.1

Purity by UPLC (214 nm): 95.5%

### Example A54: DOTA-K(Z360)-G-S-G-f[CYwKTC]T-OH (I-54)

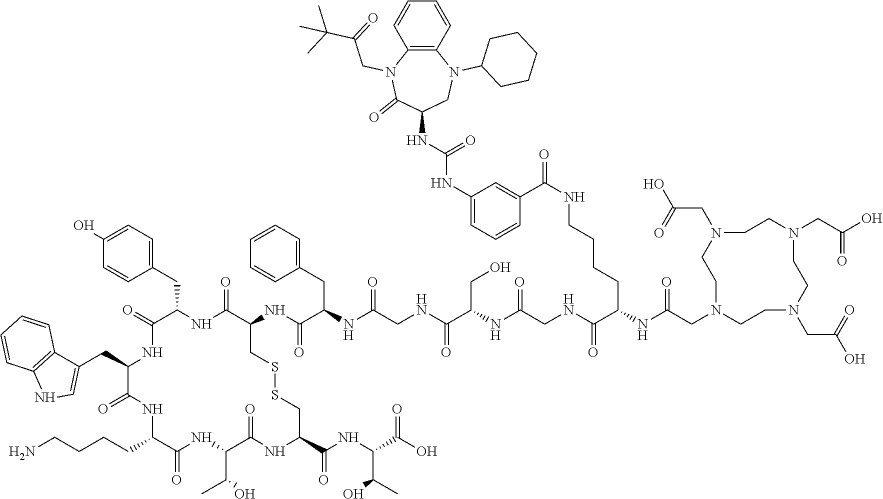 [SMILES: C[C@@H](O)[C@H](NC(=O)[C@@H]1CSSC[C@H](NC(=O)[C@@H](Cc2ccccc2)NC(=O)CNC(=O)[C@H](CO)NC(=O)CNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2267.6 g/mol

Determined by LC-MS: (M+2H)2+: 1134.2; (M+3H)_{3+: 756.6}; (M+4H)4+: 567.8

Purity by UPLC (214 nm): 98.9%

### Example A55: DOTA-K(Z360)-G-p-G-p-G-f[CYwKTC]T-OH (I-55)

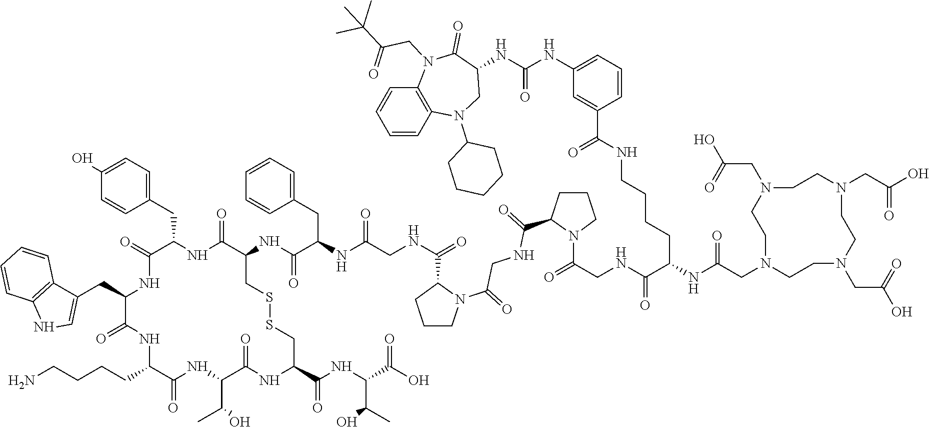 [SMILES: C[C@@H](O)[C@H](NC(=O)[C@@H]1CSSC[C@H](NC(=O)[C@@H](Cc2ccccc2)NC(=O)CNC(=O)[C@H]2CCCN2C(=O)CNC(=O)[C@H]2CCCN2C(=O)CNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2431.8 g/mol

Determined by LC-MS: (M+2H)2+: 1216.6; (M+3H)_{3+: 811.3}; (M+4H)4+: 608.8

Purity by UPLC (214 nm): >99.0%

### Example A56: DOTA-G-p-G-K(Z360)-f[CYwKTC]T-OH (I-56)

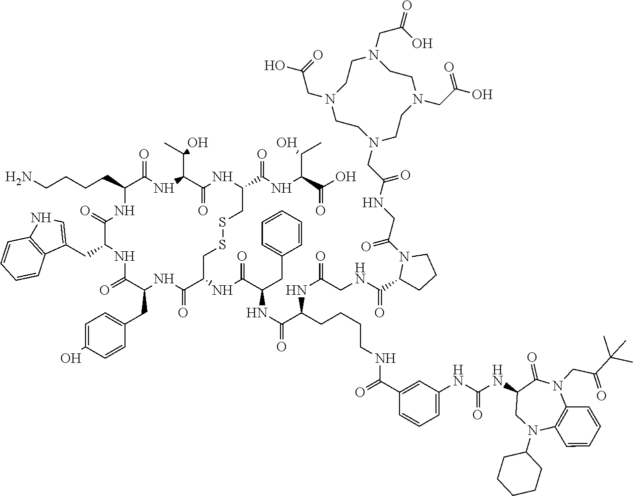 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CNC(=O)[C@H]2CCCN2C(=O)CNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2277.7 g/mol

Determined by LC-MS: (M+2H)2+: 1139.4; (M+3H)_{3+: 759.8}; (M+4H)4+: 570.2

Purity by UPLC (214 nm): >99.0%

### Example A57: DOTA-p-G-p-G-K(Z360)-f[CYwKTC]T-OH (I-57)

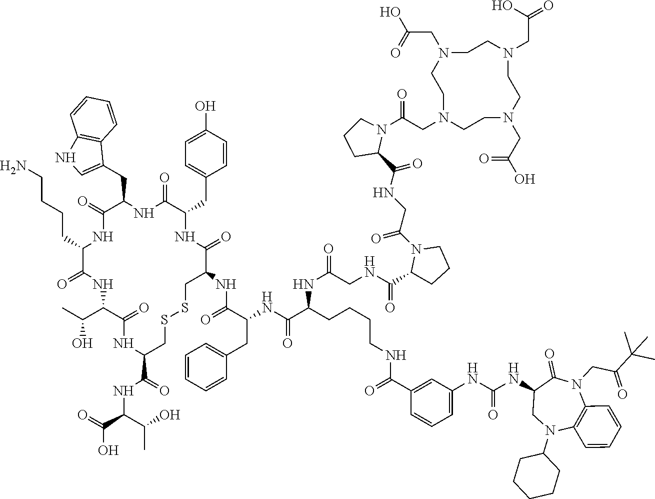 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CNC(=O)[C@H]2CCCN2C(=O)CNC(=O)[C@H]2CCCN2C(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2374.8 g/mol

Determined by LC-MS: (M+2H)2+: 1187.8; (M+3H)_{3+: 792.3}; (M+4H)4+: 594.7

Purity by UPLC (214 nm): >99.0%

### Example A58: DOTA-G-S-K(Z360)-G-S-f[CYwKTC]T-OH (I-58)

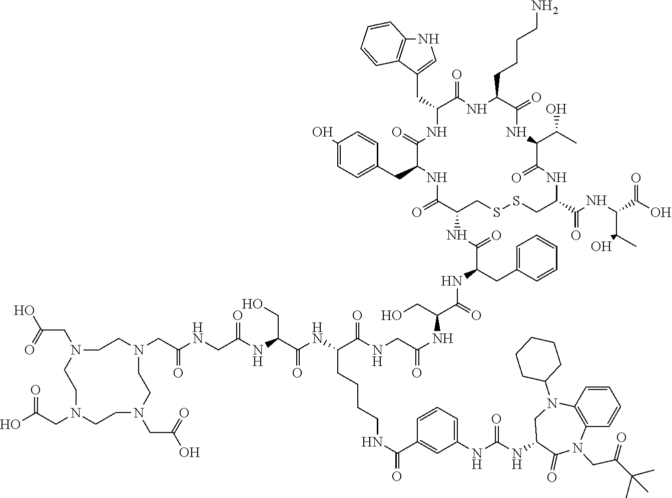 [SMILES: C[C@@H](O)[C@H](NC(=O)[C@@H]1CSSC[C@H](NC(=O)[C@@H](Cc2ccccc2)NC(=O)[C@H](CO)NC(=O)CNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)[C@H](CO)NC(=O)CNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2354.7 g/mol

Determined by LC-MS: (M+2H)2+: 1177.5; (M+3H)_{3+: 785.6}; (M+4H)4+: 589.6

Purity by UPLC (214 nm): 95.6%

### Example A59: DOTA-Aoc-K(Z360)-f[CYwKTC]T-OH (I-59)

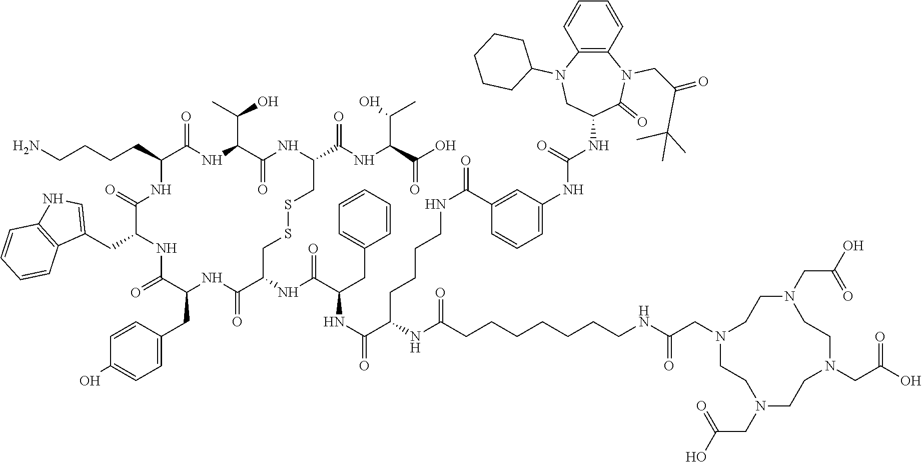 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CCCCCCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2207.6 g/mol

Determined by LC-MS: (M+2H)2+: 1104.2; (M+3H)_{3+: 736.7}; (M+4H)4+: 552.8

Purity by UPLC (214 nm): 96.5%

### Example A60: DOTA-K(-G-G-G-Z360)-f[CYwKTC]T-OH (I-60)

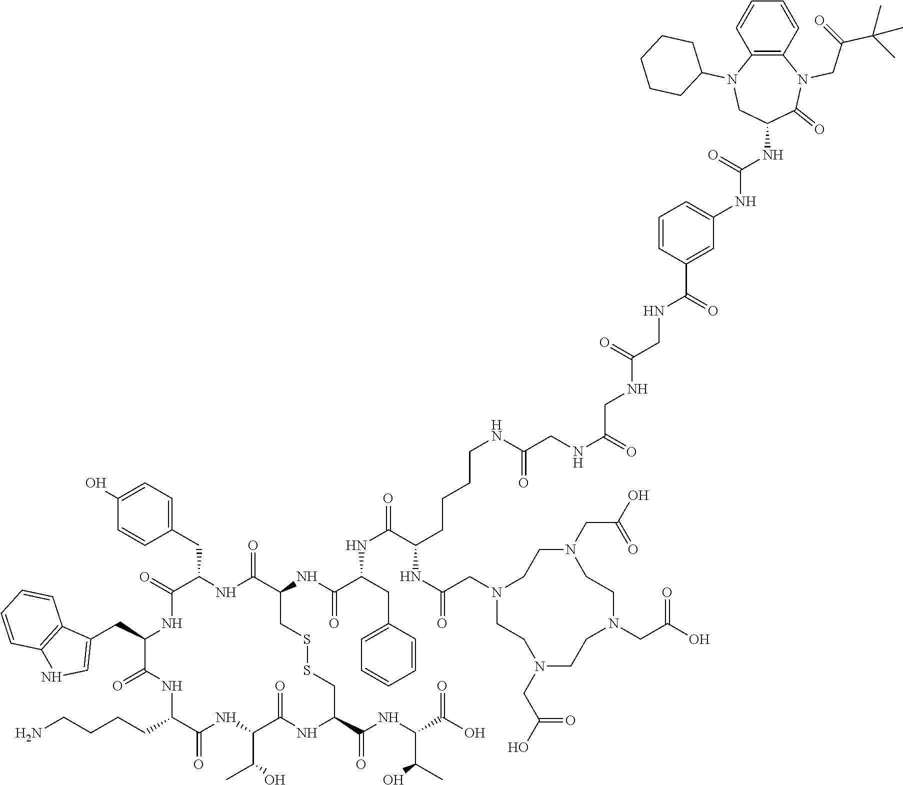 [SMILES: C[C@@H](O)[C@H](NC(=O)[C@@H]1CSSC[C@H](NC(=O)[C@@H](Cc2ccccc2)NC(=O)[C@H](CCCCNC(=O)CNC(=O)CNC(=O)CNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2237.6 g/mol

Determined by LC-MS: (M+2H)2+: 1119.2; (M+3H)_{3+: 746.7}; (M+4H)4+: 560.4

Purity by UPLC (214 nm): 92.4%

### Example A61: DOTA-K(Z360)-Aoc-f[CYwKTC]T-OH (I-61)

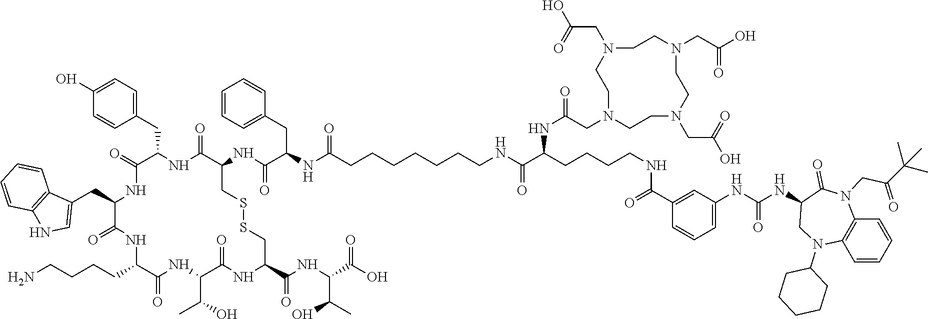 [SMILES: C[C@@H](O)[C@H](NC(=O)[C@@H]1CSSC[C@H](NC(=O)[C@@H](Cc2ccccc2)NC(=O)CCCCCCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2207.6 g/mol

Determined by LC-MS: (M+2H)2+: 1104.3; (M+3H)_{3+: 736.5}; (M+4H)4+: 552.7

Purity by UPLC (214 nm): 92.6%

### Example A62: DOTA-R-K(Z360)-f[CYwKTC]T-OH (I-62)

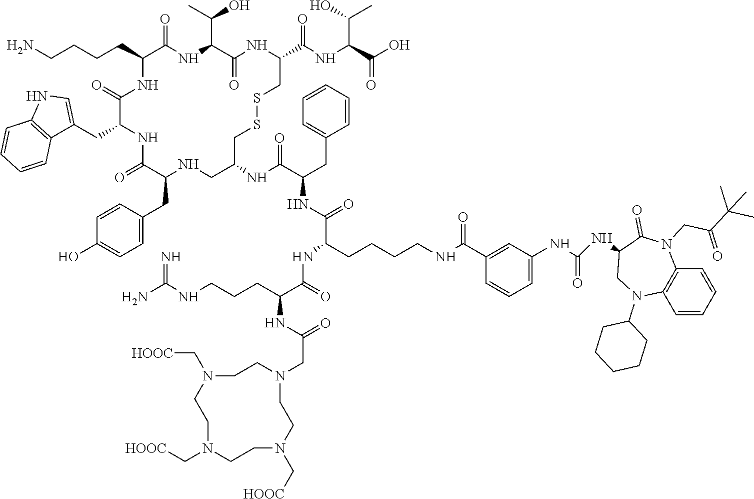 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)[C@H](CCCNC(=N)N)NC(=O)CN2CCN(CC(=O)O)CCN(CC(=O)O)CCN(CC(=O)O)CC2)CN[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2222.6 g/mol

Determined by LC-MS: (M+2H)2+: 1111.9; (M+3H)_{3+: 741.6}; (M+4H)4+: 556.6

Purity by UPLC (214 nm): 97.5%

### Example A63: DOTA-K(Z360)-R-f[CYwKTC]T-OH (I-63)

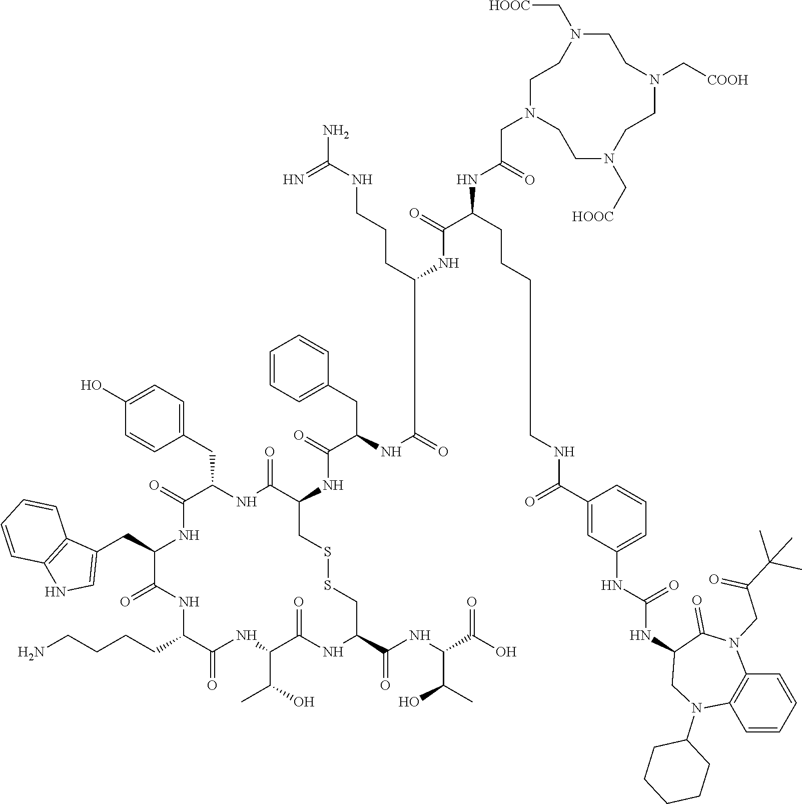 [SMILES: C[C@@H](O)[C@H](NC(=O)[C@@H]1CSSC[C@H](NC(=O)[C@@H](Cc2ccccc2)NC(=O)[C@H](CCCNC(=N)N)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 2222.6 g/mol

Determined by LC-MS: (M+2H)2+: 1111.9; (M+3H)_{3+: 741.7}; (M+4H)4+: 556.6

Purity by UPLC (214 nm): >99.0%

### Example A64: DOTA-K(-OEG-Z360)-f[CYwKTC]T-NH₂ (1-64)

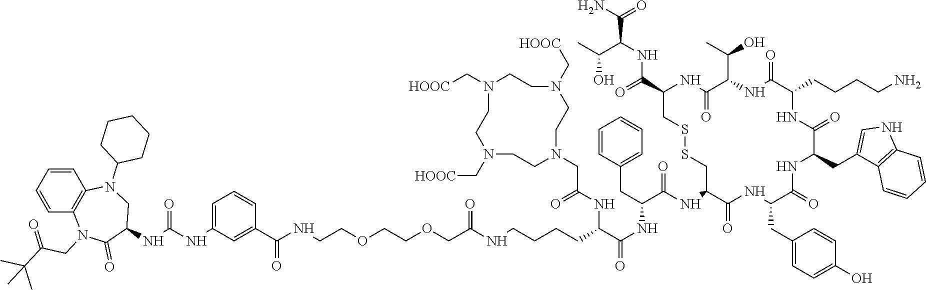 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(N)=O]

Molecular weight (average) calculated: 2210.6 g/mol

Determined by LC-MS: (M+2H)2+: 1105.9; (M+3H)_{3+: 737.6}; (M+4H)4+: 553.6

Purity by UPLC (214 nm): >99.0%

### Example A65: DOTA-eK-eK-OEG-OEG-K(-gE-G-Z360)-OEG-f[CYwKTC]T-OH (I-65)

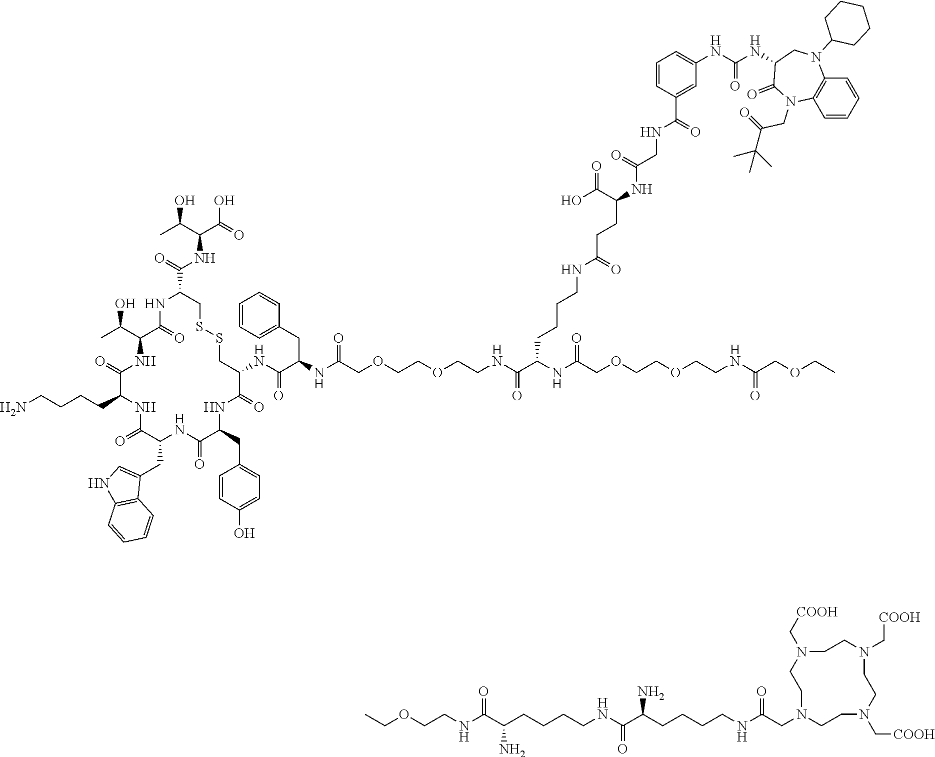 [SMILES: CCOCC(=O)NCCOCCOCC(=O)N[C@@H](CCCCNC(=O)CC[C@H](NC(=O)CNC(=O)c1cccc(NC(=O)N[C@@H]2CN(C3CCCCC3)c3ccccc3N(CC(=O)C(C)(C)C)C2=O)c1)C(=O)O)C(=O)NCCOCCOCC(=O)N[C@H](Cc1ccccc1)C(=O)N[C@H]1CSSC[C@@H](C(=O)N[C@H](C(=O)O)[C@@H](C)O)NC(=O)[C@H]([C@@H](C)O)NC(=O)[C@H](CCCCN)NC(=O)[C@@H](Cc2c[nH]c3ccccc23)NC(=O)[C@H](Cc2ccc(O)cc2)NC1=O ; CCOCCNC(=O)[C@@H](N)CCCCNC(=O)[C@@H](N)CCCCNC(=O)CN1CCN(CC(=O)O)CCN(CC(=O)O)CCN(CC(=O)O)CC1]

Molecular weight (average) calculated: 2944.4 g/mol

Determined by LC-MS: (M+2H)2+: 1472.9; (M+3H)_{3+: 982.1}; (M+4H)4+: 737.0; (M+5H)5+: 589.9

Purity by UPLC (214 nm): 96.3%

### Example A66: DOTA-eK-eK-eK-OEG-K(-gE-G-Z360)-OEG-f[CYwKTC]T-OH (I-66)

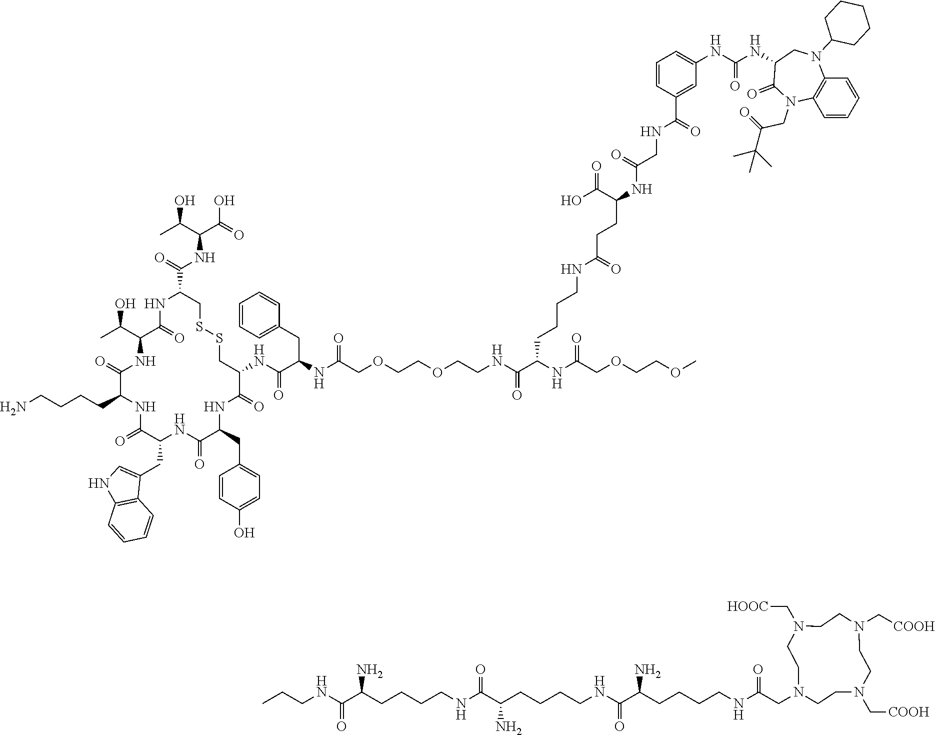 [SMILES: COCCOCC(=O)N[C@@H](CCCCNC(=O)CC[C@H](NC(=O)CNC(=O)c1cccc(NC(=O)N[C@@H]2CN(C3CCCCC3)c3ccccc3N(CC(=O)C(C)(C)C)C2=O)c1)C(=O)O)C(=O)NCCOCCOCC(=O)N[C@H](Cc1ccccc1)C(=O)N[C@H]1CSSC[C@@H](C(=O)N[C@H](C(=O)O)[C@@H](C)O)NC(=O)[C@H]([C@@H](C)O)NC(=O)[C@H](CCCCN)NC(=O)[C@@H](Cc2c[nH]c3ccccc23)NC(=O)[C@H](Cc2ccc(O)cc2)NC1=O ; CCCNC(=O)[C@@H](N)CCCCNC(=O)[C@@H](N)CCCCNC(=O)[C@@H](N)CCCCNC(=O)CN1CCN(CC(=O)O)CCN(CC(=O)O)CCN(CC(=O)O)CC1]

Molecular weight (average) calculated: 2927.4 g/mol

Determined by LC-MS: (M+3H)_{3+: 976.3}; (M+4H)4+: 732.6; (M+5H)5+: 586.5

Purity by UPLC (214 nm): >99.0%

### Example A67: DOTAGA-K(Z360)-OEG-f[CYwKTC]T-OH (I-67)

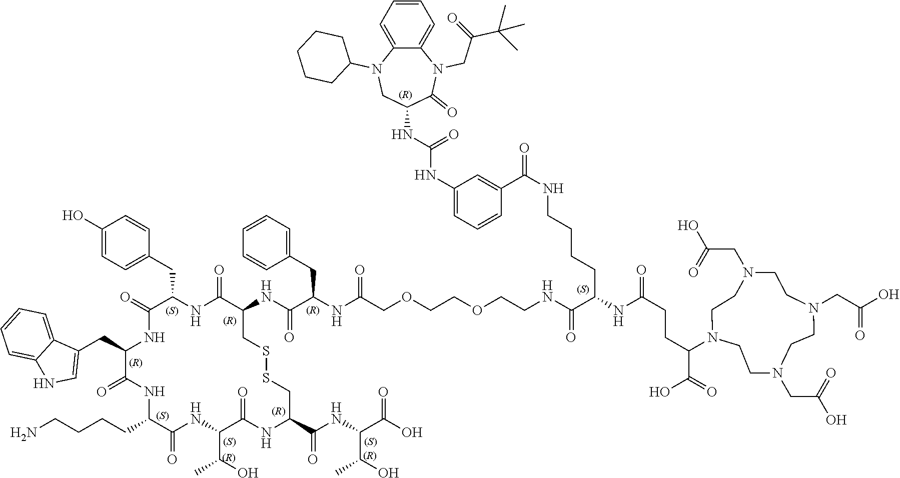

Molecular weight (average) calculated: 2283.64 g/mol

Determined by LC-MS: 1142.1(M+2H⁺); 761.8(M+3H⁺); 571.8(M+4H⁺)

Purity by UPLC (214 nm): 100%

### Example A68: DOTAGA-K(-Glu-Z360)-OEG-f[CYwKTC]T-OH (I-68)

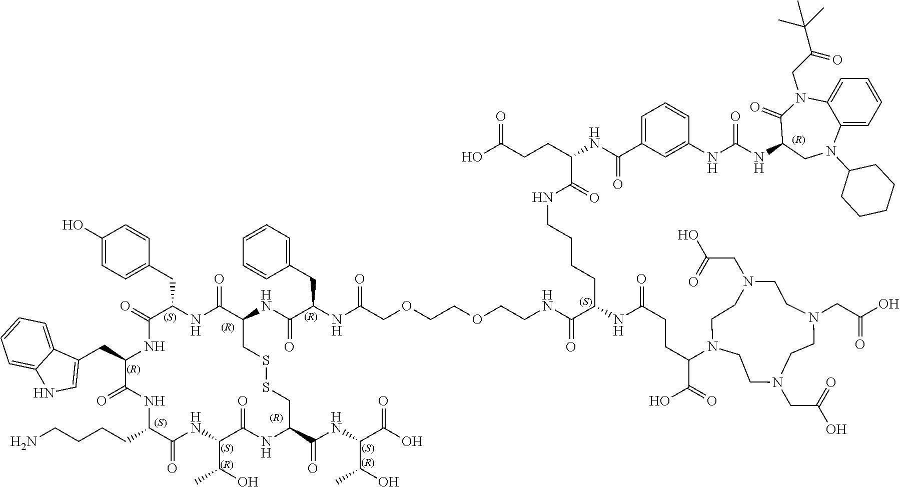

Molecular weight (average) calculated: 2412.76 g/mol PGPI-187,TI

Determined by LC-MS: 1206.8(M+2H+); 805.0(M+i3H+); 604.0(M+4H+)

Purity by UPLC (214 nm): 96%

### Example A69: DOTAGA-K(-Glu-Z360)-f[CYwKTC]T-OH (I-69)

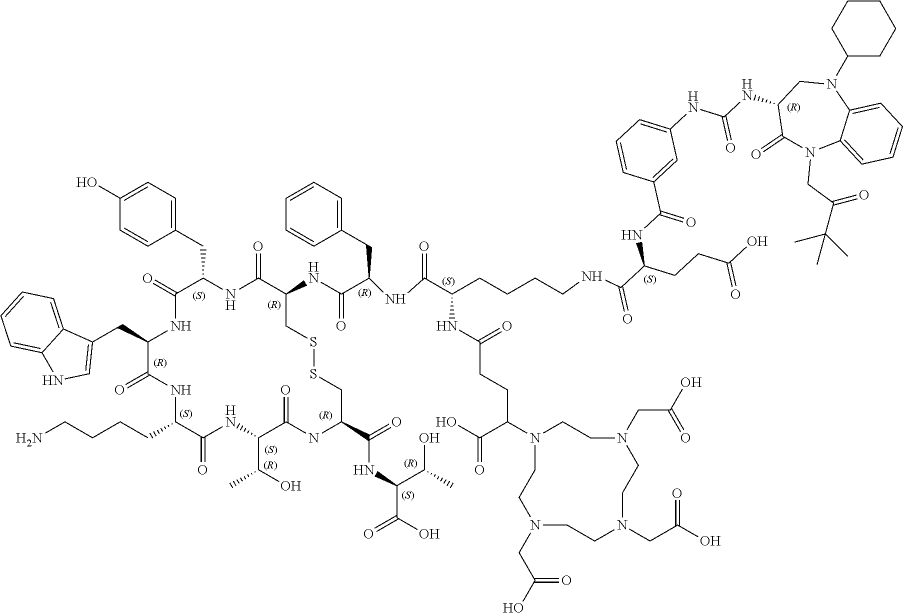

Molecular weight (average) calculated: 2267.60 g/mol

Determined by LC-MS: 1134.1(M+2H⁺); 756.7(M+3H⁺); 567.8(M+4H⁺)

Purity by UPLC (214 nm): 97%

### Example A70: DOTA-K(-Glu-Z360)-Ahx-f[CYwKTC]T-OH (I-70)

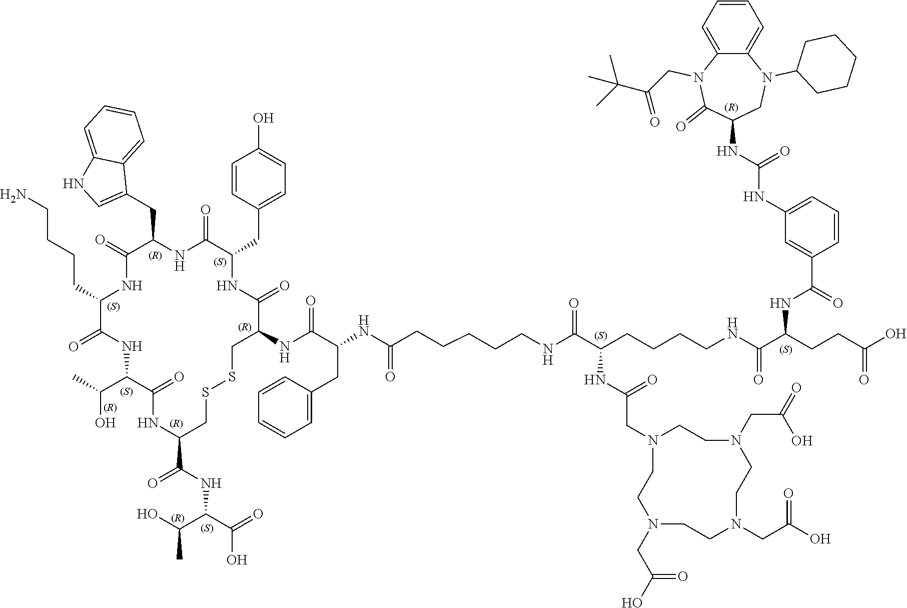

Molecular weight (average) calculated: 2308.70 g/mol

Determined by LC-MS: 1154.8(M+2H⁺); 770.2(M+3H⁺); 578.0(M+4H⁺)

Purity by UPLC (214 nm): 96%

### Example A71: DOTAGA-K(-Glu-Z360)-Ahx-f[CYwKTC]T-OH (I-71)

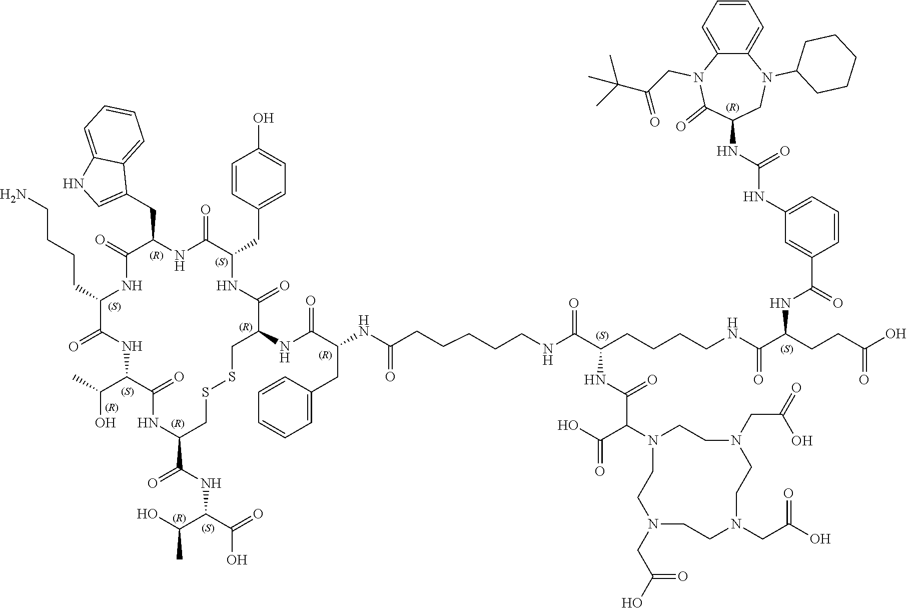

Molecular weight (average) calculated: 2380.76 g/mol

Determined by LC-MS: 1190.8(M+2H⁺); 794.3(M+3H⁺); 596.0(M+4H⁺)

Purity by UPLC (214 nm): 94%

### Example A72: DOTAGA-K(-Glu-Z360)-Ahx-Ahx-f[CYwKTC]T-OH (I-72)

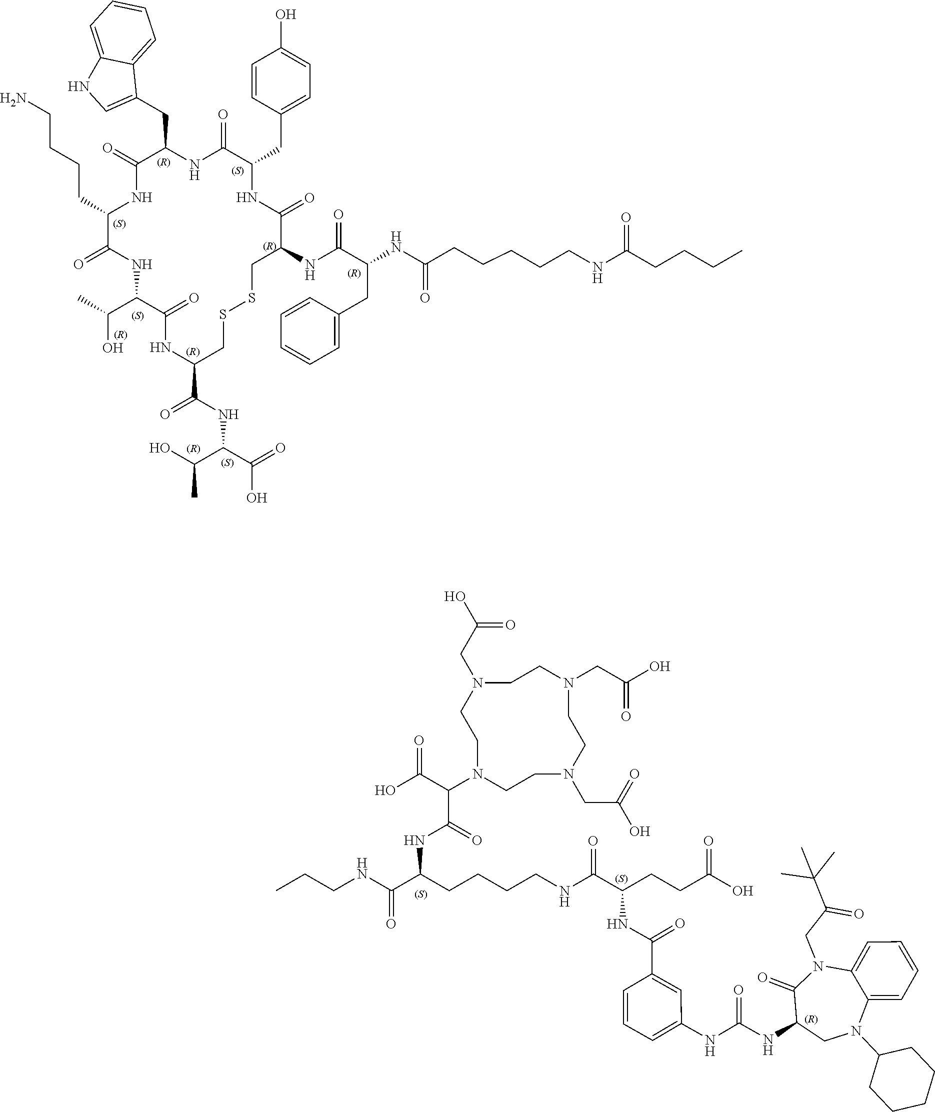

Molecular weight (average) calculated: 2493.92 g/mol

Determined by LC-MS: 1247.5(M+2H⁺); 831.9(M+3H⁺); 624.4(M+4H⁺)

Purity by UPLC (214 nm): 100%

### Example A73: DOTAGA-K(-Glu-Z360)-Aoc-Aoc-f[CYwKTC]T-OH (I-73)

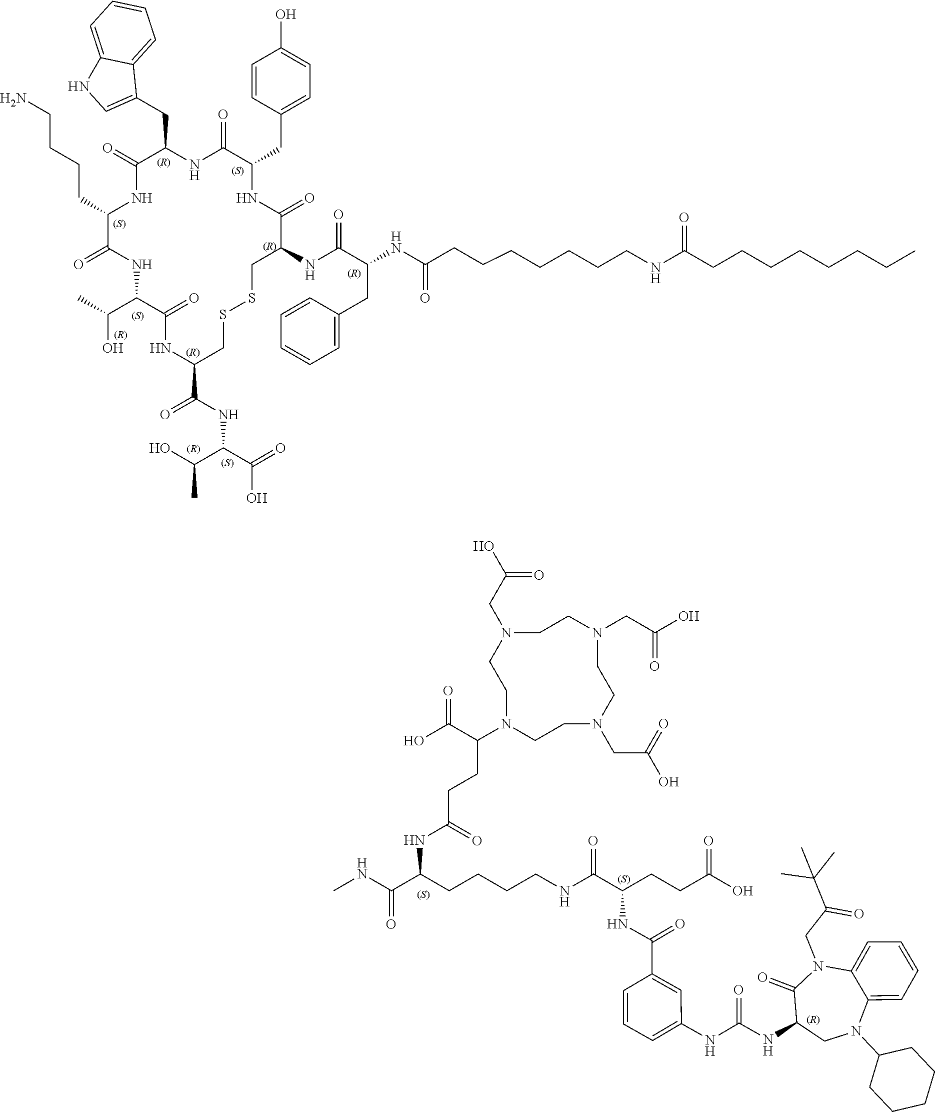

Molecular weight (average) calculated: 2550.03 g/mol

Determined by LC-MS: 1275.4(M+2H+); 850.8(M+3H+); 638.4(M+4H+)

Purity by UPLC (214 nm): 100%

### Example A74: DOTA-K(Z360)-gGlu-OEG-OEG-f[CYwKTC]T-OH (I-74)

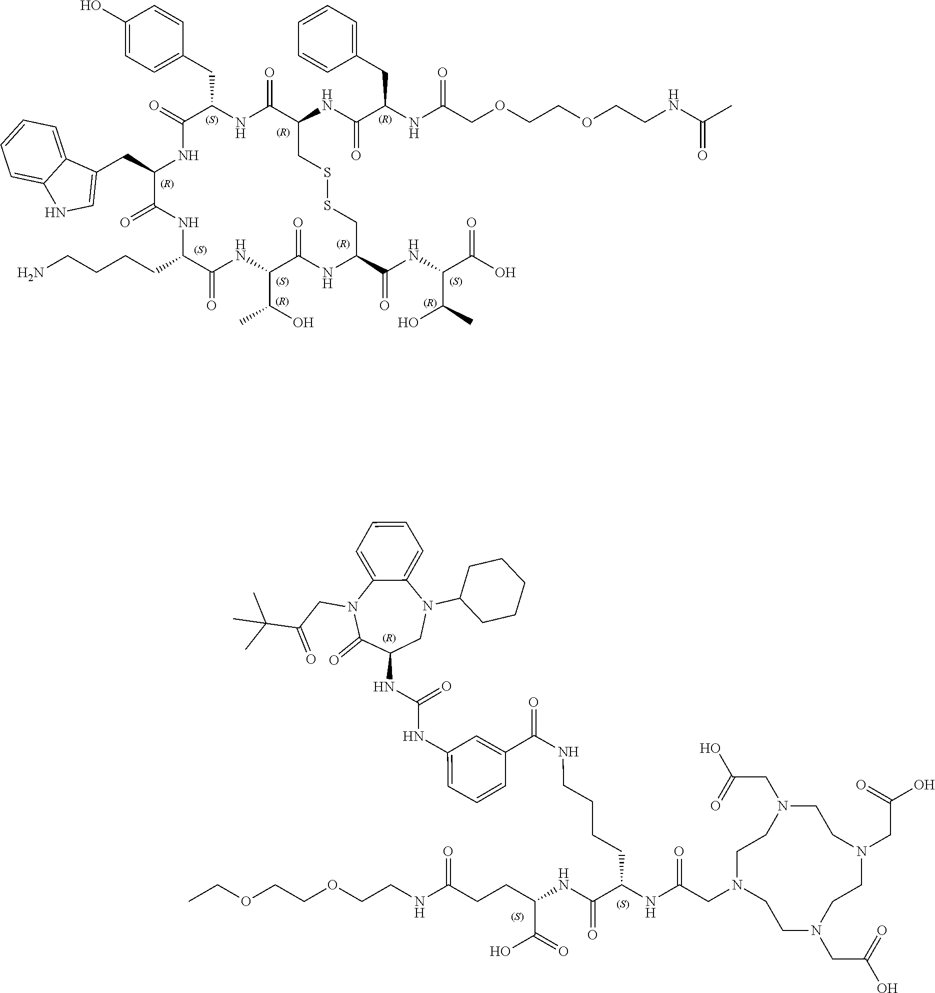

Molecular weight (average) calculated: 2485.85 g/mol

Determined by LC-MS: 1243.8(M+2H⁺); 829.3(M+3H⁺); 622.3(M+4H⁺)

Purity by UPLC (214 nm): 100%

### Example A75: DOTA-K(Z360)-Glu-gGlu-OEG-OEG-f[CYwKTC]T-OH (I-75)

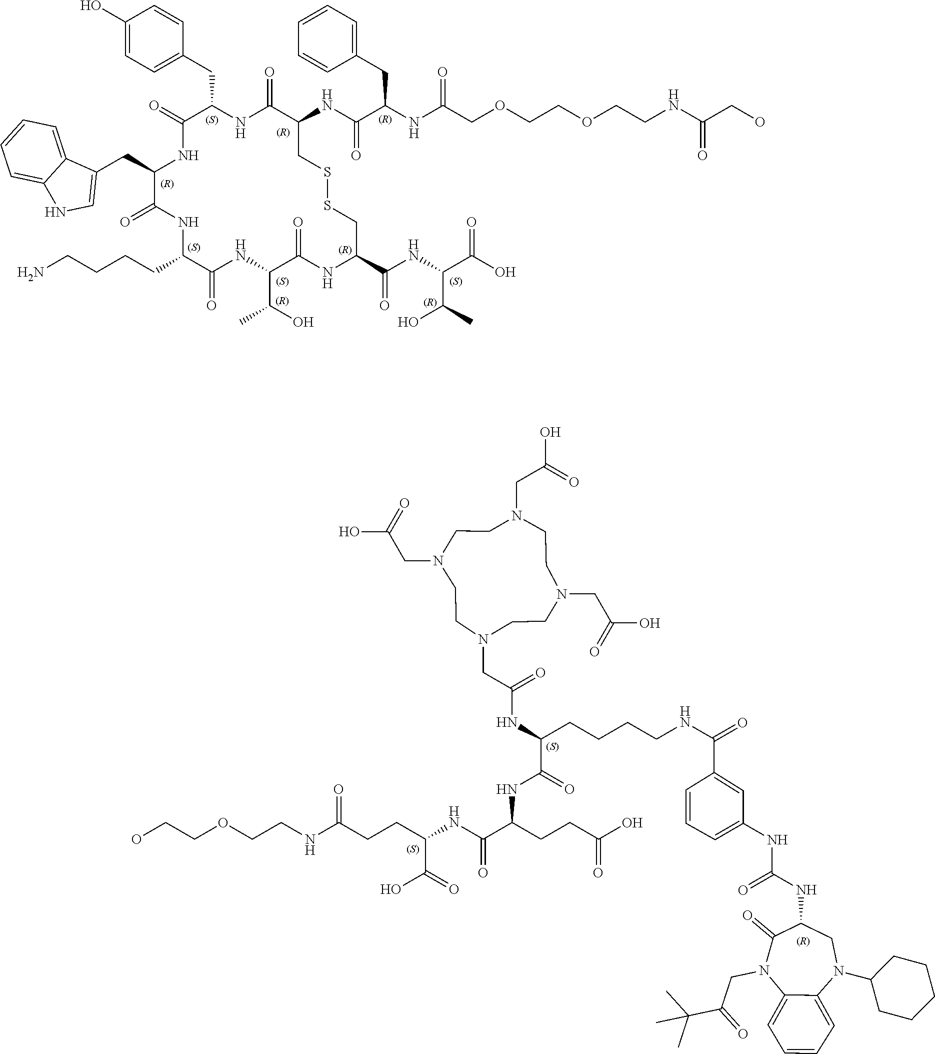

Molecular weight (average) calculated: 2614.97 g/mol PP14T

Determined by LC-MS: 872.3(M+3H⁺); 654.5(M+4H⁺)

Purity by UPLC (214 nm): 100%

### Example A 76: DOT A-K(-OEG-OEG-Glu-Z360)-OEG-f[CYwKTC]T-OH (1-76)

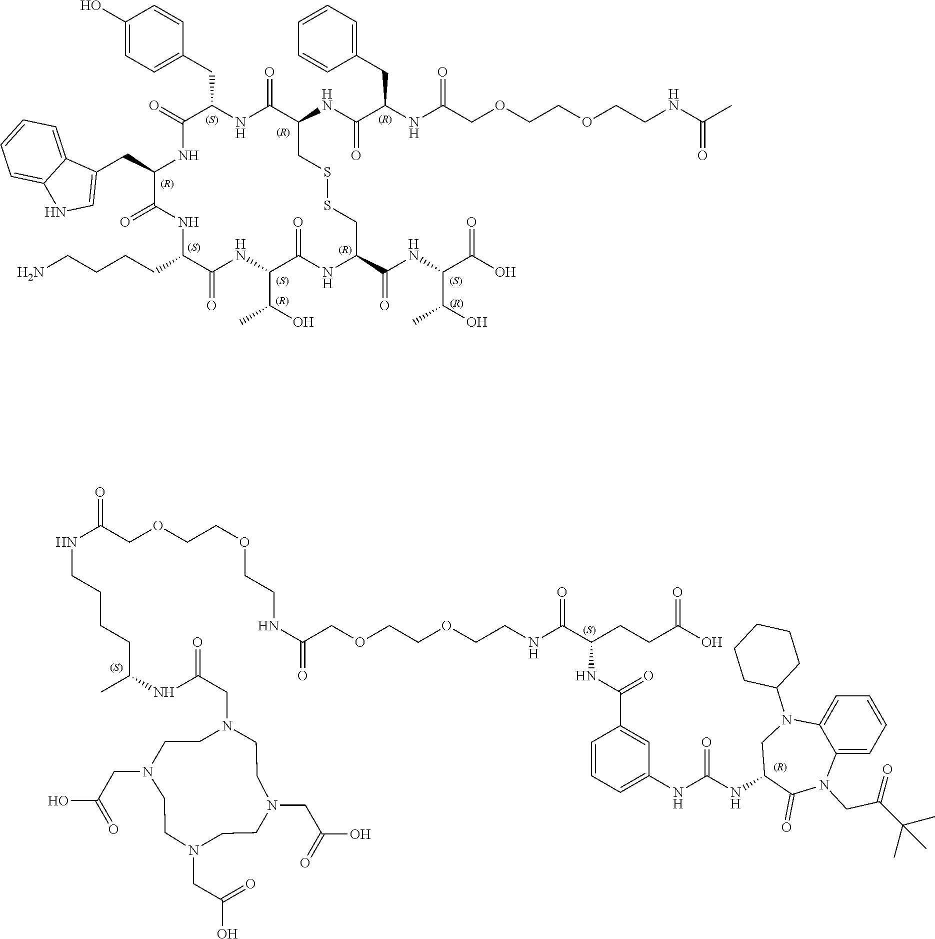

Molecular weight (average) calculated: 26314.97 g/mol

Determined by LC-MS: 1315(M+2H⁺); 87.6MH); 5.5(M+4H⁺)

Purity by UPLC (214 nm): 100%

### Example A77: DOTA-K(-OEG-gGlu-Ser-Z360)-OEG-f[CYwKTC]T-OH (I-77)

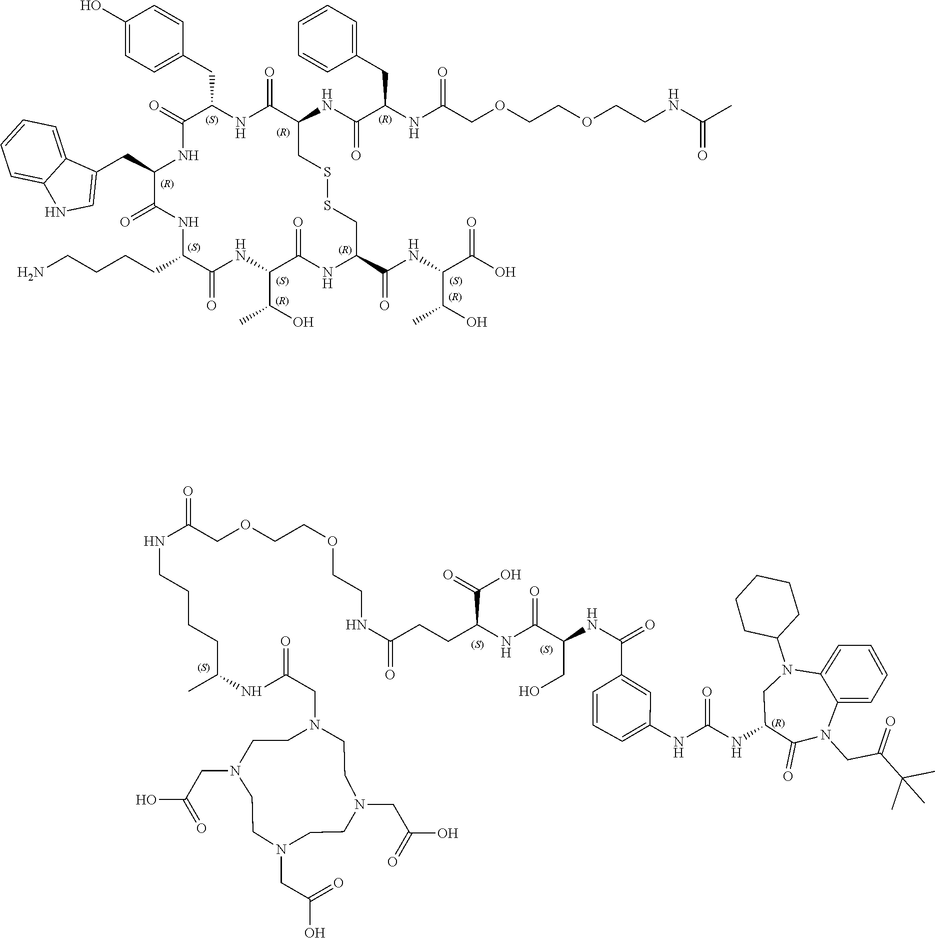

Molecular weight (average) calculated: 2572.93 g/mol

Determined by LC-MS: 1286.9(M+2H); 858.2(M+3H⁺); 644.0(M+4H+)

Purity by UPLC (214 nm): 100%

### Example A78: DOTA-K(-OEG-HGlu-Dap7Z360)-OEG-f[CYwKTC]T-OH (I-78)

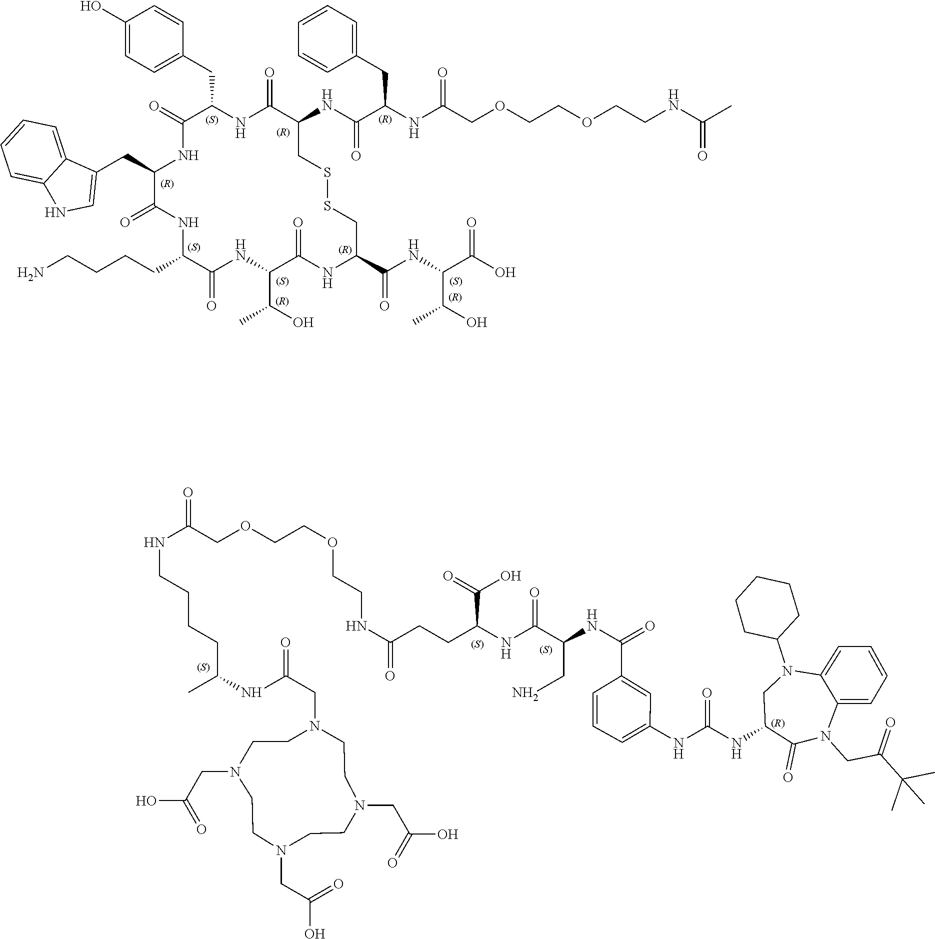

Molecular weight (average) calculated: 2571.95 g/mol

Determined by LC-MS: 857.9(M+3H+); 643.8(M+4H); 515.3(M+5H+)

Purity by UPLC (214 nm): 100%

### Example A79: DOTA-K(-OEG-His-Glu-Z360)-OEG-f[CYwKTC]T-OH (1-79)

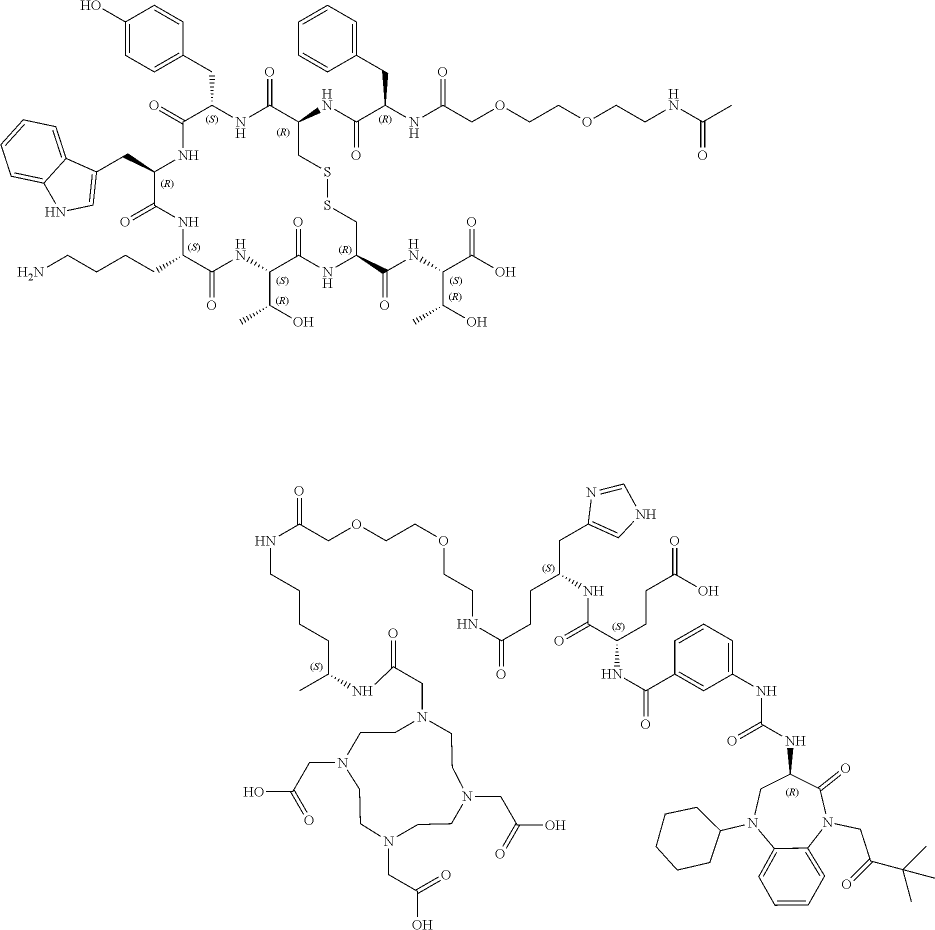

Molecular weight (average) calculated: 2608.97 g/mol

Determined by LC-MS: 874.9(M+3H+); 656.5(M+4H+); 525.5(M+5H+)

Purity by UPLC (214 nm): 100%

### Example A80: DOTA-K(-OEG-OEG-Cbp-Z360)-OEG-f[CYwKTC]T-OH (I-80)

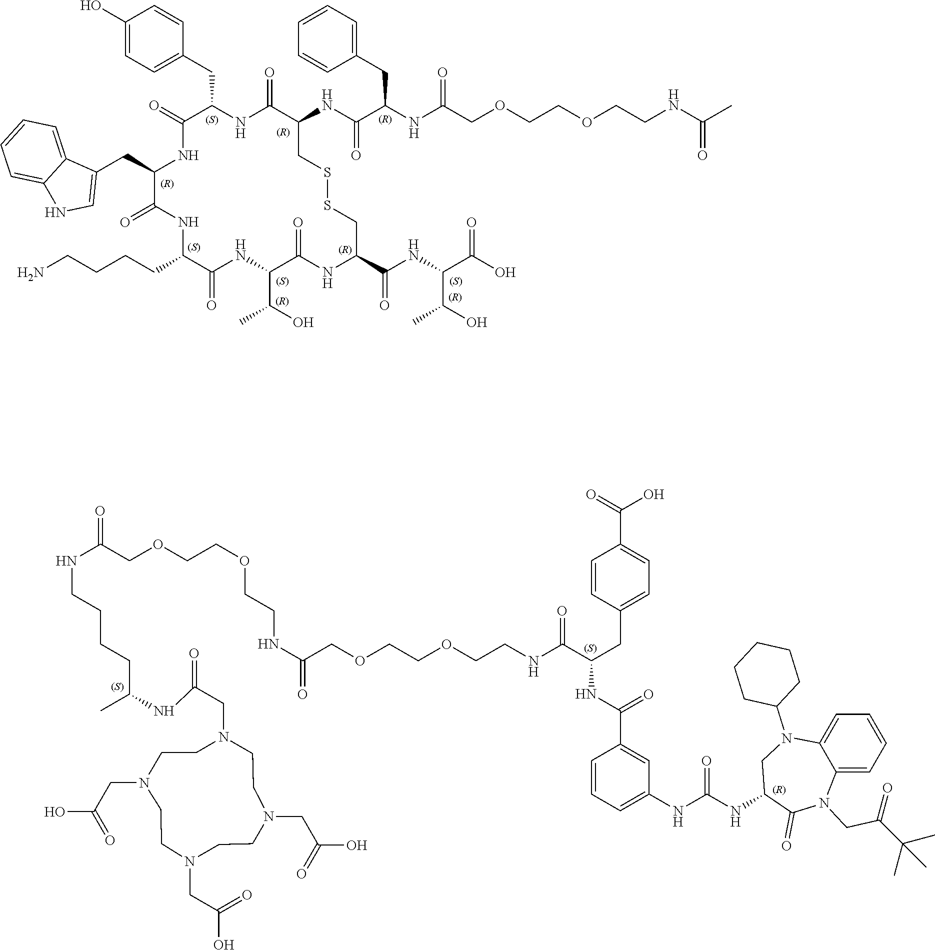

Molecular weight (average) calculated: 2693.08 g/mol

Determined by LC-MS: 898.2(M+3H⁺); 674.0(M+4H⁺)

Purity by UPLC (214 nm): 98%

### Comparative Compounds

The following comparative compounds were synthesized using the above methods.

### Comparator 1: DOTA-f[CYwKTC]T-OH (C-1)

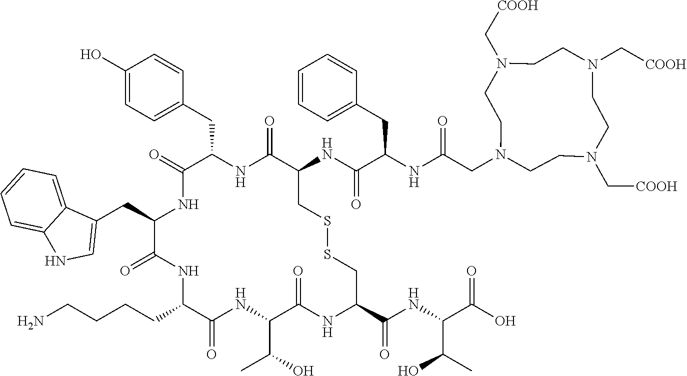 [SMILES: C[C@@H](O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O]

Molecular weight (average) calculated: 1435.6 g/mol

Determined by LC-MS: (M+2H)2+: 718.4; (M+3H)_{3+: 479.4}

Purity by UPLC (214 nm): 93.7%

### Comparator 2: DO TA-e-e-e-e-e-e-AYGW-Nle-DF-NH2 (C-2)

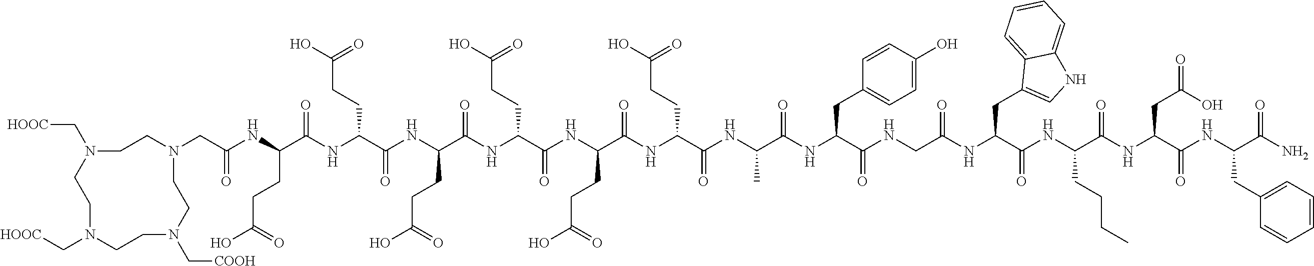 [SMILES: CCCC[C@H](NC(=O)[C@H](Cc1c[nH]c2ccccc12)NC(=O)CNC(=O)[C@H](Cc1ccc(O)cc1)NC(=O)[C@H](C)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)CN1CCN(CC(=O)O)CCN(CC(=O)O)CCN(CC(=O)O)CC1)C(=O)N[C@@H](CC(=O)O)C(=O)N[C@@H](Cc1ccccc1)C(N)=O]

Molecular weight (average) calculated: 2031.1 g/mol

Determined by LC-MS: (M+2H)2+: 1016.4; (M+3H)_{3+: 677.8}

Purity by UPLC (214 nm): 95.5%

### Comparator 3: Ac-Tyr-Gly-e-e-e-e-e-e-AYGW-NIe-DF-NH₂ (C-3)

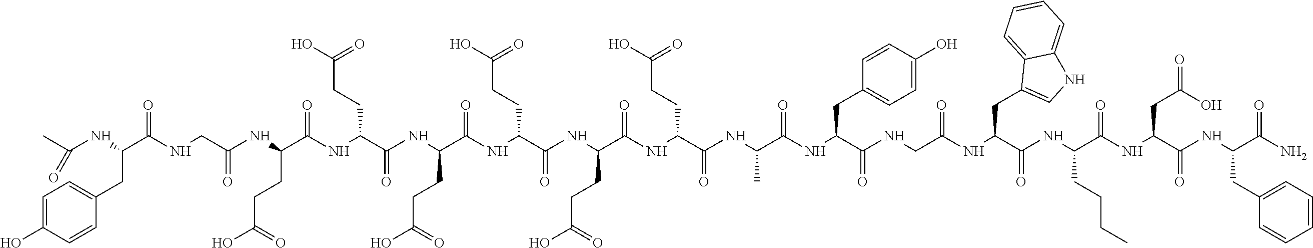 [SMILES: CCCC[C@H](NC(=O)[C@H](Cc1c[nH]c2ccccc12)NC(=O)CNC(=O)[C@H](Cc1ccc(O)cc1)NC(=O)[C@H](C)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)[C@@H](CCC(=O)O)NC(=O)CNC(=O)[C@H](Cc1ccc(O)cc1)NC(C)=O)C(=O)N[C@@H](CC(=O)O)C(=O)N[C@@H](Cc1ccccc1)C(N)=O]

Molecular weight (average) calculated: 1906.9 g/mol

Determined by LC-MS: (M+2H)2+: 953.9

Purity by UPLC (214 nm): 94.6%

### Comparator 4: Z360-gE-gE-gE-K(-OEG-OEG-DOTAGA)-NH₂ (C-4)

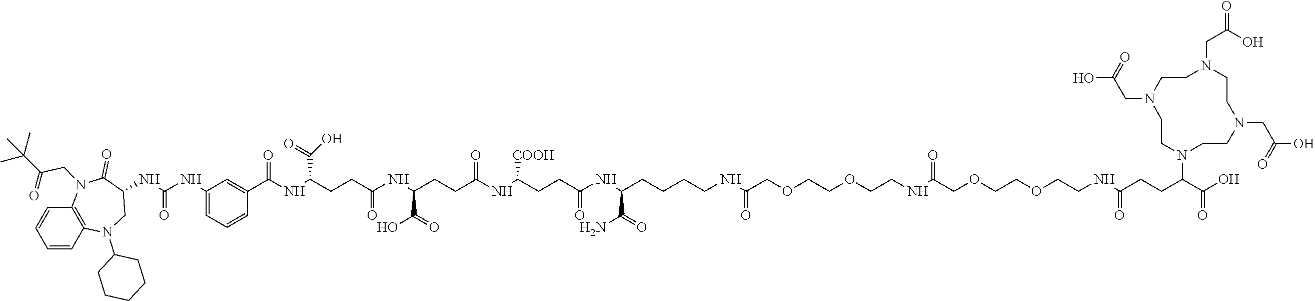 [SMILES: CC(C)(C)C(=O)CN1C(=O)[C@H](NC(=O)Nc2cccc(C(=O)N[C@@H](CCC(=O)N[C@@H](CCC(=O)N[C@@H](CCC(=O)N[C@@H](CCCCNC(=O)COCCOCCNC(=O)COCCOCCNC(=O)CCC(C(=O)O)N3CCN(CC(=O)O)CCN(CC(=O)O)CCN(CC(=O)O)CC3)C(N)=O)C(=O)O)C(=O)O)C(=O)O)c2)CN(C2CCCCC2)c2ccccc21]

Molecular weight (average) calculated: 1783.9 g/mol

Determined by LC-MS: (M+2H)2+:892.4; (M+3H)_{3+: 595.5}

Purity by UPLC (214 nm): 96.2%

### Comparator 5: DOTA-e-e-e-e-e-e-Ala-Tyr-Gly-Trp-Nle-Asp-Phe-NH2 (C-5)

Molecular weight (average) calculated: 2031.1 g/mol

Determined by LC-MS: (M+2H)2+:1016.4

Purity by UPLC (214 nm): 95%

### B: Biological Data

### Example B1: Cell-Based Binding Affinity of the Exemplary Compounds of the Present Application

AR42J cells (ATCC) were maintained in RPMI-1640 medium (Gibco) supplemented with 15% fetal bovine serum (Gibco) and 1% penicillin-streptomycin (BI) at 37° C. in a humidified incubator with 5% CO₂. The culture medium was replaced with fresh medium every 2-3 days. Experiments were performed with cells at 70-80% confluence.

### Binding Affinity Tested by Using ¹²⁵I-Labeled Competitive Ligands.

The binding affinities of test compounds were determined by a competitive cell-binding assay using ¹²⁵I-C1 and ¹²⁵I-C3 as the competitive ligand. The suspended AR42J cells at a density of 1~3×10⁶ cells/mL in binding buffer (RPMI-1640 medium supplemented with 0.25% bovine serum albumin) were transferred to a MultiScreen-DV Filter Plate (Millipore) with 100 μL per well except the blank group where the binding buffer was used instead. The cells were then incubated with ¹²⁵I-C1 and ¹²⁵I-C3 (0.02 μCi/well) in the presence of increasing concentrations (0-10000 nM) of test compounds at 37° C. for 1 h (n=3). The final volume in each well was maintained at 200 μL and insufficient volume was adjusted by the binding buffer. After the 1 h incubation, unbound ¹²⁵I-competitive ligands were removed by filtration using a Multiscreen vacuum manifold (Millipore) followed by rinses with the binding buffer (3 times). The filters were collected, and their radioactivity were individually measured by γ counter (2480 WIZARD2, PerkinElmer). The best-fit IC₅₀value (inhibitory concentration when 50% of the bound ¹²⁵I-C1 and ^{125l I-C}3 on cells were displaced) of test compounds were calculated by fitting the data with nonlinear regression using GraphPad Prism 8.0.1.

The results are shown in Table 1. The relative binding affinity of exemplary compounds of the application based on the mean value of the IC₅₀number is shown in Table 2.

The exemplary compounds of the present application all displayed binding ability to SSTR2 and C/K2R with a 10₅₀ value in the range of about 100 μM to about 1.0 μM in the above described radioligand competitive binding assays. Most bifunctional compounds demonstrated comparable or stronger activities on SSTR2 and CCK2R receptors when compared with their respective monofunctional comparators C-1 and C-2 respectively.

TABLE 1
IC₅₀ of selected compounds in radioligand
competitive binding assay using ¹²⁵I-C1
and ¹²⁵I-C3 as the competitive ligands.

| | CCK2R/IC⁵⁰ | SSTR2/IC⁵⁰ |
|---|---|---|
| | (M) | (M) |
| C-1 | ND | 2.81E−09 |
| C-2 | 1.7E−08 | >10000 |
| I-13 | 1.6E−09 | 1.23E−08 |
| I-20 | 1.4E−09 | 7.81E−10 |
| I-36 | 4.2E−09 | 1.60E−09 |
| I-37 | 4.2E−09 | 3.01E−09 |
| I-39 | 4.9E−09 | 3.67E−10 |
| I-45 | 9.9E−09 | 2.07E−09 |
| I-65 | 9.4E−09 | 1.36E−10 |

Table 2 shows the relative binding affinity of exemplary compounds of the application based on the mean value of the respective IC₅₀ number.

TABLE 2
Relative activity of exemplary compounds of the application based on the
mean value of the respective IC50 number.

| | | Relative | Relative |
|---|---|---|---|
| | | Binding | Binding |
| | | Affinity | Affinity |
| ID | Sequence | CCK2R | SSTR2 |
| C-1 | DOTA-f[CYwKTC]T-OH | ND | 1.00 |
| C-2 | DOTA-e-e-e-e-e-e-AYGW-Nle-DF-NH₂ | 1.00 | <0.001 |
| C-3 | Ac-YG-e-e-e-e-e-e-AYGW-Nle-DF-NH₂ | ND | ND |
| C-4 | Z360-gE-gE-gE-K(-OEG-OEG-DOTAGA)-NH₂ | 21.9 | ND |
| I-1 | DOTA-K(-gE-G-Z360)-OEG-f[CYwKTC]T-OH | 82.0 | 1.21 |
| I-2 | DOTA-K(-gE-G-Z360)-OEG-OEG-f[CYwKTC]T-OH | ND | 2.94 |
| I-3 | DOTA-K(-E-Z360)-OEG-f[CYwKTC]T-NH₂ | 5.3 | 0.50 |
| I-4 | DOTA-K(-E-Z360)-f[CYwKTC]T-NH₂ | 53.1 | 0.90 |
| I-5 | DOTA-K(-E-Z360)-OEG-f[CYwKTC]-Thr(ol) | 216 | 0.10 |
| I-6 | DOTA-K(-E-Z360)-f[CYwKTC]-Thr(ol) | 19.7 | 0.20 |
| I-7 | DOTA-gE-gE-gE-gE-OEG-K(-gE-G-Z360)-OEG-f[CYwKTC]T- | ND | 1.29 |
| | OH | | |
| I-8 | DOTA-gE-gE-gE-OEG-Lys(-gE-G-Z360)-OEG-f[CYwKTC]T- | 165 | 0.30 |
| | OH | | |
| I-9 | DOTA-gE-K(-gE-G-Z360)-gE-f[CYwKTC]T-OH | 13.5 | 2.85 |
| I-10 | DOTA-gE-gE-K(-gE-G-Z360)-OEG-f[CYwKTC]T-OH | 275 | 0.78 |
| I-11 | DOTA-gE-gE-OEG-K(-E-Z360)-OEG-f[CYwKTC]T-OH | 248 | 0.36 |
| I-12 | (Z360)-E-gE-OEG-gE-K(DOTA)-OEG-f[CYwKTC]T-OH | 255 | 0.61 |
| I-13 | (Z360)-gE-gE-OEG-K(DOTAGA)-OEG-f[CYwKTC]T-OH | 10.8 | 0.23 |
| I-14 | DOTA-gE-gE-OEG -K(-gE-G-Z360)-OEG-f[CYwKTC]T-OH | 3.80 | 3.50 |
| I-15 | DOTA-gE-gE-OEG-OEG-K(-gE-G-Z360)-OEG-f[CYwKTC]T- | 71.1 | 0.29 |
| | OH | | |
| I-16 | DOTA-gE-gE-OEG-OEG-K(-gE-G-Z360)-OEG-OEG- | 3.7 | 4.01 |
| | f[CYwKTC]T-OH | | |
| I-17 | (Z360)-gE-gE-OEG-OEG-K(DOTAGA)-OEG-OEG- | 17.3 | 0.16 |
| | f[CYwKTC]T-OH | | |
| I-18 | DOTA-gE-OEG-OEG-OEG-OEG-K(-gE-G-Z360)-OEG-gE- | 6.8 | 0.34 |
| | OEG-OEG-f[CYwKTC]T-OH | | |
| I-19 | DOTAGA-OEG-K(-gE-G-Z360)-f[CYwKTC]T-OH | 270 | 0.46 |
| I-20 | DOTAGA-OEG-K(-gE-G-Z360)-OEG-f[CYwKTC]T-OH | 12.1 | 3.60 |
| I-21 | DOTA-gE-OEG-OEG-K(-gE-G-Z360)-OEG-f[CYwKTC]T-OH | ND | 2.40 |
| I-22 | DOTA-gE-OEG-OEG-OEG-OEG-OEG-K(-gE-G-Z360)-OEG- | 2.7 | 2.31 |
| | f[CYwKTC]T-OH | | |
| I-23 | DOTA-OEG-OEG-OEG-K(-gE-G-Z360)-OEG-gE-OEG-OEG- | 12.0 | 13.7 |
| | f[CYwKTC]T-OH | | |
| I-24 | DOTA-gE-OEG-OEG-OEG-OEG-K(-gE-G-Z360)-OEG-OEG- | 5.0 | 0.38 |
| | OEG-OEG-f[CYwKTC]T-OH | | |
| I-25 | DOTA-K(-E-Z360)-f[CYwKTC]T-OH | ND | ND |
| I-26 | DOTA-K(-gE-G-Z360)-f[CYwKTC]T-OH | 6.0 | 1.09 |
| I-27 | DOTA-K(-E-Z360)-OEG-f[CYwKTC]T-OH | ND | 0.51 |
| I-28 | DOTA-K(-OEG-E-Z360)-f[CYwKTC]T-OH | 80.5 | 0.43 |
| I-29 | DOTA-K(-OEG-OEG-E-Z360)-f[CYwKTC]T-OH | 13.4 | 0.4 |
| I-30 | DOTA-K(-OEG-E-Z360)-OEG-f[CYwKTC]T-OH | 4.6 | 1.2 |
| I-31 | Z360-OEG-K(DOTAGA)-OEG-f[CYwKTC]T-OH | 8.8 | 4.75 |
| I-32 | Z360-G-gE-OEG-OEG-OEG-K(DOTA)-OEG-f[CYwKTC]T-OH | 6.4 | 1.15 |
| I-33 | DOTA-OEG-OEG-OEG-OEG-OEG-OEG-K(-gE-G-Z360)- | 3.3 | 3.20 |
| | OEG-f[CYwKTC]T-OH | | |
| I-34 | DOTA-K(Z360)-f[CYwKTC]T-OH | 5.9 | 1.49 |
| I-35 | DOTA-OEG-K(Z360)-f[CYwKTC]T-OH | ND | ND |
| I-36 | DOTA-K(-OEG-Z360)-f[CYwKTC]T-OH | 4.0 | 1.76 |
| I-37 | DOTA-K(Z360)-OEG-f[CYwKTC]T-OH | 4.0 | 0.93 |
| I-38 | DOTA-K(Z360)-PEG3-f[CYwKTC]T-OH | ND | ND |
| I-39 | DOTA-K(Z360)-OEG-OEG-f[CYwKTC]T-OH | 3.4 | 7.67 |
| I-40 | DOTA-K(Z360)-PEG6-f[CYwKTC]T-OH | ND | ND |
| I-41 | DOTA-OEG-OEG-K(Z360)-f[CYwKTC]T-OH | ND | ND |
| I-42 | DOTA-K(-OEG-OEG-Z360)-f[CYwKTC]T-OH | 115 | 0.54 |
| I-43 | DOTA-K(-OEG-Z360)-OEG-f[CYwKTC]T-OH | 4.0 | 0.61 |
| I-44 | DOTA-OEG-K(-OEG-Z360)-f[CYwKTC]T-OH | ND | ND |
| I-45 | DOTA-K(Z360)-OEG-OEG-OEG-f[CYwKTC]T-OH | 1.7 | 1.36 |
| I-46 | DOTA-K(-OEG-OEG-OEG-Z360)-f[CYwKTC]T-OH | 50.2 | 0.43 |
| I-47 | DOTA-eK-OEG-OEG-OEG-K(-gE-G-Z360)-OEG-f[CYwKTC]T- | 8.6 | 9.09 |
| | OH | | |
| I-48 | DOTA-PEG3-K(Z360)-f[CYwKTC]T-OH | ND | ND |
| I-49 | DOTA-G-G-K(Z360)-f[CYwKTC]T-OH | ND | ND |
| I-50 | DOTA-S-S-K(Z360)-f[CYwKTC]T-OH | ND | ND |
| I-51 | DOTA-PEG1-K(Z360)-f[CYwKTC]T-OH | ND | ND |
| I-52 | DOTA-OEG-K(Z360)-OEG-f[CYwKTC]T-OH | ND | ND |
| I-53 | DOTA-K(Z360)-OEG-G-f[CYwKTC]T-OH | ND | ND |
| I-54 | DOTA-K(Z360)-G-S-G-f[CYwKTC]T-OH | ND | ND |
| I-55 | DOTA-K(Z360)-G-p-G-p-G-f[CYwKTC]T-OH | ND | ND |
| I-56 | DOTA-G-p-G-K(Z360)-f[CYwKTC]T-OH | ND | ND |
| I-57 | DOTA-p-G-p-G-K(Z360)-f[CYwKTC]T-OH | ND | ND |
| I-58 | DOTA-G-S-K(Z360)-G-S-f[CYwKTC]T-OH | ND | ND |
| I-59 | DOTA-Aoc-K(Z360)-f[CYwKTC]T-OH | ND | ND |
| I-60 | DOTA-K(-G-G-G-Z360)-f[CYwKTC]T-OH | ND | ND |
| I-61 | DOTA-K(Z360)-Aoc-f[CYwKTC]T-OH | ND | ND |
| I-62 | DOTA-R-K(Z360)-f[CYwKTC]T-OH | ND | ND |
| I-63 | DOTA-K(Z360)-R-f[CYwKTC]T-OH | ND | ND |
| I-64 | DOTA-K(-OEG-Z360)-f[CYwKTC]T-NH₂ | ND | ND |
| I-65 | DOTA-eK-eK-OEG-OEG-K(-gE-G-Z360)-OEG-f[CYwKTC]T- | 1.8 | 20.7 |
| | OH | | |
| I-66 | DOTA-eK-eK-eK-OEG-K(-gE-G-Z360)-OEG-f[CYwKTC]T-OH | 26.1 | 34.6 |

Abbreviations:
T: Threonine; C: Cysteine; K: Lysine; w: D-Tryptophan; Y: Tyrosine; f: D-Phenylalanine; A: Alanine; G: Glycine; W: Tryptophan; Nle: Non-leucine; D: Aspartic acid; S: Serine; p: D-proline; R: Arginine:
gE: gamma-Glutamic acid; eK: epsilon-Lysine
-OH: C-terminal acid; - NH₂: C-terminal amide; Thr(ol): C-terminal alcohol
[CXXXXC]: Disulfide bond
DOTA: 1,4,7,10-Tetraazacyclododecane-1,4,7,10-tetraacetic acid;
DOTAGA: 2-[1,4,7,10-Tetraazacyclododecane-4,7,10-trisacetic acid]-pentanedioic acid
OEG: H₂N-[CH₂CH₂O]₂-CH₂CO₂H
PEG1: H₂N-CH₂CH₂O-CH₂CH₂CO₂H
PEG3: H₂N-[CH₂CH₂O]₃-CH₂CH₂CO₂H
PEG6: H₂N-[CH₂CH₂O]₆-CH₂CH₂CO₂H
Aoc: 8-amino-octanoic acid
Z360: Nastorazepide; CAS NO. 343326-69-2
ND: not determined

### Binding Affinity Tested by Using Cy5-Labled Competitive Ligands.

HEK 293T cells were transfected with SSTR2 or CCK2R-expressing plasmid by Lipofectamine 2000 reagent. 24 hours after transfection, cells were harvested and suspended with assay buffer (1% OVA in DMEM). Then cells were seeded into 96-well plate with Cy5-labled DOTA-TATE (for SSTR2, Seq: Cy5-OEG-Lys(DOTA)-f[CYwKTC]T-OH) and Cy5-labeled Z360 (for CCK2R, Seq: Z360-Glu-Glu-Glu-Lys(-OEG-Cy5)-NH2) as competitive ligands for compounds I-26, I-1, I-21, I-37, I-28, I-29, I-30, I-25, or the cells were seeded into 96-well plate with Cy5-labled DOTA-TATE (for SSTR2, Seq: Cy5-OEG-Lys(DOTA)-f[CYwKTC]T-OH) and Cy5-labeled F11N derivative (for CCK2R, Seq: Cy5-OEG-Lys(DOTA)-OEG-e-Ala-Tyr-Gly-Trp-Nle-Asp-Phe-NH2) as competitive ligands for compounds I-72, I-73, I-74, I-75, I-76, I-77, I-78, I-79, I-80. The testing compound was further supplemented into the wells at different concentrations. After incubation for 1 hour at dark, cells were washed and suspended in FACS buffer (1% OVA in DPBS). The fluorescent signal was detected by FACS machine and the binding affinity of the testing compound was evaluated by calculation of IC₅₀ for competitive binding of reference compound.

As shown in Table 3 and Table 4, the exemplary compounds of the present application all displayed binding ability to SSTR2 and CCK2R with a IC₅ value in the range of about 100 μM to about 1.0 μM in the assay as described.

TABLE 3
IC₅₀ of exemplary compounds using Cy5-labled DOTA-
TATE and Cy5-labled Z360 as competitive ligands

| | | CCK2R | SSTR2 |
|---|---|---|---|
| | | IC₅₀ | IC₅₀ |
| ID | Sequence | (nM) | (nM) |
| I-26 | DOTA-K(-gGlu-Gly-Z360)- | 2.67 | 2.13 |
| | f[CYwKTC]T-OH | | |
| I-1 | DOTA-K(-gGlu-Gly-Z360)-OEG- | 7.32 | 3.19 |
| | f[CYwKTC]T-OH | | |
| I-21 | DOTA-gGlu-OEG-OEG-K(-gGlu- | 7.13 | 5.95 |
| | Gly-Z360)-OEG-f[CYwKTC]T- | | |
| | OH | | |
| I-37 | DOTA-K(Z360)-OEG- | 10.68 | 0.96 |
| | f[CYwKTC]T-OH | | |
| I-28 | DOTA-K(-OEG-Glu-Z360)- | 4.84 | 1.73 |
| | f[CYwKTC]T-OH | | |
| I-29 | DOTA-K(-OEG-OEG-Glu-Z360)- | 0.46 | 2.13 |
| | f[CYwKTC]T-OH | | |
| I-30 | DOTA-K(-OEG-Glu-Z360)-OEG- | 1.28 | 1.81 |
| | f[CYwKTC]T-OH | | |
| I-25 | DOTA-K(-Glu-Z360)- | 1.36 | 0.98 |
| | f[CYwKTC]T-OH | | |

Abbreviations:
T: Threonine; C: Cysteine; K: Lysine; w: D-Tryptophan; Y: Tyrosine; f: D-Phenylalanine; A: Alanine; G: Glycine; W: Tryptophan; Nle: Non-leucine; D: Aspartic acid; S: Serine; p: D-proline; R: Arginine:
gE: gamma-Glutamic acid; ek: epsilon-Lysine
-OH: C-terminal acid; - NH₂: C-terminal amide; Thr(ol): C-terminal alcohol
[CXXXXC]: Disulfide bond
DOTA: 1,4,7,10-Tetraazacyclododecane-1,4,7,10-tetraacetic acid;
DOTAGA: 2-[1,4,7,10-Tetraazacyclododecane-4,7,10-trisacetic acid]-pentanedioic acid
OEG: H₂N-[CH₂CH₂O]₂-CH₂CO₂H
PEG1: H₂N-CH₂CH₂O-CH₂CH₂CO₂H
PEG3: H₂N-[CH₂CH₂O]₃-CH₂CH₂CO₂H
PEG6: H₂N-[CH₂CH₂O]₆-CH₂CH₂CO₂H
Aoc: 8-amino-octanoic acid
Z360: Nastorazepide; CAS NO. 343326-69-2
ND: not determined

TABLE 4
IC₅₀ of exemplary compounds using Cy5-labled DOTA-
TATE and Cy5-labled F11N derivative as competitive
ligands

| | | CCK2R | SSTR2 |
|---|---|---|---|
| | | IC₅₀ | IC₅₀ |
| ID | Sequence | (nM) | (nM) |
| C1 | DOTA-f[CYwKTC]T-OH | ND | 7.64 |
| C5 | DOTA-e-e-e-e-e-e-Ala-Tyr-Gly- | 25.71 | ND |
| | Trp-Nle-Asp-Phe-NH2 | | |
| I-72 | DOTAGA-K(-Glu-Z360)-Ahx- | 5.53 | 2.74 |
| | Ahx-f[CYwKTC]T-OH | | |
| I-73 | DOTAGA-K(-Glu-Z360)-Aoc- | 12.95 | 4.45 |
| | Aoc-f[CYwKTC]T-OH | | |
| I-74 | DOTA-K(Z360)-gGlu-OEG- | 56.73 | 5.53 |
| | OEG-f[CYwKTC]T-OH | | |
| I-75 | DOTA-K(Z360)-Glu-gGlu-OEG- | 126.20 | 9.36 |
| | OEG-f[CYwKTC]T-OH | | |
| I-76 | DOTA-K(-OEG-OEG-Glu-Z360)- | 11.17 | 5.44 |
| | OEG-f[CYwKTC]T-OH | | |
| I-77 | DOTA-K(-OEG-gGlu-Ser-Z360)- | 19.42 | 4.77 |
| | OEG-f[CYwKTC]T-OH | | |
| I-78 | DOTA-K(-OEG-gGlu-Dap-Z360)- | 30.48 | 6.13 |
| | OEG-f[CYwKTC]T-OH | | |
| I-79 | DOTA-K(-OEG-His-Glu-Z360)- | 16.50 | 6.75 |
| | OEG-f[CYwKTC]T-OH | | |
| I-80 | DOTA-K(-OEG-OEG-Cbp- | 24.21 | 5.03 |
| | Z360)-OEG-f[CYwKTC]T-OH | | |

Abbreviations:
T: Threonine; C: Cysteine; K: Lysine; w: D-Tryptophan; Y: Tyrosine; f: D-Phenylalanine; A: Alanine; G: Glycine; W: Tryptophan; Nle: Non-leucine; D: Aspartic acid; S: Serine; p: D-proline; R: Arginine, H: Histidine, E: Glutamic acid:
gE: gamma-Glutamic acid; eK: epsilon-Lysine
-OH: C-terminal acid; - NH2: C-terminal amide; Thr(ol): C-terminal alcohol
[CXXXXC]: Disulfide bond
DOTA: 1,4,7,10-Tetraazacyclododecane-1,4,7,10-tetraacetic acid;
DOTAGA: 2-[1,4,7, 10-Tetraazacyclododecane-4,7,10-trisacetic acid]-pentanedioic acid
OEG: H₂N-[CH₂CH₂O]₂-CH₂CO₂H
PEG1: H₂N-CH₂CH₂O-CH₂CH₂CO₂H
PEG3: H₂N-[CH₂CH₂O]₃-CH₂CH₂CO₂H
PEG6: H₂N-[CH₂CH₂O]₆-CH₂CH₂CO₂H
Aoc: 8-amino-octanoic acid
Cbp: 4-Carboxy-L-phenylalanine
Dap: 2,3-diaminoproprionic acid
Z360: Nastorazepide; CAS NO. 343326-69-2
ND: not determined

### Example B2: Biodistribution Study

All animal care and experimental procedure were performed by following the animal protocols approved by the ethics committee of China Institute of Radiation Protection. The BALB/c nude mice (Charles River, Beijing) were utilized for these studies. Mice were below 5 per cage in sterile, microisolator cages under temperature- and humidity-controlled conditions with a 12 h light/12 h dark schedule and fed irradiated rodent chow and reverse osmosis (RO) sterile water ad libitum. In preparation for tumor cell inoculations, mice were anesthetized with isoflurane (RWD Life Science Inc.) at an induction rate of 4% and maintained at a rate of 2.5% with 0.4 L oxygen delivered via precision vaporizer and a non-rebreathing apparatus. These mice received subcutaneous rear flank injections of approximately 4×10⁶ AR42J cells (human pancreatic cancer cell line) suspended in 200 μL of phosphate-buffered saline (PBS) and Matrigel (Corning) (1/1). Xenografted tumors were allowed to grow for ~2-4 weeks post-inoculation and ranged in mass from 0.05 to 0.50 g (average tumor size, 0.20 g). Biodistribution studies in nude mice were performed by the tail vein injection of each test compound, ~10-50 μCi (~0.37-1.85 MBq) with specific activity 50-250 μCi/nmol, delivered in 100 μL of 0.9% NaCl. The mice were euthanized, and the tissues and organs were excised from the animals at 4, 24 and 72 h post-injection. The tissues and organs were weighed, counted in a PerkinElmer 2480 WIZARD2 γ counter, and the percent injected dose (% ID) and % ID/g of each organ or tissue were calculated.

All tested exemplary compounds demonstrated measurable tumor uptake of the radionuclide. The uptake of radionuclide in normal organs including kidney and liver were found to be dependent on the net charge of the compound-radionuclide complex as well as the length and chemical properties of the linkers.

Tables 5-15 show the biodistribution of various compounds in AR42J tumor-bearing mice.

Table 5 shows the impact of net charge number and total combined linker length on the biodistribution: tumor and organ uptake of ¹⁷⁷Lu-labeled compounds of Formula I at 24 hours after dosing and the respective tumor-to-organ ratios.

Charge is presented as the ¹⁷⁷Lu chelated state of the exemplary compound of Formula I at physiological pH. For example, when E is a DOTA moiety (a chelating group derived from DOTA), which comprises of three free carboxylic acid groups, the DOTA moiety when complexed to ¹⁷⁷Lu would have a 0 net charge. Similarly, when E is a DOTAGA moiety, which comprises four free carboxylic acid groups, the DOTAGA moiety when complexed to Lu-17 would have a −1 net charge. Further, C-terminal and side chains groups having free carboxyl groups, including, for example, glutamic acid or gamma-glutamic acid, would be counted as contributing −1 charge for each free carboxyl group; and N-terminal and side chain groups having a free amine (e.g,. epsilon amine of lysine or the guanidine group of arginine) would be counted as contributing a +1 charge for each free amine.

TABLE 5
The uptake (% ID/g) of ¹⁷⁷Lu-labeled compounds at 24 hours after dosing
in tumor and normal organs and the respective tumor-to-organ ratios.

| | | Linker | | | | | | | | | | | |
|---|---|---|---|---|---|---|---|---|---|---|---|---|---|
| | Net | length | | | | | | | T/ | T/ | T/ | T/ | T/ |
| ID | Charge | (atoms) | Kidney | Tumor | Liver | Spleen | Stomach | Pancreas | K | L | P | Sp | St |
| I-9 | −3 | 18 | 42.2 | 10.9 | 1.31 | 1.03 | 3.22 | 2.89 | 0.3 | 8.3 | 3.8 | 11 | 3.4 |
| I-13 | −3 | 28 | 46.7 | 14.8 | 1.21 | 0.47 | 2.19 | 1.21 | 0.3 | 12 | 12 | 32 | 6.8 |
| I-14 | −3 | 36 | 68.6 | 14.9 | 2.24 | 0.56 | 2.25 | 3.37 | 0.2 | 6.7 | 4.4 | 27 | 6.6 |
| I-22 | −2 | 67 | 21.9 | 12.7 | 1.10 | 1.1 | 1.7 | 2.6 | 0.6 | 12 | 5.0 | 12 | 7.5 |
| I-19 | −2 | 17 | 51.6 | 31.3 | 0.96 | 0.68 | 7.24 | 2.07 | 0.6 | 35 | 15 | 46 | 4.3 |
| I-27 | −1 | 9 | 21.0 | 28.5 | 1.11 | 0.44 | 4.51 | 1.79 | 1.4 | 26 | 16 | 64 | 6.3 |
| I-1 | −1 | 17 | 14.0 | 17.1 | 2.86 | 1.60 | 2.53 | 1.60 | 1.2 | 6.0 | 11 | 11 | 6.8 |
| I-34 | zero | 0 | 6.1 | 22.7 | 19.8 | 9.5 | 5.7 | 3.2 | 3.7 | 1.1 | 7.0 | 2.4 | 4.0 |
| I-35 | zero | 9 | 6.1 | 17.8 | 5.06 | 0.47 | 3.09 | 0.96 | 2.9 | 3.5 | 19 | 38 | 5.8 |
| I-48 | zero | 13 | 10.6 | 18.9 | 7.02 | 1.02 | 4.75 | 1.35 | 1.8 | 2.7 | 14 | 19 | 4.0 |
| I-36 | zero | 9 | 7.4 | 21.6 | 5.0 | 0.6 | 2.0 | 1.6 | 2.9 | 4.3 | 13 | 36 | 11 |
| I-37 | zero | 9 | 7.79 | 23.1 | 2.40 | 0.20 | 2.58 | 0.68 | 3.0 | 9.6 | 34 | 115 | 8.9 |
| I-38 | zero | 13 | 7.9 | 14.3 | 2.3 | 0.2 | 3.4 | 0.8 | 1.8 | 6.1 | 18 | 72 | 4.2 |
| I-39 | zero | 18 | 12.0 | 27.5 | 2.78 | 0.31 | 4.63 | 0.94 | 2.3 | 9.9 | 29 | 89 | 5.9 |
| I-40 | zero | 22 | 8.9 | 17.9 | 3.1 | 0.3 | 1.8 | 0.3 | 2.0 | 5.7 | 59 | 53 | 10 |
| I-41 | zero | 18 | 9.7 | 16.1 | 1.5 | 0.3 | 4.0 | 0.7 | 1.7 | 10 | 24 | 55 | 4.0 |
| I-52 | zero | 18 | 14.0 | 12.4 | 5.7 | 0.3 | 5.1 | 0.9 | 0.9 | 2.2 | 14 | 36 | 2.4 |
| I-42 | zero | 18 | 17.8 | 18.7 | 5.6 | 0.3 | 2.1 | 1.6 | 1.1 | 3.3 | 12 | 62 | 8.8 |
| I-49 | zero | 6 | 5.93 | 8.97 | 5.92 | 0.62 | 2.77 | 0.92 | 1.5 | 1.5 | 9.8 | 14 | 3.2 |
| I-53 | zero | 12 | 7.15 | 16.2 | 2.57 | 0.31 | 3.36 | 0.91 | 2.3 | 6.3 | 18 | 52 | 4.8 |
| I-55 | zero | 15 | 20.8 | 23.0 | 9.21 | 0.67 | 3.13 | 1.66 | 1.1 | 2.5 | 14 | 34 | 7.3 |
| I-54 | zero | 9 | 10.5 | 25.8 | 9.60 | 0.71 | 4.10 | 2.65 | 2.4 | 2.7 | 9.7 | 36 | 6.3 |
| I-61 | zero | 9 | 1.53 | 5.41 | 53.3 | 28.3 | 1.21 | 0.34 | 3.5 | 0.1 | 16 | 0.2 | 4.5 |
| I-3 | zero | 12 | 16.5 | 13.6 | 1.16 | 0.30 | 3.24 | 1.05 | 0.8 | 12 | 13 | 45 | 4.2 |
| I-62 | +1 | 3 | 14.7 | 10.8 | 32.4 | 0.82 | 1.68 | 1.78 | 0.7 | 0.3 | 6.1 | 13 | 6.4 |
| I-66 | +2 | 47 | 9.35 | 1.35 | 1.20 | 0.05 | 0.37 | 0.30 | 0.1 | 1.1 | 4.4 | 26 | 3.6 |

T/K: Tumor/Kidney; T/L: Tumor/Liver; T/Sp: Tumor/Spleen; T/St: Tumor/Stomach; T/P: Tumor/Pancreas.

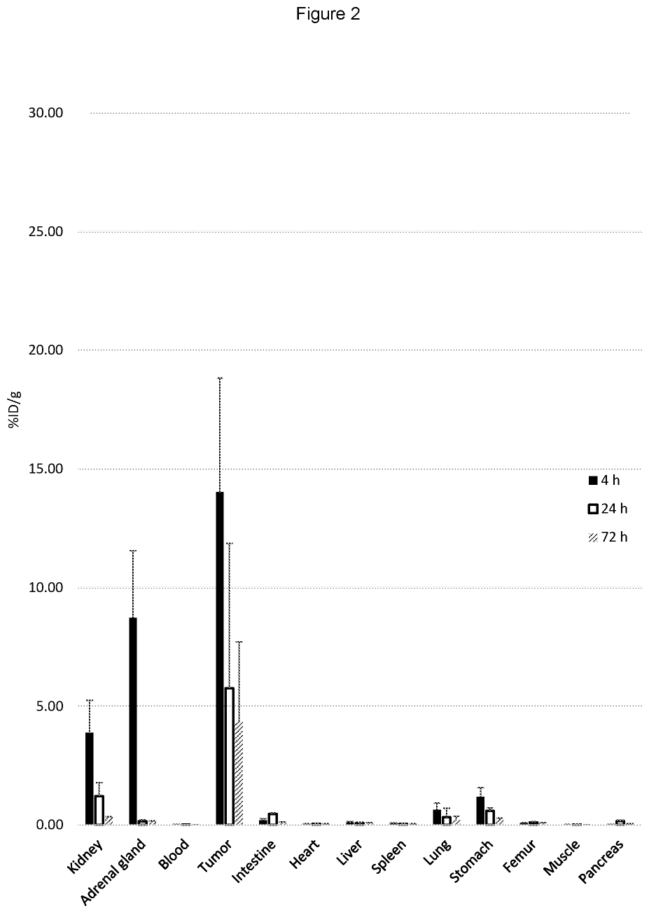

TABLE 6 and FIG. 2 shows the biodistribution of ¹⁷⁷Lu-C-1 in AR42J tumor-bearing mice (n=3)

TABLE 6

| | | | | 4 h | 24 h | 72 h |
|---|---|---|---|---|---|---|
| % ID/g | 4 h | 24 h | 72 h | STDEV | STDEV | STDEV |
| Kidney | 3.88 | 1.21 | 0.33 | 1.37 | 0.57 | 0.02 |
| Adrenal gland | 8.73 | 0.15 | 0.13 | 2.82 | 0.07 | 0.05 |
| Blood | 0.02 | 0.01 | 0.01 | 0.00 | 0.00 | 0.00 |
| Tumor | 14.02 | 5.76 | 4.33 | 4.82 | 6.10 | 3.39 |
| Intestine | 0.20 | 0.46 | 0.11 | 0.07 | 0.06 | 0.02 |
| Heart | 0.06 | 0.05 | 0.04 | 0.00 | 0.00 | 0.01 |
| Liver | 0.10 | 0.07 | 0.06 | 0.05 | 0.04 | 0.04 |
| Spleen | 0.08 | 0.04 | 0.05 | 0.01 | 0.02 | 0.01 |
| Lung | 0.63 | 0.32 | 0.21 | 0.30 | 0.38 | 0.15 |
| Stomach | 1.18 | 0.59 | 0.21 | 0.39 | 0.13 | 0.08 |
| Femur | 0.07 | 0.11 | 0.08 | 0.03 | 0.03 | 0.02 |
| Muscle | 0.01 | 0.02 | 0.01 | 0.01 | 0.02 | 0.00 |
| Pancreas | 0.02 | 0.16 | 0.03 | 0.01 | 0.05 | 0.03 |
| Fat | ND | ND | ND | ND | ND | ND |

TABLE 7 shows the biodistribution of ¹⁷⁷Lu-C-2 in AR42J tumor-bearing mice (n=3)

TABLE 7

| | | | 4 h | 24 h |
|---|---|---|---|---|
| % ID/g | 4 h | 24 h | STDEV | STDEV |
| Kidney | 2.72 | 0.71 | 1.19 | 0.10 |
| Adrenal gland | ND | ND | ND | ND |
| Blood | 0.08 | 0.01 | 0.01 | 0.00 |
| Tumor | 1.07 | 0.82 | 0.18 | 0.47 |
| Intestine | 0.06 | 0.05 | 0.01 | 0.02 |
| Heart | 0.05 | 0.04 | 0.00 | 0.01 |
| Liver | 0.15 | 0.12 | 0.03 | 0.04 |
| Spleen | 0.06 | 0.05 | 0.03 | 0.01 |
| Lung | 0.06 | 0.11 | 0.04 | 0.11 |
| Stomach | 0.21 | 0.17 | 0.04 | 0.06 |
| Femur | 0.12 | 0.24 | 0.06 | 0.08 |
| Muscle | 0.06 | 0.01 | 0.07 | 0.01 |
| Pancreas | 0.01 | 0.02 | 0.00 | 0.00 |
| Fat | ND | ND | ND | ND |

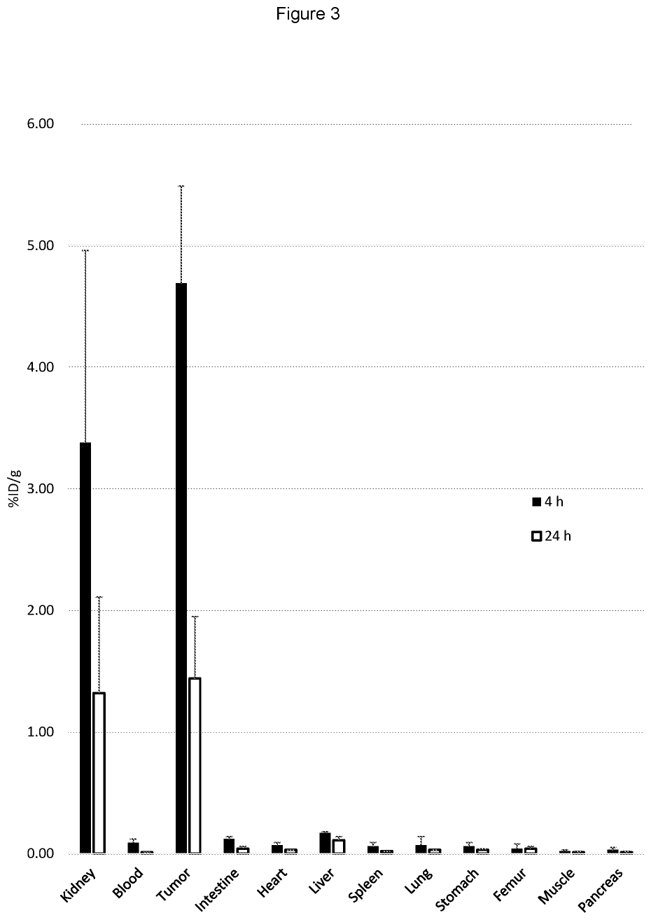

TABLE 8 and FIG. 3 shows the biodistribution of ¹⁷⁷Lu-C-4 in AR42J tumor-bearing mice (n=3)

TABLE 8

| | | | 4 h | 24 h |
|---|---|---|---|---|
| % ID/g | 4 h | 24 h | STDEV | STDEV |
| Kidney | 3.38 | 1.32 | 1.58 | 0.79 |
| Adrenal gland | ND | ND | ND | ND |
| Blood | 0.09 | 0.01 | 0.03 | 0.00 |
| Tumor | 4.69 | 1.44 | 0.80 | 0.51 |
| Intestine | 0.12 | 0.04 | 0.02 | 0.02 |
| Heart | 0.07 | 0.03 | 0.02 | 0.00 |
| Liver | 0.17 | 0.11 | 0.01 | 0.03 |
| Spleen | 0.06 | 0.02 | 0.03 | 0.00 |
| Lung | 0.07 | 0.03 | 0.07 | 0.00 |
| Stomach | 0.06 | 0.03 | 0.03 | 0.01 |
| Femur | 0.04 | 0.04 | 0.04 | 0.02 |
| Muscle | 0.02 | 0.01 | 0.01 | 0.01 |
| Pancreas | 0.03 | 0.01 | 0.02 | 0.01 |
| Fat | ND | ND | ND | ND |

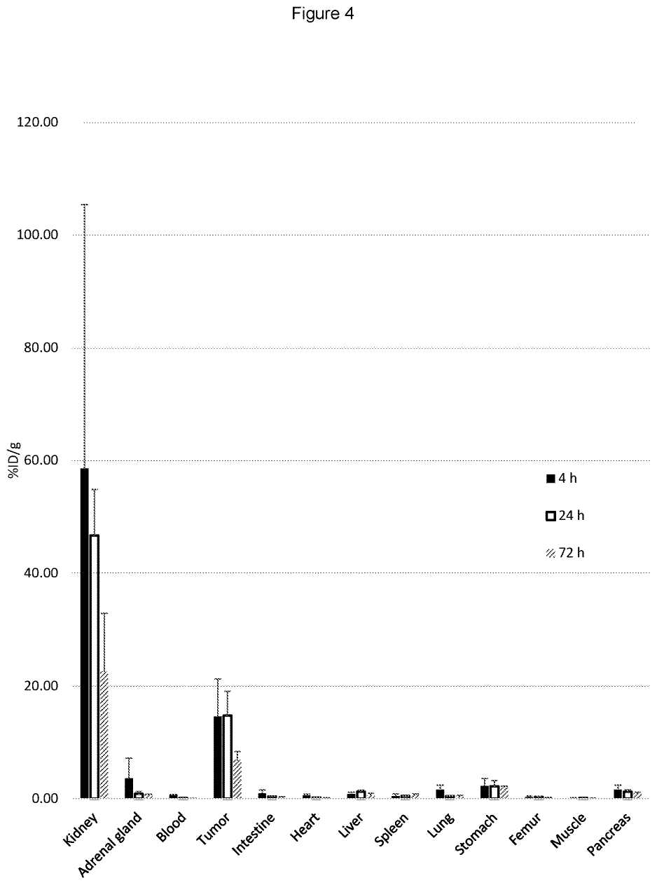

TABLE 9 and FIG. 4 shows the biodistribution of ¹⁷⁷Lu-1-13 in AR42J tumor-bearing mice (n=3).

TABLE 9

| | | | | 4 h | 24 h | 72 h |
|---|---|---|---|---|---|---|
| % ID/g | 4 h | 24 h | 72 h | STDEV | STDEV | STDEV |
| Kidney | 58.56 | 46.68 | 22.52 | 46.86 | 8.16 | 10.40 |
| Adrenal gland | 3.60 | 0.81 | 0.73 | 3.57 | 0.41 | 0.06 |
| Blood | 0.64 | 0.09 | 0.03 | 0.07 | 0.02 | 0.00 |
| Tumor | 14.56 | 14.75 | 6.83 | 6.69 | 4.32 | 1.52 |
| Intestine | 0.89 | 0.37 | 0.22 | 0.67 | 0.06 | 0.10 |
| Heart | 0.54 | 0.19 | 0.11 | 0.22 | 0.05 | 0.02 |
| Liver | 0.79 | 1.21 | 0.52 | 0.32 | 0.28 | 0.40 |
| Spleen | 0.43 | 0.47 | 0.64 | 0.36 | 0.08 | 0.18 |
| Lung | 1.57 | 0.43 | 0.38 | 0.82 | 0.02 | 0.23 |
| Stomach | 2.23 | 2.19 | 1.95 | 1.35 | 1.01 | 0.24 |
| Femur | 0.21 | 0.27 | 0.14 | 0.24 | 0.12 | 0.05 |
| Muscle | 0.08 | 0.08 | 0.05 | 0.05 | 0.02 | 0.03 |
| Pancreas | 1.59 | 1.21 | 0.91 | 0.84 | 0.36 | 0.25 |
| Fat | ND | ND | ND | ND | ND | ND |

TABLE 10 shows the biodistribution of ¹⁷⁷Lu-1-14 in AR42J tumor-bearing mice (n=3)

TABLE 10

| | | | | 4 h | 24 h | 72 h |
|---|---|---|---|---|---|---|
| % ID/g | 4 h | 24 h | 72 h | STDEV | STDEV | STDEV |
| Kidney | 48.22 | 68.61 | 24.31 | 17.67 | 54.33 | 6.05 |
| Adrenal gland | 3.55 | 2.72 | 1.44 | 0.35 | 1.95 | 0.67 |
| Blood | 1.32 | 0.09 | 0.02 | 0.26 | 0.02 | 0.01 |
| Tumor | 30.87 | 14.91 | 17.96 | 5.58 | 11.44 | 16.01 |
| Intestine | 0.60 | 1.25 | 0.37 | 0.19 | 1.18 | 0.23 |
| Heart | 0.80 | 0.17 | 0.19 | 0.15 | 0.09 | 0.00 |
| Liver | 3.31 | 2.24 | 1.28 | 1.46 | 0.63 | 0.37 |
| Spleen | 0.64 | 0.56 | 2.17 | 0.17 | 0.03 | 0.67 |
| Lung | 1.32 | 0.72 | 0.95 | 0.31 | 0.11 | 0.09 |
| Stomach | 3.68 | 2.25 | 2.99 | 0.63 | 0.69 | 0.52 |
| Femur | 0.49 | 0.40 | 0.31 | 0.20 | 0.28 | 0.15 |
| Muscle | 0.17 | 0.17 | 0.11 | 0.07 | 0.04 | 0.04 |
| Pancreas | 2.11 | 3.37 | 3.14 | 0.36 | 0.48 | 0.12 |
| Fat | ND | ND | ND | ND | ND | ND |

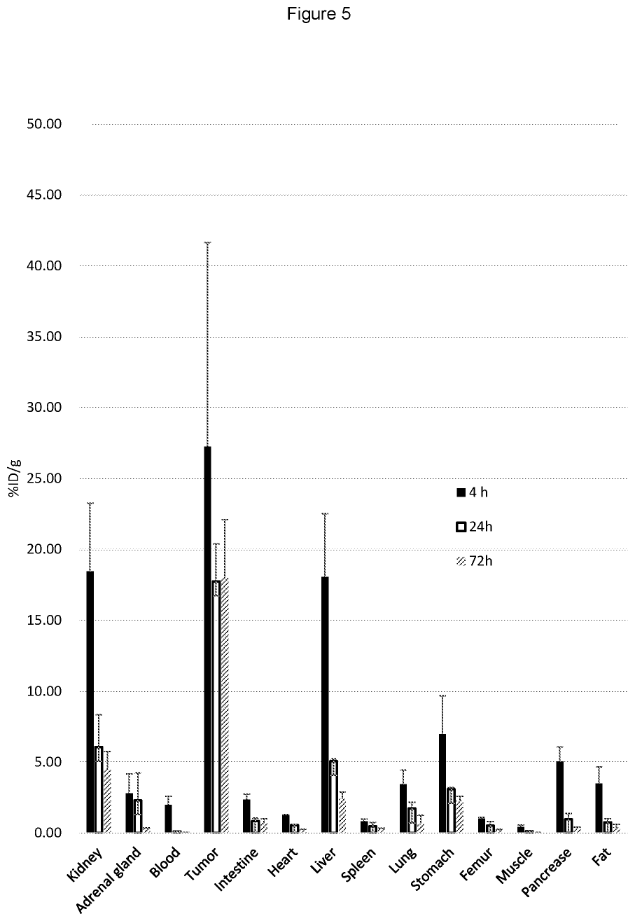

TABLE 11 and FIG. 5 shows the biodistribution of ¹⁷⁷Lu-I-35 in AR42J tumor-bearing mice (n=3)

TABLE 11

| | | | | 4 h | 24 h | 72 h |
|---|---|---|---|---|---|---|
| % ID/g | 4 h | 24 h | 72 h | STDEV | STDEV | STDEV |
| Kidney | 18.46 | 6.06 | 4.51 | 4.80 | 2.31 | 1.25 |
| Adrenal gland | 2.80 | 2.30 | 0.34 | 1.36 | 1.95 | 0.04 |
| Blood | 1.98 | 0.11 | 0.03 | 0.62 | 0.01 | 0.01 |
| Tumor | 27.25 | 17.75 | 18.00 | 14.40 | 2.65 | 4.11 |
| Intestine | 2.36 | 0.84 | 0.72 | 0.39 | 0.22 | 0.29 |
| Heart | 1.26 | 0.56 | 0.25 | 0.04 | 0.07 | 0.03 |
| Liver | 18.06 | 5.06 | 2.41 | 4.47 | 0.18 | 0.48 |
| Spleen | 0.82 | 0.47 | 0.28 | 0.16 | 0.27 | 0.07 |
| Lung | 3.44 | 1.73 | 0.66 | 1.01 | 0.45 | 0.59 |
| Stomach | 6.98 | 3.09 | 2.29 | 2.70 | 0.13 | 0.30 |
| Femur | 1.01 | 0.52 | 0.21 | 0.10 | 0.28 | 0.07 |
| Muscle | 0.42 | 0.12 | 0.03 | 0.13 | 0.01 | 0.01 |
| Pancreas | 5.03 | 0.96 | 0.34 | 1.04 | 0.42 | 0.08 |
| Fat | 3.49 | 0.75 | 0.41 | 1.17 | 0.26 | 0.22 |

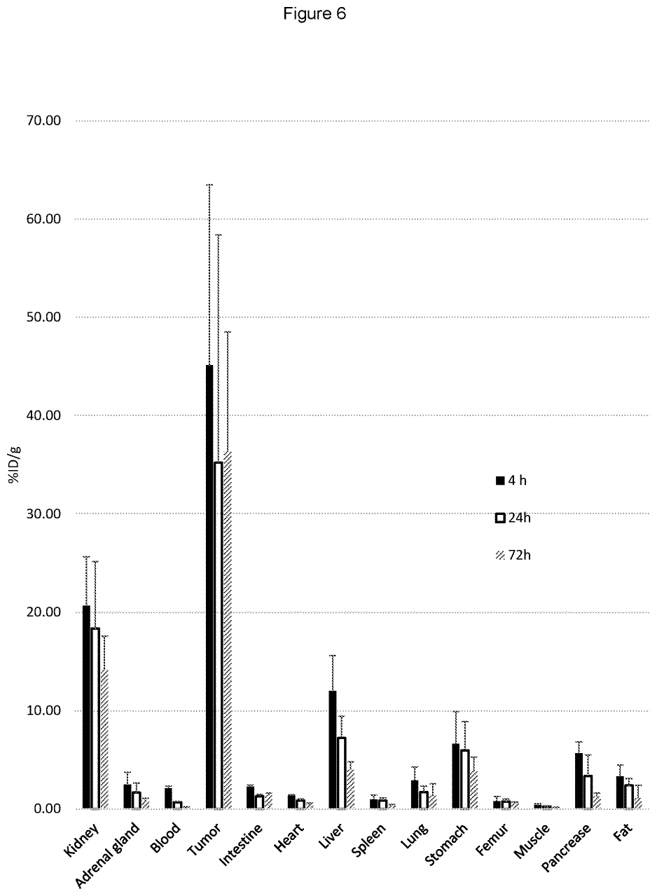

TABLE 12 and FIG. 6 shows the biodistribution of ¹⁷⁷Lu-I-36 in AR42J tumor-bearing mice (n=3) TABLE 12

TABLE 12

| | | | | 4 h | 24 h | 72 h |
|---|---|---|---|---|---|---|
| % ID/g | 4 h | 24 h | 72 h | STDEV | STDEV | STDEV |
| Kidney | 20.70 | 18.38 | 14.19 | 4.95 | 6.78 | 3.39 |
| Adrenal gland | 2.48 | 1.67 | 0.87 | 1.26 | 0.97 | 0.22 |
| Blood | 2.09 | 0.70 | 0.21 | 0.21 | 0.08 | 0.03 |
| Tumor | 45.13 | 35.22 | 36.39 | 18.38 | 23.19 | 12.13 |
| Intestine | 2.27 | 1.29 | 1.39 | 0.15 | 0.19 | 0.23 |
| Heart | 1.36 | 0.85 | 0.47 | 0.10 | 0.16 | 0.12 |
| Liver | 12.00 | 7.22 | 3.99 | 3.61 | 2.23 | 0.81 |
| Spleen | 0.97 | 0.85 | 0.36 | 0.43 | 0.29 | 0.12 |
| Lung | 2.89 | 1.72 | 1.36 | 1.37 | 0.62 | 1.21 |
| Stomach | 6.60 | 5.96 | 3.80 | 3.32 | 2.94 | 1.46 |
| Femur | 0.79 | 0.76 | 0.59 | 0.46 | 0.25 | 0.12 |
| Muscle | 0.41 | 0.25 | 0.13 | 0.14 | 0.06 | 0.07 |
| Pancreas | 5.66 | 3.34 | 1.28 | 1.15 | 2.16 | 0.36 |
| Fat | 3.32 | 2.39 | 1.12 | 1.13 | 0.71 | 1.30 |

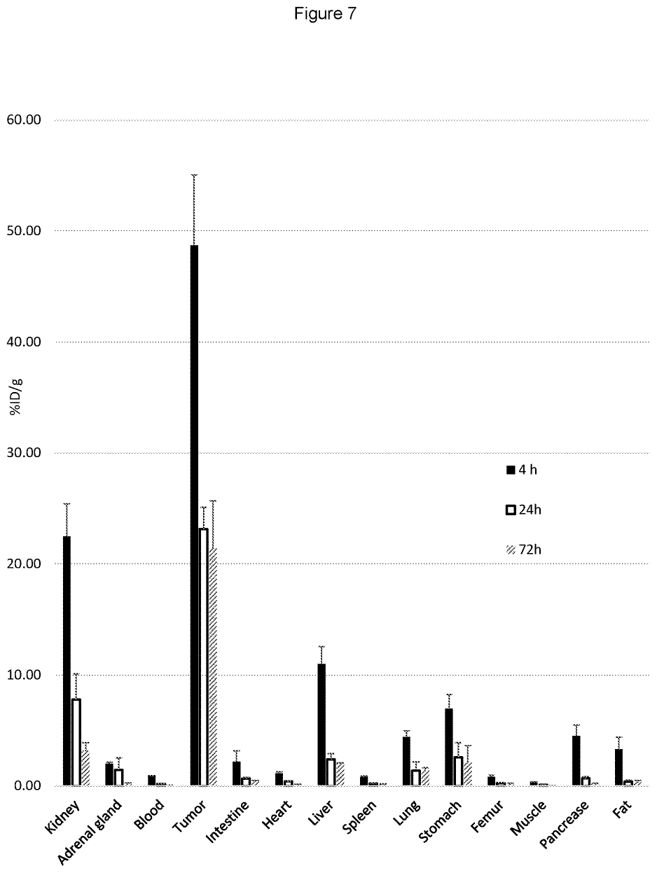

TABLE 13 and FIG. 7 shows the biodistribution of ¹⁷⁷Lu-I-37 in AR42J tumor-bearing mice (n=3)

TABLE 13

| | | | | 4 h | 24 h | 72 h |
|---|---|---|---|---|---|---|
| % ID/g | 4 h | 24 h | 72 h | STDEV | STDEV | STDEV |
| Kidney | 22.48 | 7.79 | 3.16 | 2.94 | 2.32 | 0.75 |
| Adrenal gland | 2.01 | 1.43 | 0.22 | 0.12 | 1.13 | 0.07 |
| Blood | 0.94 | 0.18 | 0.05 | 0.00 | 0.02 | 0.00 |
| Tumor | 48.69 | 23.14 | 21.39 | 6.34 | 1.97 | 4.31 |
| Intestine | 2.17 | 0.67 | 0.50 | 0.99 | 0.10 | 0.02 |
| Heart | 1.12 | 0.40 | 0.15 | 0.15 | 0.06 | 0.01 |
| Liver | 11.00 | 2.40 | 2.03 | 1.58 | 0.52 | 0.05 |
| Spleen | 0.80 | 0.20 | 0.13 | 0.11 | 0.08 | 0.08 |
| Lung | 4.42 | 1.38 | 1.41 | 0.53 | 0.80 | 0.24 |
| Stomach | 6.95 | 2.58 | 2.13 | 1.31 | 1.34 | 1.48 |
| Femur | 0.83 | 0.26 | 0.18 | 0.12 | 0.06 | 0.06 |
| Muscle | 0.38 | 0.10 | 0.03 | 0.01 | 0.02 | 0.02 |
| Pancreas | 4.50 | 0.68 | 0.19 | 1.00 | 0.17 | 0.07 |
| Fat | 3.28 | 0.40 | 0.32 | 1.12 | 0.13 | 0.17 |

TABLE 14 shows the biodistribution of ¹⁷⁷Lu-I-39 in AR42J tumor-bearing mice (n=3)

TABLE 14

| | | | | 4 h | 24 h | 72 h |
|---|---|---|---|---|---|---|
| % ID/g | 4 h | 24 h | 72 h | STDEV | STDEV | STDEV |
| Kidney | 25.69 | 12.03 | 3.20 | 3.31 | 1.02 | 0.90 |
| Adrenal gland | 1.33 | 1.06 | 0.30 | 0.63 | 0.12 | 0.05 |
| Blood | 0.91 | 0.33 | 0.06 | 0.33 | 0.06 | 0.01 |
| Tumor | 41.84 | 27.46 | 9.48 | 11.85 | 6.28 | 6.05 |
| Intestine | 1.80 | 0.76 | 0.24 | 0.33 | 0.26 | 0.12 |
| Heart | 0.84 | 0.38 | 0.12 | 0.23 | 0.15 | 0.01 |
| Liver | 6.35 | 2.78 | 1.24 | 1.08 | 0.88 | 0.63 |
| Spleen | 0.76 | 0.31 | 0.07 | 0.29 | 0.08 | 0.01 |
| Lung | 3.81 | 1.85 | 0.87 | 0.40 | 0.01 | 0.50 |
| Stomach | 6.28 | 4.63 | 1.26 | 1.62 | 0.29 | 0.60 |
| Femur | 0.90 | 0.42 | 0.10 | 0.19 | 0.04 | 0.06 |
| Muscle | 0.30 | 0.13 | 0.02 | 0.09 | 0.01 | 0.00 |
| Pancreas | 3.71 | 0.94 | 0.15 | 0.52 | 0.20 | 0.02 |
| Fat | 3.38 | 1.07 | 0.27 | 1.44 | 0.19 | 0.13 |

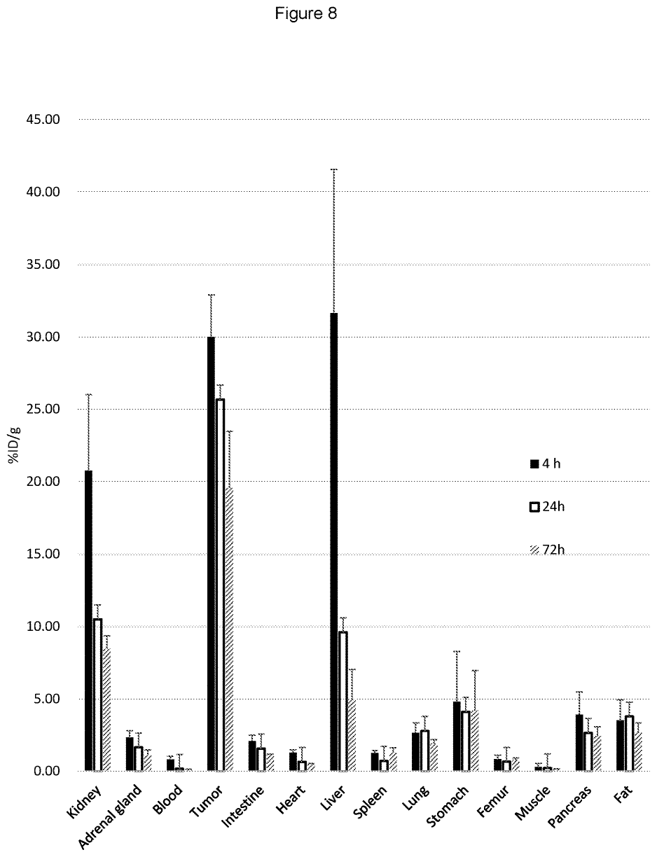

TABLE 15 and FIG. 8 shows the biodistribution of ¹⁷⁷Lu-I-54 in AR42J tumor-bearing mice (n=3)

TABLE 15

| | | | | 4 h | 24 h | 72 h |
|---|---|---|---|---|---|---|
| % ID/g | 4 h | 24 h | 72 h | STDEV | STDEV | STDEV |
| Kidney | 20.74 | 10.50 | 8.45 | 5.27 | 3.62 | 0.90 |
| Adrenal gland | 2.34 | 1.64 | 1.12 | 0.47 | 0.60 | 0.37 |
| Blood | 0.81 | 0.16 | 0.09 | 0.23 | 0.00 | 0.05 |
| Tumor | 30.00 | 25.67 | 19.56 | 2.89 | 8.46 | 3.91 |
| Intestine | 2.07 | 1.56 | 1.14 | 0.43 | 0.08 | 0.04 |
| Heart | 1.29 | 0.65 | 0.50 | 0.19 | 0.03 | 0.04 |
| Liver | 31.63 | 9.60 | 5.01 | 9.94 | 3.40 | 2.03 |
| Spleen | 1.25 | 0.71 | 1.28 | 0.19 | 0.27 | 0.33 |
| Lung | 2.65 | 2.77 | 1.91 | 0.70 | 0.30 | 0.27 |
| Stomach | 4.81 | 4.10 | 4.20 | 3.48 | 1.11 | 2.77 |
| Femur | 0.83 | 0.66 | 0.79 | 0.29 | 0.19 | 0.12 |
| Muscle | 0.30 | 0.21 | 0.16 | 0.24 | 0.16 | 0.01 |
| Pancreas | 3.91 | 2.65 | 2.42 | 1.57 | 0.47 | 0.65 |
| Fat | 3.50 | 3.78 | 2.66 | 1.45 | 0.64 | 0.67 |

### DISCUSSION

Based on structure-activity relationship studies, a trifunctional radioligand, consisting of a SSTR2 binding group, a CCK2R binding group and a radionuclide chelating group has been constructed in a way that resulted in the radioligand having dual targeting vectors each with good binding affinity to the original binding sites of their respective receptors.

Subsequently, in vivo biodistribution and imaging profiles of exemplary dual receptor targeting radioligand was optimized. Chemical and biophysical properties, such as molecular size, hydrophobicity, net charge, charge distribution, and hydrophobic patch distribution were examined to determine factors which contributed to the biodistribution profile. Such studies unexpectedly revealed that subtle changes of these psychochemical properties can have an impact on the biodistribution profile of the radioligand. For example, it was found that some radioligands bearing high negative net charge when complexed with the radionuclide resulted in high kidney uptake while radioligands having a net charge of, for example, +2 to −3 when complexed to the radionuclide provided a good overall biodistribution profile. In addition, it was observed that radioligands having a hydrophobic divalent linker or sometimes no divalent linker caused higher radionuclide uptake in the liver, while radioligands having longer hydrophilic linkers could compromise the tumor/kidney ratio. Some exemplary radioligands that provided high tumor uptake and minimal normal organ accumulation were found to consist of short hydrophilic linkers and a net charge of 0 or −1 when complexed with the radionuclide, e.g ¹⁷⁷Lu.

Accordingly, novel SSTR2 and CCK2R dual targeting radioligands have been identified. The radioligands of the application have been shown to possess potent and balanced binding affinity toward both the SSTR2 and CCK2R receptors. Further, when chelated to ¹⁷⁷Lu, the radioligands of the application were shown to demonstrate a favorable in vivo biodistribution profile in the AR42J tumor-bearing mice model. The radioligands of the application showed high tumor uptake and both good tumor/kidney biodistribution ratios as well as good tumor/liver biodistribution ratios. Accordingly, such radioligands, having this unexpected favorable distribution profile would have an acceptable safety window as well as ideal therapeutic efficacy in cancer patients wherein either SSTR2 or CCK2R is overexpressed.

### FULL CITATIONS FOR DOCUMENTS REFERRED TO IN THE SPECIFICATION

A number of publications are cited herein. Full citations for these references are provided below. Each of these references is incorporated herein by reference in its entirety into the present disclosure, to the same extent as if each individual reference was specifically and individually indicated to be incorporated by

### REFERENCE

1. Lehman J. M., and aMassion P. P., Somatostatin receptor 2 targeting in small cell lung carcinoma: perspectives, Oncotarget. 2019 Jul. 30; 10(46): 4727-4730. PMID:31413814.
2. Parghane R. V., et al., Clinical utility of ¹⁷⁷Lu-DOTATATE PRRT in somatostatin receptor-positive metastatic medullary carcinoma of thyroid patients with assessment of efficacy, survival analysis, prognostic variables, and toxicity, Head Neck, 2020 March; 42(3):401-416
3. Klingler M et al., Cholecystokinin-2 Receptor Targeting with Radiolabeled Peptides: Current Status and Future Directions. Curr Med Chem. 2020; 27(41):7112-7132.
4. Rottenburger C, et al, Cholecystokinin 2 Receptor Agonist 177Lu-PP-F11 N for Radionuclide Therapy of Medullary Thyroid Carcinoma: Results of the Lumed Phase Oa Study. J Nucl Med. 2020 April; 61(4):520-526.
5. Aloj L et al., Comparison of the binding and internalization properties of 12 DOTA-coupled and ¹¹¹In-labelled CCK2/gastrin receptor binding peptides: a collaborative project under COST Action BM0607. Eur J Nucl Med Mol Imaging. 2011 August; 38(8):1417-25.
6. Laverman P. et al., Comparative biodistribution of 12 ¹¹¹In-labelled gastrin/CCK2 receptor-targeting peptides. Eur J Nucl Med Mol Imaging. 2011 August; 38(8):1410-6.
7. Kaloudi A. et al., [99mTc]Tc-DGA1, a Promising CCK2R-Antagonist-Based Tracer for Tumor Diagnosis with Single-Photon Emission Computed Tomography. Mol Pharm. 2020 Aug. 3; 17(8):3116-3128.
8. Wayua C et al., Evaluation of a nonpeptidic ligand for imaging of cholecystokinin 2 receptor-expressing cancers. J Nucl Med. 2015 January; 56(1):113-9.

1. A compound of Formula I or a pharmaceutically acceptable salt, solvate and/or prodrug thereof, 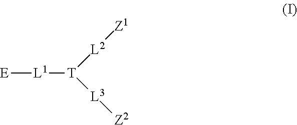
wherein
E is a chelating group;
T is a trivalent branching group;
Z¹ is a cholecystokinin-2 receptor (CCK2R) binding group;
Z² is a somatostatin receptor 2 (SSTR2) binding group;
L¹, L² and L³ are each independently a direct bond or a divalent linker.

2. The compound of claim 1, wherein the CCK2R binding group is a benzodiazepine moiety of Formula II, 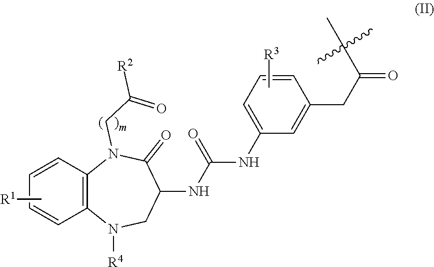
wherein
R¹ is selected from H, halo and C₁₋₆alkyl;
R² is selected from C₁₋₆alkyl, NH₂, NH(C₁₋₆alkyl) and N(C₁₋₆alkyl)₂;
R³ is selected from H, halo and C₁₋₆alkyl;
R⁴ is selected from C₅₋₆cycloalkyl and phenyl; and
m is 0, 1, 2 or 3; and
n is 0, 1, 2 or 3.

3. The compound of claim 2, wherein R¹ and R³ are independently selected from H, F, Cl, CH₃, CH₂CH₃, CH₂CH₂CH₃, CH₂CH₂CH₂CH₃, CH(CH₃)₂ and C(CH₃)₃.

4. The compound of claim 3, wherein both R¹ and R³ are H.

5. The compound of any one of claims 2 to 4, wherein R² is selected from CH₃, CH₂CH₃, CH₂CH₂CH₃, CH₂CH₂CH₂CH₃, CH(CH₃)₂, C(CH₃)₃, NH₂, NH(CH₃) and N(CH₃)₂.

6. The compound of claim 5, wherein R² is C(CH₃)₃.

7. The compound of any one of claims 2 to 6, wherein R⁴ is selected from cyclopentyl, cyclohexyl and phenyl.

8. The compound of claim 7, wherein R⁴ is cyclohexyl.

9. The compound of any one of claims 2 to 8, wherein m is 1 and n is 0.

10. The compound of any one of claims 1 to 9, wherein the somatostatin receptor 2 (SSTR2) binding group is a moiety of Formula III, 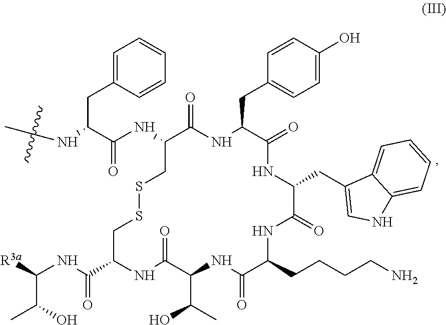
wherein:
R^{3a} is selected from CH₂OH, CO₂H and CONH₂.

11. The compound of claim 10, wherein R^{3a} is selected from CH₂OH and CONH₂.

12. The compound of claim 11, wherein R^{3a} is CO₂H.

13. The compound of any one of claims 2 to 8, wherein the compound of Formula I or a pharmaceutically acceptable salt, solvate and/or prodrug thereof when complexed to a radionuclide comprises a net charge of positive 2 (+2) to negative five (−5).

14. The compound of any one of claims 2 to 8, wherein the compound of Formula I or a pharmaceutically acceptable salt, solvate and/or prodrug thereof when complexed to a radionuclide comprises net charge of zero (0) or negative one (−1).

15. The compound of any one of claims 1 to 14, wherein combined length of L¹, L² and L³ is 0 atoms to 80 atoms.

16. The compound of any one of claims 1 to 14, wherein the combined length of L¹, L² and L³ is 0 atoms to 47 atoms, 0 atoms to 46 atoms, 0 atoms to 43 atoms, 0 atoms to 41 atoms, 0 atoms to 40 atoms, 0 atoms to 36 atoms, 0 atoms to 37 atoms, 0 atoms to 33 atoms, 0 atoms to 30 atoms or 0 atoms to 27 atoms.

17. The compound of any one of claims 1 to 16, wherein E is a chelating group derived from a chelating agent selected from a cyclic or an acyclic bifunctional chelating agent capable of binding with and/or complexing one or more radionuclides.

18. The compound of claim 17, wherein E a chelating group derived from a chelating agent selected from 1,4,7-Triazacyclononane (TACN); 1,4,7-triazacyclononane-triacetic acid (NOTA); 1,4,7-triazacyclononane-N-succinic acid-N′,N″-diacetic acid (NOTASA); 1,4,7-triazacyclononane-N-glutamic acid-N′,N″-diacetic acid (NODAGA); 1,4,7-triazacyclononane-N,N′,N″-tris (methylenephosphonic) acid (NOTP); 1,4,7,10-tetraazacyclododecane ([12]aneN4) (cyclen); 1,4,7,10-tetraazacyclotridecane ([13]aneN4); 1,4,7,11-tetraazacyclotetradecane (iso-cyclam); 1,4,7,10-tetraazacyclododecane-1,4,7,10-tetraacetic acid (DOTA); 2-(1,4,7,10-tetraazacyclododecan-1-yl)acetate (DO1A); 2,2′-(1,4,7,10-tetraazacyclododecane-1,7-diyl) diacetic acid (DO2A); 2,2′,2″-(1,4,7,10-tetraazacyclododecane-1,4,7-triyl) triacetic acid (DO3A); 1,4,7,10-tetraazacyclododecane-1,4,7,10-tetra(methanepnosphonic acid) (DOTP); 1,4,7,10-tetraazacyclododecane-1,7-di(methanephosphoriic acid) (DO2P); 1,4,7,10-tetraazacyclododecane-1,4,7-tri(methanephosphonic acid) (DO3P); 1,4,7,10-tetraazacyclo-decane-1-glutamic acid-4,7,10-triacetic acid (DOTAGA); 1,4,7,10-tetraazacyclodecane-1-succinic acid-4,7,10-triacetic acid (DOTASA); 1,4,8,11-tetraazacyclotetradecane ([14]aneN4) (cyclam); 1,4,8,12-tetraazacyclopentadecane ([15]aneN4); 1,5,9,13-tetraazacyclohexadecane ([16]aneN4); 1,4-ethano-1,4,8,11-tetraazacyclo-tetradecane (et-cyclam); 1,4,8,11-tetraazacyclotetradecane-1,4,8,11-tetraacetic acid (TETA); 2-(1,4,8,11-tetraazacyclotetradecane-1-yl) acetic acid (TE1A); 2,2′-(1,4,8,11-tetraazacyclotetradecane-1,8-diyl) diacetic acid (TE2A); 4,11-bis(carboxy methyl)-1,4,8,11-tetraazabicyclo[6.6.2]-hexadecane (CB-TE2A); 3,6,10,13,16,19-hexaazabicyclo[6.6.6]icosane (Sar); 1,4,7,10-tetra-(2-carbamoyl-methyl)-cyclododecane (TCMC); N,N′-bis[(6-carboxy-2-pyridil)methyl]-4,13-diaza-18-crown-6 (macropa), phthalocyanines; porphyrins; PCTA (3,6,9,15-tetraazabicyclo[9.3.1]pentadeca-1(15), 11,13-triene-3,6,9-triacetic acid); DEPA (7-[2-(biscarboxymethylamino)ethyl]-4,10-biscarboxymethyl-1,4,7,10-tetraazacyclododec-1-yl-acetic acid); DTPA (1,1,4,7,7-diethylenetriaminepentaacetic acid); CHX-DTPA (cyclohexane-1,2-diamineN,N,N′,N′-tetraacetate); BATPA (1,2-bis[2-aminophenoxy]ethane-N,N,N′,N′-tetraacetic acid); TTHA (triethylenetetramineN,N,N′,N″,N″′,N″′-hexaacetic acid); HBED (N,N′-bis[2-hydroxybenzyl]ethylenediamine-N,N′-diacetic acid); EGTA (ethylene glycol bis[2-aminoethyl ether]-N,N,N′,N′-tetraacetic acid); EDTMP (ethylenediamine tetra-[methylene phosphonic acid]); TRAP (triazacyclononate phosphinic acids); SHBED (N,N′-bis[2-hydroxy-5-sulfobenzyl]ethylenediaminediacetic acid); H6Sbbpen(N,N′-bis-[2-hydroxy-5-sulfonylbenzyl]-N,N′-bis[2-methylpyridyl]ethylenediamine); THP (Tris(3,4-hydroxypyridinone); DFO (deferoxamine); FSC (Fusarinine); 6SS (N,N′-bis[2,2-dimethyl-2-mercaptoethyl]ethylenediamine-N,N′-diacetic acid); ECC (ethylenecysteamine cysteine); ECD (ethyl cysteinate dimer); NETA ([2-{4,7-biscarboxymethyl(1,4,7)triazacyclonona-1-yl-ethyl}carbonylmethylamino]acetic acid; THPN (Tetrakis(3-Hydroxy-4-Pyridinone)); H2dedpa (1,2-[{6-(carboxylato-)pyridin-2-yl}methylamino]-ethane); H4octapa (N,N′-bis[6-carboxy-2-pyridylmethyl]-ethylenediamine-N,N′-diacetic acid); H2bispa2 (6,6′-[{9-hydroxy-1,5-bis-(methoxycarbonyl)-2,4-di(pyridin-2-yl)-3,7-diazabicyclo[3.3.1]nonane-3,7-diyl}bis(methylene)]dipicolinic acid); DOTMP(1,4,7,10-Tetraazacyclododecane-1,4,7,10-tetrayl-tetrakis(methylphosphonic acid)); PEPA (1,4,7,10,13-pentaazocyclopentadecane pentaacetic acid); HEHA (1,4,7,10,13,16-hexaazocyclooctadecane hexaacetic acid); H2hox; H2CHXhox; H2octox; H2pyhox; H4neunopa; TETPA; H4pypa; H4py4pa; DTPAm; EGTAm; ampam; Me-3,2-HOPO;
3,4,3-(LI-1,2-HOPO); macrocyclic tetrapthalimide; or any derivatives thereof.

19. The compound of claim 18, wherein the chelating agent is selected from DOTA and DOTAGA.

20. The compound of any one of claims 17 to 19, wherein the one or more radionuclides is a radioactive isotope of C, N, O, F, P, S, Cl, Br, I, As, Se, At, K, Sc, Ti, Cr, Mn, Fe, Co, Ni, Cu, Ga, Ge, Rb, Sr, Y, Zr, Nb, Tc, Rh, Pd, In, Sn, Sb, Zn, Ta, W, Re, Os, Ir, Pt, Au, Hg, TI, Pb, Bi, Po, Fr, Pm, a lanthanide (such as La, Ce, Pr, Nd, Pm, Sm, Eu, Gd, Tb, Dy, Ho, Er, Tm, Yb, Lu), an actinide (such as Ac, Th, U), Mg, Al, Ca, Cd, or Ba.

21. The compound of claim 20, wherein the lanthanide isLu, Sm, Ho, or Tb.

22. The compound of claim 20 or claim 21, wherein the one or more radionuclides are selected from ¹⁴C, ¹⁵N, ¹⁸F, ⁷⁵Br, ⁷⁶Br, ⁷⁷Br, ¹²³I, ¹²⁴I, ¹²⁵I, ¹³¹I, ³⁵S, ⁹⁹Tc, ^{99m}Tc, ¹⁸⁸Re, ¹⁸⁶Re, ¹⁵³Sm, ⁶⁶Ga, ⁶⁷Ga, ⁶⁸Ga, ¹¹¹In, ¹²³In, ⁵⁹Fe, ⁶³Zn, ⁵²Fe, ⁵²Mn, ⁴⁵Ti ⁶⁰Cu, ⁶¹Cu, ⁶⁷Cu ⁶⁴Cu ⁶²Cu ⁸²Rb, ^{19m}Pt, ^{191m}Pt, ^{193m}Pt, ^{117m}Sn, ⁸⁹Zr, ¹⁷⁷Lu, ¹⁸F, ¹⁸⁸Re, ¹⁸⁶Re, ¹⁵³Sm, ⁶⁶Ho, ⁸⁶Y, ⁸⁷Y, ⁹⁰Y, ⁸⁹Sr, ¹⁵³Gd, ¹⁵⁹Gd, ²²⁵Ac, ²¹²Bi, ²¹³Bi, ²¹¹At, ¹⁹⁸Au, ¹⁹⁹Au, ^{193m}Pt, ¹⁹⁷Pt, ¹⁰³Pd, ¹⁰⁹Pd, ¹⁰⁵Rh, ^{101m}Rh, ^{103m}Rh, ²²³Ra, ²²⁴Ra, ⁹⁷Ru, ²²⁷Th, ²²⁹Th, ³²p, ¹⁶¹Tb, ³³p ¹⁴⁹Tb, ¹²⁵I, ²⁰³Pb, ²¹²Pb, ²⁰¹T1, ¹¹⁹Sb, ⁵⁸mCo, 55 Co, ⁵⁷Co, ⁴⁷Sc, ¹⁴⁹Pm ¹⁴²Pr ¹⁶¹Ho, ¹⁶⁶Ho, ¹⁷⁵Yb, or ⁵¹Cr.

23. The compound of any one of claims 17 to 22, wherein the one or more radionuclides are for use in imaging or for use in therapy.

24. The compound of any one of claims 17 to 23, wherein the one or more radionuclides is ¹⁷⁷Lu.

25. The compound of any one of claims 1 to 24, wherein T is a trivalent branching group comprising at least a first terminal functional group, a second terminal functional group and a third terminal functional group, which are the same or different and bind to a complementary functional groups on L¹ (or alternatively E), L² (or alternatively Z¹) and L³ (or alternatively Z²), respectively.

26. The compound of claim 25, wherein T is selected from an amino acid residue derived from lysine, ornithine, homo-lysine, 2,3-diaminopropionic acid (Dap), 2,4-diaminobutyric acid (Dab), cysteine, homo-cysteine, glutamate or glutamine.

27. The compound of claim 26, wherein T is an amino acid residue derived from lysine,

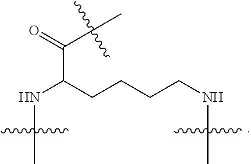 [SMILES: *NCCCCC(N*)C(*)=O]

28. The compound of any one of claims 1 to 27, wherein L¹, L² and L³ are each independently a direct bond or a divalent linker, and the divalent linkers combined comprise a total of 1 to 15 groups selected from amino acid residues, W^{a}, R^{a}, R^{a}-W^{a}, W^{a}-R^{b}, R^{a}-W^{a}-R^{b} and W^{a}-R^{b}-W^{b},
wherein
each W^{a} and W^{b} is independently selected from O, S, S(O), S(O)₂, NR⁵, C(O), C(S), NR⁵C(O), NR⁵C(S), C(O)NR⁵, C(S)NR⁵, (C₁₋₆alkyleneY)ₚ and Y-(C₁₋₆alkyleneY′)ₚ,
each R^{a} and R^{b} is independently selected from C₁₋₂₀alkylene, C₂₋₂₀alkenylene, and C₂₋₂₀alkynylene;
each Y and Y′ is independently selected from O, S, C(O) and NR⁶;
each R⁵ is independently selected from H and C₁₋₆alkyl;
each R⁶ is independently selected from H and C₁₋₃alkyl; and
p is an integer selected from 1 to 8.

29. The compound of claim 28, wherein each R^{a} and R^{b} is independently selected from C₁₋₁₀alkylene, C₂₋₁₀alkenylene and C₂-10alkynylene.

30. The compound of claim 28, wherein each R^{a} and R^{b} is independently selected from C₁₋₂₀alkylene.

31. The compound of any one of claims 28 to 30, wherein Y and Y′ is independently selected from O, S, C(O) and NR⁶.

32. The compound of claim 31, wherein each Y and Y′ is independently selected from O, C(O) and NR⁶.

33. The compound of any one of claims 28 to 32, each W^{a} and W^{b} is independently selected from O, S, S(O), SO₂, NR⁵, C(O), C(S), C(S)NR⁵, NR⁵C(S), C(O)NR⁵, NR⁵C(O), (C₁₋₆alkyleneO)ₚ, (C₁₋₆alkyleneNR⁶)ₚ, (C₁₋₆alkyleneC(O))ₚ, O—(C₁₋₆alkyleneO)ₚ, O—(C₁₋₆alkyleneNR⁶)ₚ, O—(C₁₋₆alkyleneC(O))ₚ, NR⁶—(C₁₋₆alkyleneO)ₚ, NR⁶—(C₁₋₆alkyleneNR⁶)ₚ, NR⁶(C₁₋₆alkyleneC(O))ₚ, C(O)—(C₁₋₆alkyleneO)ₚ, C(O)—(C₁₋₆alkyleneNR⁶)ₚ, and C(O)—(C₁₋₆alkyleneC(O)ₚ.

34. The compound of any one of claims 30 to 33, wherein the divalent linkers combined comprise a combined total of 1 to 15 groups selected from amino acid residues, O, S, S(O), SO₂, NR⁵, C(O), C(O)NR⁵, NR⁵C(O), C₁₋₂₀alkylene, OC₁₋₂₀alkyleneO, OC₁₋₂₀alkyleneNR⁵, OC₁₋₂₀alkyleneC(O), NR⁵C₁₋₂₀alkyleneO, NR⁵C₁₋₂₀alkyleneNR⁵, NR⁵C₁₋₂₀alkyleneC(O), C(O)C₁₋₂₀alkyleneO, C(O)—C₁₋₂₀alkyleneC(O), C(O)C₁₋₂₀alkyleneNR⁵, O—(C₁₋₆alkyleneC(O))ₚC₁₋₂₀alkyleneO, O—(C₁₋₆alkyleneC(O))ₚC₁₋₂₀alkyleneNR⁵, O—(C₁₋₆alkyleneC(O))ₚC₁₋₂₀alkyleneC(O), O—(C₁₋₆alkyleneNR⁶)ₚC₁₋₂₀alkyleneO, O—(C₁₋₆alkyleneNR⁶)ₚC₁₋₂₀alkyleneNR⁵, O—(C₁₋₆alkyleneNR⁶)ₚC₁₋₂₀alkyleneC(O), O—(C₁₋₆alkyleneC(O))ₚC₁₋₂₀alkyleneO, O—(C₁₋₆alkyleneC(O))ₚC₁₋₂₀ alkyleneNR⁵, O—(C₁₋₆alkyleneC(O))ₚC₁₋₂₀alkyleneC(O), NR⁶—(C₁₋₆alkyleneO)ₚC₁₋₂₀ alkyleneO, NR⁶—(C₁₋₆alkyleneO)ₚC₁₋₂₀alkyleneNR⁵, NR⁶—(C₁₋₆alkyleneO)ₚC₁₋₂₀alkyleneC(O), NR⁶—(C₁₋₆alkyleneNR⁶)ₚC₁₋₂₀alkyleneO, NR⁶—(C₁₋₆alkyleneNR⁶)ₚC₁₋₂₀alkyleneNR⁵, NR⁶—(C₁₋₆alkyleneNR⁶)ₚC₁₋₂₀alkyleneC(O), NR⁶(C₁₋₆alkyleneC(O))ₚC₁₋₂₀alkyleneO, NR⁶(C₁₋₆alkyleneC(O))ₚC₁₋₂₀alkyleneNR⁵, NR⁶(C₁₋₆alkyleneC(O))ₚC₁₋₂₀alkyleneC(O), C(O)—(C₁₋₆alkyleneO)ₚC₁₋₂₀alkyleneO, C(O)—(C₁₋₆alkyleneO)ₚC₁₋₂₀alkyleneNR⁵, C(O)—(C₁₋₆alkyleneO)ₚC₁₋₂₀alkyleneC(O), C(O)—(C₁₋₆alkyleneNR⁶)ₚC₁₋₂₀alkyleneO, C(O)—(C₁₋₆alkylene NR⁶)ₚC₁₋₂₀alkyleneNR⁵, C(O)—(C₁₋₆alkyleneNR⁶)ₚC₁₋₂₀alkyleneC(O), C(O)—(C₁₋₆alkyleneC(O))ₚ C₁₋₂₀alkyleneO, C(O)—(C₁₋₆alkyleneC(O))ₚC₁₋₂₀alkyleneNR⁵ and C(O)—(C₁₋₆alkyleneC(O))ₚC₁₋₂₀alkyleneC(O).

35. The compound of claim 34, wherein the divalent linkers combined comprise 1 to 15 groups independently selected from amino acid residues, NR⁵C₁₋₁₂alkyleneC(O), C(O)—C₁₋₁₂alkyleneC(O), C(O)C₁₋₁₂alkyleneNR⁵, NR⁵C₁₋₁₂alkyleneNR⁵, NR⁶—(C₁₋₆alkyleneO)ₚC₁₋₆alkyleneNR⁵, NR⁶—(C₁₋₆alkyleneO)ₚC₁₋₆alkyleneC(O), NR⁶—(C₁₋₆alkyleneNR⁶)ₚC₁₋₆alkyleneNR⁵, NR⁶—(C₁₋₆alkyleneNR⁶)ₚC₁₋₆alkyleneC(O), NR⁶(C₁₋₆alkyleneC(O))ₚC₁₋₆alkyleneNR⁵, NR⁶(C₁₋₆alkyleneC(O))ₚC₁₋₆alkyleneC(O), C(O)—(C₁₋₆alkyleneO)ₚC₁₋₆alkyleneNR⁵, C(O)—(C₁₋₆alkyleneO)ₚC₁₋₆alkyleneC(O), C(O)—(C₁₋₆alkyleneNR⁶)ₚC₁₋₆alkyleneNR⁵, C(O)—(C₁₋₆alkyleneNR⁶)ₚC₁₋₂₀alkyleneC(O), C(O)—(C₁₋₆ alkyleneC(O))ₚC₁₋₆alkyleneNR⁵ and C(O)—(C₁₋₆alkyleneC(O))ₚ C₁₋₆alkyleneC(O).

36. The compound of claim 35, wherein the divalent linkers combined comprise 1 to groups independently selected from amino acid residues, NR⁵C₁₋₁₂alkyleneC(O), C(O)C₁₋₁₂alkyleneNR⁵ and NR⁶—(C₁₋₆alkyleneO)ₚC₁₋₆alkyleneC(O).

37. The compound of any one of claims 27 to 35, wherein the amino acid residues are amino acid residues derived from naturally occurring amino acids, naturally occurring amino acids that have been modified to provide modified amino acids, D enantiomers of the naturally occurring amino acid residues or the modified amino acid residues and amino acid residues derived from a p-amino acid or a γ-amino acid.

38. The compound of any one of claims 28 to 37, wherein the amino acid residues comprise zero charged amino acid residues.

39. The compound of any one of claims 28 to 38, wherein the amino acid residues comprise at least one negatively charged amino acid residue selected from D-Asp, L-Asp, D-Glu, L-Glu, D-γGlu and L-γGlu.

40. The compound of any one of claims 28 to 39, wherein the amino acid residues comprise positively charged amino acid residues selected from D-His, L-His, D-Arg, L-Arg, D-Lys, L-Lys, D-εLys, and L-εLys.

41. The compound of any one of claims 28 to 40, wherein the amino acid residues comprise one or more hydrophilic amino acid residues selected from D-Ser, and L-Ser.

42. The compound of any one of claims 28 to 41, wherein the amino acid residues comprise one or more neutral amino acid residues selected from Gly, D-Pro and L-Pro.

43. The compound of claim 36 or claim 37, wherein the divalent linkers comprise a combined total of 1 to 15 groups independently selected from 4-carboxy-L-phenylalaninel (Cbp), 2,3-diaminopropionic acid (Dap), Gly, D-His, L-His, D-Ala, L-Ala, D-Val, L-Val, D-Leu, L-Leu, D-Phe, L-Phe, D-Pro, L-Pro, D-Met, L-Met, D-Trp, L-Trp, D-Thr, L-Thr, D-Tyr, L-Tyr, D-Nle, L-Nle, D-Ser, L-Ser, D-Arg, L-Arg, D-Lys, L-Lys, D-εLys, L-εLys, D-Asp, L-Asp, D-Glu, L-Glu, D-γGlu, L-γGlu, NR⁵C₁₋₁₂alkyleneC(O), C(O)C₁₋₁₂ alkyleneNR⁵ and NR⁶—(C₁₋₆alkyleneO)ₚC₁₋₆alkyleneC(O).

44. The compound of claim 43, wherein the divalent linkers comprise a combined total of 1 to 15 groups selected from Cbp, Dap, Gly, D-His, L-His D-Ala, L-Ala, D-Val, L-Val, D-Ser, L-Ser, D-Pro, L-Pro, D-Arg, L-Arg, D-Lys, L-Lys, D-εLys, L-εLys, D-Asp, L-Asp, D-Glu, L-Glu, D-γGlu, L-γGlu, NR⁵C₂₋₈alkyleneC(O), C(O)C₂₋₈alkyleneNR⁵ and NR⁶—(C₁₋₃alkyleneO)ₚC₁₋₃alkyleneC(O).

45. The compound of claim 44, wherein the divalent linkers comprise a combined total of 1 to 15 groups independently selected from Cbp, Dap, Gly, D-Ala, D-His, L-His L-Ala, D-Val, L-Val, D-Ser, L-Ser, D-Pro, L-Pro, D-Arg, L-Arg, D-Lys, L-Lys, D-εLys, L-εLys, D-Asp, L-Asp, D-Glu, L-Glu, D-γGlu, L-γGlu, NR⁵C₅alkyleneC(O) (Ahx), NR⁵C₇alkyleneC(O) (Aoc), NR⁵C₁₀alkyleneC(O) (Aun), NR⁶—(C₂alkyleneO)ₚC₁alkyleneC(O) and NR⁶—(C₂alkyleneO)ₚC₂alkyleneC(O).).

46. The compound of claim 45, wherein p is an integer selected from 1 to 6 and the divalent linkers comprise a combined total of 1 to 15 groups independently selected from Cbp, Dap, Gly, D-His, L-His, D-Ala, L-Ala, D-Ser, L-Ser, D-Pro, L-Pro, D-Arg, L-Arg, D-Lys, L-Lys, D-εLys, L-εLys, D-Asp, L-Asp, D-Glu, L-Glu, D-γGlu, L-γGlu, NR⁶—(C₂alkyleneO)₂C₁alkyleneC(O) (OEG), NR⁶—(C₂alkyleneO)C₂alkyleneC(O) (PEG1), NR⁶—(C₂alkyleneO)₃C₂alkyleneC(O) (PEG3), NR⁶—(C₂alkyleneO)eC₂alkyleneC(O) (PEG6), NR⁵C₇alkyleneC(O) (Aoc), NR⁵C₁₀alkyleneC(O) (Aun) and NR⁵C₅alkyleneC(O) (Ahx).

47. The compound of claim 46, wherein the divalent linkers comprise a combined total of 1 to 15 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx.

48. The compound of any one claims 13 to 47, wherein the compound of Formula I or a pharmaceutically acceptable salt, solvate and/or prodrug thereof when complexed to a radionuclide comprises a net charge of negative five (−5) and the divalent linkers comprise a combined total 4 to 11 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, wherein at least 4 or 5 groups are selected from D-Glu, L-Glu, D-γGlu and L-γGlu.

49. The compound of any one claims 13 to 47, wherein the compound of Formula I or a pharmaceutically acceptable salt, solvate and/or prodrug thereof when complexed to a radionuclide comprises a net charge of negative four (−4) and the divalent linkers combined comprise 3 to 11 groups independently selected from 3 to 11 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, wherein 3 or 4 groups are selected from D-Glu, L-Glu, D-γGlu and L-γGlu.

50. The compound of any one claims 13 to 47, wherein the compound of Formula I or a pharmaceutically acceptable salt, solvate and/or prodrug thereof when complexed to a radionuclide comprises a net charge of negative three (−3) and the divalent linkers comprise a combined total of 2 to 9 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, wherein at least 2 or 3 groups are selected from D-Glu, L-Glu, D-γGlu and L-γGlu.

51. The compound of any one claims 13 to 47, wherein the compound of Formula I or a pharmaceutically acceptable salt, solvate and/or prodrug thereof when complexed to a radionuclide comprises a net charge of negative two (−2) and the divalent linkers comprise a combined total of 1 to 9 groups independently selected from Cbp, Dap, Gly, D-His, L-His, D-Pro, L-Pro, D-Ser, L-Ser, D-Arg, L-Arg, D-Lys, L-Lys, D-εLys, L-εLys, D-Glu, L-Glu, D-γGlu, L-γGlu, OEG, PEG1, PEG3, PEG6, Aoc, Aun and Ahx, wherein at least 1 or 2 groups are selected from D-Glu, L-Glu, D-γGlu and L-γGlu.

52. The compound of any one claims 13 to 47, wherein the compound of Formula I or a pharmaceutically acceptable salt, solvate and/or prodrug thereof when complexed to a radionuclide comprises a net charge of negative one (−1) and the divalent linkers comprise a combined total of 1 to 9 groups independently selected from Cbp, Dap, Gly, D-His, L-His, D-Pro, L-Pro, D-Ser, L-Ser, D-Arg, L-Arg, D-Lys, L-Lys, D-CLys, L-CLys, D-Glu, L-Glu, D-γGlu, L-γGlu, OEG, PEG1, PEG3, PEG6, Aoc, Aun and Ahx, wherein 0 or 1 groups are selected from D-Glu, L-Glu, D-γGlu and L-γGlu.

53. The compound of any one of claims 13 to 47, wherein E is a chelating group derived from DOTA, the divalent linkers combined comprise 2 to 9 groups independently selected from Gly, L-Pro, L-Glu, L-γGlu, OEG, PEG1, PEG3, PEG6, and Aoc wherein at least 1 group is selected from L-Glu and L-γGlu and the compound of Formula I or a pharmaceutically acceptable salt, solvate and/or prodrug thereof when complexed to ¹⁷⁷Lu comprises a net charge of negative one (−1).

54. The compound of any one claims 13 to 47, wherein the compound of Formula I or a pharmaceutically acceptable salt, solvate and/or prodrug thereof when complexed to a radionuclide comprises a net charge of zero (0) and the divalent linkers comprise a combined total of 1 to 9 groups independently selected from Cbp, Dap, Gly, D-His, L-His, D-Pro, L-Pro, D-Arg, L-Arg, D-Ser, L-Ser, D-Lys, L-Lys, D-CLys, L-CLys, D-Glu, L-Glu, D-γGlu, L-γGlu, OEG, PEG1, PEG3, PEG6, Aoc Aun and Ahx, wherein 0 or 1 groups are selected from D-Lys, L-Lys, D-CLys and L-CLys.

55. The compound of claim 47, wherein the divalent linkers comprise a combined total of 2 to 5 groups independently selected from Gly and L-Pro.

56. The compound of claim 47, wherein the divalent linkers comprise a combined total of 1 to 4 groups independently selected from Gly and L-Ser.

57. The compound of claim 47, wherein the divalent linkers comprise a combined total of 1 to 4 groups and the 1 to 4 groups are Gly.

58. The compound of claim 47, wherein the divalent linkers comprise a combined total of 1 to 2 groups selected from PEG1, PEG3 or PEG6.

59. The compound of claim 47, wherein the divalent linkers comprise a combined total of 1 to 2 and the 1 to 2 groups are Aoc.

60. The compound of claim 47, wherein the divalent linkers comprise a combined total of 1 to 4 groups and the 1 to 4 groups are OEG.

61. The compound of claim 47, wherein the divalent linkers comprise a combined total of 1 group and the 1 group is OEG.

62. The compound of claim 47, wherein the divalent linkers comprise a combined total of 1 to 7 groups independently selected from Gly, D-γGlu, L-γGlu, OEG and Gly.

63. The compound of claim 47, wherein the divalent linkers comprise a combined total of 1 to 7 groups independently selected from Gly, D-γGlu, L-γGlu, D-Lys, L-Lys, D-εLys and L-εLys and OEG.

64. The compound of claim 47, wherein, the divalent linkers comprise a combined total of 1 to 7 groups independently selected from Cbp, Dap, Gly, D-γGlu, L-γGlu, D-Ser, L-Ser, D-His, L-His and OEG.

65. The compound of any one of claims 1 to 64 wherein one of L¹, L² and L³ is a direct bond.

66. The compound of any one of claims 1 to 64 wherein L¹ is a direct bond.

67. The compound of any one of claims 1 to 64, wherein two of L¹, L² and L³ are direct bonds.

68. The compound of any one of claims 1 to 28, wherein L¹, L² and L³ are all direct bonds.

69. The compound of any one claims 1 to 47, wherein one or two of L¹, L² and L³ are divalent linkers, and the divalent linkers comprise a combined total of 1 to 15 groups selected from Cbp, Dap, Gly, D-His, L-His, D-Pro, L-Pro, D-Arg, L-Arg D-Ser, L-Ser, D-Lys, L-Lys, D-εLys, L-εLys, D-Glu, L-Glu, D-γGlu, L-γGlu, OEG, PEG1, PEG3, PEG6, Aoc, Aun and Ahx, and the other two or one of L¹, L² and L³ respectively are direct bonds.

70. The compound of any one of claims 1 to 47, wherein one of L¹, L² and L³ is a divalent linker, and the divalent linker comprises 1 to 3 OEG groups, and the other two of L¹, L² and L³ are direct bonds.

71. The compound of any one of claims 1 to 47, wherein one of L¹, L² and L³ is a divalent linker, and the divalent linker comprises 1 OEG group, and the other two of L¹, L² and L³ are direct bonds.

72. The compound of any one of claims 1 to 47, wherein E is a chelating group derived from DOTA, one of L¹, L² and L³ is a divalent linker, and the divalent linker comprises 1 OEG group, and the other two of L¹, L² and L³ are direct bonds and the compound of Formula I or a pharmaceutically acceptable salt, solvate and/or prodrug thereof when complexed to ¹⁷⁷Lu comprises a net charge of zero (0).

73. The compound of any one of claims 13 to 72, wherein each R⁵ is independently selected from H and CH₃.

74. The compound of any one of claims 13 to 73, wherein each R⁶ is independently selected from H and OH₃.

75. The compound of claim 1, wherein the compound of Formula I is selected from the following list of compounds:

| | Compound | |
|---|---|---|
| I.D | Name | Structure |
| I-1 | DOTA-K(- gE-G- Z360)- OEG- f[CYwKTC] T-OH | 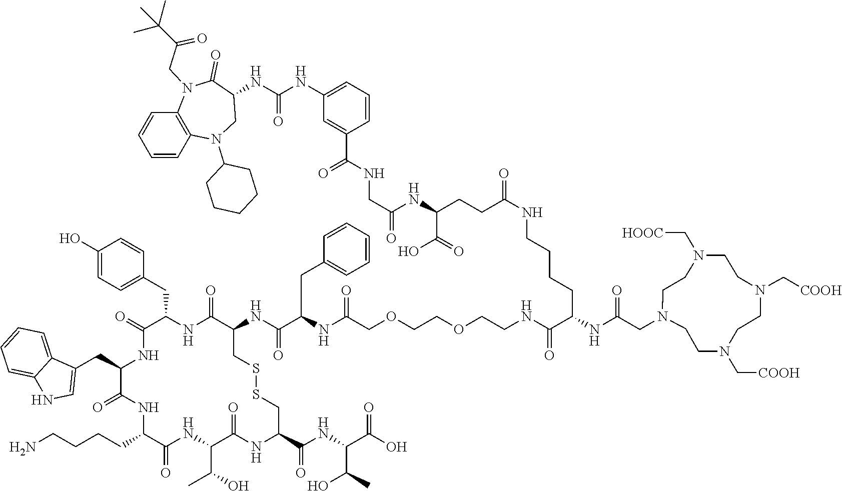 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-2 | DOTA-K(- gE-G- Z360)- OEG-OEG- f[CYwKTC] T-OH | 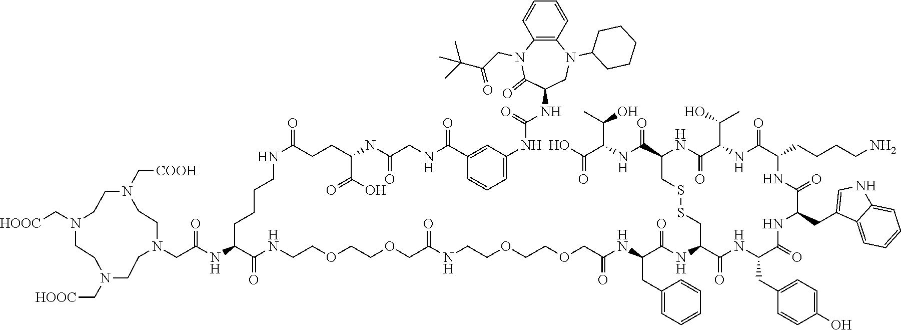 |
| I-3 | DOTA-K(- E-Z360)- OEG- f[CYwKTC] T-NH₂ | 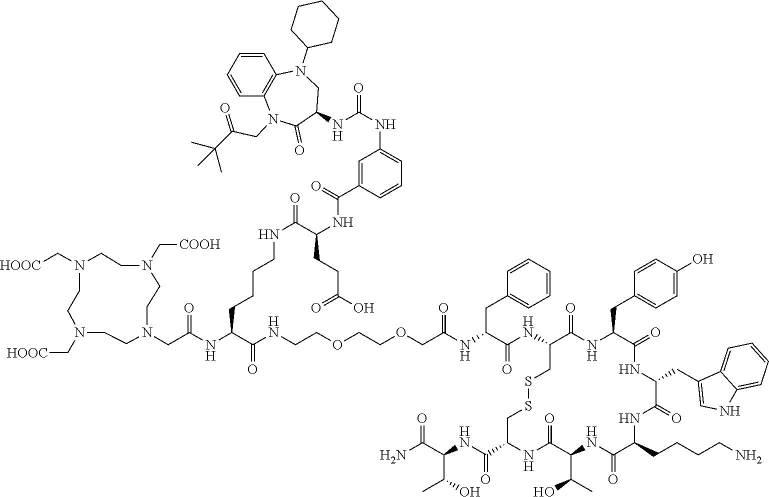 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(N)=O] |
| I-4 | DOTA-K(- E-Z360)- f[CYwKTC] T-NH₂ | 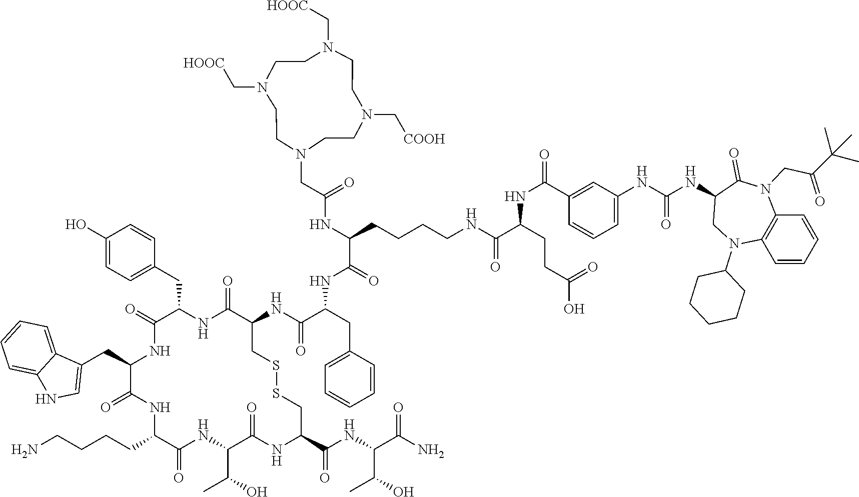 [SMILES: C[C@@H](O)[C@H](NC(=O)[C@@H]1CSSC[C@H](NC(=O)[C@@H](Cc2ccccc2)NC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(N)=O] |
| I-5 | DOTA-K(- E-Z360)- OEG- f[CYwKTC]- Thr(ol) | 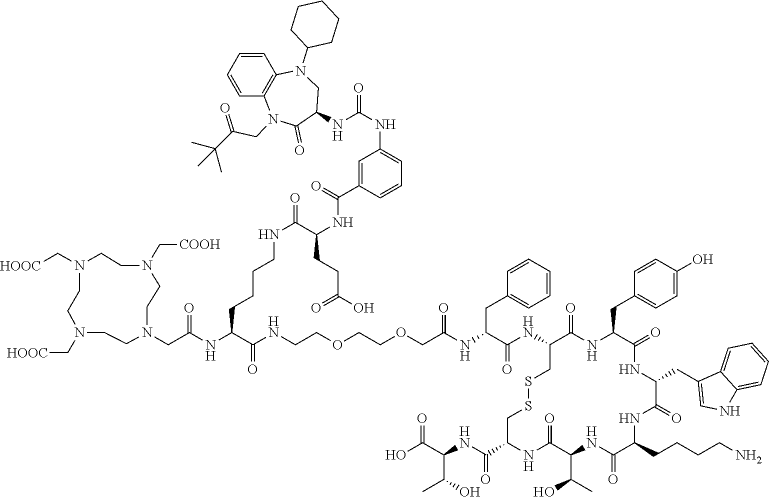 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-6 | DOTA-K(- E-Z360)- f[CYwKTC]- Thr(ol) | 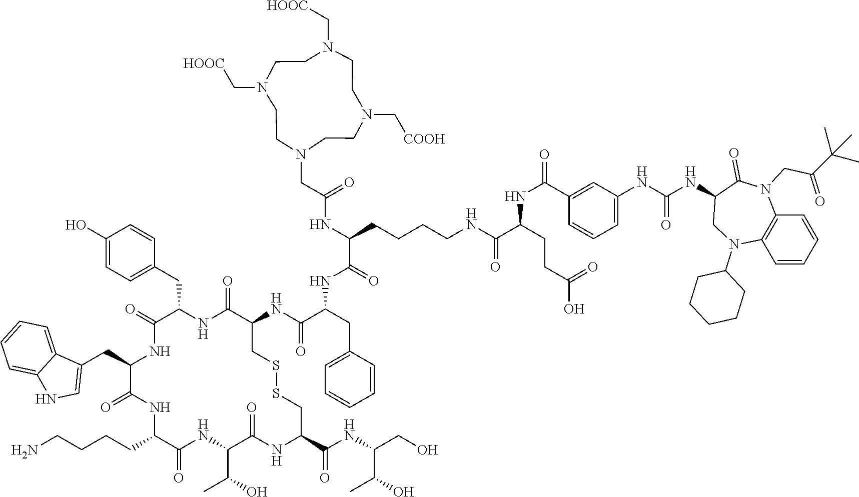 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)CSSC[C@@H](C(=O)N[C@H](CO)[C@@H](C)O)NC1=O] |
| I-7 | DOTA-gE- gE-gE-gE- OEG-K(- gE-G- Z360)- OEG- f[CYwKTC] T-OH) | 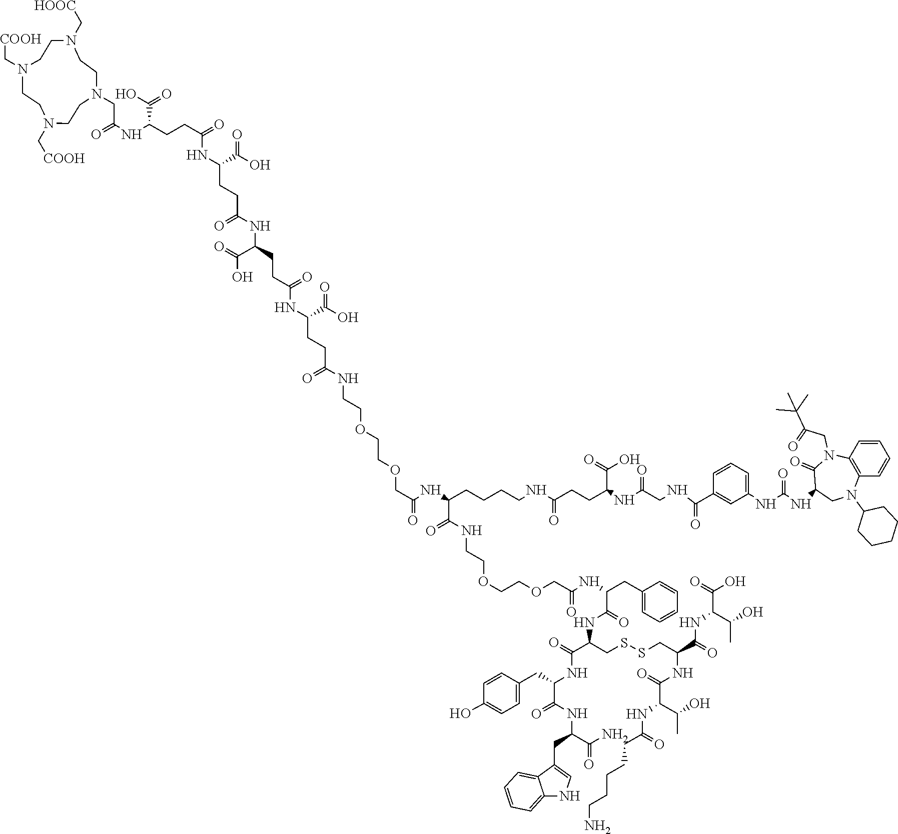 |
| I-8 | DOTA-gE- gE-gE- OEG-K(- gE-G- Z360)- OEG- f[CYwKTC] T-OH | 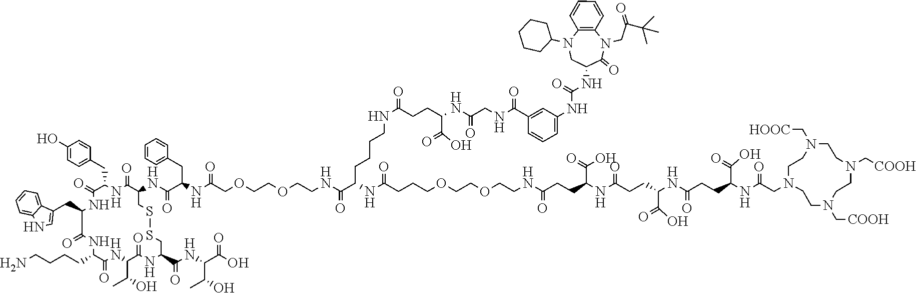 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CCCOCCOCCNC(=O)CC[C@H](NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-9 | DOTA-gE- K(-gE-G- Z360)-gE- f[CYwKTC] T-OH | 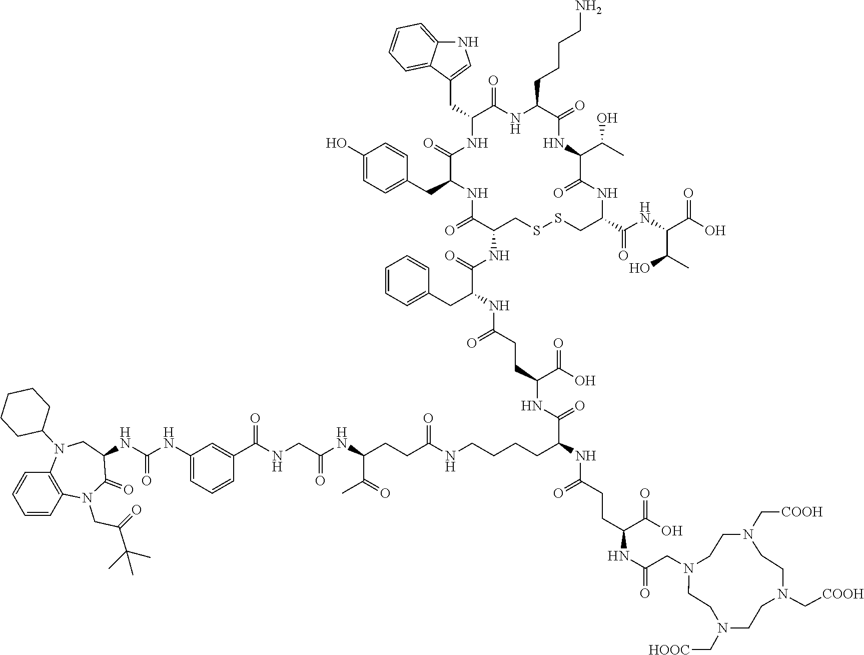 [SMILES: CC(=O)[C@H](CCC(=O)NCCCC[C@H](NC(=O)CC[C@H](NC(=O)CN1CCN(CC(=O)O)CCN(CC(=O)O)CCN(CC(=O)O)CC1)C(=O)O)C(=O)N[C@@H](CCC(=O)N[C@H](Cc1ccccc1)C(=O)N[C@H]1CSSC[C@@H](C(=O)N[C@H](C(=O)O)[C@@H](C)O)NC(=O)[C@H]([C@@H](C)O)NC(=O)[C@H](CCCCN)NC(=O)[C@@H](Cc2c[nH]c3ccccc23)NC(=O)[C@H](Cc2ccc(O)cc2)NC1=O)C(=O)O)NC(=O)CNC(=O)c1cccc(NC(=O)N[C@@H]2CN(C3CCCCC3)c3ccccc3N(CC(=O)C(C)(C)C)C2=O)c1] |
| I-10 | DOTA-gE- gE-K(-gE- G-Z360)- OEG- f[CYwKTC] T-OH | 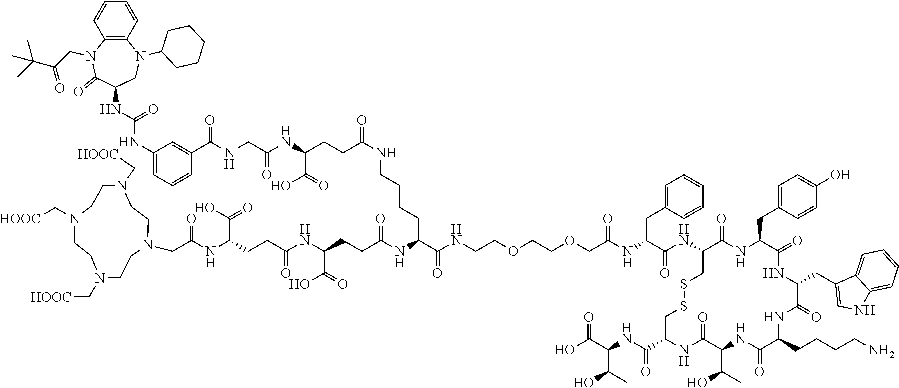 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-11 | DOTA-gE- gE-OEG- K(-E- Z360)- OEG- f[CYwKTC] T-OH | 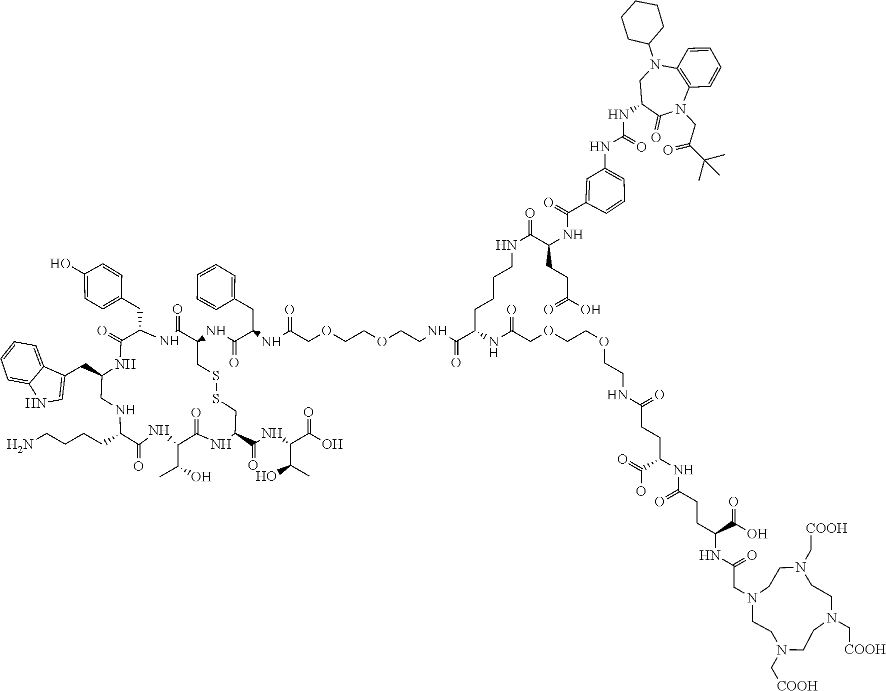 |
| I-12 | (Z360)-E- gE-OEG- gE- K(DOTA)- OEG- f[CYwKTC] T-OH | 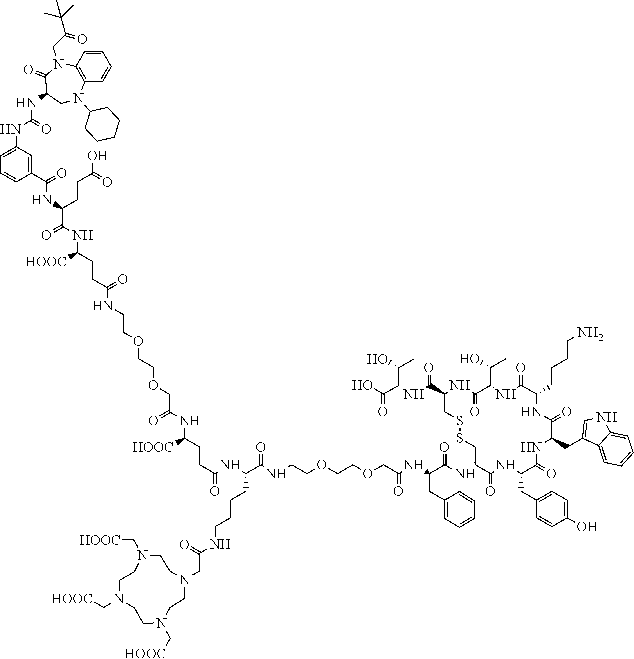 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CN2CCN(CC(=O)O)CCN(CC(=O)O)CCN(CC(=O)O)CC2)NC(=O)CC[C@H](NC(=O)COCCOCCNC(=O)CC[C@H](NC(=O)[C@H](CCC(=O)O)NC(=O)c2cccc(NC(=O)N[C@@H]3CN(C4CCCCC4)c4ccccc4N(CC(=O)C(C)(C)C)C3=O)c2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-13 | (Z360)-gE- gE-OEG- K(DOTAGA)- OEG- f[CYwKTC] T-OH | 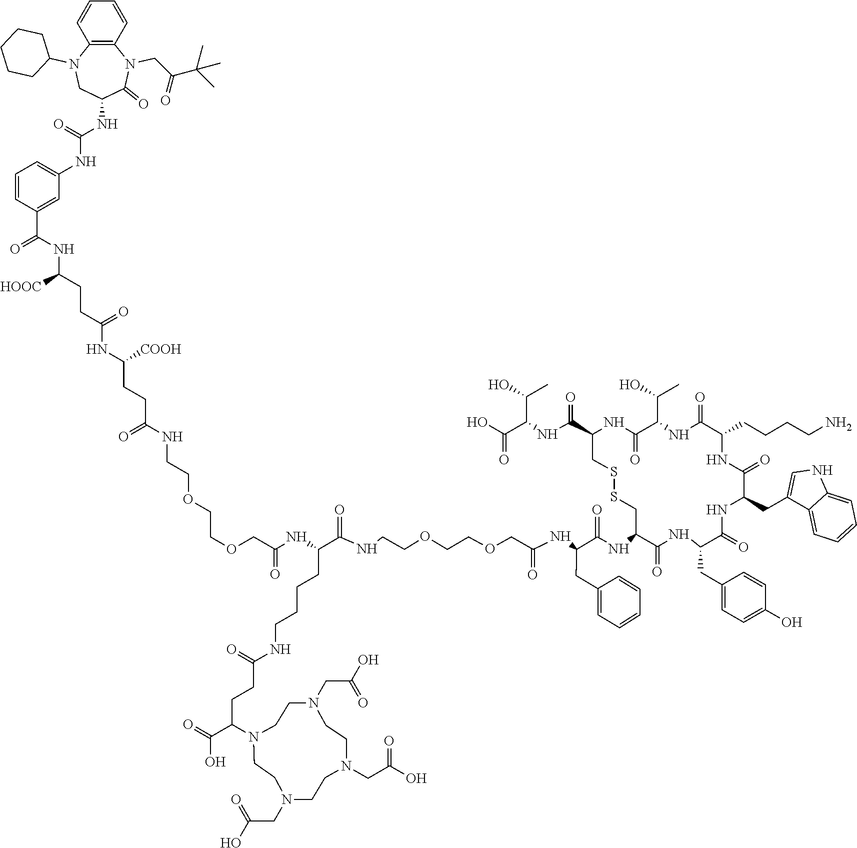 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CCC(C(=O)O)N2CCN(CC(=O)O)CCN(CC(=O)O)CCN(CC(=O)O)CC2)NC(=O)COCCOCCNC(=O)CC[C@H](NC(=O)CC[C@H](NC(=O)c2cccc(NC(=O)N[C@@H]3CN(C4CCCCC4)c4ccccc4N(CC(=O)C(C)(C)C)C3=O)c2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-14 | DOTA-gE- gE-OEG- K(-gE-G- Z360)- OEG- f[CYwKTC] T-OH | 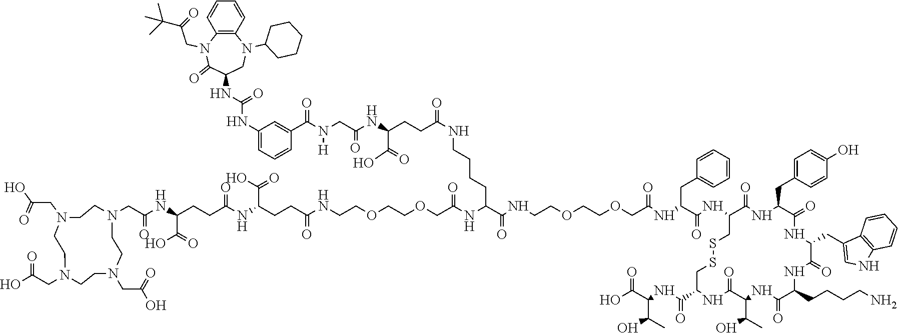 [SMILES: C[C@@H](O)[C@H](NC(=O)[C@@H]1CSSC[C@H](NC(=O)[C@@H](Cc2ccccc2)NC(=O)COCCOCCNC(=O)C(CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-15 | DOTA-gE- gE-OEG- OEG-K(- gE-G- Z360)- OEG- f[CYwKTC] T-OH | 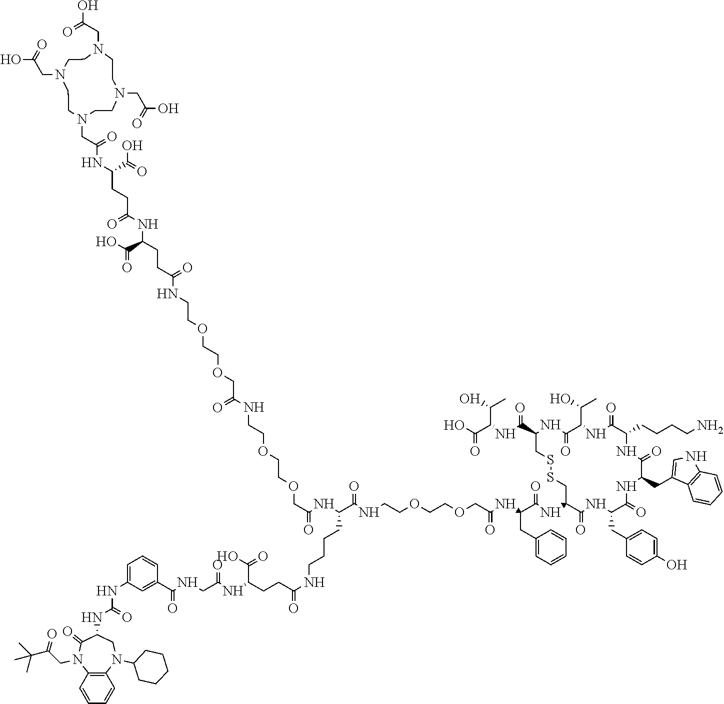 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-16 | DOTA-gE- gE-OEG- OEG-K(- gE-G- Z360)- OEG-OEG- f[CYwKTC] T-OH | 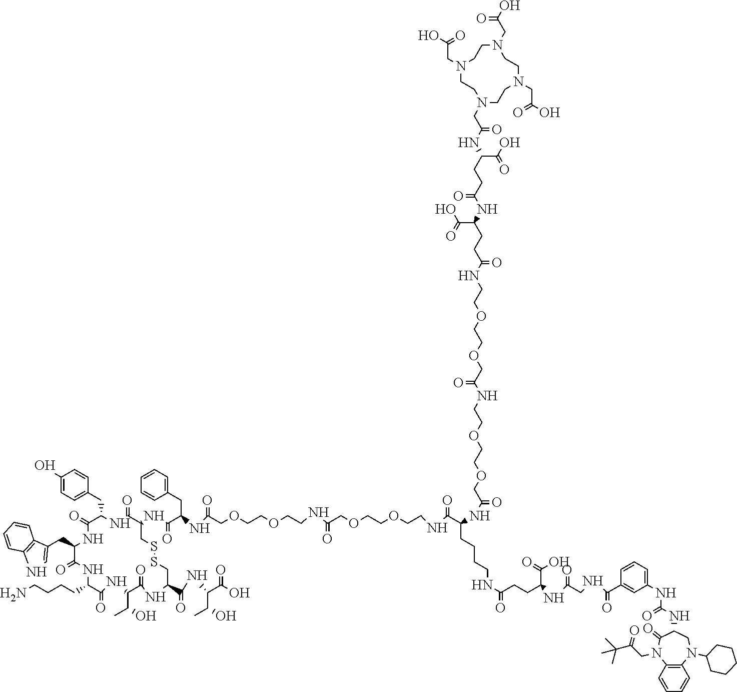 [SMILES: C[C@@H](O)[C@H](NC(=O)[C@@H]1CSSC[C@H](NC(=O)[C@@H](Cc2ccccc2)NC(=O)COCCOCCNC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-17 | (Z360)-gE- gE-OEG- OEG- K(DOTAG A)-OEG- OEG- f[CYwKTC] T-OH ( | 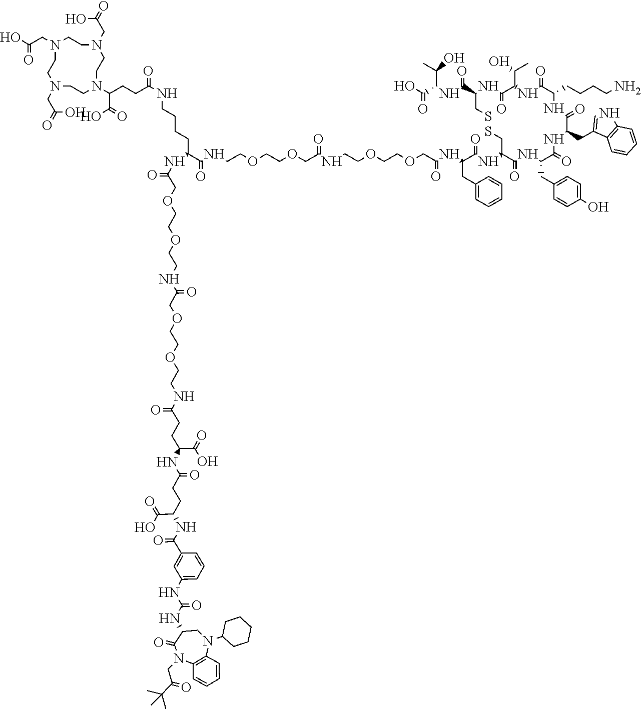 [SMILES: C[C@@H](O)[C@H](NC(=O)[C@@H]1CSSC[C@H](NC(=O)[C@@H](Cc2ccccc2)NC(=O)COCCOCCNC(=O)COCCOCCNC(=O)[C@H](CCCCNC(=O)CCC(C(=O)O)N2CCN(CC(=O)O)CCN(CC(=O)O)CCN(CC(=O)O)CC2)NC(=O)COCCOCCNC(=O)COCCOCCNC(=O)CC[C@H](NC(=O)CC[C@H](NC(=O)c2cccc(NC(=O)N[C@@H]3CN(C4CCCCC4)c4ccccc4N(CC(=O)C(C)(C)C)C3=O)c2)C(=O)O)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-18 | DOTA-gE- OEG-OEG- OEG-OEG- K(-gE-G- Z360)- OEG-gE- OEG-OEG- f[CYwKTC] T-OH | 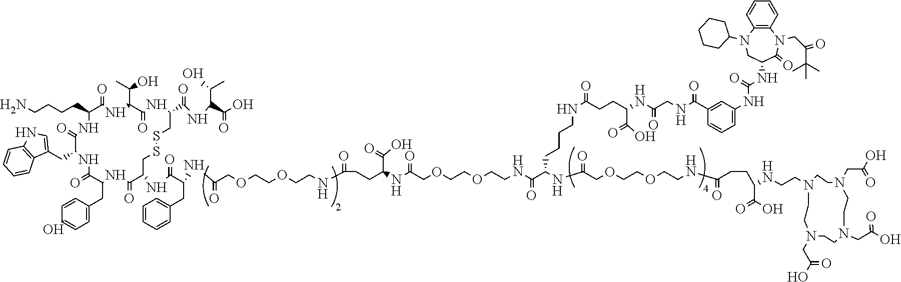 |
| I-19 | DOTAGA- OEG-K(- gE-G- Z360)- f[CYwKTC] T-OH | 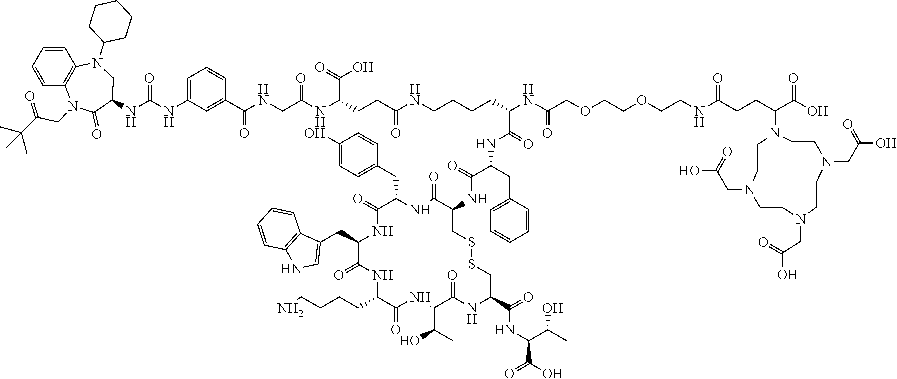 [SMILES: C[C@@H](O)[C@H](NC(=O)[C@@H]1CSSC[C@H](NC(=O)[C@@H](Cc2ccccc2)NC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CCC(C(=O)O)N2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-20 | DOTAGA- OEG-K(- gE-G- Z360)- OEG- f[CYwKTC] T-OH | 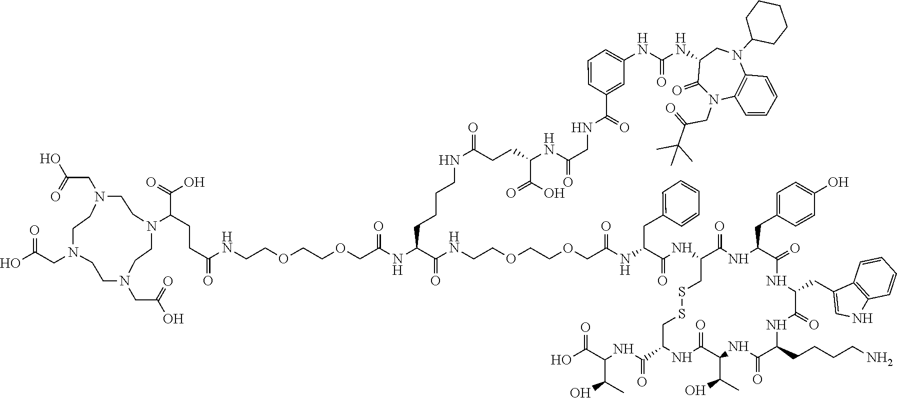 [SMILES: C[C@@H](O)C(NC(=O)[C@@H]1CSSC[C@H](NC(=O)[C@@H](Cc2ccccc2)NC(=O)COCCOCCNC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)CCC(C(=O)O)N2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-21 | DOTA-gE- OEG-OEG- K(-gE-G- Z360)- OEG- f[CYwKTC] T-OH | 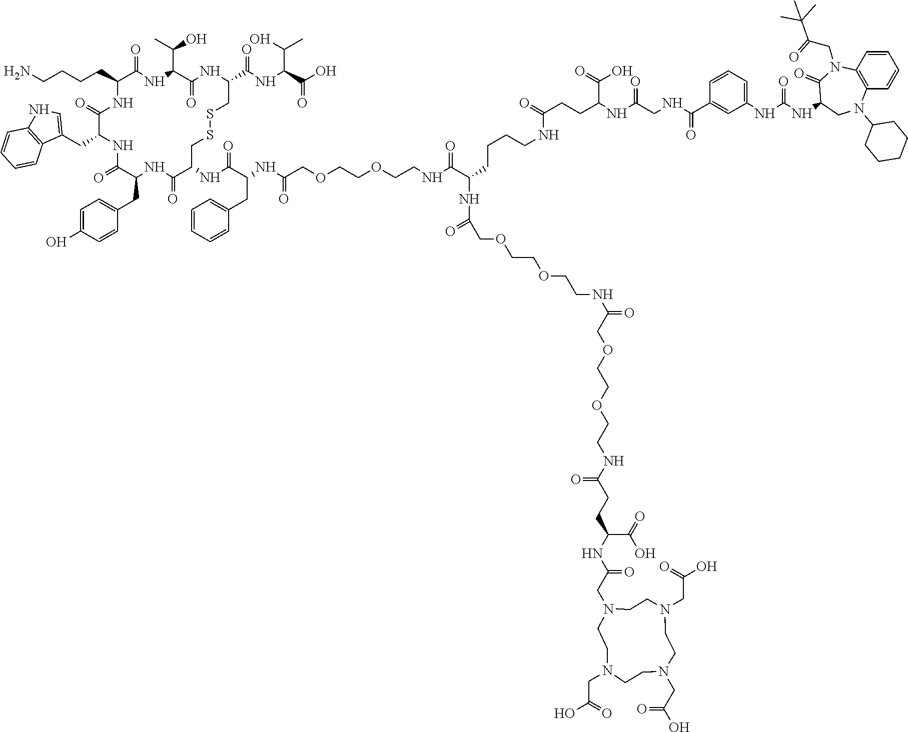 [SMILES: CC(O)[C@H](NC(=O)[C@@H]1CSSC[C@H](NC(=O)[C@@H](Cc2ccccc2)NC(=O)COCCOCCNC(=O)[C@H](CCCCNC(=O)CCC(NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)CC[C@H](NC(=O)CN2CCN(CC(=O)O)CCN(CC(=O)O)CCN(CC(=O)O)CC2)C(=O)O)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-22 | DOTA-gE- OEG-OEG- OEG-OEG- OEG-K(- gE-G- Z360)- OEG- f[CYwKTC] T-OH) | 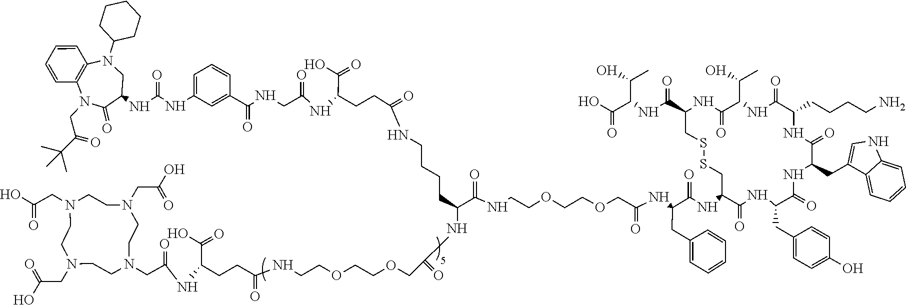 |
| I-23 | DOTA- OEG-OEG- OEG-K(- gE-G- Z360)- OEG-gE- OEG-OEG- f[CYwKTC] T-OH | 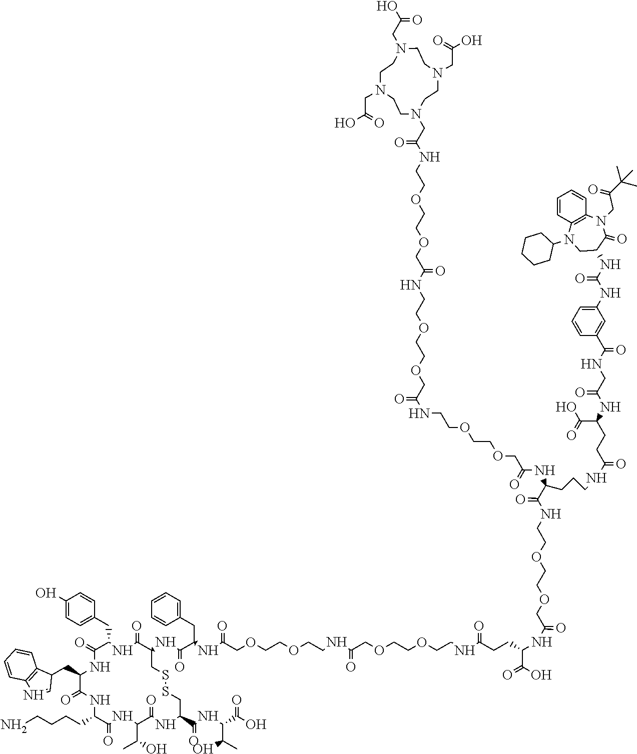 [SMILES: C[C@@H](O)C1NC(=O)[C@H](CCCCN)NC(=O)[C@@H](CC2C=Nc3ccccc32)NC(=O)[C@H](Cc2ccc(O)cc2)NC(=O)[C@@H](NC(=O)[C@@H](Cc2ccccc2)NC(=O)COCCOCCNC(=O)COCCOCCNC(=O)CC[C@H](NC(=O)COCCOCCNC(=O)[C@H](CCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)COCCOCCNC(=O)COCCOCCNC(=O)COCCOCCNC(=O)CN2CCN(CC(=O)O)CCN(CC(=O)O)CCN(CC(=O)O)CC2)C(=O)O)CSSC[C@@H](C(=O)N[C@H](C(=O)O)[C@@H](C)O)NC1=O] |
| I-24 | DOTA-gE- OEG-OEG- OEG-OEG- K(-gE-G- Z360)- OEG-OEG- OEG-OEG- f[CYwKTC] T-OH | 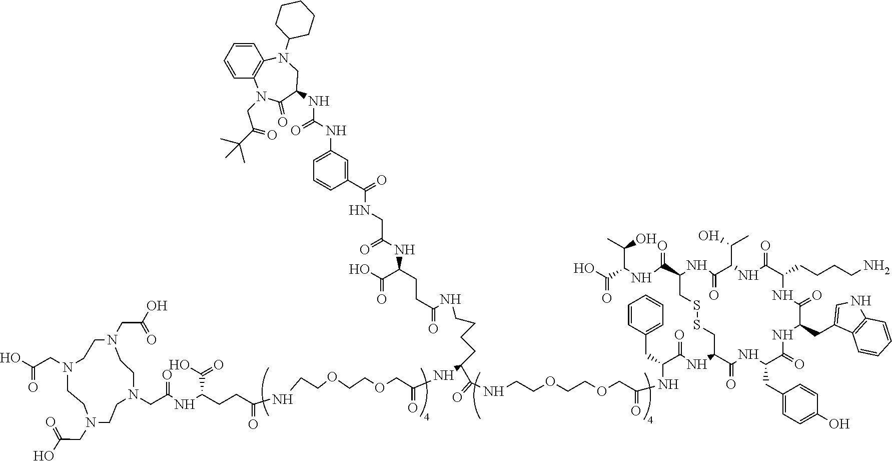 |
| I-25 | DOTA-K(- E-Z360)- f[CYwKTC] T-OH | 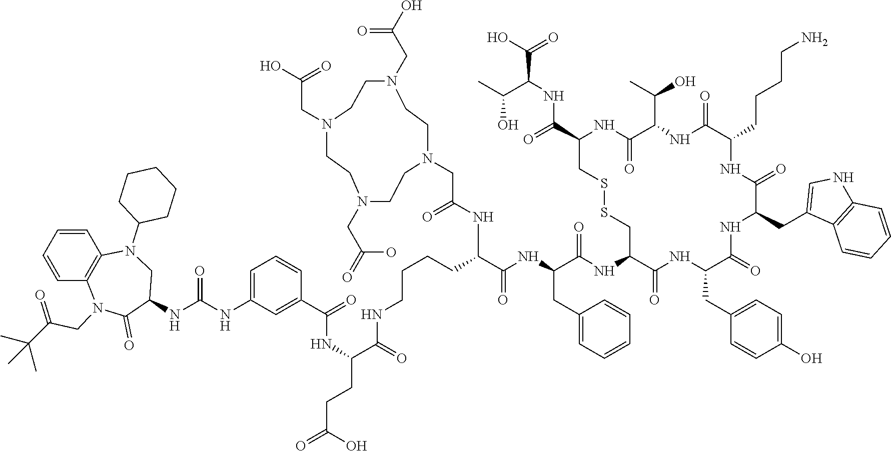 |
| I-26 | DOTA-K(- gE-G- Z360)- f[CYwKTC] T-OH | 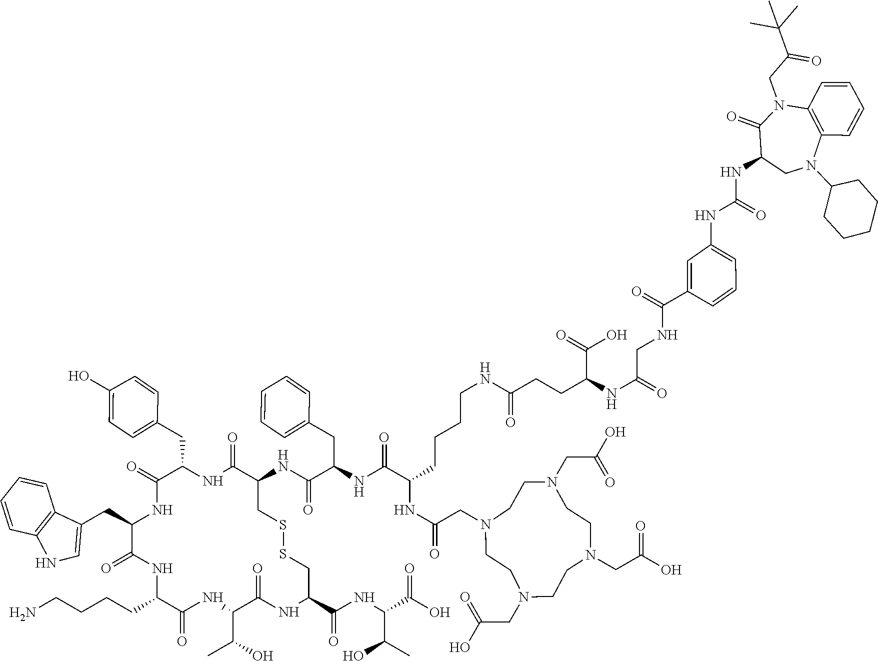 [SMILES: C[C@@H](O)[C@H](NC(=O)[C@@H]1CSSC[C@H](NC(=O)[C@@H](Cc2ccccc2)NC(=O)[C@H](CCCCNC(=O)CC[C@H](NC(=O)CNC(=O)c2cccc(NC(=O)N[C@@H]3CN(C4CCCCC4)c4ccccc4N(CC(=O)C(C)(C)C)C3=O)c2)C(=O)O)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-27 | DOTA-K(- E-Z360)- OEG- f[CYwKTC] T-OH | 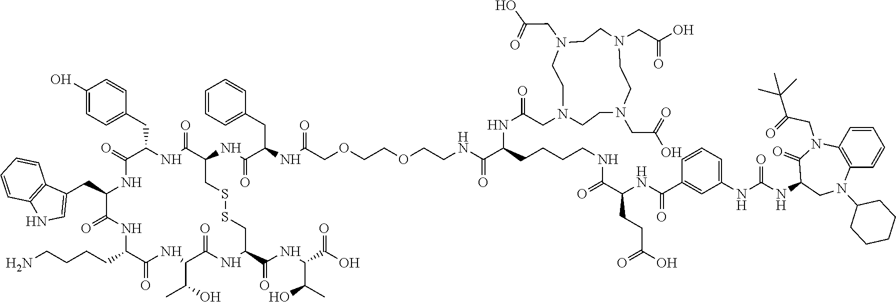 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-28 | DOTA-K(- OEG-E- Z360)- f[CYwKTC] T-OH | 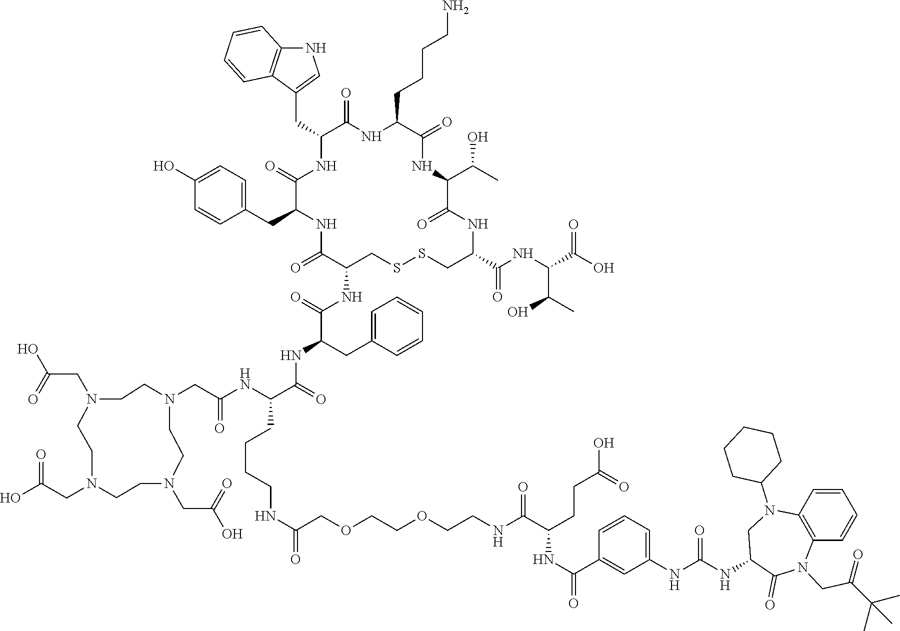 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-29 | DOTA-K(- OEG-OEG- E-Z360)- f[CYwKTC] T-OH | 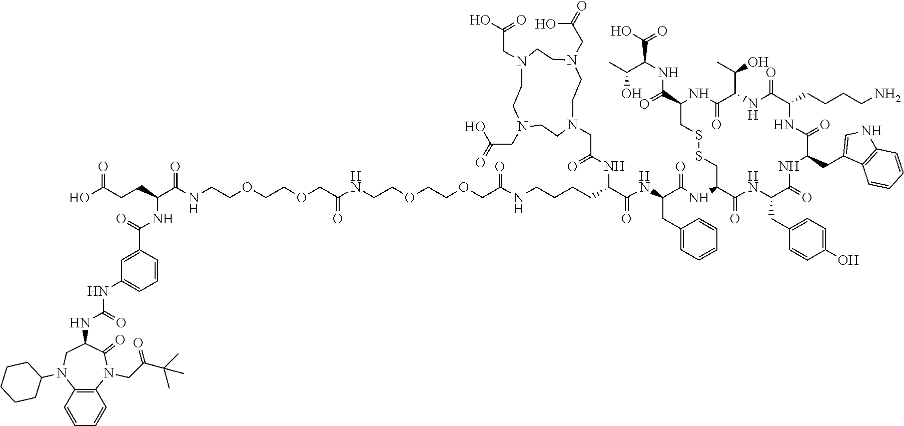 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)COCCOCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-30 | DOTA-K(- OEG-E- Z360)- OEG- f[CYwKTC] T-OH | 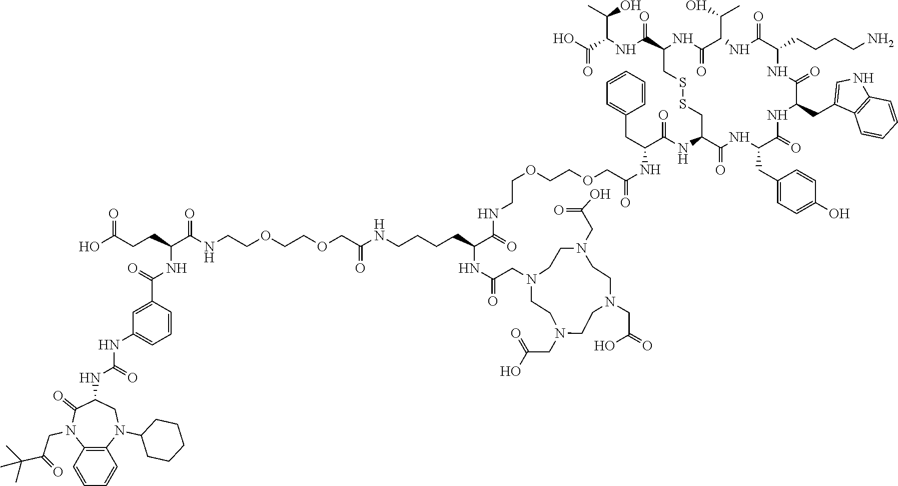 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)COCCOCCNC(=O)[C@H](CCC(=O)O)NC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-31 | Z360-OEG- K(DOTAG A)-OEG- f[CYwKTC] T-OH | 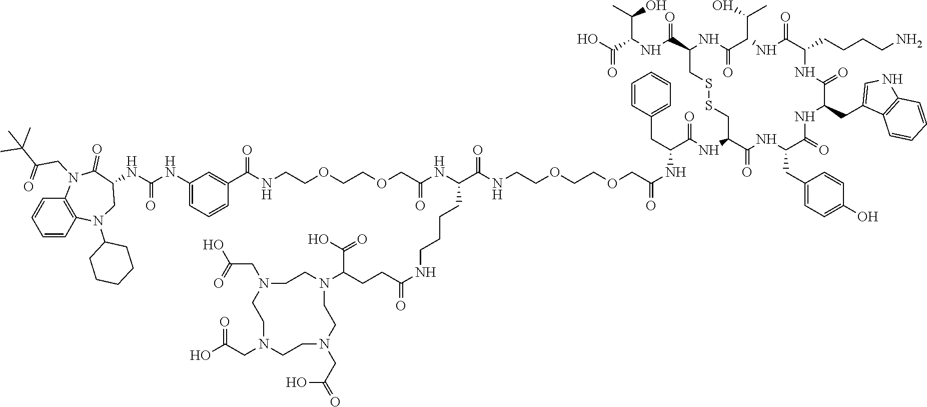 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)CCC(C(=O)O)N2CCN(CC(=O)O)CCN(CC(=O)O)CCN(CC(=O)O)CC2)NC(=O)COCCOCCNC(=O)c2cccc(NC(=O)N[C@@H]3CN(C4CCCCC4)c4ccccc4N(CC(=O)C(C)(C)C)C3=O)c2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-32 | Z360-G- gE-OEG- OEG-OEG- K(DOTA)- OEG- f[CYwKTC] T-OH | 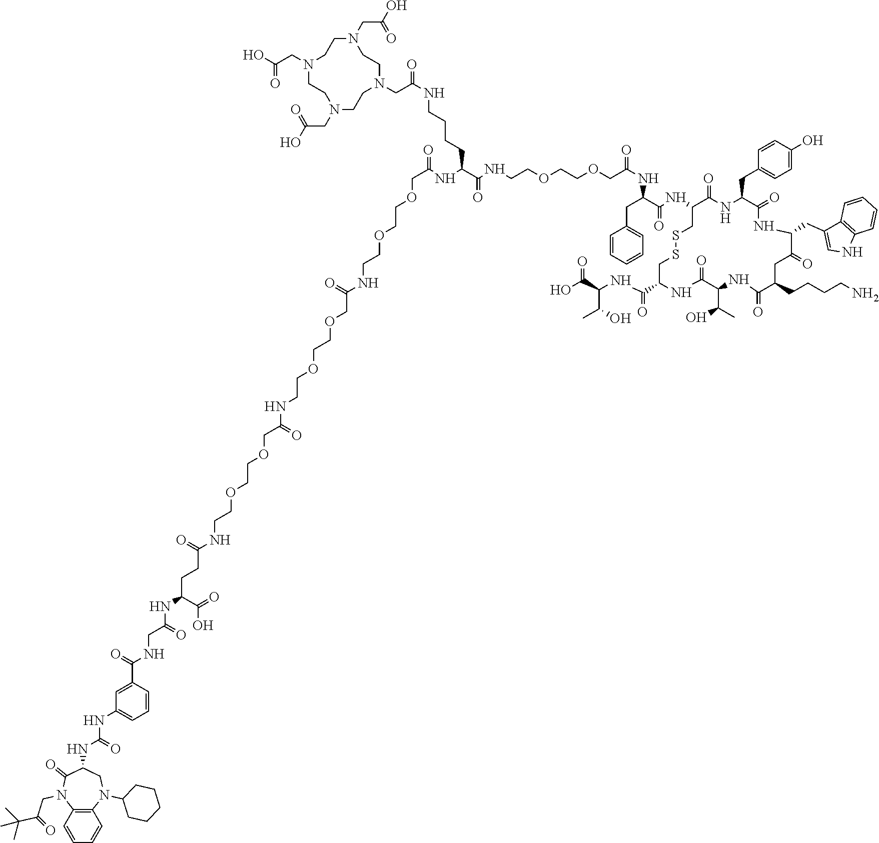 |
| I-33 | DOTA- OEG-OEG- OEG-OEG- OEG-OEG- K(-gE-G- Z360)- OEG- f[CYwKTC] T-OH | 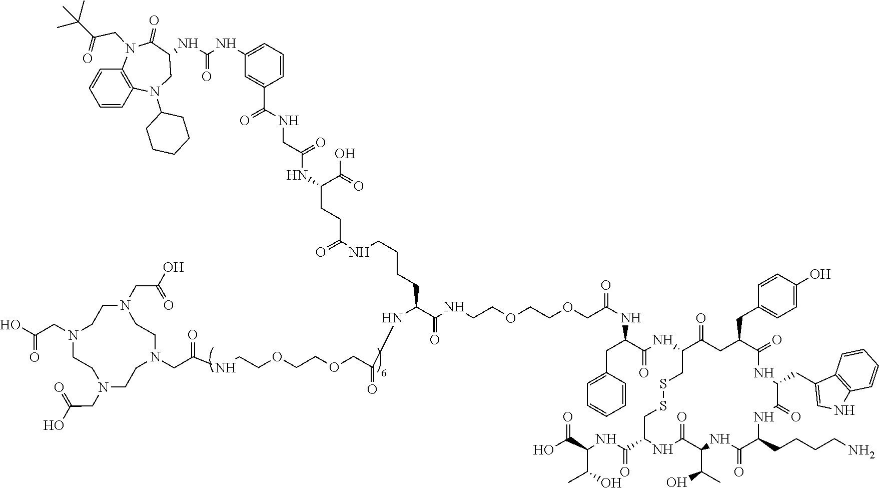 |
| I-34 | DOTA- K(Z360)- f[CYwKTC] T-OH | 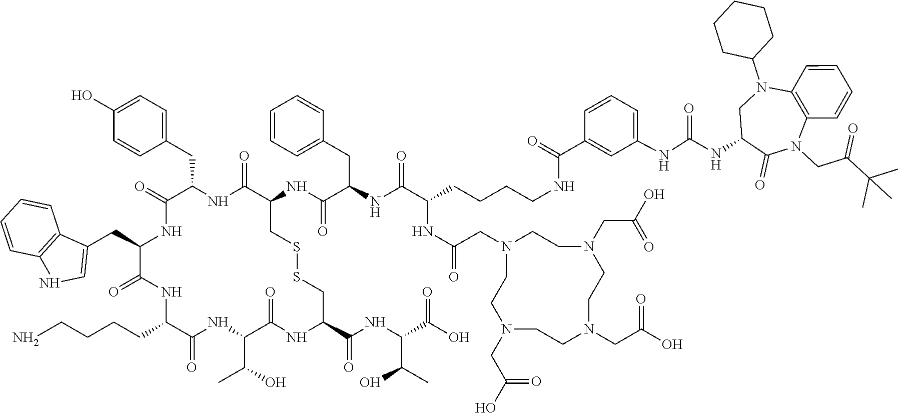 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-35 | DOTA- OEG- K(Z360)- f[CYwKTC] T-OH | 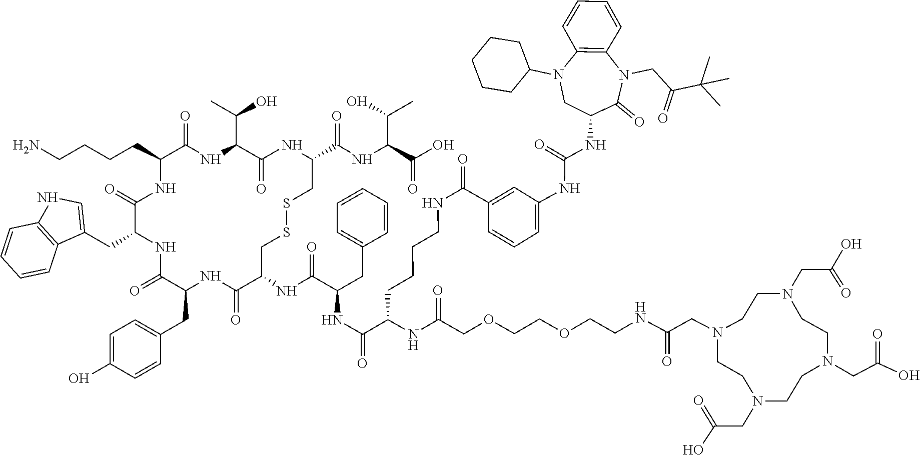 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)COCCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-36 | DOTA-K(- OEG- Z360)- f[CYwKTC] T-OH | 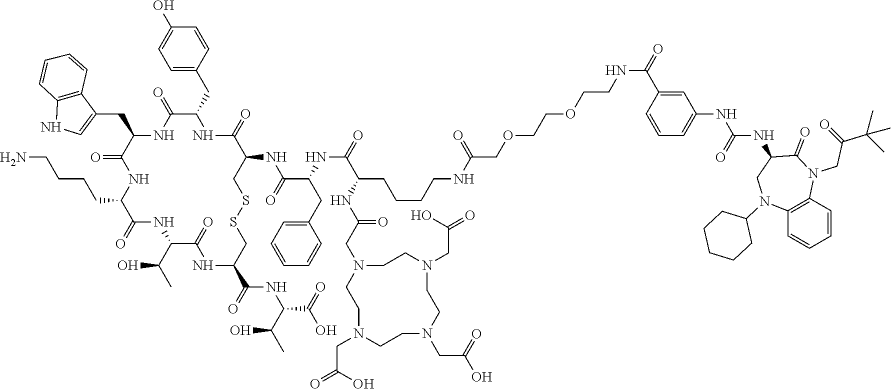 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-37 | DOTA- K(Z360)- OEG- f[CYwKTC] T-OH | 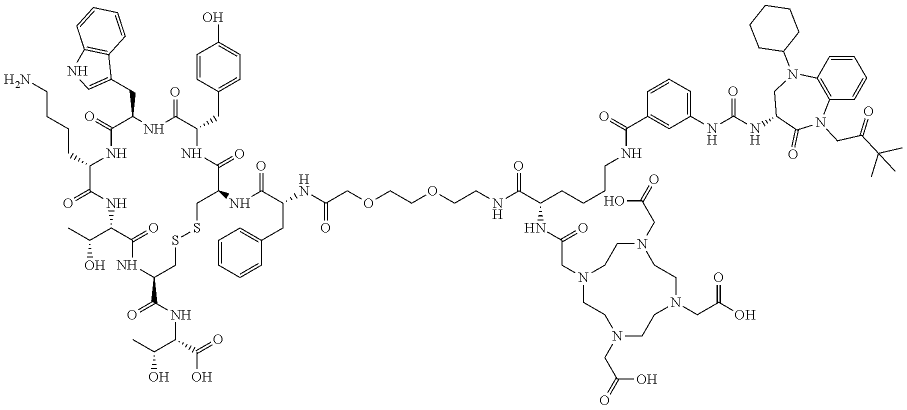 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-38 | DOTA- K(Z360)- PEG3- f[CYwKTC] T-OH | 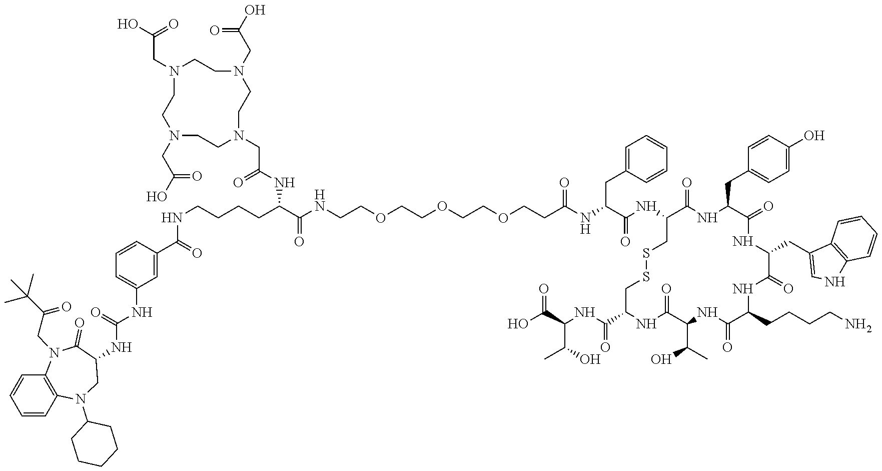 [SMILES: C[C@@H](O)[C@H](NC(=O)[C@@H]1CSSC[C@H](NC(=O)[C@@H](Cc2ccccc2)NC(=O)CCOCCOCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-39 | DOTA- K(Z360)- OEG-OEG- f[CYwKTC] T-OH | 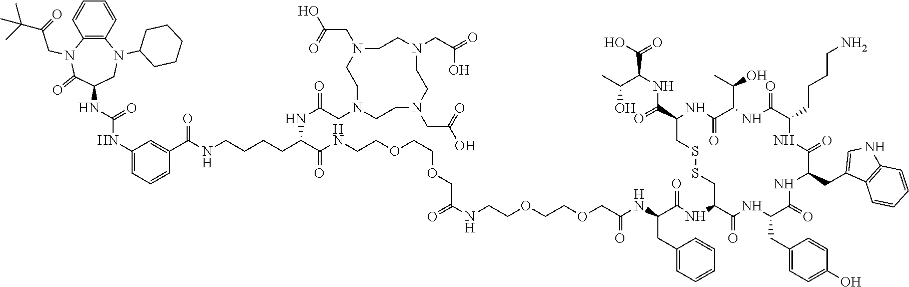 [SMILES: C[C@@H](O)[C@H](NC(=O)[C@@H]1CSSC[C@H](NC(=O)[C@@H](Cc2ccccc2)NC(=O)COCCOCCNC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-40 | DOTA- K(Z360)- PEG6- f[CYwKTC] T-OH | 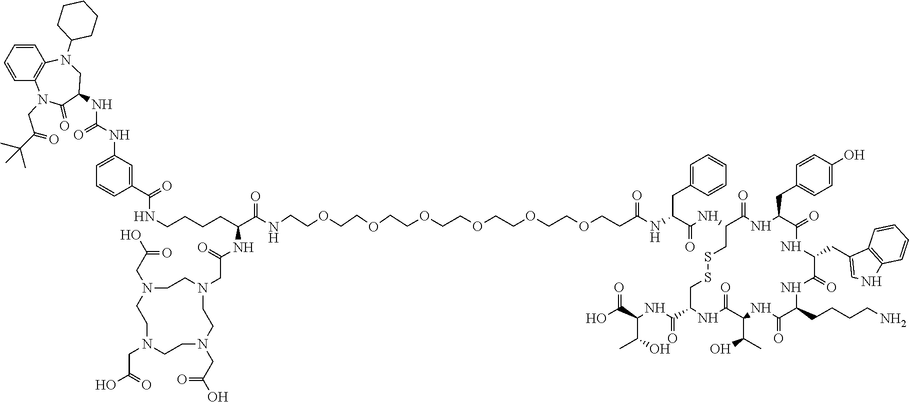 [SMILES: C[C@@H](O)[C@H](NC(=O)[C@@H]1CSSC[C@H](NC(=O)[C@@H](Cc2ccccc2)NC(=O)CCOCCOCCOCCOCCOCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-41 | DOTA- OEG-OEG- K(Z360)- f[CYwKTC] T-OH | 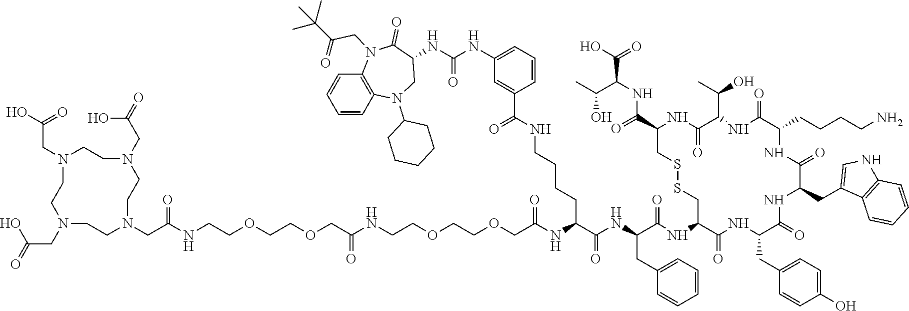 |
| I-42 | DOTA-K(- OEG-OEG- Z360)- f[CYwKTC] T-OH | 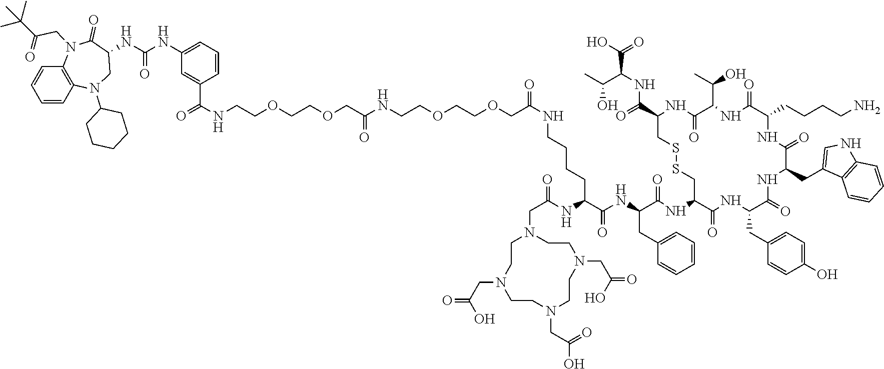 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-43 | DOTA-K(- OEG- Z360)- OEG- f[CYwKTC] T-OH | 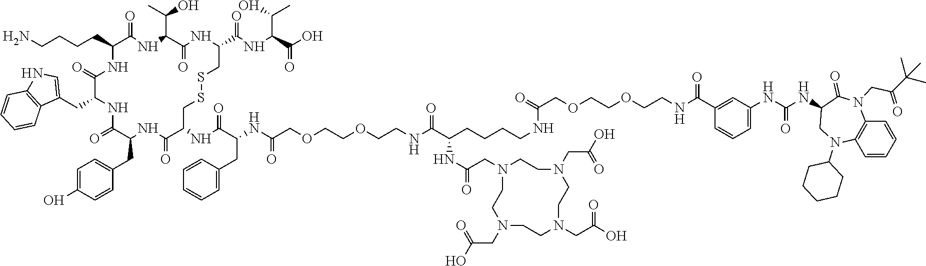 [SMILES: C[C@@H](O)[C@H](NC(=O)[C@@H]1CSSC[C@H](NC(=O)[C@@H](Cc2ccccc2)NC(=O)COCCOCCNC(=O)[C@H](CCCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-44 | DOTA- OEG-K(- OEG- Z360)- f[CYwKTC] T-OH | 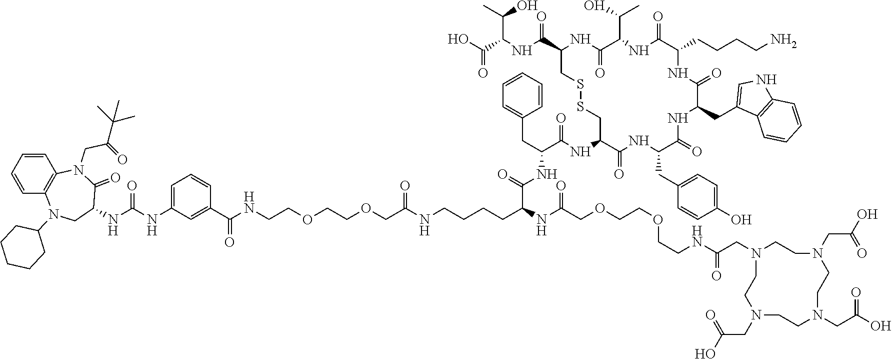 [SMILES: C[C@@H](O)[C@H](NC(=O)[C@@H]1CSSC[C@H](NC(=O)[C@@H](Cc2ccccc2)NC(=O)[C@H](CCCCNC(=O)COCCOCCNC(=O)c2cccc(NC(=O)N[C@@H]3CN(C4CCCCC4)c4ccccc4N(CC(=O)C(C)(C)C)C3=O)c2)NC(=O)COCCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-45 | DOTA- K(Z360)- OEG-OEG- OEG- f[CYwKTC] T-OH | 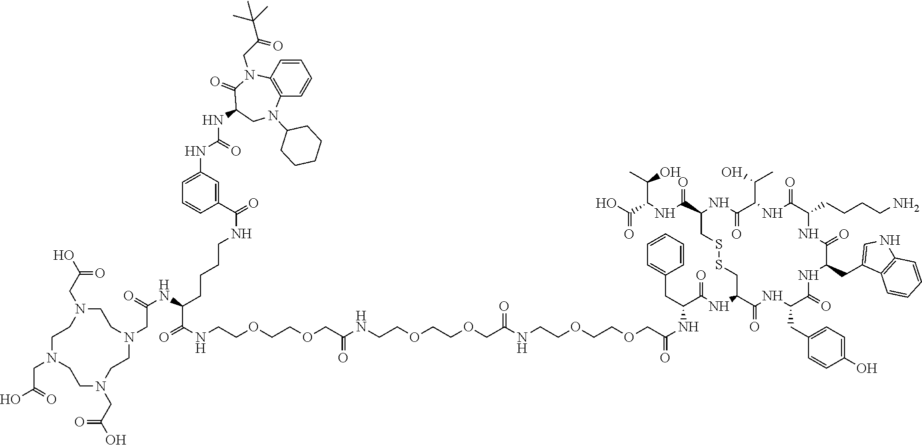 [SMILES: C[C@@H](O)[C@H](NC(=O)[C@@H]1CSSC[C@H](NC(=O)[C@@H](Cc2ccccc2)NC(=O)COCCOCCNC(=O)COCCOCCNC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-46 | DOTA-K(- OEG-OEG- OEG- Z360)- f[CYwKTC] T-OH | 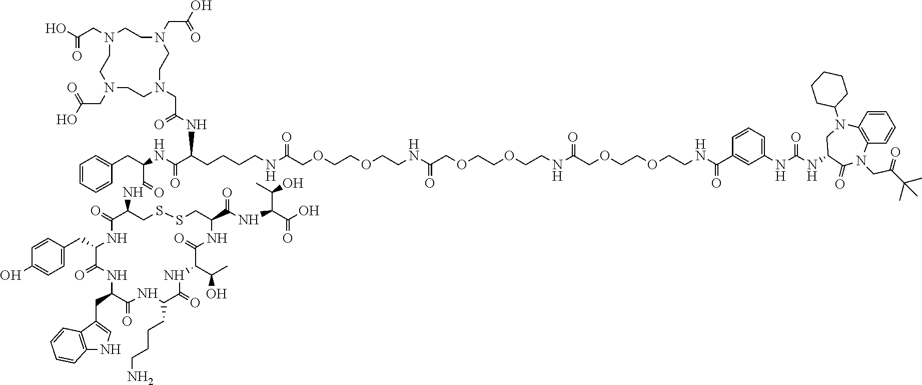 |
| I-47 | DOTA-eK- OEG-OEG- OEG-K(- gE-G- Z360)- OEG- f[CYwKTC] T-OH | 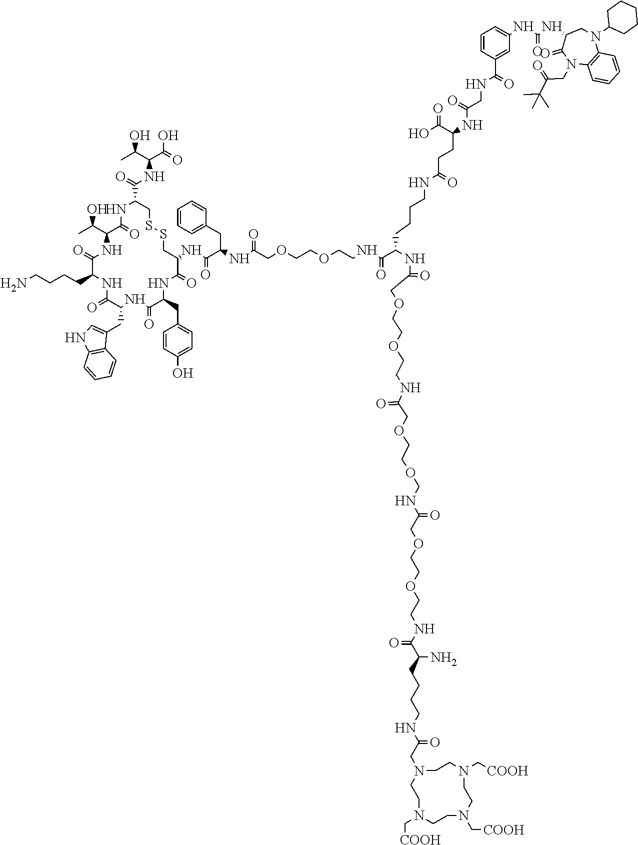 |
| I-48 | DOTA- PEG3- K(Z360)- f[CYwKTC] T-OH | 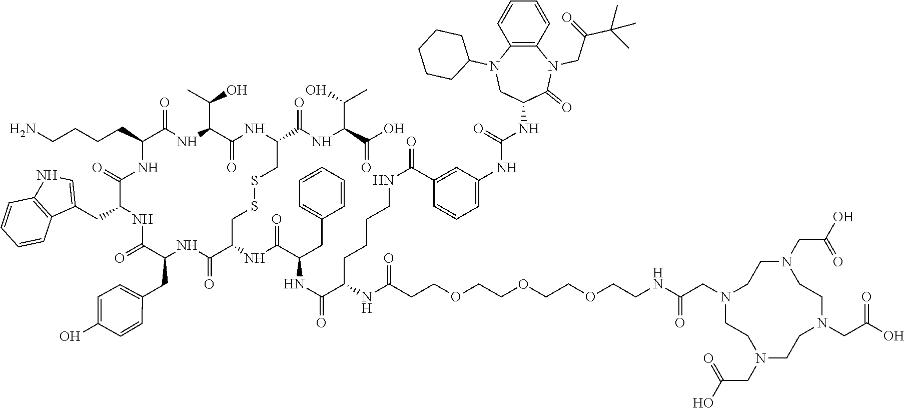 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CCOCCOCCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-49 | DOTA-G- G-K(Z360)- f[CYwKTC] T-OH | 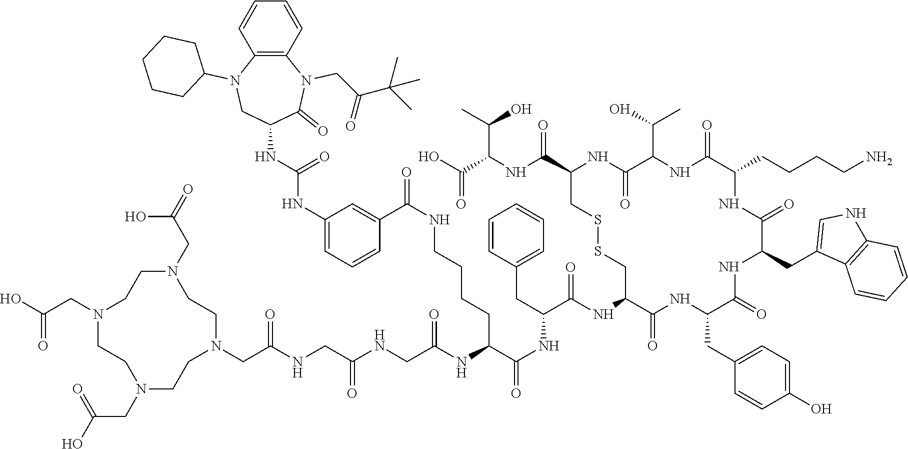 [SMILES: C[C@@H](O)C1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CNC(=O)CNC(=O)CN2CCN(CC(=O)O)CCN(CC(=O)O)CCN(CC(=O)O)CC2)CSSC[C@@H](C(=O)N[C@H](C(=O)O)[C@@H](C)O)NC1=O] |
| I-50 | DOTA-S-S- K(Z360)- f[CYwKTC] T-OH | 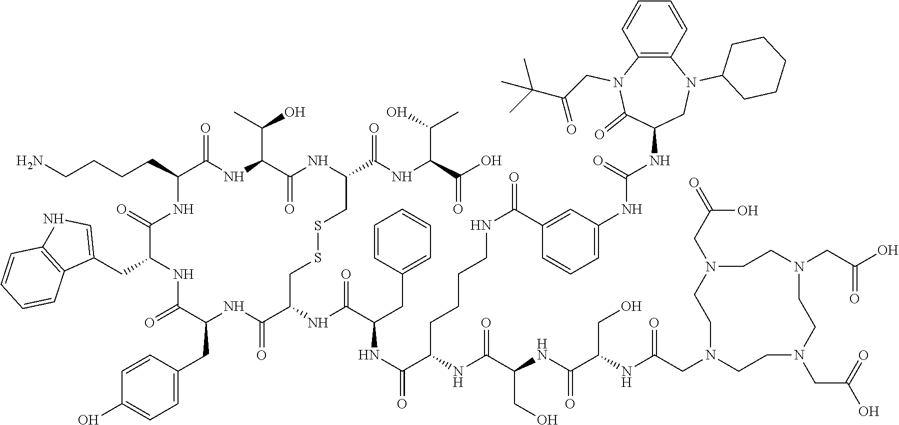 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)[C@H](CO)NC(=O)[C@H](CO)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-51 | DOTA- PEG1- K(Z360)- f[CYwKTC] T-OH | 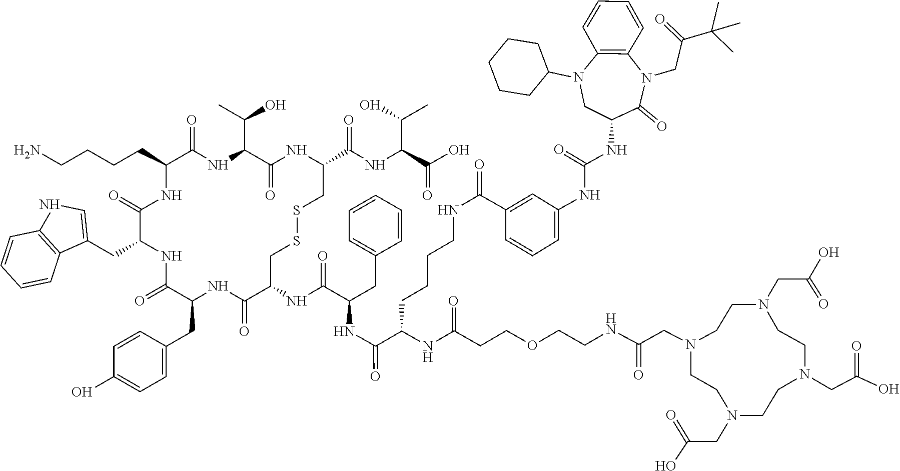 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CCOCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-52 | DOTA- OEG- K(Z360)- OEG- f[CYwKTC] T-OH | 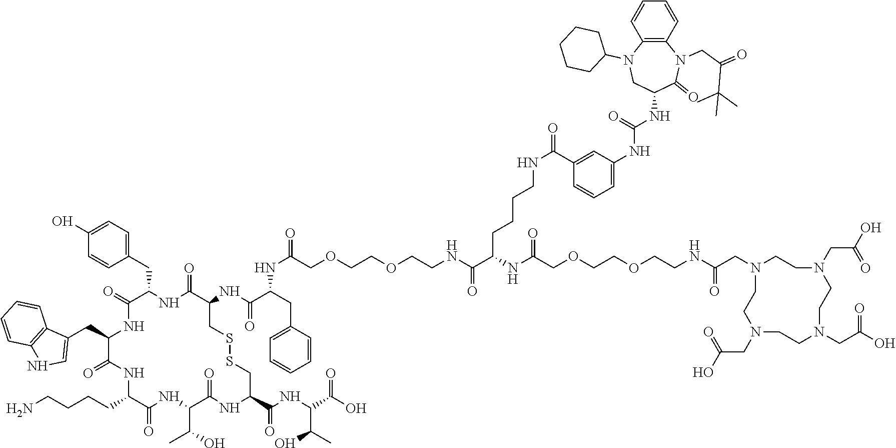 |
| I-53 | DOTA- K(Z360)- OEG-G- f[CYwKTC] T-OH | 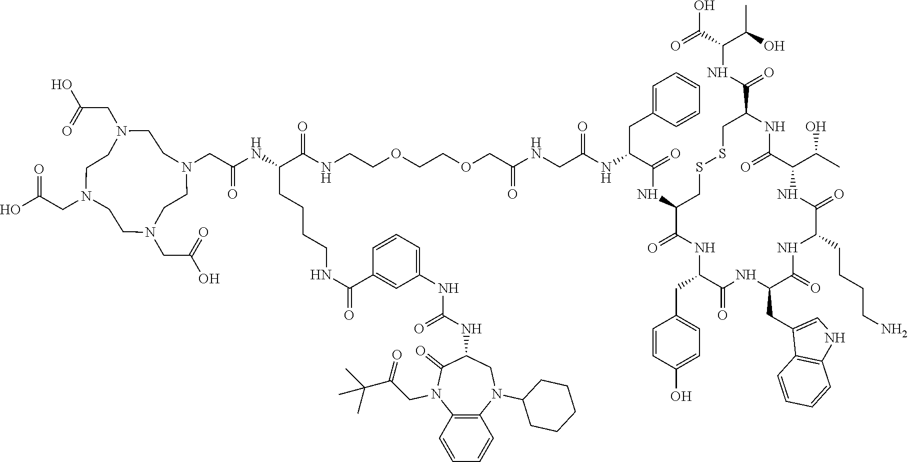 [SMILES: C[C@@H](O)[C@H](NC(=O)[C@@H]1CSSC[C@H](NC(=O)[C@@H](Cc2ccccc2)NC(=O)CNC(=O)COCCOCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-54 | DOTA- K(Z360)-G- S-G- f[CYwKTC] T-OH | 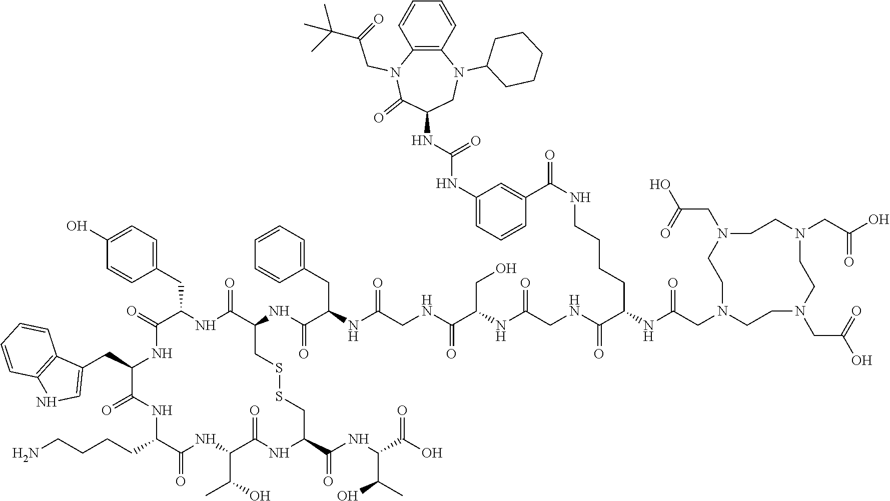 [SMILES: C[C@@H](O)[C@H](NC(=O)[C@@H]1CSSC[C@H](NC(=O)[C@@H](Cc2ccccc2)NC(=O)CNC(=O)[C@H](CO)NC(=O)CNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-55 | DOTA- K(Z360)-G- p-G-p-G- f[CYwKTC] T-OH | 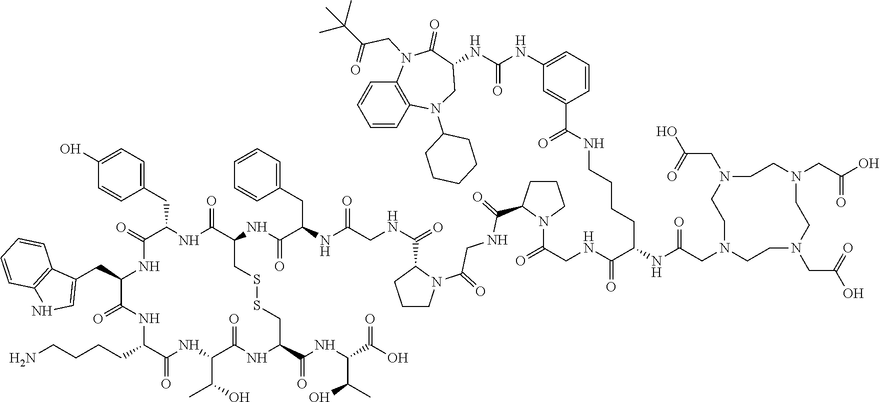 [SMILES: C[C@@H](O)[C@H](NC(=O)[C@@H]1CSSC[C@H](NC(=O)[C@@H](Cc2ccccc2)NC(=O)CNC(=O)[C@H]2CCCN2C(=O)CNC(=O)[C@H]2CCCN2C(=O)CNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-56 | DOTA-G-p- G-K(Z360)- f[CYwKTC] T-OH | 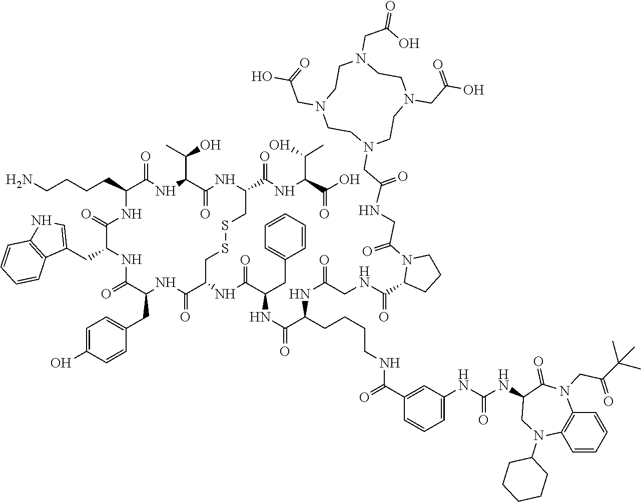 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CNC(=O)[C@H]2CCCN2C(=O)CNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-57 | DOTA-p-G- p-G- K(Z360)- f[CYwKTC] T-OH | 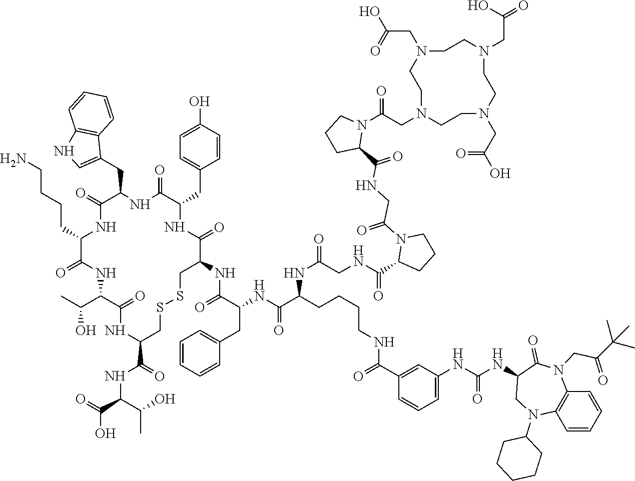 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CNC(=O)[C@H]2CCCN2C(=O)CNC(=O)[C@H]2CCCN2C(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-58 | DOTA-G- S-K(Z360)- G-S- f[CYwKTC] T-OH | 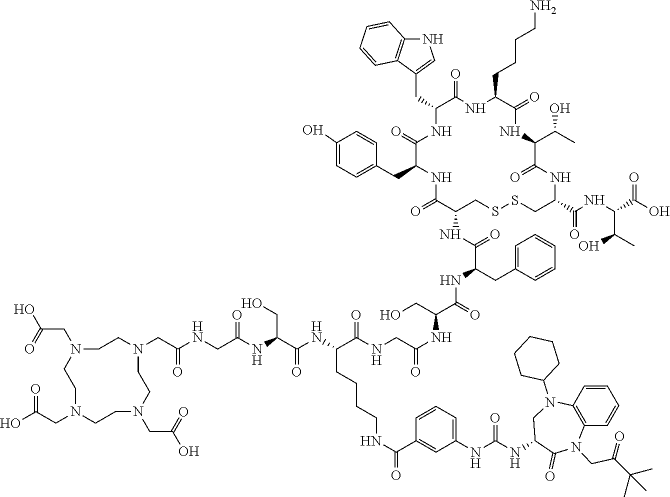 [SMILES: C[C@@H](O)[C@H](NC(=O)[C@@H]1CSSC[C@H](NC(=O)[C@@H](Cc2ccccc2)NC(=O)[C@H](CO)NC(=O)CNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)[C@H](CO)NC(=O)CNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-59 | DOTA-Aoc- K(Z360)- f[CYwKTC] T-OH | 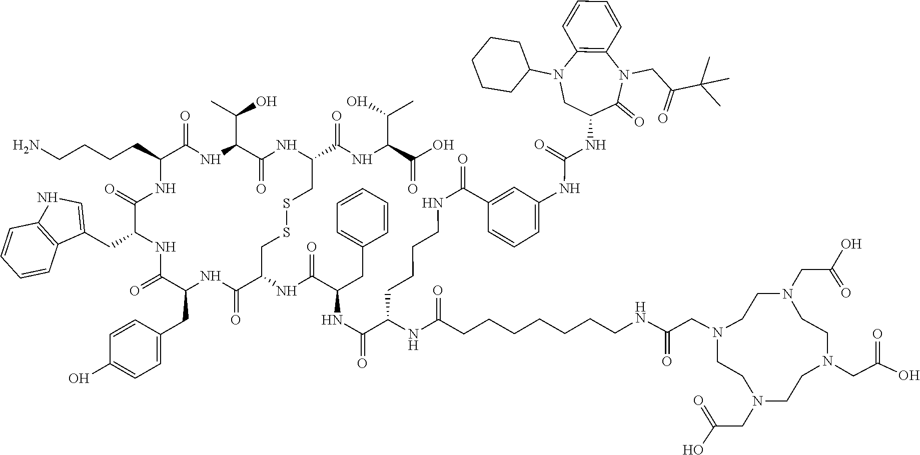 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CCCCCCCNC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-60 | DOTA-K(- G-G-G- Z360)- f[CYwKTC] T-OH | 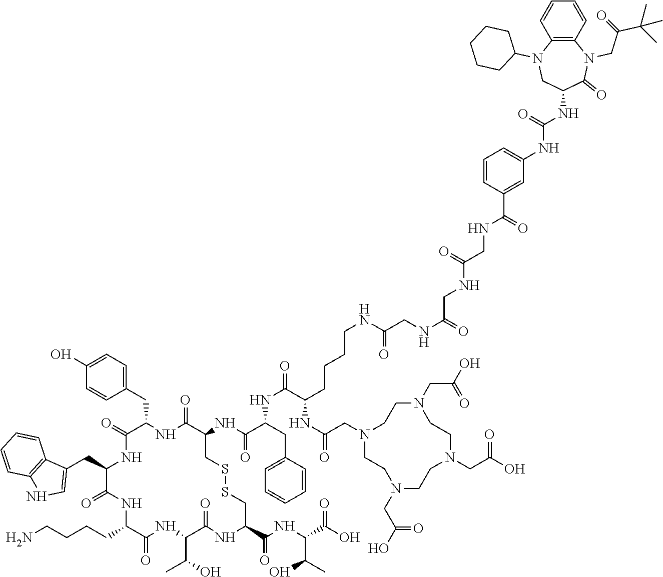 [SMILES: C[C@@H](O)[C@H](NC(=O)[C@@H]1CSSC[C@H](NC(=O)[C@@H](Cc2ccccc2)NC(=O)[C@H](CCCCNC(=O)CNC(=O)CNC(=O)CNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-61 | DOTA- K(Z360)- Aoc- f[CYwKTC] T-OH | 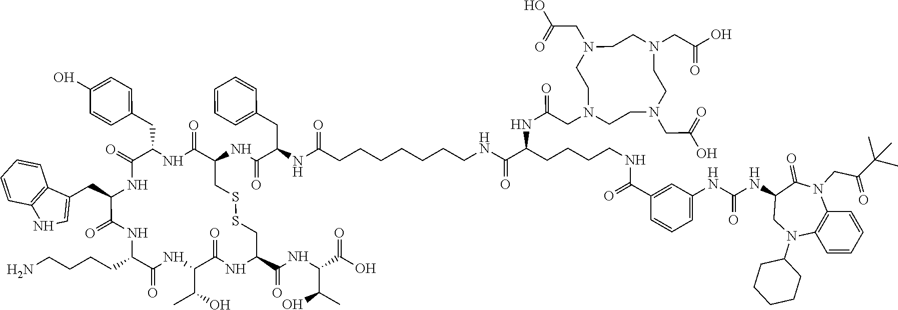 [SMILES: C[C@@H](O)[C@H](NC(=O)[C@@H]1CSSC[C@H](NC(=O)[C@@H](Cc2ccccc2)NC(=O)CCCCCCCNC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-62 | DOTA-R- K(Z360)- f[CYwKTC] T-OH | 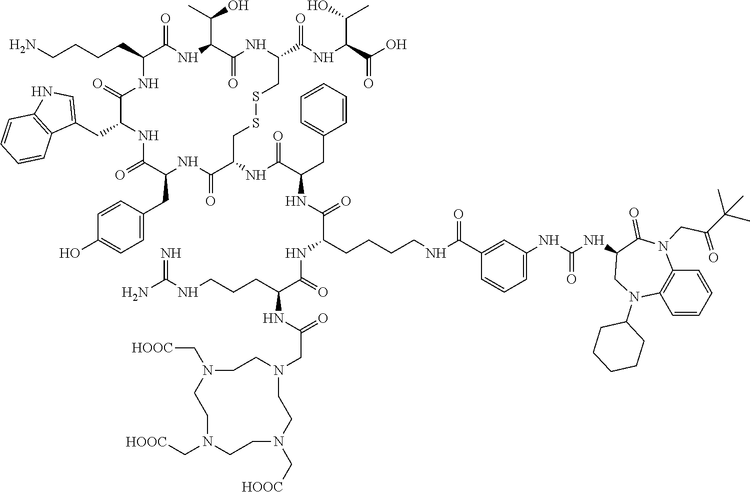 [SMILES: C[C@@H](O)[C@H](NC(=O)[C@@H]1CSSC[C@H](NC(=O)[C@@H](Cc2ccccc2)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)[C@H](CCCNC(=N)N)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-63 | DOTA- K(Z360)-R- f[CYwKTC] T-OH | 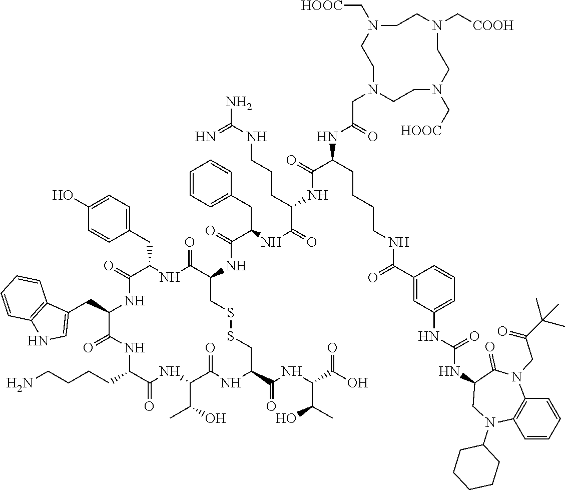 [SMILES: C[C@@H](O)[C@H](NC(=O)[C@@H]1CSSC[C@H](NC(=O)[C@@H](Cc2ccccc2)NC(=O)[C@H](CCCNC(=N)N)NC(=O)[C@H](CCCCNC(=O)c2cccc(NC(=O)N[C@@H]3CN(C4CCCCC4)c4ccccc4N(CC(=O)C(C)(C)C)C3=O)c2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H]([C@@H](C)O)C(=O)N1)C(=O)O] |
| I-64 | DOTA-K(- OEG- Z360)- f[CYwKTC] T-NH₂ | 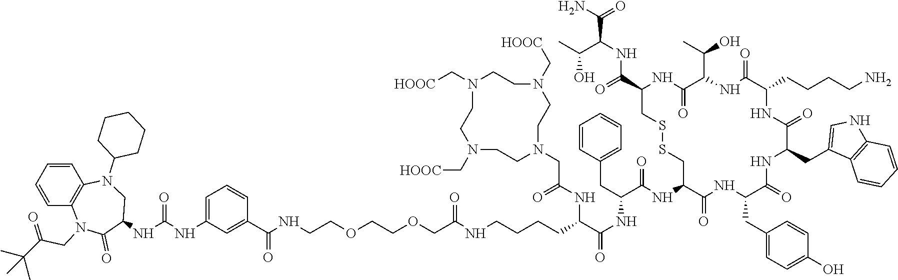 |
| I-65 | DOTA-eK- eK-OEG- OEG-K(- gE-G- Z360)- OEG- f[CYwKTC] T-OH | 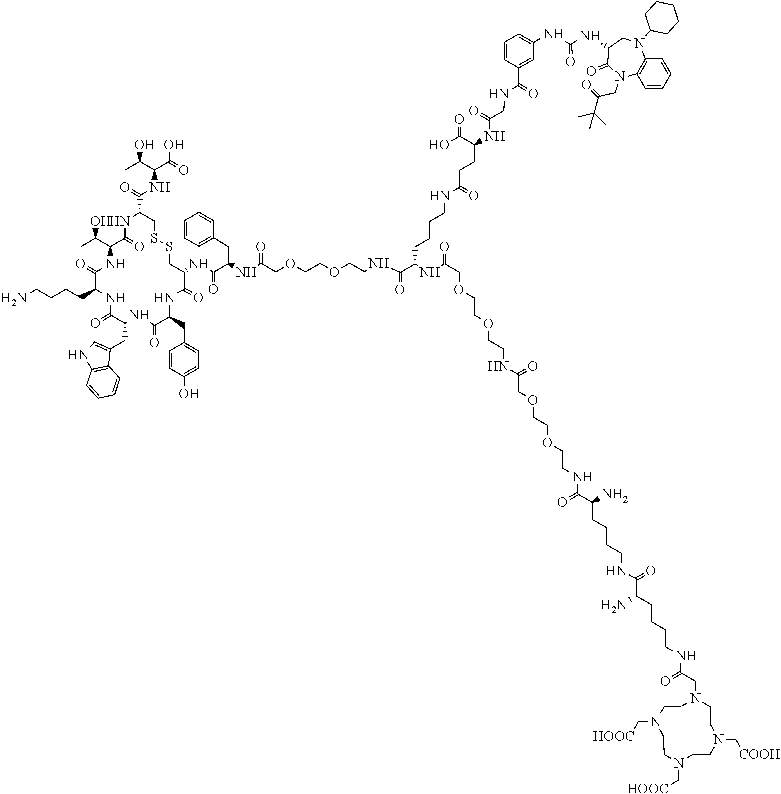 |
| I-66 | DOTA-eK- eK-eK- OEG-K(- gE-G- Z360)- OEG- f[CYwKTC] T-OH | 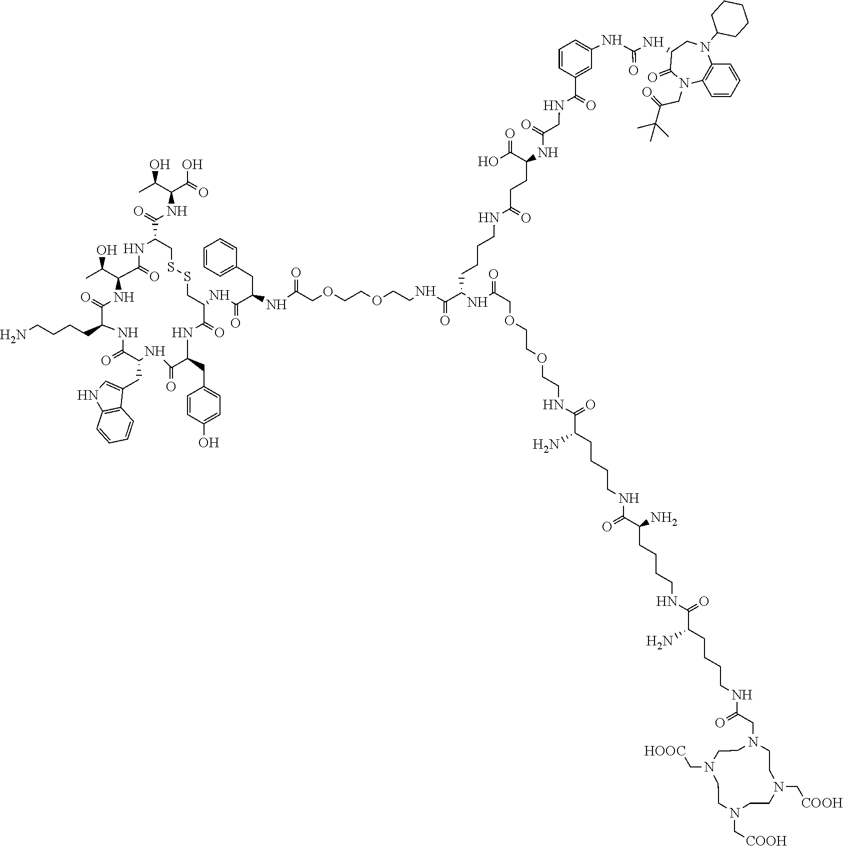 |
| I-67 | DOTAGA- K(Z360)- OEG- f[CYwKTC] T-OH | 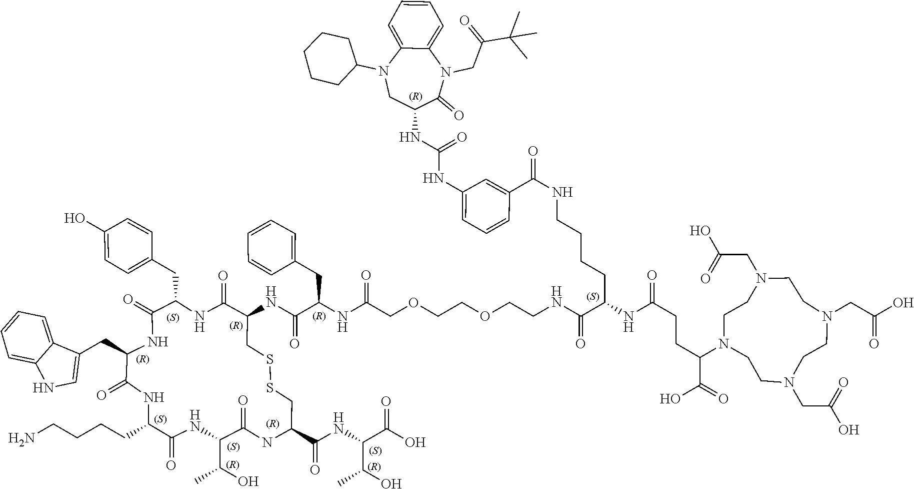 |
| I-68 | DOTAGA- K(-Glu- Z360)- OEG- f[CYwKTC] T-OH | 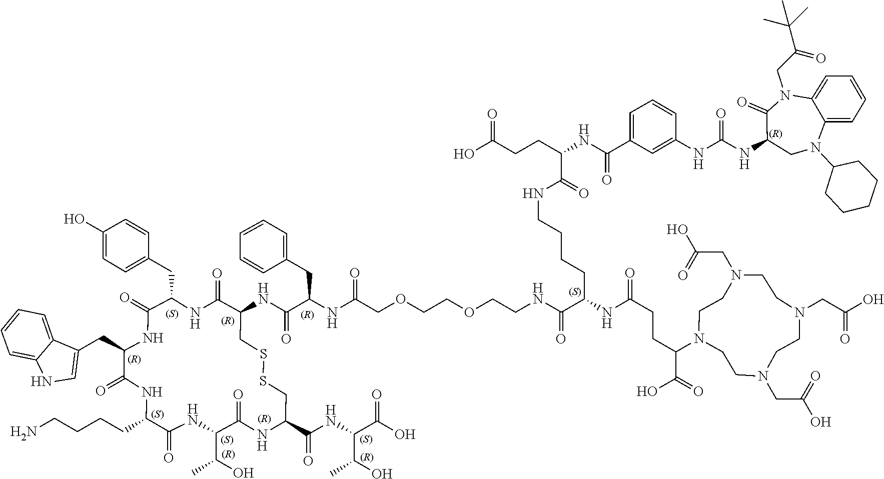 |
| I-69 | DOTAGA- K(-Glu- Z360)- f[CYwKTC] T-OH | 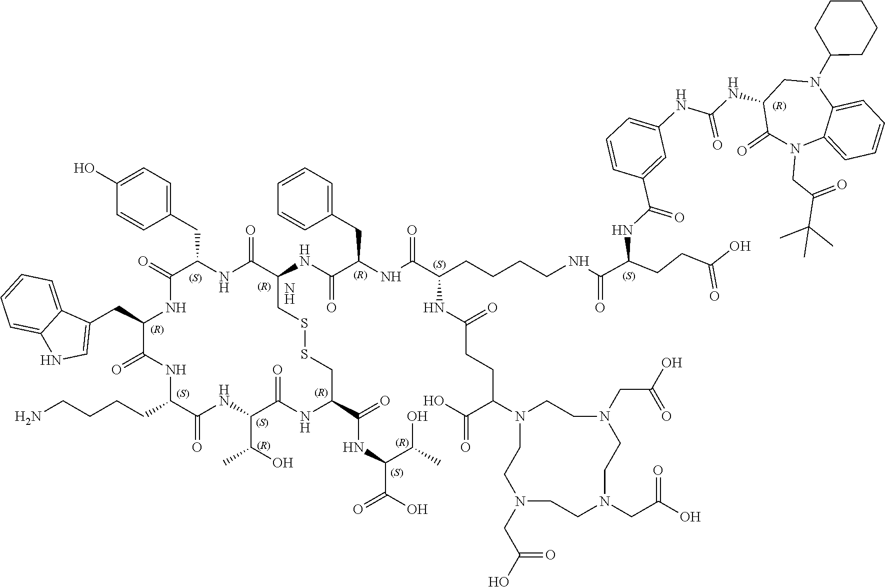 |
| I-70 | DOTA-K(- Glu-Z360)- Ahx- f[CYwKTC] T-OH | 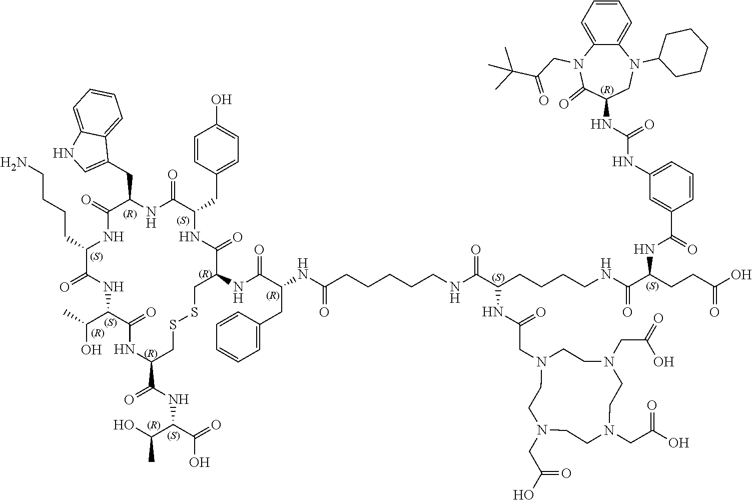 |
| I-71 | DOTAGA- K(-Glu- Z360)-Ahx- f[CYwKTC] T-OH | 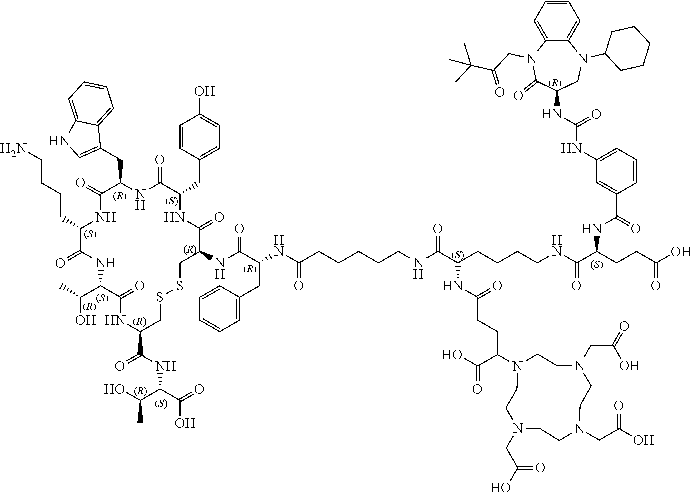 |
| I-72 | DOTAGA- K(-Glu- Z360)-Ahx- Ahx- f[CYwKTC] T-OH | 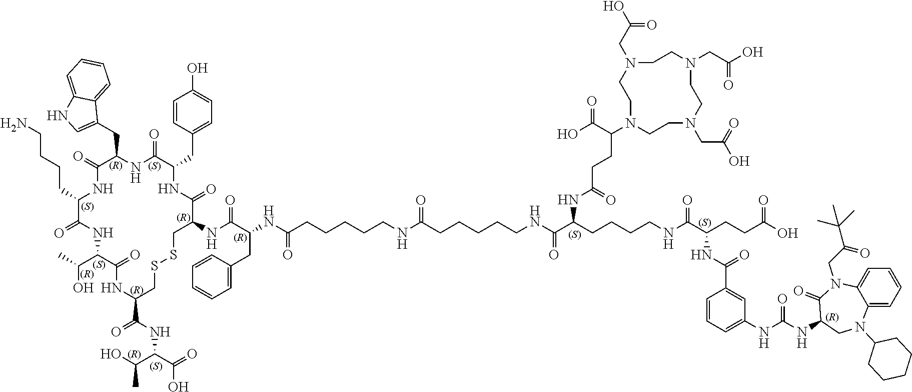 |
| I-73 | DOTAGA- K(-Glu- Z360)-Aoc- Aoc- f[CYwKTC] T-OH | 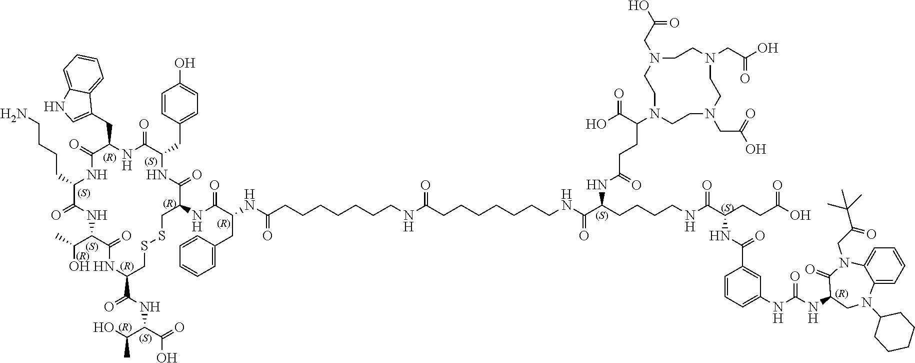 |
| I-74 | DOTA- K(Z360)- gGlu-OEG- OEG- f[CYwKTC] T-OH | 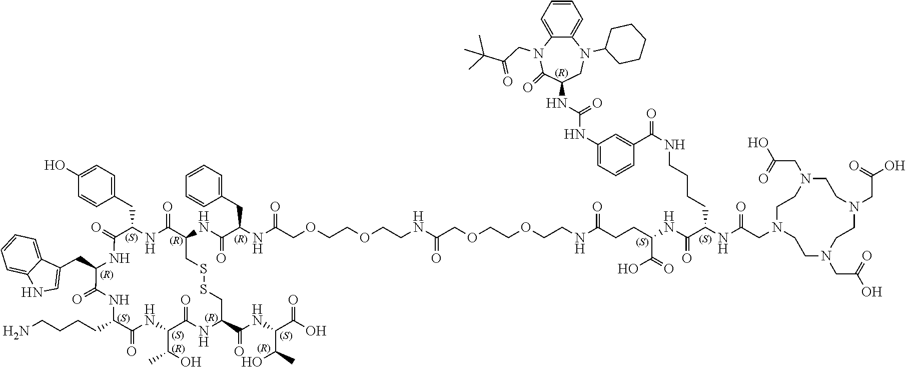 |
| I-75 | DOTA- K(Z360)- Glu-gGlu- OEG-OEG- f[CYwKTC] T-OH | 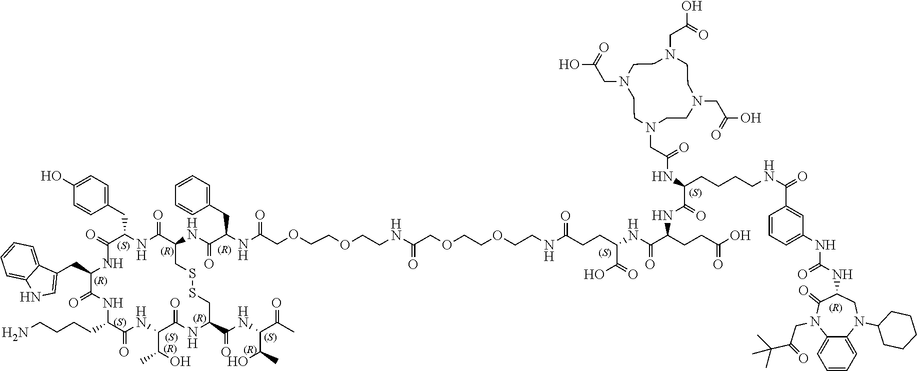 |
| I-76 | DOTA-K(- OEG-OEG- Glu-Z360)- OEG- f[CYwKTC] T-OH | 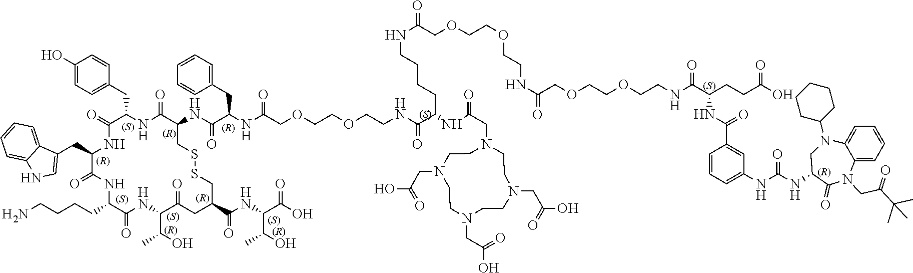 |
| I-77 | DOTA-K(- OEG-gGlu- Ser-Z360)- OEG- f[CYwKTC] T-OH | 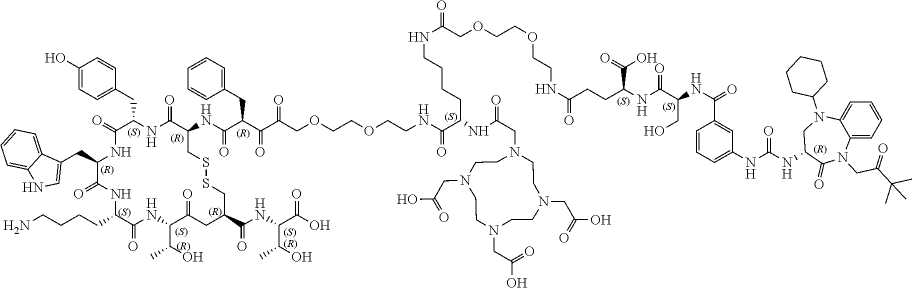 |
| I-78 | DOTA-K(- OEG-gGlu- Dap-Z360)- OEG- f[CYwKTC] T-OH | 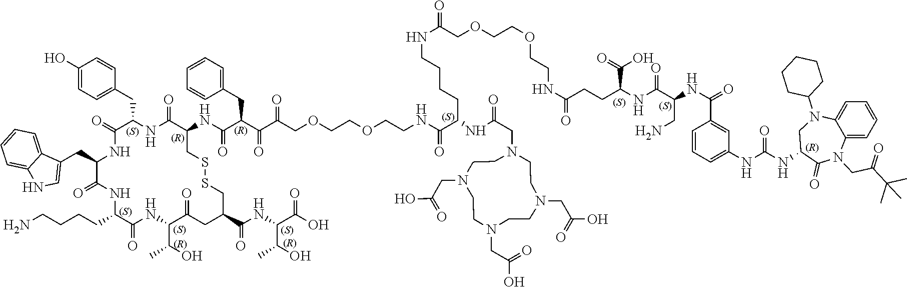 |
| I-79 | DOTA-K(- OEG-His- Glu-Z360)- OEG- f[CYwKTC] T-OH | 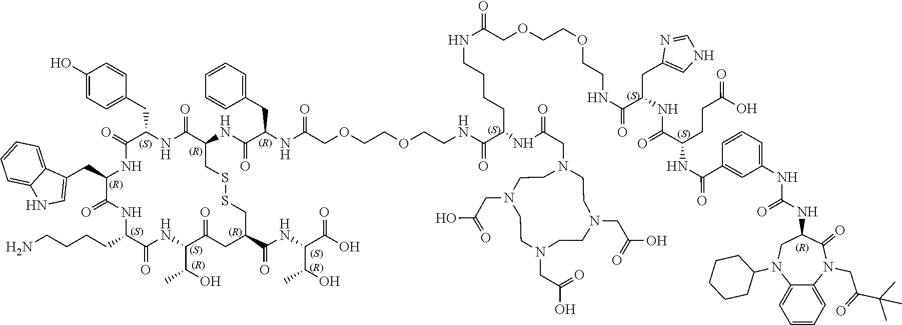 |
| I-80 | DOTA-K(- OEG-OEG- Cbp-Z360)- OEG- f[CYwKTC] T-OH | 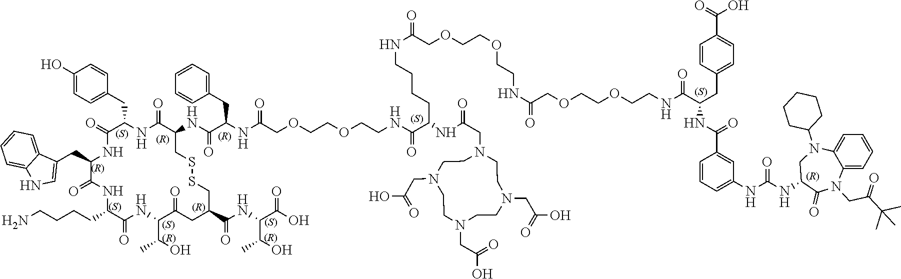 |

or a pharmaceutically acceptable salt and/or solvate thereof.

76. A radionuclide complex or a pharmaceutically acceptable salt and/or solvate thereof, comprising a compound of any one of claims 1 to 75 or a pharmaceutically acceptable salt and/or solvate thereof, and one or more radionuclides.

77. The radionuclide complex of claim 76, wherein the one or more radionuclides are selected from ¹⁴C, ¹⁵N, ¹⁸F, ⁷⁵Br, ⁷⁶Br, ⁷⁷Br, ¹²³I ¹²⁴I, ¹²⁵I, ¹³¹I, ³⁵S, ⁹⁹Tc, ^{99m}Tc, ¹⁸⁸Re, ¹⁸⁶Re, ¹⁵³Sm, ⁶⁶Ga, ⁶⁷Ga, ⁶⁸Ga, ¹¹¹In, ¹²³In, 59Fe, ⁶³Zn, ⁵²Fe, ⁵²Mn, ⁴⁵Ti, ⁶⁰Cu, ⁶¹Cu, ⁶⁷Cu ⁶⁴Cu ⁶²Cu ⁸²Rb, ^{19m}Pt, ^{191m}Pt, ^{193m}Pt, ^{117m}Sn, ⁸⁹Zr, ¹⁷⁷Lu, ¹⁸F, ¹⁸⁸Re, ¹⁸⁶Re, ¹⁵³Sm, ⁶⁶Ho, ⁸⁶Y, ⁸⁷Y, ⁹⁰Y, ⁸⁹Sr, ¹⁵³Gd, ¹⁵⁹Gd, ²²⁵Ac, ²¹²Bi, ²¹³Bi, ²¹¹At, ¹⁹⁸Au, ¹⁹⁹Au, ^{193m}Pt, ¹⁹⁷Pt, ¹⁰³Pd, ¹⁰⁹Pd, ¹⁰⁵Rh, ^{101m}Rh, ^{103m}Rh, ²²³Ra, ²²⁴Ra, ⁹⁷Ru, ²²⁷Th, ²²⁹Th, ³²P, ¹⁶¹Tb, ³³p ¹⁴⁹Tb, ¹²⁵I, ²⁰³Pb, ²¹²Pb, ²⁰¹Tl, ¹¹⁹Sb, ^{58m}Co, 55 Co, 57Co ⁴⁷Sc, ¹⁴⁹Pm ¹⁴²Pr ¹⁶¹Ho, ¹⁶⁶Ho, ¹⁷⁵Yb, or ⁵¹Cr.

78. The radionuclide complex of claim 76 or claim 77, wherein the one or more radionuclides are for use in imaging or diagnosing, or for use in therapy.

79. The radionuclide complex of claim 78, wherein the radionuclides is ¹⁷⁷Lu or ²²⁵Ac.

80. A composition comprising one or more compounds of any one of claims 1 to 75, or one or more complexes of any one of claims 76 to 79 and a carrier.

81. A kit comprising
one or more compounds of Formula I of any one of claims 1 to 75, or a pharmaceutically acceptable salt and/or solvate thereof, and
instructions for administration of the one or more compounds of Formula I, or a pharmaceutically acceptable salt and/or solvate thereof, to a subject in need thereof.

82. A kit comprising
one or more compounds of Formula I of any one of claims 1 to 75, or a pharmaceutically acceptable salt and/or solvate thereof, and
one or more radioisotope as defined above, and
optionally instructions for administration of the one or more compounds of Formula I, or a pharmaceutically acceptable salt and/or solvate thereof, to a subject in need thereof and the radioisotope to a subject in need thereof.

83. A kit comprising
one or more complexes of any one of claims 76 to 79, as defined above, or a pharmaceutically acceptable salt and/or solvate thereof, and
instructions for administration of the one or more compounds complexes to a subject in need thereof.

84. A method of treating a disease or disorder comprising administering a therapeutically effective amount of one or more compounds of any one of claims 1 to 71, or one or more complexes of any one of claims 76 to 79 to a subject in need thereof.

85. The method of claim 84, wherein the disease or disorder is cancer.

86. The method of claim 84, wherein the cancer is a cancer that overexpresses SSTR2, CCK2R or both.

87. The method of claim 85, wherein the cancer is medullary thyroid carcinoma (MTC) or a small cell lung cancer (SCLC) or a neuroendocrine tumor.

88. A method of inhibiting proliferative activity in a cell, comprising administering a therapeutically effective amount of one or more compounds of any one of claims 1 to 75, or one or more complexes of any one of claims 76 to 79 to the cell.

89. A method of imaging a tissue in a subject by administering an imaging effective amount of one or more compounds of any one of claims 1 to 75, or one or more complexes of any one of claims 76 to 79 for use in imaging to a subject in need thereof and applying an imaging technique to detect emitted gamma rays.

90. A method of diagnosing cancer in subject by administering a diagnostic effective amount of one or more compounds of any one of claims 1 to 75, or one or more complexes of any one of claims 76 to 79 to a subject in need thereof and applying an imaging technique to detect emitted gamma rays.
